(19) United States
(12) Patent Application Publication
(10) Pub. No.: US 2024/0150830 A1
(43) Pub. Date: May 9, 2024

(54) PHASED GENOME SCALE EPIGENETIC MAPS AND METHODS FOR GENERATING MAPS

(71) Applicants: Erez Lieberman Aiden, Houston, TX (US); Galina Aglyamova, Houston, TX (US); Ivan Bochkov, Houston, TX (US); Olga Dudchenko, Houston, TX (US); Saul Godinez, Houston, TX (US); Huiya Gu, Houston, TX (US); Ragini Mahajan, Houston, TX (US); Suhas Rao, Stanford, CA (US); Andreas Gnirke, Cambridge, MA (US); Elena Stamenova, Cambridge, MA (US)

(72) Inventors: Erez Lieberman Aiden, Houston, TX (US); Galina Aglyamova, Houston, TX (US); Ivan Bochkov, Houston, TX (US); Olga Dudchenko, Houston, TX (US); Saul Godinez, Houston, TX (US); Huiya Gu, Houston, TX (US); Ragini Mahajan, Houston, TX (US); Suhas Rao, Stanford, CA (US); Andreas Gnirke, Cambridge, MA (US); Elena Stamenova, Cambridge, MA (US)

(21) Appl. No.: 18/501,637

(22) Filed: Nov. 3, 2023

Related U.S. Application Data
(60) Provisional application No. 63/422,414, filed on Nov. 3, 2022.

(51) Int. Cl. C12Q 1/6874; C12Q 1/6806; C12Q 1/6876; G16B 45/00

(52) U.S. Cl. CPC C12Q 1/6874; C12Q 1/6876; G16B 45/00; C12Q 1/6806; C12Q 2600/154; C12Q 2600/156; C12Q 2600/16

(57) ABSTRACT

Disclosed are methods for obtaining genome scale and fully phased epigenetic maps in a cell. The method enables maintaining intact chromatin structure and interrogating chromatin structure using chromatin accessibility maps. DNA contacts are used to fully phase the epigenetic and chromatin contact maps.

### CROSS-REFERENCE TO RELATED APPLICATIONS

This application claims the benefit of U.S. Provisional Application No. 63/422,414, filed Nov. 3, 2022. The entire contents of the above-identified application are hereby fully incorporated herein by reference.

### STATEMENT REGARDING FEDERALLY SPONSORED RESEARCH

This invention was made with government support under Grant No. OD008540 awarded by the National Institutes of Health, and Grant No. PHY1427654 awarded by the National Science Foundation. The government has certain rights in the invention.

### REFERENCE TO AN ELECTRONIC SEQUENCE LISTING

The contents of the electronic sequence listing (“BROD-5735US_ST26.xml”; Size is 515,606 bytes and it was created on Nov. 3, 2023) is herein incorporated by reference in its entirety.

### TECHNICAL FIELD

The subject matter disclosed herein is generally directed to genome scale and fully phased epigenetic maps of chromatin structure and methods for generating the maps.

### BACKGROUND

It has been suggested that the three-dimensional structure of nucleic acids in a cell may be involved in complex biological regulation, for example compartmentalizing the nucleus and bringing widely separated functional elements into close spatial proximity. Understanding how nucleic acids interact, and perhaps more importantly how this interaction, or lack thereof, regulates cellular processes, presents a new frontier of exploration. For example, understanding chromosomal folding and the patterns therein can provide insight into the complex relationships between chromatin structure, gene activity, and the functional state of the cell.

Typically, deoxyribonucleic acid (DNA) is viewed as a linear molecule, with little attention paid to the three-dimensional organization. However, chromosomes are not rigid, and while the linear distance between two genomic loci indeed may be vast, when folded, the special distance may be small (i.e., looping). For example, while regions of chromosomal DNA may be separated by many megabases, they also can be immediately adjacent in 3-dimensional space. Much the same way a protein can fold to bring sequence elements together to form an active site, from the standpoint of gene regulation, long-range interactions between genomic loci may form active centers. For example, gene enhancers, silencers, and insulator elements might function across vast genomic distances.

Current methods of determining 3D architecture cannot map all the chromatin loops and cannot associate each loop with a single DNA element because of inadequate resolution. Current methods suffer from the problem that regulatory loops seem absent, looping elements are localized to 15 kb, which is far worse than linear epigenetics assays. Regarding epigenetics proteins associated with each loop need to be identified. Current problems are that the identity of looping proteins cannot be determined. This requires two separate assays using different populations of cells, ChIP-Seq and Dnase-Seq. These datasets are inaccurate and often shallow. For example, ⅔ of CTCF loop anchors lack an annotated Dnase footprint. Regarding genetics there is a need to be able to predict the effect of every single variant on protein binding, loop formation, and gene expression, but there is no way to link variants to function. This requires external, phased SNP data and it is hard to link variants to protein binding or looping. In situ Hi-C in nuclei improves 3D genome mapping but only up to a point because peaks are diffuse at 1 kb resolution, even with an order of magnitude more reads (see, e.g., Rao S S, Huntley M H, Durand N C, et al. A 3D map of the human genome at kilobase resolution reveals principles of chromatin looping. Cell. 2014; 159(7):1665-1680). In the case of oncogenes and other disease-associated genes, identification of long-range genetic regulators would be of great use in identifying the genomic variants responsible for the disease state and the process by which the disease state is brought about.

Citation or identification of any document in this application is not an admission that such a document is available as prior art to the present invention.

### SUMMARY

In one aspect, the present invention provides for a phased genome scale nuclease sensitivity or chromatin accessibility map for a cell, wherein the nuclease cut sites are determined with 1000, 500, 200, 100, 50, 10 or 1 base pair resolution, or any values in between. In another aspect, the present invention provides for a phased genome scale DNA methylation map for a cell, wherein the DNA methylation sites are determined with 1000, 500, 200, 100, 50, 10 or 1 base pair resolution, or any values in between. In another aspect, the present invention provides for a phased genome scale DNA protein-binding map for a cell, wherein the sequence bound by a chromatin protein or chromatin modification is determined with 1000, 500, 200, 100, 50, 10 or 1 base pair resolution, or any values in between.

In another aspect, the present invention provides for a phased genome scale nuclease sensitivity or chromatin accessibility map for a cell obtained by a method comprising: enzymatically fragmenting intact chromatin in a cell; performing proximity ligation of the fragmented chromatin; sequencing ligation junctions of the ligated chromatin fragments obtained by proximity ligation to determine DNA contacts in the cell and chromatin cut sites; phasing the sequenced chromatin fragments onto individual homologs in the cell based on DNA contacts; and phasing the cut sites from the fragmenting step onto the individual homologs to generate a phased genome scale nuclease sensitivity map.

In another aspect, the present invention provides for a phased genome scale DNA methylation map for a cell obtained by a method comprising: enzymatically fragmenting intact chromatin in a cell; performing proximity ligation of the fragmented chromatin; converting the ligated chromatin fragments by a method that distinguishes between unmodified and modified cytosines, wherein modified cytosines are selected from the group consisting of methylated cytosines (mC) and hydroxymethylated cytosines (hmC); sequencing ligation junctions of the converted ligated chromatin fragments obtained by proximity ligation to determine DNA contacts in the cell, DNA methylation sites, and chromatin cut sites; phasing the sequenced chromatin fragments onto individual homologs in the cell based on DNA contacts; and phasing the DNA methylation sites onto the individual homologs to generate a phased genome scale DNA methylation map. In certain embodiments, the method that distinguishes between unmodified and modified cytosines is selected from the group consisting of (i) bisulfite conversion, (ii) Tet-assisted bisulfite conversion, (iii) Tet-assisted conversion with a substituted borane reducing agent, and (iv) protection of hmC followed by Tet-assisted conversion with a substituted borane reducing agent.

In another aspect, the present invention provides for a phased genome scale DNA protein-binding map for a cell obtained by a method comprising: enzymatically fragmenting intact chromatin in a cell; performing proximity ligation of the fragmented chromatin; performing a method that detects protein binding to the ligated chromatin fragments or chromatin modifications on the ligated chromatin fragments, optionally, with an antibody specific for the chromatin protein or chromatin modification; sequencing ligation junctions of the ligated chromatin fragments obtained by proximity ligation and immunoprecipitation to determine DNA contacts in the cell, chromatin cut sites, and DNA sites bound by the chromatin protein or having the chromatin modification; phasing the sequenced chromatin fragments onto individual homologs in the cell based on DNA contacts; and phasing the DNA sites bound by the chromatin protein or having the chromatin modification onto the individual homologs to generate a phased genome scale DNA protein-binding map. In certain embodiments, the method that detects protein binding or chromatin modification is selected from the group consisting of (i) chromatin immunoprecipitation (ChTP) with an antibody specific for the chromatin protein or chromatin modification, (ii) fusion of a methyltransferase with a protein in vivo in order to modify nearby DNA bases (such as DAMid); (iii) antibody-mediated DNA modification or cleavage, such as Cut & Run; and (iv) other methods for marking sites bound by a specific protein.

In another aspect, the present invention provides for a method for obtaining a phased genome scale nuclease sensitivity map for a cell comprising: enzymatically fragmenting intact chromatin in a cell; performing proximity ligation of the fragmented chromatin; sequencing ligation junctions of the ligated chromatin fragments obtained by proximity ligation to determine DNA contacts in the cell and chromatin cut sites; phasing the sequenced chromatin fragments onto individual homologs in the cell based on DNA contacts; and phasing the cut sites from the fragmenting step onto the individual homologs to generate a phased genome scale nuclease sensitivity map.

In another aspect, the present invention provides for a method for obtaining a phased genome scale DNA methylation map for a cell comprising: enzymatically fragmenting intact chromatin in a cell; performing proximity ligation of the fragmented chromatin; converting the ligated chromatin fragments by a method that distinguishes between unmodified and modified cytosines, wherein modified cytosines are selected from the group consisting of methylated cytosines (mC) and hydroxymethylated cytosines (hmC); sequencing ligation junctions of the converted ligated chromatin fragments obtained by proximity ligation to determine DNA contacts in the cell, DNA methylation sites, and chromatin cut sites; phasing the sequenced chromatin fragments onto individual homologs in the cell based on DNA contacts; and phasing the DNA methylation sites onto the individual homologs to generate a phased genome scale DNA methylation map. In certain embodiments, the method that distinguishes between unmodified and modified cytosines is selected from the group consisting of (i) bisulfite conversion, (ii) Tet-assisted bisulfite conversion, (iii) Tet-assisted conversion with a substituted borane reducing agent, and (iv) protection of hmC followed by Tet-assisted conversion with a substituted borane reducing agent.

In another aspect, the present invention provides for a method for obtaining a phased genome scale DNA protein-binding map for a cell comprising: enzymatically fragmenting intact chromatin in a cell; performing proximity ligation of the fragmented chromatin; performing a method that detects protein binding to the ligated chromatin fragments or chromatin modifications on the ligated chromatin fragments, optionally, with an antibody specific for a chromatin protein or chromatin modification; sequencing ligation junctions of the ligated chromatin fragments obtained by proximity ligation and immunoprecipitation to determine DNA contacts in the cell, chromatin cut sites, and DNA sites bound by the chromatin protein or having the chromatin modification; phasing the sequenced chromatin fragments onto individual homologs in the cell based on DNA contacts; and phasing the DNA sites bound by the chromatin protein or having the chromatin modification onto the individual homologs to generate a phased genome scale DNA protein-binding map.

In certain embodiments, the method further comprises identifying the state of the chromatin fragmented or confirming that the chromatin fragmented was intact, optionally, wherein only fragments from confirmed intact chromatin are used to generate the phased genome scale map.

In another aspect, the present invention provides for a method for detecting spatial proximity relationships between genomic DNA in a cell comprising: enzymatically fragmenting intact chromatin in a cell; performing proximity ligation of the fragmented chromatin; sequencing ligation junctions of the ligated chromatin fragments obtained by proximity ligation to determine DNA contacts in the cell and chromatin cut sites; phasing the sequenced chromatin fragments onto individual homologs in the cell based on DNA contacts; phasing the cut sites from the fragmenting step onto the individual homologs to generate a phased genome scale nuclease sensitivity map; and identifying the state of the chromatin fragmented using the genome scale nuclease sensitivity map. In certain embodiments, fragments from the least denatured chromatin are used to detect spatial proximity relationships. In certain embodiments, only fragments from confirmed intact chromatin are used to detect spatial proximity relationships. In certain embodiments, the cell was obtained from a sample treated with one or more agents or conditions that causes chromatin to be destabilized, such as agents, radiation, osmotically swelling of cells. In certain embodiments, the cell was obtained from a deceased organism, such as dead for more than 3 days or fossilized.

In another aspect, the present invention provides for a phased genome scale DNA methylation map for a cell obtained by a method comprising: enzymatically fragmenting intact chromatin in a cell; performing proximity ligation of the fragmented chromatin; sequencing ligation junctions of the converted ligated chromatin fragments obtained by proximity ligation using a sequencer that can detect DNA methylation to determine DNA contacts in the cell, DNA methylation sites, and chromatin cut sites; phasing the sequenced chromatin fragments onto individual homologs in the cell based on DNA contacts; and phasing the DNA methylation sites onto the individual homologs to generate a phased genome scale DNA methylation map.

In another aspect, the present invention provides for a method for obtaining a phased genome scale DNA methylation map for a cell comprising: enzymatically fragmenting intact chromatin in a cell; performing proximity ligation of the fragmented chromatin; sequencing ligation junctions of the converted ligated chromatin fragments obtained by proximity ligation using a sequencer that can detect DNA methylation to determine DNA contacts in the cell, DNA methylation sites, and chromatin cut sites; phasing the sequenced chromatin fragments onto individual homologs in the cell based on DNA contacts; and phasing the DNA methylation sites onto the individual homologs to generate a phased genome scale DNA methylation map.

In certain embodiments, the method further comprises an annotation of DNA elements located on each homolog of each chromosome of a cell as determined using the map or method.

In certain embodiments, the chromatin is enzymatically fragmented with any nuclease, such as DNase I, micrococcal nuclease (MNase), benzonase, or cyanase, or a restriction enzyme, or a transposase complex. In certain embodiments, the method further comprises identifying chromatin sites bound by a protein on the phased genome using the chromatin cut sites to identify sites protected by bound proteins. In certain embodiments, the method further comprises determining known DNA motifs in the chromatin sites bound by proteins to determine the proteins bound at the chromatin sites in the diploid genome. In certain embodiments, the method further comprises determining unknown DNA motifs bound by proteins. In certain embodiments, the method further comprises isolating proteins specific to the unknown DNA motifs by isolating proteins that bind to the DNA motif sequences. In certain embodiments, intact chromatin is enzymatically fragmented in an isolated nuclei from the cell. In certain embodiments, the cell is crosslinked. In certain embodiments, the sequencing is ligation junction sequencing. In certain embodiments, ligation junction sequencing comprises selecting and sequencing approximately 250 base pair fragments using paired end sequencing. In certain embodiments, ligation junction sequencing comprises selecting and sequencing approximately 300 base pair fragments from a single end. In certain embodiments, the method further comprises identifying sequence variants on a phased genome. In certain embodiments, the method further comprises determining a phased whole genome sequence for the cell based on the determined sequence information.

In certain embodiments, the method is used to determine which DNA elements tend to be in physical proximity of other DNA elements. In certain embodiments, the method is combined with single cell sequencing in order to map accessibility, methylation, or protein binding on a single chromosomal molecule or homolog rather than in a single cell.

In certain embodiments, chromatin is maintained intact using one or methods comprising: (1) not using SDS or other detergents prior to ligation; (2) crosslinking for an extended period of time with formaldehyde, using multiple crosslinkers, or not crosslinking at all; (3) avoiding high-temperature steps; and (4) performing in reactions in buffers with physiologic ion concentrations.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A-1B—Intact Hi-C improves 3D genome mapping with no dependence on digestion strategy. FIG. 1A. In situ Hi-C maps compared to intact Hi-C maps at 500 kb, 50 kb, 5 kb and 1 kb. FIG. 1B. Aggregate Peak Analysis (APA) plots show the aggregate signal at the same peak using intact-Hi-C and in situ Hi-C with the indicated digestion strategies.

FIG. 2—Intact Hi-C allows for increased resolution (i.e., zooming). Intact Hi-C maps and APA plots at 1 kb, 200 bp and 50 bp resolution.

FIG. 3—Intact Hi-C preserves high resolution structure at the base pair scale. APA plots obtained with Intact-Hi-C and in situ Hi-C with the indicated fragmentation (DNase, quadRE (MboI, MseI, NlaIII, Csp6I) and MNase) and resolution.

FIG. 4—Intact Hi-C peaks line up precisely with ChIP-Seq peaks. Intact Hi-C maps and APA plots at 1 kb, 200 bp and 50 bp resolution lined up with ChIP-seq peaks at the same genomic loci.

FIG. 5—Intact Hi-C enables localization at 1-10 bp resolution purely from Hi-C data. APA plot showing localizations in relation to the center of a convergent CTCF motif pair. Heatmap of localization density relative to the motif pair is shown. Motif orientations are indicated. CTCF ChIP-seq peaks are also shown.

FIG. 6—Intact Hi-C detects over 350K loops, including extensive promoter-enhancer looping. Intact-Hi-C and in situ Hi-C contact maps lined up with ChIP-seq peaks for the indicated proteins and histone modifications. APA plots show peaks in boxed regions. Venn Diagram shows loops identified with Intact Hi-C, in situ Hi-C and overlapping loops. Plot showing enrichment of indicated proteins or chromatin modifications at new (intact Hi-C) and old loop anchors (in situ Hi-C).

FIG. 7—Saturation of loop anchors with Intact Hi-C. Graph showing the number of loops and loop anchors identified as compared to sequencing depth.

FIG. 8—Intact Hi-C localizes most loop anchors to ˜10 bp and can identify causal proteins by de novo motif calling. DNA Motif Sequence Logos identified by intact Hi-C and corresponding DNA binding proteins associated with the motifs found. Also shown are ChIP binding of DNA binding proteins to the center of the identified motifs.

FIG. 9—Nuclease cleavage patterns revealed by intact Hi-C can be used to identify motifs. Top panel shows CTCF Chip-seq at the locus. Next panel shows H3K27ac ChIP-seq at the locus. Next panel shows cut sites as observed in intact Hi-C. Next panel shows genes at the locus. Next panel shows DNase hypersensitivity sites at the locus. Next panel shows motifs at the locus (CTCF motif).

FIG. 10—Anchor footprinting with Intact Hi-C. Footprints of cut sites for forward and reverse CTCF anchors.

FIG. 11—Loop anchor localization can be improved by finding the DNAse footprint. (left) Footprints around Hi-C localizations for CTCF anchors. (right) Footprints around the motifs associated with Hi-C localizations for CTCF anchors.

FIG. 12—Hi-C resequencing pipeline can be used to call SNPs. Comparison between whole genome sequencing and intact Hi-C for calling SNPs.

FIG. 13—Loop resolution diploid Hi-C contact maps can be obtained for every intact Hi-C experiment. Unphased and phased Hi-C maps.

FIG. 14—Intact Hi-C enables homolog-specific accessibility profiles. Cut sites for the maternal and paternal chromosomes are shown. In addition, CTCF ChIP-seq data showing binding of CTCF is shown.

FIG. 15A-15B—Examples of SNPs in CTCF loop anchor motifs. FIG. 15A. Maternal homolog has a SNP and there is no loop. FIG. 15B. Paternal homolog has a SNP in one of two motifs and there is no loop.

FIGS. 16A-16B—Identifying causal sequence motifs via allele specific analysis. FIG. 16A. Intact Hi-C for the maternal and paternal chromosomes are shown. FIG. 16B. Cut sites for the maternal and paternal chromosomes are shown and CTCF ChIP-seq data.

FIG. 17—Genes downregulated after cohesin loss lose promoter-enhancer loops detected by intact Hi-C. Graph showing fraction of genes downregulated for genes having the indicated number of cohesin-dependent loops to the promoter.

FIG. 18—Degradation of POLR2A at 24 hours leads to loss specifically of P-E loops, while degradation of CTCF at 24 hours leads to loss specifically of CTCF loops. Intact Hi-C maps in untreated, RAD21 degron degraded, CTCF degron degraded, and POLR2A degron degraded. ChIP-seq for CTCF, histone modifications and RAD21 are also shown.

FIG. 19A-19C—Superenhancer links with intact Hi-C. FIG. 19A-C. Superenhancers shown using intact Hi-C and in situ Hi-C. ChIP-seq data is also shown.

FIGS. 20—In the absence of FACT, promoters colocalize. Intact Hi-C maps with FACT and in the absence of FACT. ChIP-seq data and RefSeq genes are also shown.

FIG. 21—Intact Hi-C can predict which enhancers regulate which genes using looping and elucidate networks of regulatory interaction. Intact Hi-C and in situ Hi-C maps at the PPIF transcription start site in GM12878 cells.

FIG. 22A-22B—Lower depth intact Hi-C still efficiently detects functional promoter-enhancer loops validated by CRISPRi. FIG. 22A. Intact Hi-C and in situ Hi-C maps. CRISPRi data from Reilly et al (Reilly S K, Gosai S J, Gutierrez A, et al. Direct characterization of cis-regulatory elements and functional dissection of complex genetic associations using HCR-FlowFISH [published correction appears in Nat Genet. 2021 October; 53(10):1517]. Nat Genet. 2021; 53(8):1166-1176). Positive values on the CRISPRi tracks indicate that CRISPRi repression at that locus caused downregulation of the target gene. FIG. 22B. Intact Hi-C and in situ Hi-C maps. CRISPRi data from Fulco et al 2016 (Fulco C P, Munschauer M, Anyoha R, et al. Systematic mapping of functional enhancer-promoter connections with CRISPR interference. Science. 2016; 354(6313):769-773).

FIG. 23—Intact Hi-C protocol flowchart.

FIG. 24—Intact Hi-C has bp resolution. Shown are Intact Hi-C maps showing increasing resolution.

FIG. 25A-25B—Intact Hi-C-derived nuclease accessibility data reveals motifs with bp resolution. FIG. 25A. Shown are CTCF ChTP data, nuclease accessibility data and Intact Hi-C maps and aggregate peak analysis (APA). FIG. 25B. Nuclease footprints of cut sites for CTCF anchor.

FIG. 26—Intact Hi-C enables phasing Hi-C maps and Hi-C-based accessibility tracks. Maternal and paternal Hi-C accessibility and Hi-C contact maps shows that CTCF binds to the maternal homolog.

FIG. 27—Intact Hi-C enables phasing Hi-C maps and Hi-C-based accessibility tracks. Maternal and paternal Hi-C accessibility and Hi-C contact maps shows that CTCF binds to the paternal homolog.

FIG. 28—Intact Hi-C protocol can be used to build an atlas of the loops in every human tissue. Representative intact Hi-C maps are shown for the indicated tissues.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a” “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure, or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

Reference is made to U.S. patent application Ser. Nos. 15/532,353, 15/753,318, 16/308,386, 16/247,502, and 16/753,718; and International Patent Applications PCT/US2015/063272, PCT/US2016/047644, PCT/US2017/036649, PCT/US2018/054476, PCT/US2020/033436, PCT/US2020/064704.

All publications, published patent documents, and patent applications cited herein are hereby incorporated by reference to the same extent as though each individual publication, published patent document, or patent application was specifically and individually indicated as being incorporated by reference.

### Overview

A major goal in modern biology is defining the interactions between different biological actors in vivo. Over the past few decades, major advances have been made in developing methods to identify the molecular interactions with any given protein. With nucleic acids and in particular genomic DNA it is difficult to determine the interactions in a cell in part because of enormity, at the sequence level, of genomic DNA in a cell. It is believed that genomic DNA adopts a fractal globule state in which the DNA organized in three dimensions such that functionally related genomic elements, for example enhancers and their target genes, are directly interacting or are located in very close spatial proximity. Such close physical proximity between such elements is further believed to play a role in genome biology both in normal development and homeostasis and in disease. During the cell cycle the particular proximity relationships change, further complicating the study of genome dynamics. Understanding, and perhaps controlling, these tertiary interactions at the nucleic acid level has enormous potential to further our understating of the complexities cellular dynamics and perhaps fostering the development of new classes of therapeutics. Thus, methods are needed to investigate these interactions (e.g., a wiring diagram of a cell). This disclosure meets those needs.

In order to build a wiring diagram of a eukaryotic cell the following must be known. The functional DNA elements, including genes and distal elements. Which elements are physically linked to one another, such as with a map of loops. How strong each link is. How strong is the resulting upregulation/downregulation. Which proteins are responsible for each link. Which DNA bases are essential for each link and what is the effect of mutating these bases. The following invention provides novel methods for building a wiring diagram for any cell and provides novel detailed maps. The diagrams can then be used for therapeutic, diagnostic and genome engineering applications. For example, specific proteins or DNA sequences can be targeted, detected, or modified.

Applicants provide for Intact Hi-C plus confirmation and novel computational tools to address the issues above. Intact Hi-C as disclosed herein combines DNA-DNA proximity ligation in non-denatured chromatin with high throughput sequencing in order to measure how frequently positions in the human genome come into close physical proximity. The disclosed method can simultaneously map substantially all of the interactions of DNAs in a cell, including spatial arrangements of DNA. Intact Hi-C as described herein minimizes protein denaturation and better preserves architecture. Intact Hi-C captures ligation junctions to determine sites of cutting and ligation with up to single base pair resolution (e.g., less than 2 bp, 10 bp, 50 bp resolution). Intact Hi-C can exploit new sequencing technologies to generate maps with >100B reads. Intact Hi-C can use standard crosslinkers and cutters. Intact Hi-C can map all loops and can associate each loop with a single DNA element.

Embodiments disclosed herein provide for genome scale and fully phased epigenetic assay maps (e.g., any map of chromatin structure). As used herein, epigenetic assay refers to any assay that provides information regarding chromosomes and chromatin beyond or above the DNA sequence of a genome. For example, DNase I hypersensitivity assays provide for DNA that is protected from DNase I due to chromatin folding or protein binding, chromatin modification assays, such as histone modifications on individual chromosomes, assays for determining protein or protein complex binding to chromatin, such as transcription factors or chromatin architectural proteins (e.g., cohesin complex), chromatin looping assays, chromatin accessibility assays, and DNA methylation assays. As used herein, genome scale refers to assaying genomic DNA up to and including the entire genome or a substantial portion of the entire genome, such as greater than 25, 30, 35, 40, 45, 50, 55, 60, 65, 70, 75, 80, 85, 90, or 95% of the genome. As used herein, fully phased refers to separating substantially all sequencing reads based on parental chromosome (e.g., greater than 75, 80, 85, 90, 95, or 99% of the sequencing reads). For example, in diploid organisms, phasing an assembly means separating the maternally and paternally inherited copies of each chromosome, known as haplotypes. Each phased contig, or haplotig, is made up of reads from the same parental chromosome. In certain embodiments, phasing requires determining DNA contacts with resolution much greater than 1 kb (i.e., 200, 150, 100, 75, 50, 25, 15, 10, 5 or 1 base pair resolution) to be able to assign short chromatin fragments to individual chromosomes (e.g., fragments less than 500 base pairs, preferably, about 250-300 base pairs).

Embodiments disclosed herein provide for epigenetic maps in a cell at resolution up to single base pair resolution (e.g., 100, 50, 10 or 1 base pair resolution) because the maps are obtained under conditions that maintain the native conformation of proteins. As used herein the chromatin obtained under these conditions are referred to as “intact chromatin.” Intact chromatin maintains the DNA contacts in the nuclei. As used herein “intact chromatin” also refers to chromatin that has not been denatured. Partially or fully denatured chromatin will not maintain protein binding at all DNA fragments resulting in loss of the proximity of DNA fragments, loss of DNA protection, and decreased resolution. As used herein “intact chromatin” also refers to chromatin that is bound by non-denatured proteins, such that DNA bound by a protein is protected from being cut. As used herein “intact chromatin” also refers to chromatin that displays a consistent or sharp nuclease fragmentation pattern or chromatin accessibility pattern for any specific chromatin sequence. For example, a chromatin fragment originating from a single chromosome in a population of cells will have the same pattern for all of the cells. For example, the DNA protection is confined to a sharp sequence corresponding to a specific binding motif sequence. The conditions for intact chromatin do not use SDS or heat inactivation for permeabilization of nuclei. Heating in the presence of SDS reduces the loop signal. The conditions for intact chromatin also maintain protein complex integrity in the nuclei of crosslinked cells. Specific methods for keeping the chromatin intact include, but are not limited to, (1) not using SDS or other detergents prior to ligation; (2) crosslinking for an extended period of time with formaldehyde, using multiple crosslinkers, or not crosslinking at all; (3) avoiding high-temperature steps; and (4) performing in reactions in buffers with physiologic ion concentrations. Applicants note that some of these steps, e.g. the use of SDS, are widely used in other protocols and previously not recognized as very damaging to the chromatin and specifically the chromatin architecture.

Embodiments disclosed herein also provide for the epigenetic maps in a cell where it is confirmed that every region of the genome evaluated does indeed maintain native conformation and chromatin binding (i.e., intact chromatin). In all of the methods described herein chromatin is fragmented, generating a nuclease fragmentation pattern or chromatin accessibility pattern that provides for confirmation of whether the chromatin was intact or not. This confirmation can be considered a “certificate of authenticity” for every experiment performed and every map generated.

The methods described herein allow for the first time a confirmation that in every experiment chromatin was intact as shown by the nuclease sensitivity map. The nuclease sensitivity map can further show every sequence that is bound by a protein in every experiment and can show the exact sequence of the DNA bound because of the base pair resolution that Intact Hi-C provides. Further, the methods described herein can show the exact sequence of a loop anchor. Further, the methods described herein can show the orientation of bound proteins (e.g., N terminal to C terminal of the protein). For example, the nuclease sensitivity pattern can show forward and reverse CTCF motifs bound by CTCF in reverse orientations. Further, the confirmation and increased resolution allows for phasing chromosomes without the use of haplotype specific variants (SNPs). The method also can be used for whole genome sequencing (WGS) with phased SNPs. The method thus provides for fully phased genome scale chromatin assays within an individual experiment without the need for any external data or knowledge.

In example embodiments, the present invention provides for a fully phased genome scale nuclease or chromatin accessibility map for a cell. In example embodiments, determining the exact sequences protected from nuclease digestion or accessible to an enzyme requires less than 1000, 100, 50, or 10 base pair resolution.

In example embodiments, the present invention provides for a fully phased genome scale DNA methylation map for a cell. In example embodiments, ligated chromatin fragments are converted by a method that distinguishes between unmodified and modified cytosines, wherein modified cytosines are selected from the group consisting of methylated cytosines (mC) and hydroxymethylated cytosines (hmC). After sequencing individual methylated cytosines can be phased to individual chromosomes.

In example embodiments, the present invention provides for a fully phased genome scale chromatin immunoprecipitation sequencing (ChIP-seq) map for a cell (i.e., DNA protein-binding), wherein the sequence bound by a chromatin protein or chromatin modification is determined with less than 1000, 100, 50, or 10 base pair resolution. Additionally, because the method includes nuclease sensitivity maps, the exact sites of protein bound to chromatin can be determined.

Using the approach disclosed herein, it is now possible to comprehensively identify all distal regulators of all genes in a sample population of cells. The information available, will make it possible to assess the impact of candidate drugs on specific cellular circuits, hastening the process of drug discovery and for biological research in general. The information available will also enable the mapping of genomic structural and sequence variations.

The methods described herein also allow for determining the whole genome sequence of a cell simultaneously with detecting phased spatial proximity relationships between genomic DNA and phased nuclease sensitivity sites. Applicants discovered that the sequencing reads obtained for the joined fragments cover approximately the same percentage of the genome as conventional whole genome sequencing. Thus, in example embodiments, all sequence variants (e.g., SNPs) can be identified and phased. In example embodiments, the data from the disclosed methods can be used to assemble a genome de novo. In example embodiments, the sequence information determined by the disclosed methods may be used to resolve genomic structural genomic variation, including copy number variations.

In example embodiments, sequence variants associated with a phenotype can be assigned to a specific chromosome or haplotype and can be assigned to a specific gene based on enhancer/promoter contacts (see, e.g., Welter, D. et al. The NHGRI GWAS catalogue, a curated resource of SNP-trait associations. Nucleic Acids Res. 42, D1001-D1006 (2014); Wood, A. R. et al. Defining the role of common variation in the genomic and biological architecture of adult human height. Nat. Genet. 46, 1173-1186 (2014); Ripke, S. et al. Biological insights from 108 schizophrenia-associated genetic loci. Nature 511, 421-427 (2014); Okbay, A. et al. Genome-wide association study identifies 74 loci associated with educational attainment. Nature 533, 539-542 (2016); Sudlow, C. et al. UK biobank: an open access resource for identifying the causes of a wide range of complex diseases of middle and old age. PLoS Med. 12, 1-10 (2015); Bycroft et al., The UK Biobank resource with deep phenotyping and genomic data. Nature 562, 203-209 (2018); and 1000 Genomes Project Consortium. A global reference for human genetic variation. Molecular cell, 526(7571):68-74, 2015). Moreover, variants present in a loop may be assigned to a gene. The variants may be present in an enhancer and enhancers may be assigned to specific genes. Thus, the present invention provides for linking variants to genes to phenotypes (e.g., disease, age related, and health related phenotypes). Previous studies showed that disease-associated variants are enriched in specific regulatory chromatin states (see, e.g., Ernst, J. et al. Mapping and analysis of chromatin state dynamics in nine human cell types. Nature 473, 43-49 (2011)), evolutionarily conserved elements (Lindblad-Toh, K. et al. A high-resolution map of human evolutionary constraint using 29 mammals. Nature 478, 476-482 (2011)), histone marks (Trynka, G. et al. Chromatin marks identify critical cell types for fine mapping complex trait variants. Nature Genet. 45, 124-130 (2013)) and accessible regions (Maurano, M. T. et al. Systematic localization of common disease-associated variation in regulatory DNA. Science 337, 1190-1195 (2012)), thus showing the importance of assigning variants in regulatory sequences to the correct chromosomes and genes.

In example embodiments, the epigenetic states identified are correlated with a disease state or age-related state. In example embodiments, the epigenetic states identified are correlated with an environmental condition. The disclosed methods are also particularly suited to monitoring disease states, such as disease state in an organism, for example a plant or an animal subject, such as a mammalian subject, for example a human subject.

### Methods for Generating Genome Scale and Phased Epigenetic Maps

Disclosed herein are methods for generating phased genome scale epigenetic maps, such as protein binding to chromatin, histone modification, DNA methylation, and chromatin accessibility. The methods require detecting spatial proximity relationships between nucleic acid sequences in intact chromatin with an adequate resolution in order to phase sequencing reads to an individual homolog in a cell or multiple cells. The methods include providing a sample of one or more cells or nuclei isolated from the cells. In some embodiments, the spatial relationships in the cell is locked in, for example cross-linked or otherwise stabilized. For example, a sample of cells can be treated with a cross-linker to lock in the spatial information or relationship about the molecules in the cells, such as the DNA in the cell. The nucleic acids present are fragmented in situ to yield fragmented chromatin. The ends may be filled in and/or repaired in situ, for example using a DNA polymerase, such as available from a commercial source. The filled in or repaired nucleic acid fragments are thus blunt ended at the end filled 5′ end. The fragments are then end joined in situ at the filled in or repaired end, for example, by ligation using a commercially available nucleic acid ligase, or otherwise attached to another fragment that is in close physical proximity. The ligation, or other attachment procedure, for example nick translation or strand displacement, creates one or more end joined nucleic acid fragments having a junction, for example a ligation junction, wherein the site of the junction, or at least within a few bases, includes one or more labeled nucleic acids, for example, one or more fragmented nucleic acids that have had their overhanging ends filled and joined together. While this step typically involves a ligase, it is contemplated that any means of joining the fragments can be used, for example any chemical or enzymatic means. Further, it is not necessary that the ends be joined in a typical 3′-5′ ligation.

In example embodiments, to identify the created ligation junction a labeled nucleotide is used. In one example embodiment, one or more labeled nucleotides are incorporated into the ligated junction. For example, the overhanging or repaired ends may be filled in using a DNA polymerase that incorporates one or more labeled nucleotides during the filling in or repairing step described above.

In some embodiments, the nucleic acids are cross-linked, either directly, or indirectly, and the information about spatial relationships between the different DNA fragments in the cell, or cells, is maintained during the joining step, and substantially all of the end joined nucleic acid fragments formed at this step were in spatial proximity in the cell prior to the crosslinking step. Previously it was believed that the crosslinking locked in the spatial proximity of DNA sequences in the cell. However, Applicants disclose herein that denaturing conditions can still cause part of the spatial information to be lost by denaturing crosslinked protein complexes necessary to hold the DNA in a locked position. Once the DNA ends are joined the information about which sequences were in spatial proximity to other sequences in the cell is locked into the end joined fragments. It has been found that in some situations, it is not necessary to hold the nucleic acids in place using a chemical fixative or crosslinking agent. Thus, in some embodiments, no crosslinking agent is used. In still other embodiments, the nucleic acids are held in position relative to each other by the application of non-crosslinking means, such as by using agar or other polymer to hold the nucleic acids in position.

The labeled nucleotide present in the junction is used to isolate the one or more end joined nucleic acid fragments using a binding agent specific to the labeled nucleotide. The sequence is determined at the junction of the one or more end joined nucleic acid fragments, thereby detecting spatial proximity relationships between nucleic acid sequences in a cell and also detecting the cut sites in the fragmented nucleic acids. In some embodiments, based on the cut sites, the level of denaturation of the chromatin can be determined. In some embodiments, the cut sites can be phased to a homolog. In some embodiments, the cut sites can indicate DNA sequences protected from fragmentation and thus provides a map of all protected sites in the nucleic acids. In some embodiments, when the fragmentation pattern indicates that the chromatin was intact, exact sequence motifs representing protected DNA can be determined. In some embodiments, sequence motifs can be mapped to loop anchors. In some embodiments, such as for genome assembly, essentially all of the sequence of the end joined fragments is determined. In some embodiments, determining the sequence of the junction of the one or more end joined nucleic acid fragments includes nucleic acid sequencing.

In some embodiments, the ligation junctions can be treated to identify epigenetic marks. In one example embodiment, DNA methylation can be detected on phased homologs by converting the ligated chromatin with an agent that distinguishes methylated from non-methylated DNA. In one example embodiment, ligated chromatin still bound to proteins is immunoprecipitated to enrich for fragments bound by proteins or having a specific chromatin modification. In some embodiments, the chromatin accessibility data provided by the methods can be used to determine the exact sequences bound by the immunoprecipitated protein. The ligation junctions of both the enriched (bound) and non-enriched (flow-through) can be sequenced, such that spatial proximity and chromatin accessibility is obtained without significant loss. Ligation junctions bound by the protein is expected to be enriched in the bound fraction as compared to ligations junctions not enriched.

In some embodiments, determining the sequence of the junction of the one or more end joined nucleic acid fragments includes using a probe that specifically hybridizes to the nucleic acid sequences both 5′ and 3′ of the junction of the one or more end joined nucleic acid fragments, for example using an RNA probe, a DNA probe, a locked nucleic acid (LNA) probe, a peptide nucleic acid (PNA) probe, or a hybrid RNA-DNA probe. In exemplary embodiments of the disclosed method, the location is determined or identified for nucleic acid sequences both 5′ and 3′ of the ligation junction of the one or more end joined nucleic acid fragments relative to source genome and/or chromosome.

### Clinical and Research Applications

In example embodiments, the epigenetic states identified are correlated with a disease or age-related state. In example embodiments, the epigenetic states identified are correlated with an environmental condition. In example embodiments, the sequenced end joined fragments are assembled to create an assembled genome or portion thereof, such as a chromosome or sub-fraction thereof. In example embodiments, information from one or more ligation junctions derived from a sample consisting of a mixture of cells from different organisms, such as mixture of microbes, is used to identify the organisms present in the sample and their relative proportions. In some examples, the sample is derived from patient samples.

The disclosed methods are also particularly suited to monitoring disease states or age related states, such as disease state or age related state in an organism, for example a plant or an animal subject, such as a mammalian subject, for example a human subject. Certain disease states or age-related states may be caused and/or characterized by the differential epigenetic states. For example, certain epigenetic states may occur in a diseased cell but not in a normal cell. In other examples, certain epigenetic states may occur in a normal cell but not in diseased cell. Thus, using the disclosed methods a profile of epigenetic states in vivo, can be correlated with a disease state. The epigenetic states correlated with a disease can be used as a “fingerprint” to identify and/or diagnose a disease in a cell, by virtue of having a similar “fingerprint.” In addition, the profile can be used to monitor a disease state, for example to monitor the response to a therapy, disease progression and/or make treatment decisions for subjects.

The ability to obtain a genome scale phased epigenetic map allows for the diagnosis of a disease state, for example by comparison of the profile present in a sample with the correlated with a specific disease state, wherein a similarity in profile indicates a particular disease state.

Accordingly, aspects of the disclosed methods relate to diagnosing a disease state based on a profile of epigenetic states correlated with a disease state, for example cancer, or an infection, such as a viral or bacterial infection. It is understood that a diagnosis of a disease state could be made for any organism, including without limitation plants, and animals, such as humans.

Aspects of the present disclosure relate to the correlation of an environmental stress or state with an epigenetic profile, such as a sample of cells, for example a culture of cells, can be exposed to an environmental stress, such as but not limited to heat shock, osmolarity, hypoxia, cold, oxidative stress, radiation, starvation, a chemical (for example a therapeutic agent or potential therapeutic agent) and the like. After the stress is applied, a representative sample can be subjected to analysis, for example at various time points, and compared to a control, such as a sample from an organism or cell, for example a cell from an organism, or a standard value.

The disclosed methods are also particularly suited to analyzing aging. Aging-associated alterations of higher-order chromatin structures for physiologically aged tissues and cell types remain undetermined (see, e.g., Liu, et al., 2022, Deciphering aging at three-dimensional genomic resolution, Cell Insight, Volume 1, Issue 3). Prior studies used in situ Hi-C that has kilobase resolution (see, e.g., Multiscale 3D Genome Reorganization during Skeletal Muscle Stem Cell Lineage Progression and Muscle Aging. Yu Zhao, Yingzhe Ding, Liangqiang He, Yuying Li, Xiaona Chen, Hao Sun, Huating Wang, bioRxiv 2021.12.20.473464).

In example embodiments, the disclosed methods can be used to screen for agents that modulate epigenetic profiles related to disease or aging. For example, that alter the interaction profile from an aging profile to a young profile. For example that alter protein binding, DNA methylation, and/or looping. By exposing cells, or fractions thereof, tissues, or even whole animals, to different members of a library, and performing the methods described herein, different members of a library can be screened for their effect on epigenetic profiles simultaneously in a relatively short amount of time, for example using a high throughput method.

In some embodiments, screening of test agents involves testing a combinatorial library containing a large number of potential modulator compounds. A combinatorial chemical library may be a collection of diverse chemical compounds generated by either chemical synthesis or biological synthesis, by combining a number of chemical “building blocks” such as reagents. For example, a linear combinatorial chemical library, such as a polypeptide library, is formed by combining a set of chemical building blocks (amino acids) in every possible way for a given compound length (for example the number of amino acids in a polypeptide compound). Millions of chemical compounds can be synthesized through such combinatorial mixing of chemical building blocks. As used herein the term “test agent” refers to any agent that that is tested for its effects, for example its effects on a cell. In some embodiments, a test agent is a chemical compound, such as a chemotherapeutic agent, antibiotic, or even an agent with unknown biological properties.

Appropriate agents can be contained in libraries, for example, synthetic or natural compounds in a combinatorial library. Numerous libraries are commercially available or can be readily produced; means for random and directed synthesis of a wide variety of organic compounds and biomolecules, including expression of randomized oligonucleotides, such as antisense oligonucleotides and oligopeptides, also are known. Alternatively, libraries of natural compounds in the form of bacterial, fungal, plant and animal extracts are available or can be readily produced. Additionally, natural or synthetically produced libraries and compounds are readily modified through conventional chemical, physical and biochemical means, and may be used to produce combinatorial libraries. Such libraries are useful for the screening of a large number of different compounds.

The compounds identified using the methods disclosed herein can serve as conventional “lead compounds” or can themselves be used as potential or actual therapeutics. In some instances, pools of candidate agents can be identified and further screened to determine which individual or sub-pools of agents in the collective have a desired activity.

Appropriate samples for use in the methods disclosed herein include any conventional biological sample obtained from an organism or a part thereof, such as a plant, animal, and the like. In particular embodiments, the sample is a cell line. The cell line can be treated or untreated as described herein (e.g., treated with a drug candidate, compound, biologic, environmental stress, or genetic perturbation). In particular embodiments, the biological sample is obtained from an animal subject, such as a human subject. A biological sample is any solid or fluid sample obtained from, excreted by or secreted by any living organism, including without limitation, single celled organisms, such as yeast, protozoans, and amoebas among others, multicellular organisms (such as plants or animals, including samples from a healthy or apparently healthy human subject or a human patient affected by a condition or disease to be diagnosed or investigated, such as cancer). For example, a biological sample can be a biological fluid obtained from, for example, blood, plasma, serum, urine, bile, ascites, saliva, cerebrospinal fluid, aqueous or vitreous humor, or any bodily secretion, a transudate, an exudate (for example, fluid obtained from an abscess or any other site of infection or inflammation), or fluid obtained from a joint (for example, a normal joint or a joint affected by disease, such as a rheumatoid arthritis, osteoarthritis, gout or septic arthritis). A sample can also be a sample obtained from any organ or tissue (including a biopsy or autopsy specimen, such as a tumor biopsy) or can include a cell (whether a primary cell or cultured cell) or medium conditioned by any cell, tissue, or organ. Exemplary samples include, without limitation, cells, cell lysates, blood smears, cyto-centrifuge preparations, cytology smears, bodily fluids (e.g., blood, plasma, serum, saliva, sputum, urine, bronchoalveolar lavage, semen, etc.), tissue biopsies (e.g., tumor biopsies), fine-needle aspirates, and/or tissue sections (e.g., cryostat tissue sections and/or paraffin-embedded tissue sections). In other examples, the sample includes circulating tumor cells (which can be identified by cell surface markers). In particular examples, samples are used directly (e.g., fresh or frozen), or can be manipulated prior to use, for example, by fixation (e.g., using formalin) and/or embedding in wax (such as formalin-fixed paraffin-embedded (FFPE) tissue samples). It will be appreciated that any method of obtaining tissue from a subject can be utilized, and that the selection of the method used will depend upon various factors such as the type of tissue, age of the subject, or procedures available to the practitioner. Standard techniques for acquisition of such samples are available. See, for example Schluger et al., J. Exp. Med. 176:1327-33 (1992); Bigby et al., Am. Rev. Respir. Dis. 133:515-18 (1986); Kovacs et al., NEJM 318:589-93 (1988); and Ognibene et al., Am. Rev. Respir. Dis. 129:929-32 (1984).

### Proximity Ligation

Embodiments disclosed herein include any method of proximity ligation. As used herein, proximity ligation refers to any method wherein fragmented nucleic acids that are in close proximity to each other in a cell or nuclei are ligated to determine nucleic acids that are in close proximity or contact with each other. The fragments that are in close proximity or contact with each other are determined by sequencing of the ligated fragments and determining the sequences ligated together.

Over the past quarter-century, various methods have emerged to assess the three-dimensional architecture of the nucleus in vivo (Gerasimova et al., Molecular cell 6, 1025-1035, 2000; Mukherjee et al., Cell 52, 375-383, 1988), including nuclear ligation assay and chromosome conformation capture (3C), which analyze contacts made by a single locus (Cullen et al., Science 261, 203-206, 1993; Dekker et al., Science 295, 1306-1311, 2002; Murrell et al., Nature genetics 36, 889-893, 2004; Tolhuis et al., Molecular cell 10, 1453-1465, 2002), extensions such as 5C for examining several loci simultaneously (Dostie et al., Genome research 16, 1299-1309, 2006), and methods such as CHIA-PET for examining all loci bound by a specific protein (Fullwood et al., Nature 462, 58-64, 2009). Previous proximity ligation methods include Hi-C and in situ Hi-C, which combines DNA-DNA proximity ligation with high throughput sequencing to interrogate all pairs of loci across a genome (Lieberman-Aiden et al., Science 326, 289-293, 2009; and Rao S S, Huntley M H, Durand N C, et al. A 3D map of the human genome at kilobase resolution reveals principles of chromatin looping. Cell. 2014; 159(7):1665-1680).

The present invention combines proximity ligation of intact chromatin in situ (i.e., the steps are performed inside nuclei) with high-throughput sequencing and confirmation of intact chromatin to perform any epigenetic assay in a genome scale and phased format.

### Crosslinking

In example embodiments, proximity ligation is performed on crosslinked cells to preserve spatial proximity relationships in the cell. In some embodiments of the disclosed method the nucleic acids present in the cell or cells are fixed in position relative to each other by chemical crosslinking, for example by contacting the cells with one or more chemical cross linkers. This treatment locks in the spatial relationships between portions of nucleic acids in a cell. Any method of fixing the nucleic acids in their positions can be used. In some embodiments, the cells are fixed, for example with a fixative, such as an aldehyde, for example formaldehyde or gluteraldehyde. In some embodiments, a sample of one or more cells is cross-linked with a cross-linker to maintain the spatial relationships in the cell. For example, a sample of cells can be treated with a cross-linker to lock in the spatial information or relationship about the molecules in the cells, such as the DNA and RNA in the cell. In other embodiments, the relative positions of the nucleic acid can be maintained without using crosslinking agents. For example, the nucleic acids can be stabilized using spermine and spermidine (see Cullen et al., Science 261, 203 (1993), which is specifically incorporated herein by reference in its entirety). Other methods of maintaining the positional relationships of nucleic acids are known in the art. In some embodiments, nuclei are stabilized by embedding in a polymer such as agarose. In some embodiments, the cross-linker is a reversible cross-linker. In some embodiments, the cross-linker is reversed, for example after the fragments are joined and the spatial information is locked in. In specific examples, the nucleic acids are released from the cross-linked three-dimensional matrix by treatment with an agent, such as a proteinase, that degrade the proteinaceous material from the sample, thereby releasing the end ligated nucleic acids for further analysis, such as determination of the nucleic acid sequence. In specific embodiments, the sample is contacted with a proteinase, such as Proteinase K. In some embodiments of the disclosed methods, the cells are contacted with a crosslinking agent to provide the cross-linked cells. In some examples, the cells are contacted with a protein-nucleic acid crosslinking agent, a nucleic acid-nucleic acid crosslinking agent, a protein-protein crosslinking agent or any combination thereof. By this method, the nucleic acids present in the sample become resistant to special rearrangement and the spatial information about the relative locations of nucleic acids in the cell is maintained. In certain embodiments, the cells are cross linked such that the cohesin complex is not denatured. In some examples, a cross-linker is a reversible, such that the cross-linked molecules can be easily separated in subsequent steps of the method. In some examples, a cross-linker is a non-reversible cross-linker, such that the cross-linked molecules cannot be easily separated. In some examples, a cross-linker is light, such as UV light. In some examples, a cross linker is light activated. These cross-linkers include formaldehyde, disuccinimidyl glutarate, UV light, psoralens and their derivatives such as aminomethyltrioxsalen, glutaraldehyde, ethylene glycol bis[succinimidylsuccinate], bissulfosuccinimidyl suberate, 1-Ethyl-3-[3-dimethylaminopropyl]carbodiimide (EDC) bis[sulfosuccinimidyl] suberate (BS³) and other compounds known to those skilled in the art, including those described in the Thermo Scientific Pierce Crosslinking Technical Handbook, Thermo Scientific (2009) as available on the world wide web at piercenet.com/files/1601673_Crosslink_HB_Intl.pdf.

As used herein the term “contacting” refers to Placement in direct physical association, including both in solid or liquid form, for example contacting a sample with a crosslinking agent or a probe. As used herein the term “Crosslinking agent” refers to a chemical agent or even light, which facilitates the attachment of one molecule to another molecule. Crosslinking agents can be protein-nucleic acid crosslinking agents, nucleic acid-nucleic acid crosslinking agents, and protein-protein crosslinking agents. Examples of such agents are known in the art. In some embodiments, a crosslinking agent is a reversible crosslinking agent. In some embodiments, a crosslinking agent is a non-reversible crosslinking agent.

### Isolated Nuclei

In some embodiments, the cells are lysed to release the cellular contents, for example after crosslinking. In some examples the nuclei are lysed as well, while in other examples, the nuclei are maintained intact, which can then be isolated and optionally lysed, for example using a reagent that selectively targets the nuclei or other separation technique known in the art. In some examples, the sample is a sample of permeabilized nuclei, multiple nuclei, or isolated nuclei. In certain embodiments the cells are synchronized cells, (such at various points in the cell cycle, for example metaphase) before nuclei are isolated. In certain embodiments, cells are lysed under conditions that are non-denaturing, such that proteins remain folded in their native conformation and chromatin structure is maintained (e.g., intact chromatin). As used herein, chromatin structure is maintained refers to chromatin proteins remain bound to genomic DNA and does not fall off or have less stable or decreased binding as a result of being denatured. As used herein, chromatin structure is maintained also refers to minimally perturbing the spatial proximity of nucleic acids, protein folding, organelles, and/or nuclei. As used herein, chromatin structure is maintained also refers to conditions such that protein complexes do not fall apart or proteins are not denatured, for example cohesin complexes. In certain embodiments, cells are lysed under conditions that allow for cell lysis and permeabilization of the released nuclei. Chromatin structure is maintained in intact chromatin.

As used herein the term “isolated” refers to an “isolated” biological component (such as the end joined fragmented nucleic acids or nuclei as described herein) has been substantially separated or purified away from other biological components in the cell of the organism, in which the component naturally occurs, for example, extra-chromatin DNA and RNA, proteins and organelles. Nucleic acids and proteins that have been “isolated” include nucleic acids and proteins purified by standard purification methods, for example from a sample. The term also embraces nucleic acids and proteins prepared by recombinant expression in a host cell as well as chemically synthesized nucleic acids. It is understood that the term “isolated” does not imply that the biological component is free of trace contamination and can include nucleic acid molecules that are at least 50% isolated, such as at least 75%, 80%, 90%, 95%, 98%, 99%, or even 100% isolated.

### Permeabilizing Nuclei

In certain examples, the methods include permeabilizing nuclei. In certain embodiments, nuclei of the present invention can be permeabilized according to any method known in the art. In some cases, the nuclei may be permeabilized to allow access for nucleic acid processing reagents. The permeabilization may be performed in a way to minimally perturb the spatial proximity of nucleic acids, protein folding, organelles, and/or nuclei. In certain embodiments, the nuclei are permeabilized, such that protein complexes do not fall apart or proteins are not denatured. In some instances, the cells may be permeabilized using a permeabilization agent. Examples of permeabilization agents include NP40, digitonin, tween, streptolysin, exonuclease 1 buffer (NEB) and pepsin, and cationic lipids. In other instances, the cells, organelles, and/or nuclei may be permeabilized using hypotonic shock and/or ultrasonication. In other cases, the nucleic acid processing reagents e.g., enzymes such as nuclease, polymerase and/or ligase, may be highly charged, which may allow them to permeabilize through the membranes of the nuclei. Other embodiments include use of cell penetrating peptides to deliver cargo to the nuclei and allow capture of material. In certain embodiments, permeabilization steps, including pre-permeabilization are automated.

In certain embodiments, nuclei are permeabilized with a detergent. In certain embodiments, the detergent is non-ionic. In certain embodiments, the concentration of the detergent is sufficient to permeabilize the nuclei without denaturing proteins in the nuclei. In certain embodiments, NP40, digitonin, or tween is used. For example, the concentration of detergent used herein may be from 0.005% to 1%, from 0.01% to 0.8%, from 0.01% to 0.6%, from 0.01% to 0.4%, from 0.01% to 0.2%, from 0.01% to 0.1%, from 0.005% to 0.05%, from 0.01% to 0.03%, from 0.015% to 0.025%, from 0.018% to 0.022%, from 0.015% to 0.017%, from 0.016% to 0.018%, from 0.017% to 0.019%, from 0.018% to 0.02%, from 0.019% to 0.021%, from 0.02% to 0.022%, or from 0.021% to 0.023%. In some cases, the concentration of the detergent may be about 0.01%, about 0.015%, about 0.02%, about 0.025%, or about 0.03%. For example, the concentration of the detergent may be about 0.02%. In certain embodiments, SDS is used at concentrations below 0.5%, such as 0.1, 0.05, or less than 0.01%. In certain embodiments, the nuclei are not heated during permeabilization.

### Fragmenting, End-Repair, Fill-In and Ligation

In some embodiments, in order to create discrete portions of nucleic acid that can be joined together in subsequent steps of the methods, the nucleic acids present in the cells, such as cross-linked cells, are fragmented. In some embodiments, chromatin is fragmented, such that chromatin bound by proteins are protected from cleavage. Applicants have identified for the first time that chromatin fragmented by the methods described herein are protected from cleavage at sequences bound by proteins and that the methods provide information on chromatin accessibility in addition to ligation of chromatin fragments in proximity. Chromatin accessibility is only possible using intact chromatin as prior methods denatured proteins, such that protection was lost during fragmentation of chromatin that is not intact. The fragmentation can be done by a variety of methods, such as enzymatic and chemical cleavage. For example, DNA can be fragmented using any DNA cutter or combination thereof, such as, MseI and Csp6I; MboI, MseI, NlaIII and Csp6I; DNase I; micrococcal nuclease (MNase); benzonase; cyanase; another restriction enzyme; or a transposase complex. In one example, when intact chromatin is fragmented using MNase or DNase I the resulting fragmentation pattern detected after ligation is comparable to ultra-deep DNase-Seq (see, e.g., Madrigal P, Krajewski P. Current bioinformatic approaches to identify DNase I hypersensitive sites and genomic footprints from DNase-seq data. Front Genet. 2012; 3:230). In one example embodiment, accessible chromatin can be fragmented with a transposase to insert adapters into fragmented chromatin, such as in ATAC-seq (see, e.g., Buenrostro, et al., Transposition of native chromatin for fast and sensitive epigenomic profiling of open chromatin, DNA-binding proteins and nucleosome position. Nature methods 2013; 10 (12): 1213-1218). In one example embodiment, DNA can be fragmented using an endonuclease that cuts a specific sequence of DNA and leaves behind a DNA fragment with a 5′ overhang, thereby yielding fragmented DNA. In other examples, an endonuclease can be selected that cuts the DNA at random spots and yields overhangs or blunt ends. In some embodiments, fragmenting the nucleic acid present in the one or more cells comprises enzymatic digestion with an endonuclease that leaves 5′ overhanging ends. Enzymes that fragment, or cut, nucleic acids and yield an overhanging sequence are known in the art and can be obtained from such commercial sources as New England BioLabs® and Promega®. One of ordinary skill in the art can choose the restriction enzyme without undue experimentation. One of ordinary skill in the art will appreciate that using different fragmentation techniques, such as different enzymes with different sequence requirements, will yield different fragmentation patterns and therefore different nucleic acid ends. The process of fragmenting the sample can yield ends that are capable of being joined.

In certain embodiments, the ends of the fragmented DNA is repaired (e.g., end repair). Commercial reagents and protocols are available for DNA end repair. Fragmentation of polynucleotide molecules may result in fragments with a heterogeneous mix of blunt and 3′- and 5′-overhanging ends. It is therefore desirable to repair the fragment ends using methods or kits known in the art to generate ends that are optimal for ligation, for example, blunt sites of chromatin fragments. In a particular embodiment, the fragment ends of the nucleic acids are blunt ended. One method of the invention involves repairing the fragment ends with nucleotide triphosphates and a nucleic acid polymerase. The nucleotide triphosphates may contain a labeling modification, for example biotin or similar protein binding ligand, that allows selection of the end repaired fragments. The polymerase may be Klenow DNA polymerase or similar nucleic acid polymerase, that may have exonuclease activity in order to remove any 3′ overhanging ends. The reaction may be carried out with all four nucleotides, of which 0-4 may carry labeling modifications. The reaction may be carried out with a single labelled nucleoside triphosphate, and three unlabeled triphosphates, or may be carried out with two, three or four labeled nucleotides.

As used herein the term “Nucleic acid (molecule or sequence)” refers to a deoxyribonucleotide or ribonucleotide polymer including without limitation, cDNA, mRNA, genomic DNA, and synthetic (such as chemically synthesized) DNA or RNA or hybrids thereof. The nucleic acid can be double-stranded (ds) or single-stranded (ss). Where single-stranded, the nucleic acid can be the sense strand or the antisense strand. Nucleic acids can include natural nucleotides (such as A, T/U, C, and G), and can also include analogs of natural nucleotides, such as labeled nucleotides. Some examples of nucleic acids include the probes disclosed herein.

The major nucleotides of DNA are deoxyadenosine 5′-triphosphate (dATP or A), deoxyguanosine 5′-triphosphate (dGTP or G), deoxycytidine 5′-triphosphate (dCTP or C) and deoxythymidine 5′-triphosphate (dTTP or T). The major nucleotides of RNA are adenosine 5′-triphosphate (ATP or A), guanosine 5′-triphosphate (GTP or G), cytidine 5′-triphosphate (CTP or C) and uridine 5′-triphosphate (UTP or U). Nucleotides include those nucleotides containing modified bases, modified sugar moieties, and modified phosphate backbones, for example as described in U.S. Pat. No. 5,866,336 to Nazarenko et al.

Examples of modified base moieties which can be used to modify nucleotides at any position on its structure include, but are not limited to: 5-fluorouracil, 5-bromouracil, 5-chlorouracil, 5-iodouracil, hypoxanthine, xanthine, acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N˜6-sopentenyladenine, 1-methylguanine, 1-methylinosine, 2,2-dimethylguanine, 2-methyladenine, 2-methylguanine, 3-methylcytosine, 5-methyl cytosine, N6-adenine, 7-methylguanine, 5-methylaminomethyluracil, methoxyarninomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid, pseudouracil, queosine, 2-thiocytosine, 5-methyl-2-thiouracil, 2-thiouracil, 4-thiouracil, 5-methyluracil, uracil-5-oxyacetic acid methylester, uracil-S-oxyacetic acid, 5-methyl-2-thiouracil, 3-(3-amino-3-N-2-carboxypropyl) uracil, 2,6-diaminopurine and biotinylated analogs, amongst others.

Examples of modified sugar moieties which may be used to modify nucleotides at any position on its structure include, but are not limited to arabinose, 2-fluoroarabinose, xylose, and hexose, or a modified component of the phosphate backbone, such as phosphorothioate, a phosphorodithioate, a phosphoramidothioate, a phosphoramidate, a phosphordiamidate, a methylphosphonate, an alkyl phosphotriester, or a formacetal or analog thereof.

Ligation may be carried out in situ using any ligase known in the art and described further in the examples to obtain covalently linked joined DNA molecules. The ligation reaction may be carried out using any suitable ligase, for example, T3 or T4 ligase. Covalently linked: Refers to a covalent linkage between atoms by the formation of a covalent bond characterized by the sharing of pairs of electrons between atoms. In one example, a covalent link is a bond between an oxygen and a phosphorous, such as phosphodiester bonds in the backbone of a nucleic acid strand. In another example, a covalent link is one between a nucleic acid protein, another protein and/or nucleic acid that has been crosslinked by chemical means. In another example, a covalent link is one between fragmented nucleic acids.

In some embodiments, the end joined DNA that includes a labeled nucleotide is captured with a specific binding agent that specifically binds a capture moiety, such as biotin, on the labeled nucleotide. In some embodiments, the capture moiety is adsorbed or otherwise captured on a surface. In specific embodiments, the end target joined DNA is labeled with biotin, for instance by incorporation of biotin-14-CTP or other biotinylated nucleotide during the filling in of the 5′ overhang, for example with a DNA polymerase, allowing capture by streptavidin. This step can also be referred to herein as “biotin filling” or “biotin-fill-in”. In some embodiments, the step(s) of biotin filling can be completed in about 1 to about 45 minutes such as about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or about 45 minutes. Any additional biotin filing steps as discussed elsewhere herein, can also be completed in about in about 1 to about 45 minutes such as about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or about 45 minutes.

As used herein the term “biotin-14-CTP” refers to a biologically active analog of cytosine-5′-triphosphate that is readily incorporated into a nucleic acid by polymerase or a reverse transcriptase. In some examples, biotin-14-CTP is incorporated into a nucleic acid fragment that has a 3′ overhang.

As used herein the term “capture moieties” refers to molecules or other substances that when attached to a nucleic acid molecule, such as an end joined nucleic acid, allow for the capture of the nucleic acid molecule through interactions of the capture moiety and something that the capture moiety binds to, such as a particular surface and/or molecule, such as a specific binding molecule that is capable of specifically binding to the capture moiety.

Other means for labeling, capturing, and detecting nucleic acid probes include: incorporation of aminoallyl-labeled nucleotides, incorporation of sulfhydryl-labeled nucleotides, incorporation of allyl- or azide-containing nucleotides, and many other methods described in Bioconjugate Techniques (2^{nd} Ed), Greg T. Hermanson, Elsevier (2008), which is specifically incorporated herein by reference. In some embodiments the specific binding agent has been immobilized for example on a solid support, thereby isolating the target nucleic molecule of interest. By “solid support or carrier” is intended any support capable of binding a targeting nucleic acid. Well-known supports or carriers include glass, polystyrene, polypropylene, polyethylene, dextran, nylon, amylases, natural and modified celluloses, polyacrylamides, agarose, gabbros and magnetite. The nature of the carrier can be either soluble to some extent or insoluble for the purposes of the present disclosure. The support material may have virtually any possible structural configuration so long as the coupled molecule is capable of binding to targeting probe. Thus, the support configuration may be spherical, as in a bead, or cylindrical, as in the inside surface of a test tube, or the external surface of a rod. Alternatively, the surface may be flat such as a sheet or test strip. After capture, these end joined nucleic acid fragments are available for further analysis, for example to determine the sequences that contributed to the information encoded by the ligation junction, which can be used to determine which DNA sequences are close in spatial proximity in the cell, for example to map the three dimensional structure of DNA in a cell such as genomic and/or chromatin bound DNA. In some embodiments, the sequence is determined by PCR, hybridization of a probe and/or sequencing, for example by sequencing using high-throughput paired end sequencing. In some embodiments determining the sequence at the one or more junctions of the one or more end joined nucleic acid fragments comprises nucleic acid sequencing, such as short-read sequencing technologies or long-read sequencing technologies. In some embodiments, nucleic acid sequencing is used to determine two or more junctions within an end-joined concatemer simultaneously.

As used herein the term “specific binding agent” refers to an agent that binds substantially or preferentially only to a defined target such as a protein, enzyme, polysaccharide, oligonucleotide, DNA, RNA, recombinant vector or a small molecule. In an example, a “specific binding agent that specifically binds to the label” is capable of binding to a label that is covalently linked to a targeting probe.

In some embodiments, determining the sequence of a junction includes using a probe that specifically binds to the junction at the site of the two joined nucleic acid fragments. In particular embodiments, the probe specifically hybridizes to the junction both 5′ and 3′ of the site of the join and spans the site of the join. A probe that specifically binds to the junction at the site of the join can be selected based on known interactions, for example in a diagnostic setting where the presence of a particular target junction, or set of target junctions, has been correlated with a particular disease or condition. It is further contemplated that once a target junction is known, a probe for that target junction can be synthesized.

In some embodiments, the end joined nucleic acids are selectively amplified. In some examples, to selectively amplify the end joined nucleic acids, a 3′ DNA adaptor and a 5′ RNA, or conversely a 5′ DNA adaptor and a 3′ RNA adaptor can be ligated to the ends of the molecules can be used to mark the end joined nucleic acids. Using primers specific for these adaptors only end joined nucleic acids will be amplified during an amplification procedure such as PCR. In some embodiments, the target end joined nucleic acid is amplified using primers that specifically hybridize to the adaptor nucleic acid sequences present at the 3′ and 5′ ends of the end joined nucleic acids. In some embodiments, the non-ligated ends of the nucleic acids are end repaired. In some embodiments attaching sequencing adapters to the ends of the end ligated nucleic acid fragments.

As used herein the term “primers” refers to short nucleic acid molecules, such as a DNA oligonucleotide, which can be annealed to a complementary target nucleic acid molecule by nucleic acid hybridization to form a hybrid between the primer and the target nucleic acid strand. A primer can be extended along the target nucleic acid molecule by a polymerase enzyme. Therefore, primers can be used to amplify a target nucleic acid molecule, wherein the sequence of the primer is specific for the target nucleic acid molecule, for example so that the primer will hybridize to the target nucleic acid molecule under very high stringency hybridization conditions.

The specificity of a primer increases with its length. Thus, for example, a primer that includes 30 consecutive nucleotides will anneal to a target sequence with a higher specificity than a corresponding primer of only 15 nucleotides. Thus, to obtain greater specificity, probes and primers can be selected that include at least 5, 10, 15, 20, 25, 30, 35, 40, 45, 50 or more consecutive nucleotides.

In particular examples, a primer is at least 15 nucleotides in length, such as at least 5 contiguous nucleotides complementary to a target nucleic acid molecule. Particular lengths of primers that can be used to practice the methods of the present disclosure include primers having at least 5, at least 10, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 45, at least 50, or more contiguous nucleotides complementary to the target nucleic acid molecule to be amplified, such as a primer of 5-60 nucleotides, 15-50 nucleotides, 15-30 nucleotides or greater.

Primer pairs can be used for amplification of a nucleic acid sequence, for example, by PCR, or other nucleic-acid amplification methods known in the art. An “upstream” or “forward” primer is a primer 5′ to a reference point on a nucleic acid sequence. A “downstream” or “reverse” primer is a primer 3′ to a reference point on a nucleic acid sequence. In general, at least one forward and one reverse primer are included in an amplification reaction. PCR primer pairs can be derived from a known sequence, for example, by using computer programs intended for that purpose such as Primer (Version 0.5, © 1991, Whitehead Institute for Biomedical Research, Cambridge, MA).

Methods for preparing and using primers are described in, for example, Sambrook et al. (1989) Molecular Cloning: A Laboratory Manual, Cold Spring Harbor, New York; Ausubel et al. (1987) Current Protocols in Molecular Biology, Greene Publ. Assoc. & Wiley-Intersciences.

### Sequencing

In certain embodiments, the one or more end joined nucleic acid fragments are sequenced to determine the junction, cut site, and the sequence of the entire joined fragments. In certain embodiments, ligation junction sequencing is performed to ensure an accurate sequence of the ligation junction is obtained. In certain embodiments, the exact sequences with the highest contacts are determined. In a typical paired end sequencing reaction fragments are approximately 500 base pairs and the fragments are sequenced from each end. Ligation junction sequencing requires shorter fragments and/or sequencing from a single end. In certain embodiments, the nucleic acid fragments for ligation junction sequencing are between about 100 and about 400 bases in length, such as about 100, about 150, about 200, about 250, about 300, about 350, about 400, or about 450 bases in length, for example form about 100 to about 400, about 200 to about 300, about 250 to about 350, and about 250 to about 300 base pairs in length and the like. In specific examples, end joined fragments are selected for sequence determination that are between about 200 and 300 base pairs in length. In certain embodiments, end joined fragments of about 250 base pairs in length are sequenced from both ends. In certain embodiments, end joined fragments of about 300 base pairs in length are sequenced from a single end.

As used herein the term “junction” refers to a site where two nucleic acid fragments or joined, for example using the methods described herein. A junction encodes information about the proximity of the nucleic acid fragments that participate in formation of the junction. For example, junction formation between to nucleic acid fragments indicates that these two nucleic acid sequences where in close proximity when the junction was formed, although they may not be in proximity in linear nucleic acid sequence space. Thus, a junction can define long range interactions. In some embodiments, a junction is labeled, for example with a labeled nucleotide, for example to facilitate isolation of the nucleic acid molecule that includes the junction.

In some embodiments, the nucleic acids present in the ligated sample are purified, for example using ethanol precipitation. In example embodiments of the disclosed method the cell nuclei are not subjected to mechanical lysis. In some example embodiments, the sample is not subjected to RNA degradation. In specific embodiments, the sample is not contacted with an exonuclease to remove biotin from un-ligated ends. In some embodiments, the sample is not subjected to phenol/chloroform extraction.

As used herein the term “DNA sequencing” refers to the process of determining the nucleotide order of a given DNA molecule. In certain embodiments, the sequencing can be performed using automated Sanger sequencing. In certain embodiments, sequencing comprises high-throughput (formerly “next-generation”) technologies to generate sequencing reads from the one or more end joined nucleic acid fragments. In DNA sequencing, a read is an inferred sequence of base pairs (or base pair probabilities) corresponding to all or part of a single DNA fragment. A typical sequencing experiment involves fragmentation of the genome into millions of molecules or generating complementary DNA (cDNA) fragments, which are size-selected and ligated to adapters. The set of fragments is referred to as a sequencing library, which is sequenced to produce a set of reads. Methods for constructing sequencing libraries are known in the art (see, e.g., Head et al., Library construction for next-generation sequencing: Overviews and challenges. Biotechniques. 2014; 56(2): 61-77; Trombetta, J. J., Gennert, D., Lu, D., Satija, R., Shalek, A. K. & Regev, A. Preparation of Single-Cell RNA-Seq Libraries for Next Generation Sequencing. Curr Protoc Mol Biol. 107, 4 22 21-24 22 17, doi:10.1002/0471142727.mb0422s107 (2014). PMCID:4338574). A “library” or “fragment library” may be a collection of nucleic acid molecules derived from one or more nucleic acid samples, in which fragments of nucleic acid have been modified, generally by incorporating terminal adapter sequences comprising one or more primer binding sites and identifiable sequence tags. In certain embodiments, the library members (e.g., genomic DNA, cDNA) may include sequencing adaptors that are compatible with use in, e.g., Illumina's reversible terminator method, long read nanopore sequencing, Roche's pyrosequencing method (454), Life Technologies' sequencing by ligation (the SOLiD platform) or Life Technologies' Ion Torrent platform. Examples of such methods are described in the following references: Margulies et al (Nature 2005 437: 376-80); Schneider and Dekker (Nat Biotechnol. 2012 Apr. 10; 30(4):326-8); Ronaghi et al (Analytical Biochemistry 1996 242: 84-9); Shendure et al (Science 2005 309: 1728-32); Imelfort et al (Brief Bioinform. 2009 10:609-18); Fox et al (Methods Mol. Biol. 2009; 553:79-108); Appleby et al (Methods Mol. Biol. 2009; 513:19-39); and Morozova et al (Genomics. 2008 92:255-64), which are incorporated by reference for the general descriptions of the methods and the particular steps of the methods, including all starting products, reagents, and final products for each of the steps.

In certain embodiments, sequencing of the isolated end joined nucleic acid fragments results in whole genome sequencing. Whole genome sequencing (also known as WGS, full genome sequencing, complete genome sequencing, or entire genome sequencing) is the process of determining the complete DNA sequence of an organism's genome at a single time. This entails sequencing all of an organism's chromosomal DNA as well as DNA contained in the mitochondria and, for plants, in the chloroplast. “Whole genome amplification” (“WGA”) refers to any amplification method that aims to produce an amplification product that is representative of the genome from which it was amplified. Non-limiting WGA methods include Primer extension PCR (PEP) and improved PEP (I-PEP), Degenerated oligonucleotide primed PCR (DOP-PCR), Ligation-mediated PCR (LMP), T7-based linear amplification of DNA (TLAD), and Multiple displacement amplification (MDA).

In certain embodiments, the present invention includes whole exome sequencing by enriching for the one or more end joined nucleic acid fragments representative of the exome (e.g., hybrid selection, HYbrid Capture Hi-C(Hi-C2)). Exome sequencing, also known as whole exome sequencing (WES), is a genomic technique for sequencing all of the protein-coding genes in a genome (known as the exome) (see, e.g., Ng et al., 2009, Nature volume 461, pages 272-276). It consists of two steps: the first step is to select only the subset of DNA that encodes proteins. These regions are known as exons—humans have about 180,000 exons, constituting about 1% of the human genome, or approximately 30 million base pairs. The second step is to sequence the exonic DNA using any high-throughput DNA sequencing technology. In certain embodiments, whole exome sequencing is used to determine somatic mutations in genes associated with disease (e.g., cancer mutations).

In certain embodiments, the present invention includes targeted sequencing by enriching for the one or more end joined nucleic acid fragments representative of a panel of genes or sequences (e.g., hybrid selection, HYbrid Capture Hi-C(Hi-C2), discussed further herein). Targeted gene sequencing panels are useful tools for analyzing specific mutations in a given sample. Focused panels contain a select set of genes or gene regions that have known or suspected associations with the disease or phenotype under study. In certain embodiments, targeted sequencing is used to detect mutations associated with a disease in a subject in need thereof. Targeted sequencing can increase the cost-effectiveness of variant discovery and detection.

In certain embodiments, the present invention includes amplification to increase the number of copies of a nucleic acid molecule, such as one or more end joined nucleic acid fragments that includes a junction, such as a ligation junction. The resulting amplification products are called “amplicons.” Amplification of a nucleic acid molecule (such as a DNA or RNA molecule) refers to use of a technique that increases the number of copies of a nucleic acid molecule (including fragments).

An example of amplification is the polymerase chain reaction (PCR), in which a sample is contacted with a pair of oligonucleotide primers under conditions that allow for the hybridization of the primers to a nucleic acid template in the sample. The primers are extended under suitable conditions, dissociated from the template, re-annealed, extended, and dissociated to amplify the number of copies of the nucleic acid. This cycle can be repeated. The product of amplification can be characterized by such techniques as electrophoresis, restriction endonuclease cleavage patterns, oligonucleotide hybridization or ligation, and/or nucleic acid sequencing.

Other examples of in vitro amplification techniques include quantitative real-time PCR; reverse transcriptase PCR (RT-PCR); real-time PCR (rt PCR); real-time reverse transcriptase PCR (rt RT-PCR); nested PCR; strand displacement amplification (see U.S. Pat. No. 5,744,311); transcription-free isothermal amplification (see U.S. Pat. No. 6,033,881, repair chain reaction amplification (see WO 90/01069); ligase chain reaction amplification (see European patent publication EP-A-320 308); gap filling ligase chain reaction amplification (see U.S. Pat. No. 5,427,930); coupled ligase detection and PCR (see U.S. Pat. No. 6,027,889); and NASBA™ RNA transcription-free amplification (see U.S. Pat. No. 6,025,134) amongst others.

Furthermore, the methods disclosed herein can readily be combined with other techniques, such as hybrid capture after library generation (to target specific parts of the genome), chromatin immunoprecipitation after ligation (to examine the chromatin environment of regions associated with specific proteins), bisulfite treatment, (to probe the methylation state of DNA). For examples the information from one or more ligation junctions is used to infer and/or determine the three-dimensional structure of the genome. In some embodiments, the information from one or more ligation junctions is used to simultaneously map protein-DNA interactions and DNA-DNA interactions or RNA-DNA interactions and DNA-DNA interactions. In some embodiments, the information from one or more ligation junctions is used to simultaneously map methylation and three-dimensional structure. In some embodiments, the information from more than one ligation junction is used to assemble whole genomes or parts of genomes. In some embodiments, the sample is treated to accentuate interactions between contiguous regions of the genome. In some embodiments, the cells in the sample are synchronized in metaphase.

In one example embodiment, hybrid capture after library generation comprises treating a library of end joined nucleic acid fragments generated using the methods described above with an agent that isolates end joined nucleic acid fragments comprising specific nucleic acid sequence (target sequence). In certain example embodiments, the specific nucleic acid sequence is at least 50, at least 60, at least 70, at least 80, at least 90, at least 100, at least 110, at least 120, at least 130, at least 140, at least 150, at least 160, at least 170, at least 180, at least 190, or at least 200 base pairs long. In certain example embodiments, the specific nucleic acid sequence is within at least 50, at least 60, at least 70, at least, 80, at least 90, or at least 100 base pairs, in either the 5′ or 3′ direction, of a restriction site. In certain example embodiments, the specific nucleic sequence comprises less than ten repetitive bases. In certain other example embodiments, the GC content of the specific nucleic acid sequence is between 25% and 80%, between 40% and 70%, or between 50% and 60%.

In certain example embodiments, the agent that isolates the end joined nucleic acid fragments comprising the specific nucleic acid sequence is a probe. The probe may be labeled. In certain example embodiments, the probe is radiolabeled, fluorescently-labeled, enzymatically-labeled, or chemically labeled. In certain other example embodiments, the probe may be labeled with a capture moiety, such as a biotin-label. When the probe is labeled with a capture moiety, the capture moiety may be used to isolate the end joined nucleic acid fragments using techniques such as those known in the art and described previously. The exact sequence of the isolated end-joined nucleic acid fragments may then be determined, for example, by sequencing as described previously.

### Phasing

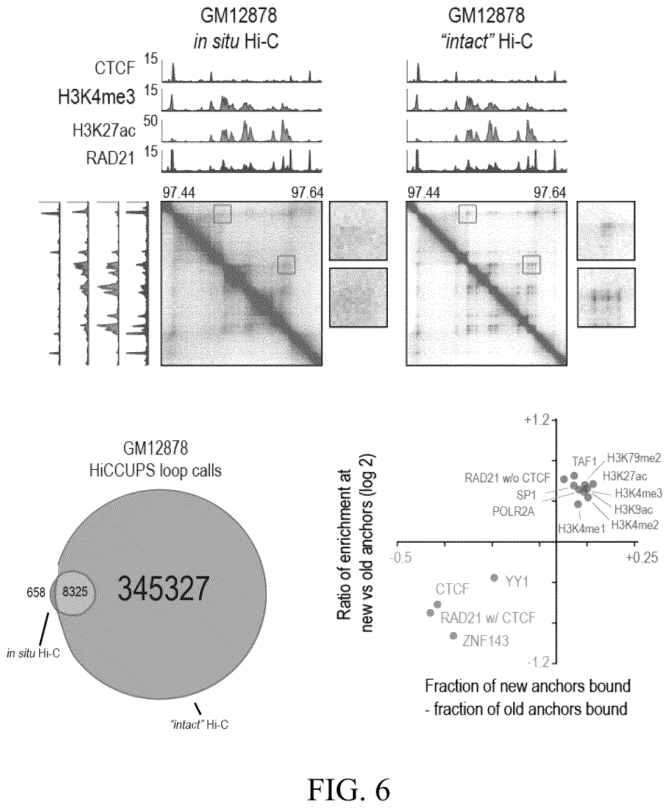

In certain embodiments, the methods described herein can provide suitable data suitable for phasing different haplotypes. In one advantageous embodiment, phasing using intact Hi-C as described herein can be performed because of the greater resolution of DNA contacts and loops that can be identified (see, e.g., FIG. 6 showing identification of 350K loops as compared to 9K loops identified with previous methods). The methods described herein do not require additional outside data. Conventional phasing methods have certain limitations. Assisted methods are limited by the requirement for sequence trios and/or the reliance of population-based inferences, which require linkage information and are useful only in the normal state. De novo methods which have long reads make it difficult to recognize SNPs and pseudo-long reads do not produce chromosome-length haploblocks. Hi-C and other DNA proximity assays, such as any of those described in greater detail elsewhere herein can provide powerful sources of linking data. Data generated from the DNA proximity assays (e.g., Hi-C and others described herein) can be used to phase a genome. Loci on the same chromosome tend to talk to each other more often than to loci on other chromosomes. This is a helpful signal for assembly to anchor contigs to chromosomes. Thus, also described herein are methods of phasing different haplotypes. In some embodiments, the method can include calculating a frequency of contact between loci containing particular variants, wherein the frequency of contact is determined using sequencing reads derived from a DNA proximity ligation assay (such as any of those described and demonstrated elsewhere herein), wherein the frequency of contact between two variants indicates if two variants are on the same molecule.

In certain example embodiments, the frequency of contact between two variants is compared to an expected model to determine whether the two variants are on the same molecule. The expected model may be determined based on a contact matrix derived from a DNA proximity ligation assay, wherein reads are represented as pixels in the contact map and wherein contact frequency is a function of distance from a diagonal of the contact matrix. In certain example embodiments, the analysis may be done in an iterative fashion and wherein in data from DNA proximity ligation experiments is used to go from one possible phasing of a variant set to another possible phasing of a variant set. The analysis of the data from the DNA proximity ligation experiments is performed using gradient descent, hill-climbing, a genetic algorithm, reducing to an instance of the Boolean satisfiability problem (SAT) and solving, or using any combinatorial optimization algorithm.

The methods disclosed herein may also be used to assist in phasing of the human genome. Phasing can be performed de novo and using population data. The 3D contact maps can be used to assess the accuracy of phasing results.

The methods disclosed herein may also be used to analyze karyotype evolution in given group of species as well as to detect karyotype polymorphisms, even at low-coverage. The karyotype data can be used to identify phylogenetic relationships, either by itself or with sequence level data.

The methods disclosed herein may also be used to substitute for inter-species chromosome painting, including at low coverage.

The methods disclosed herein may also be used to estimate the distance along the 1D sequence between any two given genomic sequences.

The methods disclosed herein may use the features of 3D contact maps. For example, identification of chromatin motifs in their proper convergent orientation can be used to properly orient other contigs in the assembly.

The methods disclosed herein can include a phasing module that utilizes a signal produced from a DNA proximity assay such as anyone described herein. The module can take as input a list of variants (.vcf) e.g. generated by realignment of data from a DNA proximity assay described herein (e.g. Intact Hi-C and others) as well as list of dedupped Hi-C alignments (Jucier mind file). Various embodiments can be capable of producing chromosome-length haploblocks solely from ENCODE data. Various embodiments can take advantage of partial phasing data such as long-read phasing, population phasing, etc.

### Nuclease Sensitivity or Chromatin Accessibility Maps

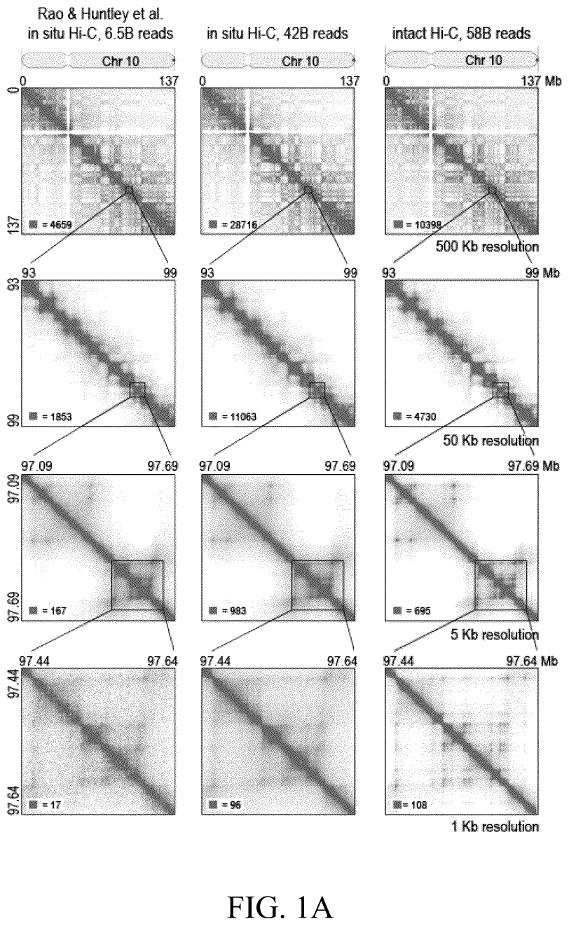

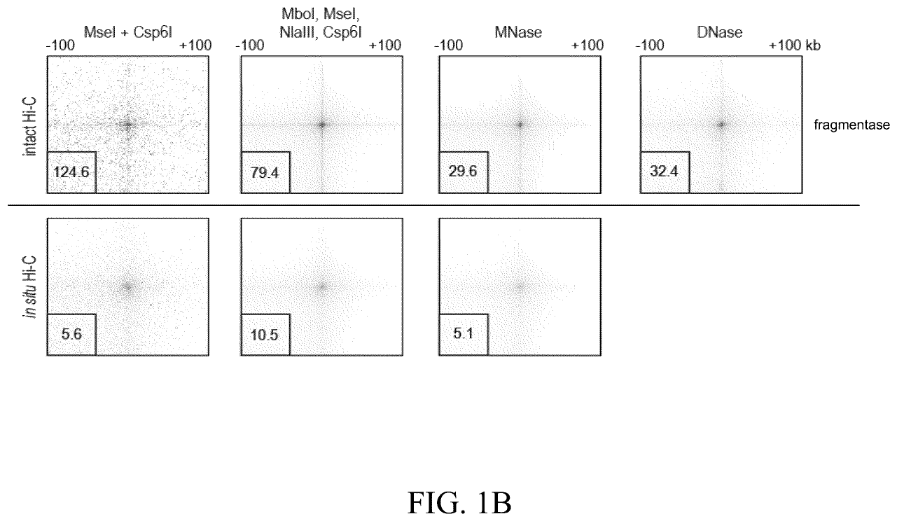

In example embodiments, every experiment includes a nuclease or chromatin accessibility map that can be used to confirm that ligated chromatin fragments were derived from intact chromatin. Additionally, the nuclease or chromatin accessibility map is phased based on the contacts between chromatin DNA and genome scale with resolution as low as single base pair resolution. Thus, the map provides for a confirmation of intact chromatin and also provides for every sequence in phased homologs that is protected from fragmentation. Generating the nuclease or chromatin accessibility map can be generated using a novel sequencing pipeline that can be incorporated into the pipeline for generating contact maps. DNase I hypersensitive sites (DHSs) are described and can be mapped in chromatin (see, e.g., FIG. 1 of Wang Y M, Zhou P, Wang L Y, Li Z H, Zhang Y N, Zhang Y X. Correlation between DNase I hypersensitive site distribution and gene expression in HeLa S3 cells. PLoS One. 2012; 7(8):e42414). Chromatin accessibility maps generated by prior methods have been described and cannot be phased (see e.g., Tsompana, M., Buck, M. J. Chromatin accessibility: a window into the genome. Epigenetics & Chromatin 7, 33 (2014)).

### DNA Methylation Maps

In example embodiments, phased DNA methylation maps can be generated by treating the ligated chromatin fragments with one or more agents that distinguish between unmodified and modified cytosines, such as methylated cytosines (mC) and hydroxymethylated cytosines (hmC). The treatment can be performed before or after ligated chromatin fragments are isolated because isolated DNA includes the methylated nucleotides. Methods for distinguishing DNA methylation include (i) bisulfite conversion, (ii) Tet-assisted bisulfite conversion, (iii) Tet-assisted conversion with a substituted borane reducing agent, and (iv) protection of hmC followed by Tet-assisted conversion with a substituted borane reducing agent (see, e.g., US patent Application No. US20210115502A1). Methylation can also be detected using methylation specific restriction enzymes or methylated DNA immunoprecipitation (MeDIP). In example embodiments, phased DNA methylation maps can be generated where methylated cytosines (mC) and hydroxymethylated cytosines (hmC) are determined by the sequencer itself and independent of one or more agents (e.g., using PacBio or Nanopore sequencers).

### DNA Protein-Binding Maps

In example embodiments, phased DNA protein-binding maps can be generated by immunoprecipitation of ligated chromatin fragments with antibodies specific for chromatin proteins or chromatin modifications, such as modified histones. Chromatin Immunoprecipitation (ChIP) is used to immunoprecipitated crosslinked chromatin to determine sequences bound by proteins or modified histones. ChIP-seq combines chromatin immunoprecipitation (ChIP) with massively parallel DNA sequencing to identify the binding sites of DNA-associated proteins (see, e.g., Nakato R, Sakata T. Methods for ChIP-seq analysis: A practical workflow and advanced applications. Methods. 2021; 187:44-53). Both methods are not capable of phasing the homolog the protein or modification is present on. Thus, patterns on a specific chromosome cannot be determined. The method of ChIP can be combined with the high resolution methods described herein to generate phased maps. Another advantage of combining ChIP-seq with the methods described herein is that precise binding sites can be determined without any outside knowledge by combining the ChIP-seq map with chromatin accessibility map.

### Spatial Proximity Maps

In example embodiments, phased DNA contact maps with nuclease sensitivity confirmation can be generated, such as a Hi-C map. As used herein a Hi-C map is a list of DNA-DNA contacts produced by a Hi-C experiment. By partitioning the linear genome into “loci” of fixed size, the Hi-C map can be represented as a “contact matrix” M, where the entry Mi,j is the number of contacts observed between locus Li and locus Lj. (A “contact” is a read pair that remains after Applicants exclude reads that do not align uniquely to the genome, that correspond to unligated fragments, or that are duplicates.) The contact matrix can be visualized as a heatmap, whose entries are called “pixels”. An “interval” refers to a (one-dimensional) set of consecutive loci; the contacts between two intervals thus form a “rectangle” or “square” in the contact matrix. “Matrix resolution” is defined as the locus size used to construct a particular contact matrix and “map resolution” as the smallest locus size such that 80% of loci have at least 1000 contacts. The map resolution describes the finest scale at which one can reliably discern local features in the data.

Applicants can identify loops by looking for pairs of loci that have significantly more contacts with one another than they do with other nearby loci. The key reason is that Applicants call peaks only when a pair of loci shows elevated contact frequency relative to the local background—that is, when the peak pixel is enriched as compared to other pixels in its neighborhood.

In example embodiments, aggregate peak analysis (APA) is performed on contact matrices. To measure the aggregate enrichment of a set of putative peaks in a contact matrix, Applicants plot the sum of a series of submatrices derived from that contact matrix. Each of these submatrices is a square centered at a single putative peak in the upper triangle of the contact matrix. The resulting APA plot displays the total number of contacts that lie within the entire putative peak set at the center of the matrix. Focal enrichment across the peak set in aggregate manifests as larger values at the center of the APA plot.

### Single Cell or Single Molecule Epigenetic Maps

The embodiments disclosed herein can also be applied to single cell or single molecule assays. For example, chromatin fragments can be tagged with cell specific barcode sequences. Methods of barcoding can include any method known in the art. The chromatin fragments can then be assigned to the cell or chromosome of origin based on the sequenced barcodes.

Nuclei may be barcoded using split pool methods of generating barcodes in intact nuclei (see, e.g., Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Rosenberg et al., “Single-cell profiling of the developing mouse brain and spinal cord with split-pool barcoding” Science 15 Mar. 2018; Vitak, et al., “Sequencing thousands of single-cell genomes with combinatorial indexing” Nature Methods, 14(3):302-308, 2017; Cao, et al., Comprehensive single-cell transcriptional profiling of a multicellular organism).

Barcoding may also include transposon specific adapters that can be used to both fragment and tag DNA fragments in nuclei, such as in single cell ATAC-seq (see, e.g., Buenrostro et al., Single-cell chromatin accessibility reveals principles of regulatory variation. Nature 523, 486-490 (2015); Cusanovich, D. A., Daza, R., Adey, A., Pliner, H., Christiansen, L., Gunderson, K. L., Steemers, F. J., Trapnell, C. & Shendure, J. Multiplex single-cell profiling of chromatin accessibility by combinatorial cellular indexing. Science. 2015 May 22; 348(6237):910-4. doi: 10.1126/science.aab1601. Epub 2015 May 7; US20160208323A1; US20160060691A1; and WO2017156336A1).

In one example embodiment, single nuclei can be fragmented by inserting universal adapter sequences by tagmentation. The single nuclei can then be merged with barcoded beads in emulsion droplets or microwells, such that barcoded beads include capture sequences specific for the universal adapter sequences. The barcodes can then be transferred to the ligated chromatin fragments. Methods of using barcoded beads have been described (see, e.g., Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. January; 12(1):44-73; Gierahn et al., “Seq-Well: portable, low-cost RNA sequencing of single cells at high throughput” Nature Methods 14, 395-398 (2017); Hughes, et al., “Highly Efficient, Massively-Parallel Single-Cell RNA-Seq Reveals Cellular States and Molecular Features of Human Skin Pathology” bioRxiv 689273; Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958; International Patent Application No. PCT/US2016/059239, published as WO2017164936 on Sep. 28, 2017; International Patent Application No. PCT/US2018/060860, published as WO/2019/094984 on May 16, 2019; International Patent Application No. PCT/US2019/055894, published as WO/2020/077236 on Apr. 16, 2020; Drokhlyansky, et al., “The enteric nervous system of the human and mouse colon at a single-cell resolution,” bioRxiv 746743; doi: doi.org/10.1101/746743; and Drokhlyansky E, Smillie C S, Van Wittenberghe N, et al. The Human and Mouse Enteric Nervous System at Single-Cell Resolution. Cell. 2020; 182(6):1606-1622.e23).

### Genome Assembly

In another aspect, the invention provides a method for reference-assisted genome assembly. Reads from DNA proximity ligation reads on a test sample may be aligned to a reference sequence derived from a control sample to generate a combined 3D contact map. The chromosomal breakpoints and/or fusions are identified between the test sample and the reference sample to create a proxy genome assembly. Variant calling may then be used to identify one or more small-scale changes, such as indels and singe nucleotide polymorphisms, between the realigned test sample and the control reference sequence. Local reassembly is then performed on the identified variants to address the one or more small-scale changes to generate a final output genome assembly. The test sample and the reference sample may be from the same or different species, or from closely related or distantly related species. The breakpoints and fusions may be identified using one of the embodiments disclosed above. In certain example embodiments, the breakage and fusion points are examined to determine regions of synteny between the test and reference samples and/or polymorphisms. The test sample may be aligned to the same or different reference sample, or multiple test samples may be aligned to many different reference sample sequences. The breakage and fusion points may be examined to infer phylogenetic relationships between samples. In certain example embodiment, multiple reference-assisted assemblies may be prepared at the same time.

As used herein the term “control” refers to a reference standard. A control can be a known value or range of values indicative of basal levels or amounts or present in a tissue or a cell or populations thereof. A control can also be a cellular or tissue control, for example a tissue from a non-diseased state and/or exposed to different environmental conditions. A difference between a test sample and a control can be an increase or conversely a decrease. The difference can be a qualitative difference or a quantitative difference, for example a statistically significant difference.

In another aspect, the invention provides a method for genome assembly, wherein proper orientation of contigs and/or scaffolds is determined, at least in part, by the relative orientation of certain DNA motifs. The motif may be a CTCF mediated loop. The proper orientation may be determined, at least in part, from DNA proximity ligation assays, which may be used to generate a 3D contact map defining one or more contact domains, loops, compartment domains, links, compartment loops, superloops, one or more compartment interactions. The 3D contact map may also define centromere and telomere regions. In certain example embodiment, the DNA proximity ligation assay is Hi-C. In certain example embodiments, wherein massively multiplex single cell Hi-C is used to identify different subpopulations with differences in scaling and long range behavior. The DNA proximity ligation assay may be performed on synchronized populations of cells. In certain example embodiments, the cells may be synchronized in metaphase. The method may be performed on one or more cell treated to modify genome folding. Modifications may include gene editing, degradation of proteins that play a role in genome folding (such as HDAC inhibitors, Degron that target CTCF, Cohesin etc.), and/or modification of transcriptional machinery. The methods may be used to assemble transcriptomes. In certain example embodiments bisulfite treatment is applied to ligation junctions derived from a proximity ligation experiment and used to analyze proximity between DNA loci in sample, including the frequency of methylation for one or more basis in a sample.

In another aspect, the invention provides a method for genome assembly wherein the proper orientation of contigs and/or scaffolds is determined, at least in part, by the relative orientation of certain DNA motifs. In certain example embodiments, the motif is a CTCF motif. In certain example embodiments, the proper orientation of the motifs is determined, at least in part, by data from a DNA proximity ligation assay.

In another aspect, the invention provides a method for estimating the linear genomic distance between sequences in a gene comprising sequencing reads derived from DNA proximity ligation assay. The distance may be determined, at least in part, based on the frequency a given sequence forms contacts with another sequence in the set. The distance may also be determined based on the relative orientation with which a given sequence forms contacts with other sequences in the set. In certain example embodiments, the contact features are determined from DNA proximity ligation assays. In certain example embodiments, a contact map generated from the DNA proximity ligation assays may be used to derive an expected model for the linear genomic distance between sequences in a genome.

In another example embodiment, the invention provides a method for quality control analysis of genome assemblies by visually examining a contact map derived from a DNA proximity ligation assay. In certain example embodiments, the visual examination may be facilitated by a computer implemented graphical user interface, wherein the graphical user interface facilitates annotation of the genome assembly. In certain example embodiments, the contig map may span a single contig or scaffold.

The methods described herein can be used to generate a personalized genome as further.

The methods disclosed herein may also be used to assemble/identify genomes in a metagenomic context. The applications include, but are not limited to, sequencing prokaryotic, eukaryotic and mixed communities from the same samples. For example, the methods may be used, among other metagenomic applications, to sequence the metagenome with the host genome, disease vectors and pathogens, and disease vectors and host etc.

### Other Applications

Various embodiments of methods described herein can be used to generate data that can be analyzed using various deep learning techniques and methods for genome wide analyses.

Considering the wealth of information that can be gained using the methods described herein, with respect to genome architecture at the primary, secondary, tertiary and beyond (see Examples below), the methods disclosed herein can be used to apply genome engineering techniques for the treatment of disease as well as the study of biological questions. In some embodiments, the organizational structure of a genome is determined using the methods disclosed herein. For example, the methods disclosed herein have been demonstrated to generate very dense contact maps. In some examples, sequences obtained using the methods disclosed herein are mapped to a genome of an organism, such as an animal, plant, fungi, or microorganism, for example, a bacterial, yeast, virus, and the like. In some examples, diploid maps corresponding to each chromosomal homolog are constructed. These maps, as well as others that can be generated using the disclosed technology provide a picture, such as a three-dimensional picture, of genomic architecture with high resolution, such as a resolution of 1 kilobase or even lower, for example less then 50 bases, in particular 1 to 10 bp resolution.

As disclosed herein, the inventors have shown that a genome is partitioned into domains that are associated with particular patterns of histone marks that segregates into sub-compartments, distinguished by unique long-range contact patterns. Using the maps, loops across the genome can be studied and their properties identified, including their strong association with gene activation.

### Detection of Junctions by Hybridization

In some embodiments of the disclosed methods, determining the identity of a nucleic acid, such as a target junction, includes detection by nucleic acid hybridization. Nucleic acid hybridization involves providing a probe and target nucleic acid under conditions where the probe and its complementary target can form stable hybrid duplexes through complementary base pairing. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids. Under low stringency conditions (e.g., low temperature and/or high salt) hybrid duplexes (e.g., DNA:DNA, PNA:DNA, RNA:RNA, or RNA:DNA) will form even where the annealed sequences are not perfectly complementary. Thus, specificity of hybridization is reduced at lower stringency. Conversely, at higher stringency (e.g., higher temperature or lower salt) successful hybridization requires fewer mismatches. One of skill in the art will appreciate that hybridization conditions can be designed to provide different degrees of stringency.

As used herein the term “target junction” refers to any nucleic acid present or thought to be present in a sample that the information of a junction between an end joined nucleic acid fragment about which information would like to be obtained, such as its presence or absence.

As used herein the term “complementary” refers to a double-stranded DNA or RNA strand consists of two complementary strands of base pairs. Complementary binding occurs when the base of one nucleic acid molecule forms a hydrogen bond to the base of another nucleic acid molecule. Normally, the base adenine (A) is complementary to thymidine (T) and uracil (U), while cytosine (C) is complementary to guanine (G). For example, the sequence 5′-ATCG-3′ of one ssDNA molecule can bond to 3′-TAGC-5′ of another ssDNA to form a dsDNA. In this example, the sequence 5′-ATCG-3′ is the reverse complement of 3′-TAGC-5′.

Nucleic acid molecules can be complementary to each other even without complete hydrogen-bonding of all bases of each molecule. For example, hybridization with a complementary nucleic acid sequence can occur under conditions of differing stringency in which a complement will bind at some but not all nucleotide positions.

In general, there is a tradeoff between hybridization specificity (stringency) and signal intensity. Thus, in one embodiment, the wash is performed at the highest stringency that produces consistent results and that provides a signal intensity greater than approximately 10% of the background intensity. Thus, the hybridized array may be washed at successively higher stringency solutions and read between each wash. Analysis of the data sets thus produced will reveal a wash stringency above which the hybridization pattern is not appreciably altered and which provides adequate signal for the particular oligonucleotide probes of interest. In some examples, RNA is detected using Northern blotting or in situ hybridization (Parker & Barnes, Methods in Molecular Biology 106:247-283, 1999); RNAse protection assays (Hod, Biotechniques 13:852-4, 1992); and PCR-based methods, such as reverse transcription polymerase chain reaction (RT-PCR) (Weis et al., Trends in Genetics 8:263-4, 1992).

As used herein the term “binding or stable binding (of an oligonucleotide)” refers to an oligonucleotide, such as a nucleic acid probe that specifically binds to a target junction in an end joined nucleic acid fragment, binds or stably binds to a target nucleic acid if a sufficient amount of the oligonucleotide forms base pairs or is hybridized to its target nucleic acid. For example, depending on the hybridization conditions, there need not be complete matching between the probe and the nucleic acid target, for example there can be mismatch, or a nucleic acid bubble. Binding can be detected by either physical or functional properties.

As used herein the term “binding site” refers to a region on a protein, DNA, or RNA to which other molecules stably bind. In one example, a binding site is the site on an end joined nucleic acid fragment.

As used herein the term “detect” refers to determining if an agent (such as a signal or particular nucleic acid or protein) is present or absent. In some examples, this can further include quantification in a sample, or a fraction of a sample, such as a particular cell or cells within a tissue.

As used herein the term “detectable label” refers to a compound or composition that is conjugated directly or indirectly to another molecule to facilitate detection of that molecule. Specific, non-limiting examples of labels include fluorescent tags, enzymatic linkages, and radioactive isotopes and other physical tags, such as biotin. In some examples, a label is attached to a nucleic acid, such as an end-joined nucleic acid, to facilitate detection and/or isolation of the nucleic acid.

As used herein the term “probe” refers to an isolated nucleic acid capable of hybridizing to a target nucleic acid (such as end joined nucleic acid fragment). A detectable label or reporter molecule can be attached to a probe. Typical labels include radioactive isotopes, enzyme substrates, co-factors, ligands, chemiluminescent or fluorescent agents, haptens, and enzymes.

Methods for labeling and guidance in the choice of labels appropriate for various purposes are discussed, for example, in Sambrook et al., Molecular Cloning: A Laboratory Manual, Cold Spring Harbor Laboratory Press (1989) and Ausubel et al., Current Protocols in Molecular Biology, Greene Publishing Associates and Wiley-Intersciences (1987).

Probes are generally at least 5 nucleotides in length, such as at least 10, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50 at least 51, at least 52, at least 53, at least 54, at least 55, at least 56, at least 57, at least 58, at least 59, at least 60, or more contiguous nucleotides complementary to the target nucleic acid molecule, such as 50-60 nucleotides, 20-50 nucleotides, 20-40 nucleotides, 20-30 nucleotides or greater.

As used herein the term “targeting probe” refers to a probe that includes an isolated nucleic acid capable of hybridizing to a junction in an end joined nucleic acid fragment, wherein the probe specifically hybridizes to the end joined nucleic acid fragment both 5′ and 3′ of the site of the junction and spans the site of the junction.

In one embodiment, the hybridized nucleic acids are detected by detecting one or more labels attached to the sample nucleic acids. The labels can be incorporated by any of a number of methods. In one example, the label is simultaneously incorporated during the amplification step in the preparation of the sample nucleic acids. Thus, for example, polymerase chain reaction (PCR) with labeled primers or labeled nucleotides will provide a labeled amplification product. In one embodiment, transcription amplification, as described above, using a labeled nucleotide (such as fluorescein-labeled UTP and/or CTP) incorporates a label into the transcribed nucleic acids.

Detectable labels suitable for use include any composition detectable by spectroscopic, photochemical, biochemical, immunochemical, electrical, optical or chemical means. Useful labels include biotin for staining with labeled streptavidin conjugate, magnetic beads (for example DYNABEADS™), fluorescent dyes (for example, fluorescein, Texas red, rhodamine, green fluorescent protein, and the like), radiolabels (for example, ³H, ¹²⁵I, ³⁵S, ¹⁴C, or ³²P), enzymes (for example, horseradish peroxidase, alkaline phosphatase and others commonly used in an ELISA), and colorimetric labels such as colloidal gold or colored glass or plastic (for example, polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

Means of detecting such labels are also well known. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and colorimetric labels are detected by simply visualizing the colored label.

The label may be added to the target (sample) nucleic acid(s) prior to, or after, the hybridization. So-called “direct labels” are detectable labels that are directly attached to or incorporated into the target (sample) nucleic acid prior to hybridization. In contrast, so-called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore will bind the biotin bearing hybrid duplexes providing a label that is easily detected (see Laboratory Techniques in Biochemistry and Molecular Biology, Vol. 24: Hybridization With Nucleic Acid Probes, P. Tijssen, ed. Elsevier, N.Y., 1993).

### Target Ligation Junctions and Probes

Also disclosed are nucleic acids made of two or more end joined nucleic acids, target junctions, produced using the disclosed methods and amplification products thereof, such as RNA, DNA or a combination thereof. An isolated target junction is an end joined nucleic acid, wherein the junction encodes the information about the proximity of the two nucleic acid sequences that make up the target junction in a cell, for example as formed by the methods disclosed herein. The presence of an isolated target junction can be correlated with a disease state or environmental condition. For example, certain disease states may be caused and/or characterized by the differential formation of certain target junctions. Similarly, isolated target junction can be correlated to an environmental stress or state, such as but not limited to heat shock, osmolarity, hypoxia, cold, oxidative stress, radiation, starvation, a chemical (for example a therapeutic agent or potential therapeutic agent) and the like.

This disclosure also relates, to isolated nucleic acid probes that specifically bind to target junction, such as a target junction indicative of a disease state or environmental condition. To recognize a target join, a probe specifically hybridizes to the target junction both 5′ and 3′ of the site of the junction and spans the site of the target junction, or specifically hybridizes to specific target sequence with the end joined nucleic acid fragments. In some example embodiments, the specific target sequence is at least 50, at least 60, at least 70, at least 80, at least 90, at least 100, at least 110, at least 120, at least 130, at least 140, at least 150, at least 160, at least 170, at least 180, at least 190, or at least 200 base pairs long. In certain example embodiments, the specific nucleic acid sequence is within at least 50, at least 60, at least 70, at least, 80, at least 90, or at least 100 base pairs, in either the 5′ or 3′ direction, of a restriction site. In certain example embodiments, the specific nucleic sequence comprises less than ten repetitive bases. In certain other example embodiments, the GC content of the specific nucleic acid sequence is between 25% and 80%, between 40% and 70%, or between 50% and 60%.

In some embodiments, the probe is labeled, such as radiolabeled, fluorescently-labeled, biotin-labeled, enzymatically-labeled, or chemically-labeled. Non-limiting examples of the probe is an RNA probe, a DNA probe, a locked nucleic acid (LNA) probe, a peptide nucleic acid (PNA) probe, or a hybrid RNA-DNA probe. Also disclosed are sets of probes for binding to target ligation junction, as well as devices, such as nucleic acid arrays for detecting a target junction.

In embodiments, the total length of the probe, including end linked PCR or other tags, is between about 10 nucleotides and 200 nucleotides, although longer probes are contemplated. In some embodiments, the total length of the probe, including end linked PCR or other tags, is at least about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190 191, 192, 193, 194, 195, 196, 197, 198, 199 or 200.

In some embodiments the total length of the probe, including end linked PCR or other tags, is less than about 2000 nucleotides in length, such as less than about 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 500, 750, 1000, 1250, 1500, 1750, 2000 nucleotides in length or even greater. In some embodiments, the total length of the probe, including end linked PCR or other tags, is between about 30 nucleotides and about 250 nucleotides, for example about 90 to about 180, about 120 to about 200, about 150 to about 220 or about 120 to about 180 nucleotides in length. In some embodiments, a set of probes is used to target a specific target junction or a set of target junctions.

In some embodiments, the probe is detectably labeled, either with an isotopic or non-isotopic label, alternatively the target junction or amplification product thereof is labeled. Non-isotopic labels can, for instance, comprise a fluorescent or luminescent molecule, biotin, an enzyme or enzyme substrate or a chemical. Such labels are preferentially chosen such that the hybridization of the probe with target junction can be detected. In some examples, the probe is labeled with a fluorophore. Examples of suitable fluorophore labels are given above. In some examples, the fluorophore is a donor fluorophore. In other examples, the fluorophore is an accepter fluorophore, such as a fluorescence quencher. In some examples, the probe includes both a donor fluorophore and an accepter fluorophore. Appropriate donor/acceptor fluorophore pairs can be selected using routine methods. In one example, the donor emission wavelength is one that can significantly excite the acceptor, thereby generating a detectable emission from the acceptor.

An array containing a plurality of heterogeneous probes for the detection of target junctions are disclosed. Such arrays may be used to rapidly detect and/or identify the target junctions present in a sample, for example as part of a diagnosis. Arrays are arrangements of addressable locations on a substrate, with each address containing a nucleic acid, such as a probe. In some embodiments, each address corresponds to a single type or class of nucleic acid, such as a single probe, though a particular nucleic acid may be redundantly contained at multiple addresses. A “microarray” is a miniaturized array requiring microscopic examination for detection of hybridization. Larger “macroarrays” allow each address to be recognizable by the naked human eye and, in some embodiments, a hybridization signal is detectable without additional magnification. The addresses may be labeled, keyed to a separate guide, or otherwise identified by location.

Any sample potentially containing, or even suspected of containing, target joins may be used. A hybridization signal from an individual address on the array indicates that the probe hybridizes to a nucleotide within the sample. This system permits the simultaneous analysis of a sample by plural probes and yields information identifying the target junctions contained within the sample. In alternative embodiments, the array contains target junctions and the array is contacted with a sample containing a probe. In any such embodiment, either the probe or the target junction may be labeled to facilitate detection of hybridization.

Within an array, each arrayed nucleic acid is addressable, such that its location may be reliably and consistently determined within the at least the two dimensions of the array surface. Thus, ordered arrays allow assignment of the location of each nucleic acid at the time it is placed within the array. Usually, an array map or key is provided to correlate each address with the appropriate nucleic acid. Ordered arrays are often arranged in a symmetrical grid pattern, but nucleic acids could be arranged in other patterns (for example, in radially distributed lines, a “spokes and wheel” pattern, or ordered clusters). Addressable arrays can be computer readable; a computer can be programmed to correlate a particular address on the array with information about the sample at that position, such as hybridization or binding data, including signal intensity. In some exemplary computer readable formats, the individual samples or molecules in the array are arranged regularly (for example, in a Cartesian grid pattern), which can be correlated to address information by a computer.

An address within the array may be of any suitable shape and size. In some embodiments, the nucleic acids are suspended in a liquid medium and contained within square or rectangular wells on the array substrate. However, the nucleic acids may be contained in regions that are essentially triangular, oval, circular, or irregular. The overall shape of the array itself also may vary, though in some embodiments it is substantially flat and rectangular or square in shape.

Examples of substrates for the phage arrays disclosed herein include glass (e.g., functionalized glass), Si, Ge, GaAs, GaP, SiO₂, SiN₄, modified silicon nitrocellulose, polyvinylidene fluoride, polystyrene, polytetrafluoroethylene, polycarbonate, nylon, fiber, or combinations thereof. Array substrates can be stiff and relatively inflexible (for example glass or a supported membrane) or flexible (such as a polymer membrane). One commercially available product line suitable for probe arrays described herein is the Microlite line of MICROTITER® plates available from Dynex Technologies UK (Middlesex, United Kingdom), such as the Microlite 1+96-well plate, or the 384 Microlite+384-well plate.

Addresses on the array should be discrete, in that hybridization signals from individual addresses can be distinguished from signals of neighboring addresses, either by the naked eye (macroarrays) or by scanning or reading by a piece of equipment or with the assistance of a microscope (microarrays).

### Systems

Also disclosed is a system wherein information from one or more ligation junctions is used to identify regions of the genome that control or modulate spatial proximity relationships between nucleic acids. In some embodiments, the genomic regions identified establish chromatin loops. In some embodiments, the genomic regions identified demarcate or establish contiguous intervals of chromatin that display elevated proximity between loci within the intervals.

Further disclosed is a system for visualizing, such as system comprising hardware and/or software, the information from one or more ligation junctions. In some examples, the information from one or more ligation junctions is represented in a matrix with entries indicating frequency of interaction. In some examples, a user can dynamically zoom in and out, viewing interactions between smaller or larger pieces of the genome. In some examples, interaction matrices and other 1-D data vectors can be viewed and compared simultaneously. In some examples, the annotations of features can be superimposed on interaction matrices. In some examples, multiple interaction matrices can be simultaneously viewer and compared.

This disclosure also provides integrated systems for high-throughput testing, or automated testing. The systems typically include a robotic armature that transfers fluid from a source to a destination, a controller that controls the robotic armature, a detector, a data storage unit that records detection, and an assay component such as a microtiter dish comprising a well having a reaction mixture for example media.

As used herein the term “high throughput technique” refers to a combination of methods, robotics, data processing and control software, liquid handling devices, and detectors that allows the rapid screening of potential reagents, conditions, or targets in a short period of time, for example in less than 24, less than 12, less than 6 hours, or even less than 1 hour.

### Kits

The nucleic acid probes, such as probes for specifically binding to a target junction, and other reagents disclosed herein for use in the disclosed methods can be supplied in the form of a kit. In such a kit, an appropriate amount of one or more of the nucleic acid probes is provided in one or more containers or held on a substrate. A nucleic acid probe may be provided suspended in an aqueous solution or as a freeze-dried or lyophilized powder, for instance. The container(s) in which the nucleic acid(s) are supplied can be any conventional container that is capable of holding the supplied form, for instance, microfuge tubes, ampoules, or bottles. The kits can include either labeled or unlabeled nucleic acid probes for use in detection, of a target junction. The amount of nucleic acid probe supplied in the kit can be any appropriate amount, and may depend on the target market to which the product is directed. A kit may contain more than one different probe, such as 2, 3, 4, 5, 6, 7, 8, 9, 10, 11, 12, 13, 14, 15, 20, 25, 50, 100, or more probes. The instructions may include directions for obtaining a sample, processing the sample, preparing the probes, and/or contacting each probe with an aliquot of the sample. In certain embodiments, the kit includes an apparatus for separating the different probes, such as individual containers (for example, microtubules) or an array substrate (such as, a 96-well or 384-well microtiter plate). In particular embodiments, the kit includes prepackaged probes, such as probes suspended in suitable medium in individual containers (for example, individually sealed EPPENDORF® tubes) or the wells of an array substrate (for example, a 96-well microtiter plate sealed with a protective plastic film). In some embodiments, kits also may include the reagents necessary to carry out methods disclosed herein. In other particular embodiments, the kit includes equipment, reagents, and instructions for the methods disclosed herein.

### Genome Engineering

In certain embodiments, a specific sequence identified on an epigenetic map according to the present invention can be targeted using a genome modifying agent (e.g., CTCF dependent or CTCF independent loops). In certain embodiments, a cell is modified to treat a disease, to model a disease, or to study a biological process. For example, a transcription factor binding site or a specific regulatory sequence (e.g., a sequence in contact with a promoter, a sequence within an enhancer, or an activator binding site). In certain embodiments, a specific variant associated with a disease is modified to treat the disease. In certain embodiments, a gene associated according to the methods described herein with a disease causing variant is modified. For example, a variant present in an enhancer or regulatory sequence that is in contact with a gene. In certain embodiments, a cell is modified in vivo, ex vivo or in vitro.

A method of the invention may be used to create a plant, an animal or cell that may be used to model and/or study genetic or epigenetic conditions of interest, such as a through a model of mutations of interest or a as a disease model. As used herein, “disease” refers to a disease, disorder, or indication in a subject. For example, a method of the invention may be used to create an animal or cell that comprises a modification in one or more nucleic acid sequences associated with a disease, or a plant, animal or cell in which the expression of one or more nucleic acid sequences associated with a disease are altered. Such a nucleic acid sequence may encode a disease associated protein sequence or may be a disease associated control sequence. Accordingly, it is understood that in embodiments of the invention, a plant, subject, patient, organism or cell can be a non-human subject, patient, organism or cell. Thus, the invention provides a plant, animal or cell, produced by the present methods, or a progeny thereof. The progeny may be a clone of the produced plant or animal or may result from sexual reproduction by crossing with other individuals of the same species to introgress further desirable traits into their offspring. The cell may be in vivo or ex vivo in the cases of multicellular organisms, particularly animals or plants. In the instance where the cell is in cultured, a cell line may be established if appropriate culturing conditions are met and preferably if the cell is suitably adapted for this purpose (for instance a stem cell). Bacterial cell lines produced by the invention are also envisaged. Hence, cell lines are also envisaged.

### Genetic Modifying Agents

In certain embodiments, the genetic modifying agent may comprise a CRISPR system, a zinc finger nuclease system, a TALEN, a meganuclease or RNAi system.

### CRISPR-Cas Modification

In some embodiments, a polynucleotide of the present invention described elsewhere herein can be modified using a CRISPR-Cas and/or Cas-based system (e.g., genomic DNA or mRNA, preferably, for a disease gene). The nucleotide sequence may be or encode one or more components of a CRISPR-Cas system. For example, the nucleotide sequences may be or encode guide RNAs. The nucleotide sequences may also encode CRISPR proteins, variants thereof, or fragments thereof.

In general, a CRISPR-Cas or CRISPR system as used herein and in other documents, such as WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g., CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g., Shmakov et al. (2015) “Discovery and Functional Characterization of Diverse Class 2 CRISPR-Cas Systems”, Molecular Cell, DOI: dx.doi.org/10.1016/j.molcel.2015.10.008.

CRISPR-Cas systems can generally fall into two classes based on their architectures of their effector molecules, which are each further subdivided by type and subtype. The two classes are Class 1 and Class 2. Class 1 CRISPR-Cas systems have effector modules composed of multiple Cas proteins, some of which form crRNA-binding complexes, while Class 2 CRISPR-Cas systems include a single, multi-domain crRNA-binding protein.

In some embodiments, the CRISPR-Cas system that can be used to modify a polynucleotide of the present invention described herein can be a Class 1 CRISPR-Cas system. In some embodiments, the CRISPR-Cas system that can be used to modify a polynucleotide of the present invention described herein can be a Class 2 CRISPR-Cas system.

### Class 1 CRISPR-Cas Systems

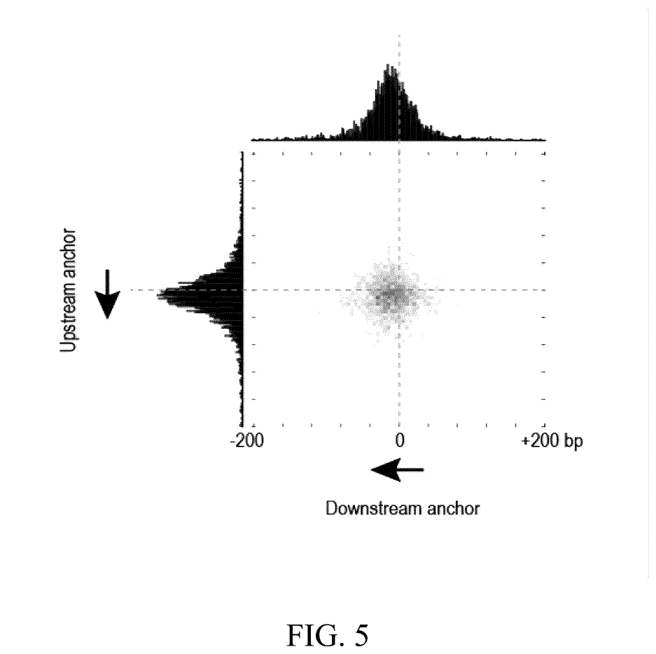

In some embodiments, the CRISPR-Cas system that can be used to modify a polynucleotide of the present invention described herein can be a Class 1 CRISPR-Cas system. Class 1 CRISPR-Cas systems are divided into Types I, II, and IV. Makarova et al. 2020. Nat. Rev. 18: 67-83., particularly as described in FIG. 1. Type I CRISPR-Cas systems are divided into 9 subtypes (I-A, I-B, I-C, I-D, I-E, I-F1, I-F2, I-F3, and IG). Makarova et al., 2020. Class 1, Type I CRISPR-Cas systems can contain a Cas3 protein that can have helicase activity. Type III CRISPR-Cas systems are divided into 6 subtypes (III-A, III-B, III-C, III-D, III-E, and III-F). Type III CRISPR-Cas systems can contain a Cas10 that can include an RNA recognition motif called Palm and a cyclase domain that can cleave polynucleotides. Makarova et al., 2020. Type IV CRISPR-Cas systems are divided into 3 subtypes. (IV-A, IV-B, and IV-C). Makarova et al., 2020. Class 1 systems also include CRISPR-Cas variants, including Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems. Peters et al., PNAS 114 (35) (2017); DOI: 10.1073/pnas.1709035114; see also, Makarova et al. 2018. The CRISPR Journal, v. 1, n5, FIG. 5.

The Class 1 systems typically use a multi-protein effector complex, which can, in some embodiments, include ancillary proteins, such as one or more proteins in a complex referred to as a CRISPR-associated complex for antiviral defense (Cascade), one or more adaptation proteins (e.g., Cas1, Cas2, RNA nuclease), and/or one or more accessory proteins (e.g., Cas 4, DNA nuclease), CRISPR associated Rossman fold (CARF) domain containing proteins, and/or RNA transcriptase.

The backbone of the Class 1 CRISPR-Cas system effector complexes can be formed by RNA recognition motif domain-containing protein(s) of the repeat-associated mysterious proteins (RAMPs) family subunits (e.g., Cas 5, Cas6, and/or Cas7). RAMP proteins are characterized by having one or more RNA recognition motif domains. In some embodiments, multiple copies of RAMPs can be present. In some embodiments, the Class I CRISPR-Cas system can include 1, 2, 3, 4, 5, 6, 7, 8, 9, 10, 11, 12 or more Cas5, Cas6, and/or Cas 7 proteins. In some embodiments, the Cas6 protein is an RNAse, which can be responsible for pre-crRNA processing. When present in a Class 1 CRISPR-Cas system, Cas6 can be optionally physically associated with the effector complex.

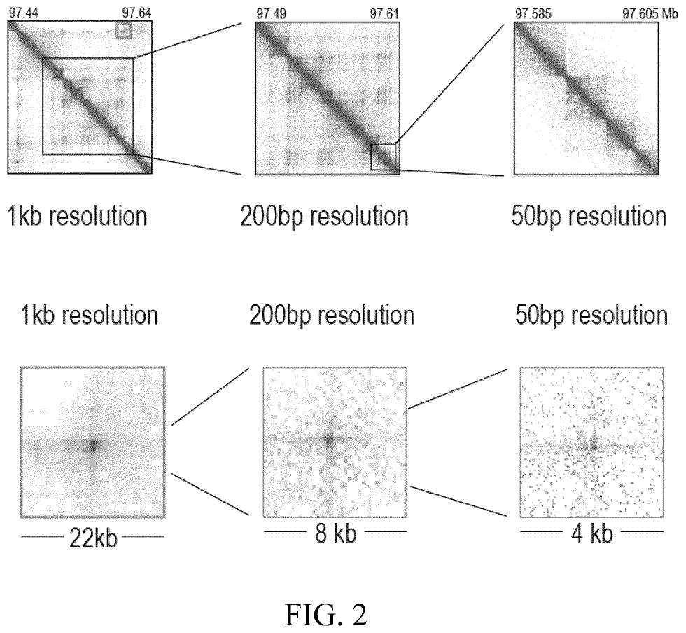

Class 1 CRISPR-Cas system effector complexes can, in some embodiments, also include a large subunit. The large subunit can be composed of or include a Cas8 and/or Cas10 protein. See, e.g., FIGS. 1 and 2. Koonin E V, Makarova K S. 2019. Phil. Trans. R. Soc. B 374: 20180087, DOI: 10.1098/rstb.2018.0087 and Makarova et al. 2020.

Class 1 CRISPR-Cas system effector complexes can, in some embodiments, include a small subunit (for example, Cas11). See, e.g., FIGS. 1 and 2. Koonin E V, Makarova K S. 2019 Origins and Evolution of CRISPR-Cas systems. Phil. Trans. R. Soc. B 374: 20180087, DOI: 10.1098/rstb.2018.0087.

In some embodiments, the Class 1 CRISPR-Cas system can be a Type I CRISPR-Cas system. In some embodiments, the Type I CRISPR-Cas system can be a subtype I-A CRISPR-Cas system. In some embodiments, the Type I CRISPR-Cas system can be a subtype I-B CRISPR-Cas system. In some embodiments, the Type I CRISPR-Cas system can be a subtype I-C CRISPR-Cas system. In some embodiments, the Type I CRISPR-Cas system can be a subtype I-D CRISPR-Cas system. In some embodiments, the Type I CRISPR-Cas system can be a subtype I-E CRISPR-Cas system. In some embodiments, the Type I CRISPR-Cas system can be a subtype I-F1 CRISPR-Cas system. In some embodiments, the Type I CRISPR-Cas system can be a subtype I-F2 CRISPR-Cas system. In some embodiments, the Type I CRISPR-Cas system can be a subtype I-F3 CRISPR-Cas system. In some embodiments, the Type I CRISPR-Cas system can be a subtype I-G CRISPR-Cas system. In some embodiments, the Type I CRISPR-Cas system can be a CRISPR Cas variant, such as a Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems as previously described.

In some embodiments, the Class 1 CRISPR-Cas system can be a Type III CRISPR-Cas system. In some embodiments, the Type III CRISPR-Cas system can be a subtype III-A CRISPR-Cas system. In some embodiments, the Type III CRISPR-Cas system can be a subtype III-B CRISPR-Cas system. In some embodiments, the Type III CRISPR-Cas system can be a subtype III-C CRISPR-Cas system. In some embodiments, the Type III CRISPR-Cas system can be a subtype III-D CRISPR-Cas system. In some embodiments, the Type III CRISPR-Cas system can be a subtype III-E CRISPR-Cas system. In some embodiments, the Type III CRISPR-Cas system can be a subtype III-F CRISPR-Cas system.

In some embodiments, the Class 1 CRISPR-Cas system can be a Type IV CRISPR-Cas-system. In some embodiments, the Type IV CRISPR-Cas system can be a subtype IV-A CRISPR-Cas system. In some embodiments, the Type IV CRISPR-Cas system can be a subtype IV-B CRISPR-Cas system. In some embodiments, the Type IV CRISPR-Cas system can be a subtype IV-C CRISPR-Cas system.

The effector complex of a Class 1 CRISPR-Cas system can, in some embodiments, include a Cas3 protein that is optionally fused to a Cas2 protein, a Cas4, a Cas5, a Cas6, a Cas7, a Cas8, a Cas10, a Cas11, or a combination thereof. In some embodiments, the effector complex of a Class 1 CRISPR-Cas system can have multiple copies, such as 1, 2, 3, 4, 5, 6, 7, 8, 9, 10, 11, 12, 13, or 14, of any one or more Cas proteins.

### Class 2 CRISPR-Cas Systems

The compositions, systems, and methods described in greater detail elsewhere herein can be designed and adapted for use with Class 2 CRISPR-Cas systems. Thus, in some embodiments, the CRISPR-Cas system is a Class 2 CRISPR-Cas system. Class 2 systems are distinguished from Class 1 systems in that they have a single, large, multi-domain effector protein. In certain example embodiments, the Class 2 system can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Each type of Class 2 system is further divided into subtypes. See Markova et al. 2020, particularly at Figure. 2. Class 2, Type II systems can be divided into 4 subtypes: II-A, II-B, II-C1, and II-C2. Class 2, Type V systems can be divided into 17 subtypes: V-A, V-B1, V-B2, V-C, V-D, V-E, V-F1, V-F1(V-U3), V-F2, V-F3, V-G, V-H, V-I, V-K (V-U5), V-U1, V-U2, and V-U4. Class 2, Type IV systems can be divided into 5 subtypes: VI-A, VI-B1, VI-B2, VI-C, and VI-D.

The distinguishing feature of these types is that their effector complexes consist of a single, large, multi-domain protein. Type V systems differ from Type II effectors (e.g., Cas9), which contain two nuclear domains that are each responsible for the cleavage of one strand of the target DNA, with the HNH nuclease inserted inside the Ruv-C like nuclease domain sequence. The Type V systems (e.g., Cas12) only contain a RuvC-like nuclease domain that cleaves both strands. Type VI (Cas13) are unrelated to the effectors of Type II and V systems and contain two HEPN domains and target RNA. Cas13 proteins also display collateral activity that is triggered by target recognition. Some Type V systems have also been found to possess this collateral activity with two single-stranded DNA in in vitro contexts.

In some embodiments, the Class 2 system is a Type II system. In some embodiments, the Type II CRISPR-Cas system is a II-A CRISPR-Cas system. In some embodiments, the Type II CRISPR-Cas system is a II-B CRISPR-Cas system. In some embodiments, the Type II CRISPR-Cas system is a II-C1 CRISPR-Cas system. In some embodiments, the Type II CRISPR-Cas system is a II-C2 CRISPR-Cas system. In some embodiments, the Type II system is a Cas9 system. In some embodiments, the Type II system includes a Cas9.

In some embodiments, the Class 2 system is a Type V system. In some embodiments, the Type V CRISPR-Cas system is a V-A CRISPR-Cas system. In some embodiments, the Type V CRISPR-Cas system is a V-B1 CRISPR-Cas system. In some embodiments, the Type V CRISPR-Cas system is a V-B2 CRISPR-Cas system. In some embodiments, the Type V CRISPR-Cas system is a V-C CRISPR-Cas system. In some embodiments, the Type V CRISPR-Cas system is a V-D CRISPR-Cas system. In some embodiments, the Type V CRISPR-Cas system is a V-E CRISPR-Cas system. In some embodiments, the Type V CRISPR-Cas system is a V-F1 CRISPR-Cas system. In some embodiments, the Type V CRISPR-Cas system is a V-F1 (V-U3) CRISPR-Cas system. In some embodiments, the Type V CRISPR-Cas system is a V-F2 CRISPR-Cas system. In some embodiments, the Type V CRISPR-Cas system is a V-F3 CRISPR-Cas system. In some embodiments, the Type V CRISPR-Cas system is a V-G CRISPR-Cas system. In some embodiments, the Type V CRISPR-Cas system is a V-H CRISPR-Cas system. In some embodiments, the Type V CRISPR-Cas system is a V-I CRISPR-Cas system. In some embodiments, the Type V CRISPR-Cas system is a V-K (V-U5) CRISPR-Cas system. In some embodiments, the Type V CRISPR-Cas system is a V-U1 CRISPR-Cas system. In some embodiments, the Type V CRISPR-Cas system is a V-U2 CRISPR-Cas system. In some embodiments, the Type V CRISPR-Cas system is a V-U4 CRISPR-Cas system. In some embodiments, the Type V CRISPR-Cas system includes a Cas12a (Cpf1), Cas12b (C2c1), Cas12c (C2c3), CasX, and/or Cas14.

In some embodiments the Class 2 system is a Type VI system. In some embodiments, the Type VI CRISPR-Cas system is a VI-A CRISPR-Cas system. In some embodiments, the Type VI CRISPR-Cas system is a VI-B1 CRISPR-Cas system. In some embodiments, the Type VI CRISPR-Cas system is a VI-B2 CRISPR-Cas system. In some embodiments, the Type VI CRISPR-Cas system is a VI-C CRISPR-Cas system. In some embodiments, the Type VI CRISPR-Cas system is a VI-D CRISPR-Cas system. In some embodiments, the Type VI CRISPR-Cas system includes a Cas13a (C2c2), Cas13b (Group 29/30), Cas13c, and/or Cas13d.

### Specialized Cas-Based Systems

In some embodiments, the system is a Cas-based system that is capable of performing a specialized function or activity. For example, the Cas protein may be fused, operably coupled to, or otherwise associated with one or more functionals domains. In certain example embodiments, the Cas protein may be a catalytically dead Cas protein (“dCas”) and/or have nickase activity. A nickase is a Cas protein that cuts only one strand of a double stranded target. In such embodiments, the dCas or nickase provide a sequence specific targeting functionality that delivers the functional domain to or proximate a target sequence. Example functional domains that may be fused to, operably coupled to, or otherwise associated with a Cas protein can be or include, but are not limited to a nuclear localization signal (NLS) domain, a nuclear export signal (NES) domain, a translational activation domain, a transcriptional activation domain (e.g. VP64, p65, MyoD1, HSF1, RTA, and SET7/9), a translation initiation domain, a transcriptional repression domain (e.g., a KRAB domain, NuE domain, NcoR domain, and a SID domain such as a SID4X domain), a nuclease domain (e.g., FokI), a histone modification domain (e.g., a histone acetyltransferase), a light inducible/controllable domain, a chemically inducible/controllable domain, a transposase domain, a homologous recombination machinery domain, a recombinase domain, an integrase domain, and combinations thereof. Methods for generating catalytically dead Cas9 or a nickase Cas9 (WO 2014/204725, Ran et al. Cell. 2013 Sep. 12; 154(6):1380-1389), Cas12 (Liu et al. Nature Communications, 8, 2095 (2017), and Cas13 (WO 2019/005884, WO2019/060746) are known in the art and incorporated herein by reference.

In some embodiments, the functional domains can have one or more of the following activities: methylase activity, demethylase activity, translation activation activity, translation initiation activity, translation repression activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity, molecular switch activity, chemical inducibility, light inducibility, and nucleic acid binding activity. In some embodiments, the one or more functional domains may comprise epitope tags or reporters. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporters include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and auto-fluorescent proteins including blue fluorescent protein (BFP).

The one or more functional domain(s) may be positioned at, near, and/or in proximity to a terminus of the effector protein (e.g., a Cas protein). In embodiments having two or more functional domains, each of the two can be positioned at or near or in proximity to a terminus of the effector protein (e.g., a Cas protein). In some embodiments, such as those where the functional domain is operably coupled to the effector protein, the one or more functional domains can be tethered or linked via a suitable linker (including, but not limited to, GlySer linkers) to the effector protein (e.g., a Cas protein). When there is more than one functional domain, the functional domains can be same or different. In some embodiments, all the functional domains are the same. In some embodiments, all of the functional domains are different from each other. In some embodiments, at least two of the functional domains are different from each other. In some embodiments, at least two of the functional domains are the same as each other.

Other suitable functional domains can be found, for example, in International Patent Publication No. WO 2019/018423.

### Split CRISPR-Cas Systems

In some embodiments, the CRISPR-Cas system is a split CRISPR-Cas system. See e.g., Zetche et al., 2015. Nat. Biotechnol. 33(2): 139-142 and WO 2019/018423, the compositions and techniques of which can be used in and/or adapted for use with the present invention. Split CRISPR-Cas proteins are set forth herein and in documents incorporated herein by reference in further detail herein. In certain embodiments, each part of a split CRISPR protein is attached to a member of a specific binding pair, and when bound with each other, the members of the specific binding pair maintain the parts of the CRISPR protein in proximity. In certain embodiments, each part of a split CRISPR protein is associated with an inducible binding pair. An inducible binding pair is one which is capable of being switched “on” or “off” by a protein or small molecule that binds to both members of the inducible binding pair. In some embodiments, CRISPR proteins may preferably split between domains, leaving domains intact. In particular embodiments, said Cas split domains (e.g., RuvC and HNH domains in the case of Cas9) can be simultaneously or sequentially introduced into the cell such that said split Cas domain(s) process the target nucleic acid sequence in the algae cell. The reduced size of the split Cas compared to the wild type Cas allows other methods of delivery of the systems to the cells, such as the use of cell penetrating peptides as described herein.

### DNA and RNA Base Editing

In some embodiments, a polynucleotide of the present invention described elsewhere herein can be modified using a base editing system. In some embodiments, a Cas protein is connected or fused to a nucleotide deaminase. Thus, in some embodiments the Cas-based system can be a base editing system. As used herein “base editing” refers generally to the process of polynucleotide modification via a CRISPR-Cas-based or Cas-based system that does not include excising nucleotides to make the modification. Base editing can convert base pairs at precise locations without generating excess undesired editing byproducts that can be made using traditional CRISPR-Cas systems.

In certain example embodiments, the nucleotide deaminase may be a DNA base editor used in combination with a DNA binding Cas protein such as, but not limited to, Class 2 Type II and Type V systems. Two classes of DNA base editors are generally known: cytosine base editors (CBEs) and adenine base editors (ABEs). CBEs convert a C·G base pair into a T·A base pair (Komor et al. 2016. Nature. 533:420-424; Nishida et al. 2016. Science. 353; and Li et al. Nat. Biotech. 36:324-327) and ABEs convert an A·T base pair to a G·C base pair. Collectively, CBEs and ABEs can mediate all four possible transition mutations (C to T, A to G, T to C, and G to A). Rees and Liu. 2018. Nat. Rev. Genet. 19(12): 770-788, particularly at FIGS. 1b, 2a-2c, 3a-3f, and Table 1. In some embodiments, the base editing system includes a CBE and/or an ABE. In some embodiments, a polynucleotide of the present invention described elsewhere herein can be modified using a base editing system. Rees and Liu. 2018. Nat. Rev. Gent. 19(12):770-788. Base editors also generally do not need a DNA donor template and/or rely on homology-directed repair. Komor et al. 2016. Nature. 533:420-424; Nishida et al. 2016. Science. 353; and Gaudeli et al. 2017. Nature. 551:464-471. Upon binding to a target locus in the DNA, base pairing between the guide RNA of the system and the target DNA strand leads to displacement of a small segment of ssDNA in an “R-loop”. Nishimasu et al. Cell. 156:935-949. DNA bases within the ssDNA bubble are modified by the enzyme component, such as a deaminase. In some systems, the catalytically disabled Cas protein can be a variant or modified Cas can have nickase functionality and can generate a nick in the non-edited DNA strand to induce cells to repair the non-edited strand using the edited strand as a template. Komor et al. 2016. Nature. 533:420-424; Nishida et al. 2016. Science. 353; and Gaudeli et al. 2017. Nature. 551:464-471. Base editors may be further engineered to optimize conversion of nucleotides (e.g. A:T to G:C). Richter et al. 2020. Nature Biotechnology. doi.org/10.1038/s41587-020-0453-z.

Other Example Type V base editing systems are described in WO 2018/213708, WO 2018/213726, PCT/US2018/067207, PCT/US2018/067225, and PCT/US2018/067307 which are incorporated by referenced herein.

In certain example embodiments, the base editing system may be a RNA base editing system. As with DNA base editors, a nucleotide deaminase capable of converting nucleotide bases may be fused to a Cas protein. However, in these embodiments, the Cas protein will need to be capable of binding RNA. Example RNA binding Cas proteins include, but are not limited to, RNA-binding Cas9s such as Francisella novicida Cas9 (“FnCas9”), and Class 2 Type VI Cas systems. The nucleotide deaminase may be a cytidine deaminase or an adenosine deaminase, or an adenosine deaminase engineered to have cytidine deaminase activity. In certain example embodiments, the RNA based editor may be used to delete or introduce a post-translation modification site in the expressed mRNA. In contrast to DNA base editors, whose edits are permanent in the modified cell, RNA base editors can provide edits where finer temporal control may be needed, for example in modulating a particular immune response. Example Type VI RNA-base editing systems are described in Cox et al. 2017. Science 358: 1019-1027, WO 2019/005884, WO 2019/005886, WO 2019/071048, PCT/US20018/05179, PCT/US2018/067207, which are incorporated herein by reference. An example FnCas9 system that may be adapted for RNA base editing purposes is described in WO 2016/106236, which is incorporated herein by reference.

An example method for delivery of base-editing systems, including use of a split-intein approach to divide CBE and ABE into reconstitutable halves, is described in Levy et al. Nature Biomedical Engineering doi.org/10.1038/s41441-019-0505-5 (2019), which is incorporated herein by reference.

### Prime Editors

In some embodiments, a polynucleotide of the present invention described elsewhere herein can be modified using a prime editing system (See e.g., Anzalone et al. 2019. Nature. 576: 149-157). Like base editing systems, prime editing systems can be capable of targeted modification of a polynucleotide without generating double stranded breaks and does not require donor templates. Further prime editing systems can be capable of all 12 possible combination swaps. Prime editing can operate via a “search-and-replace” methodology and can mediate targeted insertions, deletions, all 12 possible base-to-base conversion, and combinations thereof. Generally, a prime editing system, as exemplified by PE1, PE2, and PE3 (Id.), can include a reverse transcriptase fused or otherwise coupled or associated with an RNA-programmable nickase, and a prime-editing extended guide RNA (pegRNA) to facility direct copying of genetic information from the extension on the pegRNA into the target polynucleotide. Embodiments that can be used with the present invention include these and variants thereof. Prime editing can have the advantage of lower off-target activity than traditional CRIPSR-Cas systems along with few byproducts and greater or similar efficiency as compared to traditional CRISPR-Cas systems.

In some embodiments, the prime editing guide molecule can specify both the target polynucleotide information (e.g., sequence) and contain a new polynucleotide cargo that replaces target polynucleotides. To initiate transfer from the guide molecule to the target polynucleotide, the PE system can nick the target polynucleotide at a target side to expose a 3′hydroxyl group, which can prime reverse transcription of an edit-encoding extension region of the guide molecule (e.g., a prime editing guide molecule or peg guide molecule) directly into the target site in the target polynucleotide. See e.g., Anzalone et al. 2019. Nature. 576: 149-157, particularly at FIGS. 1b, 1c, related discussion, and Supplementary discussion.

In some embodiments, a prime editing system can be composed of a Cas polypeptide having nickase activity, a reverse transcriptase, and a guide molecule. The Cas polypeptide can lack nuclease activity. The guide molecule can include a target binding sequence as well as a primer binding sequence and a template containing the edited polynucleotide sequence. The guide molecule, Cas polypeptide, and/or reverse transcriptase can be coupled together or otherwise associate with each other to form an effector complex and edit a target sequence. In some embodiments, the Cas polypeptide is a Class 2, Type V Cas polypeptide. In some embodiments, the Cas polypeptide is a Cas9 polypeptide (e.g., is a Cas9 nickase). In some embodiments, the Cas polypeptide is fused to the reverse transcriptase. In some embodiments, the Cas polypeptide is linked to the reverse transcriptase.

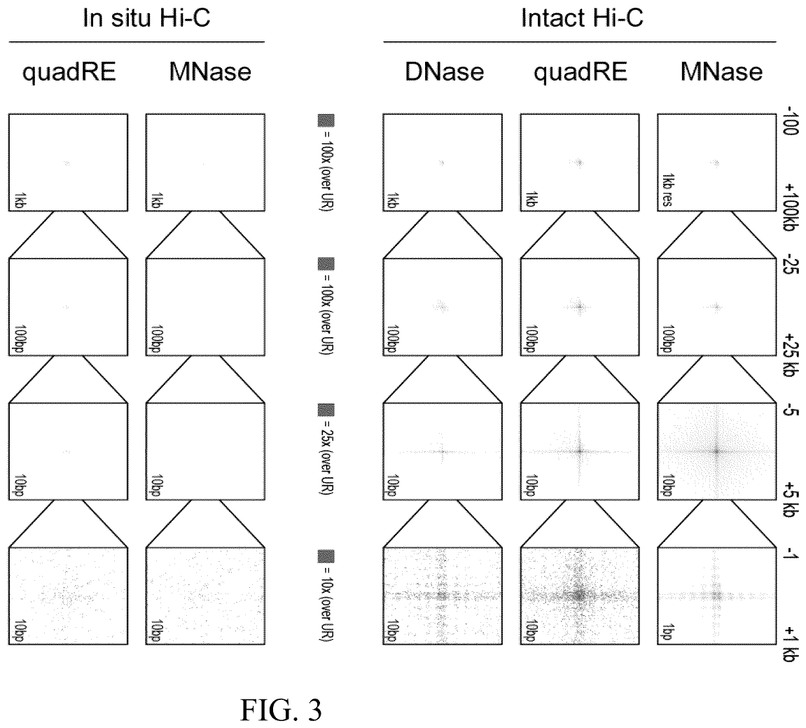

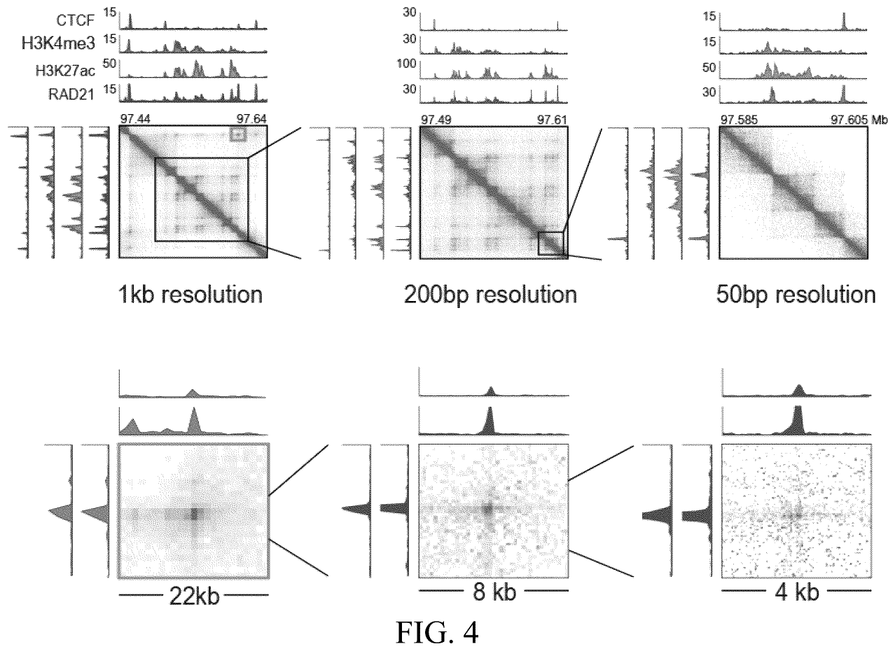

In some embodiments, the prime editing system can be a PE1 system or variant thereof, a PE2 system or variant thereof, or a PE3 (e.g., PE3, PE3b) system. See e.g., Anzalone et al. 2019. Nature. 576: 149-157, particularly at pgs. 2-3, FIGS. 2a, 3a-3f, 4a-4b, Extended data FIGS. 3a-3b, 4,

The peg guide molecule can be about 10 to about 200 or more nucleotides in length, such as 10 to/or 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 or more nucleotides in length. Optimization of the peg guide molecule can be accomplished as described in Anzalone et al. 2019. Nature. 576: 149-157, particularly at pg. 3, FIG. 2a-2b, and Extended Data FIGS. 5a-c.

### CRISPR Associated Transposase (CAST) Systems

In some embodiments, a polynucleotide of the present invention described elsewhere herein can be modified using a CRISPR Associated Transposase (“CAST”) system. CAST system can include a Cas protein that is catalytically inactive, or engineered to be catalytically active, and further comprises a transposase (or subunits thereof) that catalyze RNA-guided DNA transposition. Such systems are able to insert DNA sequences at a target site in a DNA molecule without relying on host cell repair machinery. CAST systems can be Class1 or Class 2 CAST systems. An example Class 1 system is described in Klompe et al. Nature, doi:10.1038/s41586-019-1323, which is in incorporated herein by reference. An example Class 2 system is described in Strecker et al. Science. 10/1126/science. aax9181 (2019), and PCT/US2019/066835 which are incorporated herein by reference.

### Guide Molecules

The CRISPR-Cas or Cas-Based system described herein can, in some embodiments, include one or more guide molecules. The terms guide molecule, guide sequence and guide polynucleotide, refer to polynucleotides capable of guiding Cas to a target genomic locus and are used interchangeably as in foregoing cited documents such as WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. The guide molecule can be a polynucleotide.

The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay (Qui et al. 2004. BioTechniques. 36(4)702-707). Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible and will occur to those skilled in the art.

In some embodiments, the guide molecule is an RNA. The guide molecule(s) (also referred to interchangeably herein as guide polynucleotide and guide sequence) that are included in the CRISPR-Cas or Cas based system can be any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), Clustal W, Clustal X, BLAT, Novoalign (Novocraft Technologies; available at www.novocraft.com), ELAND (Illumina, San Diego, CA), SOAP (available at soap.genomics.org.cn), and Maq (available at maq.sourceforge.net).

A guide sequence, and hence a nucleic acid-targeting guide, may be selected to target any target nucleic acid sequence. The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

In some embodiments, a nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and P A Carr and G M Church, 2009, Nature Biotechnology 27(12): 1151-62).

In certain embodiments, a guide RNA or crRNA may comprise, consist essentially of, or consist of a direct repeat (DR) sequence and a guide sequence or spacer sequence. In certain embodiments, the guide RNA or crRNA may comprise, consist essentially of, or consist of a direct repeat sequence fused or linked to a guide sequence or spacer sequence. In certain embodiments, the direct repeat sequence may be located upstream (i.e., 5′) from the guide sequence or spacer sequence. In other embodiments, the direct repeat sequence may be located downstream (i.e., 3′) from the guide sequence or spacer sequence.

In certain embodiments, the crRNA comprises a stem loop, preferably a single stem loop. In certain embodiments, the direct repeat sequence forms a stem loop, preferably a single stem loop.

In certain embodiments, the spacer length of the guide RNA is from 15 to 35 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and crRNA sequence are contained within a single transcript, such that hybridization between the two produces a transcript having a secondary structure, such as a hairpin.

In general, degree of complementarity is with reference to the optimal alignment of the sca sequence and tracr sequence, along the length of the shorter of the two sequences. Optimal alignment may be determined by any suitable alignment algorithm and may further account for secondary structures, such as self-complementarity within either the sca sequence or tracr sequence. In some embodiments, the degree of complementarity between the tracr sequence and sea sequence along the length of the shorter of the two when optimally aligned is about or more than about 25%, 30%, 40%, 50%, 60%, 70%, 80%, 90%, 95%, 97.5%, 99%, or higher.

In some embodiments, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and tracr RNA can be 30 or 50 nucleotides in length. In some embodiments, the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In some embodiments according to the invention, the guide RNA (capable of guiding Cas to a target locus) may comprise (1) a guide sequence capable of hybridizing to a genomic target locus in the eukaryotic cell; (2) a tracr sequence; and (3) a tracr mate sequence. All (1) to (3) may reside in a single RNA, i.e., an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence. Where the tracr RNA is on a different RNA than the RNA containing the guide and tracr sequence, the length of each RNA may be optimized to be shortened from their respective native lengths, and each may be independently chemically modified to protect from degradation by cellular RNase or otherwise increase stability.

Many modifications to guide sequences are known in the art and are further contemplated within the context of this invention. Various modifications may be used to increase the specificity of binding to the target sequence and/or increase the activity of the Cas protein and/or reduce off-target effects. Example guide sequence modifications are described in PCT US2019/045582, specifically paragraphs [0178]-[0333], which is incorporated herein by reference.

### Target Sequences, PAMs, and PFSs

### Target Sequences

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to an RNA polynucleotide being or comprising the target sequence. In other words, the target polynucleotide can be a polynucleotide or a part of a polynucleotide to which a part of the guide sequence is designed to have complementarity with and to which the effector function mediated by the complex comprising the CRISPR effector protein and a guide molecule is to be directed. In some embodiments, a target sequence is located in the nucleus or cytoplasm of a cell.

The guide sequence can specifically bind a target sequence in a target polynucleotide. The target polynucleotide may be DNA. The target polynucleotide may be RNA. The target polynucleotide can have one or more (e.g., 1, 2, 3, 4, 5, 6, 7, 8, 9, 10, etc. or more) target sequences. The target polynucleotide can be on a vector. The target polynucleotide can be genomic DNA. The target polynucleotide can be episomal. Other forms of the target polynucleotide are described elsewhere herein.

The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence (also referred to herein as a target polynucleotide) may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

### PAM and PFS Elements

PAM elements are sequences that can be recognized and bound by Cas proteins. Cas proteins/effector complexes can then unwind the dsDNA at a position adjacent to the PAM element. It will be appreciated that Cas proteins and systems that include them that target RNA do not require PAM sequences (Marraffini et al. 2010. Nature. 463:568-571). Instead, many rely on PFSs, which are discussed elsewhere herein. In certain embodiments, the target sequence should be associated with a PAM (protospacer adjacent motif) or PFS (protospacer flanking sequence or site), that is, a short sequence recognized by the CRISPR complex. Depending on the nature of the CRISPR-Cas protein, the target sequence should be selected, such that its complementary sequence in the DNA duplex (also referred to herein as the non-target sequence) is upstream or downstream of the PAM. In the embodiments, the complementary sequence of the target sequence is downstream or 3′ of the PAM or upstream or 5′ of the PAM. The precise sequence and length requirements for the PAM differ depending on the Cas protein used, but PAMs are typically 2-5 base pair sequences adjacent the protospacer (that is, the target sequence). Examples of the natural PAM sequences for different Cas proteins are provided herein below and the skilled person will be able to identify further PAM sequences for use with a given Cas protein.

The ability to recognize different PAM sequences depends on the Cas polypeptide(s) included in the system. See e.g., Gleditzsch et al. 2019. RNA Biology. 16(4):504-517. Table A below shows several Cas polypeptides and the PAM sequence they recognize.

TABLE A
Example PAM Sequences

| Cas Protein | PAM Sequence |
|---|---|
| SpCas9 | NGG/NRG |
| SaCas9 | NGRRT or NGRRN |
| NmeCas9 | NNNNGATT |
| CjCas9 | NNNNRYAC |
| StCas9 | NNAGAAW |
| Cas12a (Cpf1) | TTTV |
| (including LbCpf) | |
| and AsCpfl) | |
| Cas12b (C2c1) | TTT, TTA, and TTC |
| Cas12c (C2c3) | TA |
| Cas12d (CasY) | TA |
| Cas12e (CasX) | 5′-TTCN-3′ |

In a preferred embodiment, the CRISPR effector protein may recognize a 3′ PAM. In certain embodiments, the CRISPR effector protein may recognize a 3′ PAM which is 5′H, wherein H is A, C or U.

Further, engineering of the PAM Interacting (PI) domain on the Cas protein may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10.1038/nature14592. As further detailed herein, the skilled person will understand that Cas13 proteins may be modified analogously. Gao et al, “Engineered Cpf1 Enzymes with Altered PAM Specificities,” bioRxiv 091611; doi: dx.doi.org/10.1101/091611 (Dec. 4, 2016). Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.

PAM sequences can be identified in a polynucleotide using an appropriate design tool, which are commercially available as well as online. Such freely available tools include, but are not limited to, CRISPRFinder and CRISPRTarget. Mojica et al. 2009. Microbiol. 155(Pt. 3):733-740; Atschul et al. 1990. J. Mol. Biol. 215:403-410; Biswass et al. 2013 RNA Biol. 10:817-827; and Grissa et al. 2007. Nucleic Acid Res. 35:W52-57. Experimental approaches to PAM identification can include, but are not limited to, plasmid depletion assays (Jiang et al. 2013. Nat. Biotechnol. 31:233-239; Esvelt et al. 2013. Nat. Methods. 10:1116-1121; Kleinstiver et al. 2015. Nature. 523:481-485), screened by a high-throughput in vivo model called PAM-SCNAR (Pattanayak et al. 2013. Nat. Biotechnol. 31:839-843 and Leenay et al. 2016. Mol. Cell. 16:253), and negative screening (Zetsche et al. 2015. Cell. 163:759-771).

As previously mentioned, CRISPR-Cas systems that target RNA do not typically rely on PAM sequences. Instead, such systems typically recognize protospacer flanking sites (PFSs) instead of PAMs Thus, Type VI CRISPR-Cas systems typically recognize protospacer flanking sites (PFSs) instead of PAMs. PFSs represents an analogue to PAMs for RNA targets. Type VI CRISPR-Cas systems employ a Cas13. Some Cas13 proteins analyzed to date, such as Cas13a (C2c2) identified from Leptotrichia shahii (LShCAs13a) have a specific discrimination against G at the 3′end of the target RNA. The presence of a C at the corresponding crRNA repeat site can indicate that nucleotide pairing at this position is rejected. However, some Cas13 proteins (e.g., LwaCAs13a and PspCas13b) do not seem to have a PFS preference. See e.g., Gleditzsch et al. 2019. RNA Biology. 16(4):504-517.

Some Type VI proteins, such as subtype B, have 5′-recognition of D (G, T, A) and a 3′-motif requirement of NAN or NNA. One example is the Cas13b protein identified in Bergeyella zoohelcum (BzCas13b). See e.g., Gleditzsch et al. 2019. RNA Biology. 16(4):504-517.

Overall Type VI CRISPR-Cas systems appear to have less restrictive rules for substrate (e.g., target sequence) recognition than those that target DNA (e.g., Type V and type II).

### Zinc Finger Nucleases

In some embodiments, the polynucleotide is modified using a Zinc Finger nuclease or system thereof. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to FokI cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

### TALE Nucleases

In some embodiments, a TALE nuclease or TALE nuclease system can be used to modify a polynucleotide. In some embodiments, the methods provided herein use isolated, non-naturally occurring, recombinant or engineered DNA binding proteins that comprise TALE monomers or TALE monomers or half monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, “TALE monomers” or “monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X₁₋₁₁-(X₁₂×₁₃)-X₁₄₋₃₃ or 34 or 35, where the subscript indicates the amino acid position and X represents any amino acid. X₁₂×₁₃ indicate the RVDs. In some polypeptide monomers, the variable amino acid at position 13 is missing or absent and in such monomers, the RVD consists of a single amino acid. In such cases the RVD may be alternatively represented as X*, where X represents X₁₂ and (*) indicates that X₁₃ is absent. The DNA binding domain comprises several repeats of TALE monomers and this may be represented as (X₁₋₁₁-(X₁₂×₁₃)-X₁₄₋₃₃ or 34 or 35)_{z}, where in an advantageous embodiment, z is at least 5 to 40. In a further advantageous embodiment, z is at least 10 to 26.

The TALE monomers can have a nucleotide binding affinity that is determined by the identity of the amino acids in its RVD. For example, polypeptide monomers with an RVD of NI can preferentially bind to adenine (A), monomers with an RVD of NG can preferentially bind to thymine (T), monomers with an RVD of HD can preferentially bind to cytosine (C) and monomers with an RVD of NN can preferentially bind to both adenine (A) and guanine (G). In some embodiments, monomers with an RVD of IG can preferentially bind to T. Thus, the number and order of the polypeptide monomer repeats in the nucleic acid binding domain of a TALE determines its nucleic acid target specificity. In some embodiments, monomers with an RVD of NS can recognize all four base pairs and can bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011).

The polypeptides used in methods of the invention can be isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In some embodiments, polypeptide monomers having RVDs RN, NN, NK, SN, NH, KN, HN, NQ, HH, RG, KH, RH and SS can preferentially bind to guanine. In some embodiments, polypeptide monomers having RVDs RN, NK, NQ, HH, KH, RH, SS and SN can preferentially bind to guanine and can thus allow the generation of TALE polypeptides with high binding specificity for guanine containing target nucleic acid sequences. In some embodiments, polypeptide monomers having RVDs HH, KH, NH, NK, NQ, RH, RN and SS can preferentially bind to guanine and thereby allow the generation of TALE polypeptides with high binding specificity for guanine containing target nucleic acid sequences. In some embodiments, the RVDs that have high binding specificity for guanine are RN, NH RH and KH. Furthermore, polypeptide monomers having an RVD of NV can preferentially bind to adenine and guanine. In some embodiments, monomers having RVDs of H*, HA, KA, N*, NA, NC, NS, RA, and S* bind to adenine, guanine, cytosine, and thymine with comparable affinity.

The predetermined N-terminal to C-terminal order of the one or more polypeptide monomers of the nucleic acid or DNA binding domain determines the corresponding predetermined target nucleic acid sequence to which the polypeptides of the invention will bind. As used herein the monomers and at least one or more half monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases, this region may be referred to as repeat 0. In animal genomes, TALE binding sites do not necessarily have to begin with a thymine (T) and polypeptides of the invention may target DNA sequences that begin with T, A, G or C. The tandem repeat of TALE monomers always ends with a half-length repeat or a stretch of sequence that may share identity with only the first 20 amino acids of a repetitive full-length TALE monomer and this half repeat may be referred to as a half-monomer. Therefore, it follows that the length of the nucleic acid or DNA being targeted is equal to the number of full monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

An exemplary amino acid sequence of a N-terminal capping region is:

(SEQ ID NO: 1)
M D P I R S R T P S P A R E L L S G P Q
P D G V Q P T A D R G V S P P A G G P L
D G L P A R R T M S R T R L P S P P A P
S P A F S A D S F S D L L R Q F D P S L
E N T S L F D S L P P F G A H H T E A A
T G E W D E V Q S G L R A A D A P P P T
M R V A V T A A R P P R A K P A P R R R
A A Q P S D A S P A A Q V D L R T L G Y
S Q Q Q Q E K I K P K V R S T V A Q H H
E A L V G H G F T H A H I V A L S Q H P
A A L G T V A V K Y Q D M I A A L P E A
T H E A I V G V G K Q W S G A R A L E A
L L T V A G E L R G P P L Q L D T G Q L
L K I A K R G G V T A V E A V H A W R N
A L T G A P L N

An exemplary amino acid sequence of a C-terminal capping region is:

(SEQ ID NO: 2)
R P A L E S I V A Q L S R P D P A L A A
L T N D H L V A L A C L G G R P A L D A
V K K G L P H A P A L I K R T N R R I P
E R T S H R V A D H A Q V V R V L G F F
Q C H S H P A Q A F D D A M T Q F G M S
R H G L L Q L F R R V G V T E L E A R S
G T L P P A S Q R W D R I L Q A S G M K
R A K P S P T S T Q T P D Q A S L H A F
A D S L E R D L D A P S P M H E G D Q T
R A S

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain an N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full-length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full-length capping region, while fragments that include the C-terminal 20 amino acids retain greater than 50% of the efficacy of the full-length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies can be generated by any of a number of computer programs known in the art, which include but are not limited to BLAST or FASTA. Suitable computer programs for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In some embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4X domain or a Krüppel-associated box (KRAB) or fragments of the KRAB domain. In some embodiments the effector domain is an enhancer of transcription (i.e. an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination of the activities described herein.

### Meganucleases

In some embodiments, a meganuclease or system thereof can be used to modify a polynucleotide. Meganucleases, which are endodeoxyribonucleases characterized by a large recognition site (double-stranded DNA sequences of 12 to 40 base pairs). Exemplary methods for using meganucleases can be found in U.S. Pat. Nos. 8,163,514, 8,133,697, 8,021,867, 8,119,361, 8,119,381, 8,124,369, and 8,129,134, which are specifically incorporated by reference.

### Sequences Related to Nucleus Targeting and Transportation

In some embodiments, one or more components (e.g., the Cas protein and/or deaminase, Zn Finger protein, TALE, or meganuclease) in the composition for engineering cells may comprise one or more sequences related to nucleus targeting and transportation. Such sequence may facilitate the one or more components in the composition for targeting a sequence within a cell. In order to improve targeting of the CRISPR-Cas protein and/or the nucleotide deaminase protein or catalytic domain thereof used in the methods of the present disclosure to the nucleus, it may be advantageous to provide one or both of these components with one or more nuclear localization sequences (NLSs).

In some embodiments, the NLSs used in the context of the present disclosure are heterologous to the proteins. Non-limiting examples of NLSs include an NLS sequence derived from: the NLS of the SV40 virus large T-antigen, having the amino acid sequence PKKKRKV (SEQ ID NO: 3) or PKKKRKVEAS (SEQ ID NO: 4); the NLS from nucleoplasmin (e.g., the nucleoplasmin bipartite NLS with the sequence KRPAATKKAGQAKKKK (SEQ ID NO: 5)); the c-myc NLS having the amino acid sequence PAAKRVKLD (SEQ ID NO: 6) or RQRRNELKRSP (SEQ ID NO: 7); the hRNPA1 M9 NLS having the sequence NQSSNFGPMKGGNFGGRSSGPYGGGGQYFAKPRNQGGY (SEQ ID NO: 8); the sequence RMRIZFKNKGKDTAELRRRRVEVSVELRKAKKDEQILKRRNV (SEQ ID NO: 9) of the IBB domain from importin-alpha; the sequences VSRKRPRP (SEQ ID NO: 10) and PPKKARED (SEQ ID NO: 11) of the myoma T protein; the sequence PQPKKKPL (SEQ ID NO: 12) of human p53; the sequence SALIKKKKKMAP (SEQ ID NO: 13) of mouse c-abl IV; the sequences DRLRR (SEQ ID NO: 14) and PKQKKRK (SEQ ID NO: 15) of the influenza virus NS1; the sequence RKLKKKIKKL (SEQ ID NO: 16) of the Hepatitis virus delta antigen; the sequence REKKKFLKRR (SEQ ID NO: 17) of the mouse Mx1 protein; the sequence KRKGDEVDGVDEVAKKKSKK (SEQ ID NO: 18) of the human poly(ADP-ribose) polymerase; and the sequence RKCLQAGMNLEARKTKK (SEQ ID NO: 19) of the steroid hormone receptors (human) glucocorticoid. In general, the one or more NLSs are of sufficient strength to drive accumulation of the DNA-targeting Cas protein in a detectable amount in the nucleus of a eukaryotic cell. In general, strength of nuclear localization activity may derive from the number of NLSs in the CRISPR-Cas protein, the particular NLS(s) used, or a combination of these factors. Detection of accumulation in the nucleus may be performed by any suitable technique. For example, a detectable marker may be fused to the nucleic acid-targeting protein, such that location within a cell may be visualized, such as in combination with a means for detecting the location of the nucleus (e.g., a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of nucleic acid-targeting complex formation (e.g., assay for deaminase activity) at the target sequence, or assay for altered gene expression activity affected by DNA-targeting complex formation and/or DNA-targeting), as compared to a control not exposed to the CRISPR-Cas protein and deaminase protein, or exposed to a CRISPR-Cas and/or deaminase protein lacking the one or more NLSs.

The CRISPR-Cas and/or nucleotide deaminase proteins may be provided with 1 or more, such as with, 2, 3, 4, 5, 6, 7, 8, 9, 10, or more heterologous NLSs. In some embodiments, the proteins comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. In preferred embodiments of the CRISPR-Cas proteins, an NLS attached to the C-terminal of the protein.

In certain embodiments, the CRISPR-Cas protein and the deaminase protein are delivered to the cell or expressed within the cell as separate proteins. In these embodiments, each of the CRISPR-Cas and deaminase protein can be provided with one or more NLSs as described herein. In certain embodiments, the CRISPR-Cas and deaminase proteins are delivered to the cell or expressed with the cell as a fusion protein. In these embodiments one or both of the CRISPR-Cas and deaminase protein is provided with one or more NLSs. Where the nucleotide deaminase is fused to an adaptor protein (such as MS2) as described above, the one or more NLS can be provided on the adaptor protein, provided that this does not interfere with aptamer binding. In particular embodiments, the one or more NLS sequences may also function as linker sequences between the nucleotide deaminase and the CRISPR-Cas protein.

In certain embodiments, guides of the disclosure comprise specific binding sites (e.g., aptamers) for adapter proteins, which may be linked to or fused to an nucleotide deaminase or catalytic domain thereof. When such a guide forms a CRISPR complex (e.g., CRISPR-Cas protein binding to guide and target) the adapter proteins bind and, the nucleotide deaminase or catalytic domain thereof associated with the adapter protein is positioned in a spatial orientation which is advantageous for the attributed function to be effective.

The skilled person will understand that modifications to the guide which allow for binding of the adapter+nucleotide deaminase, but not proper positioning of the adapter+nucleotide deaminase (e.g., due to steric hindrance within the three-dimensional structure of the CRISPR complex) are modifications which are not intended. The one or more modified guide may be modified at the tetra loop, the stem loop 1, stem loop 2, or stem loop 3, as described herein, preferably at either the tetra loop or stem loop 2, and in some cases at both the tetra loop and stem loop 2.

In some embodiments, a component (e.g., the dead Cas protein, the nucleotide deaminase protein or catalytic domain thereof, or a combination thereof) in the systems may comprise one or more nuclear export signals (NES), one or more nuclear localization signals (NLS), or any combinations thereof. In some cases, the NES may be an HIV Rev NES. In certain cases, the NES may be MAPK NES. When the component is a protein, the NES or NLS may be at the C terminus of component. Alternatively or additionally, the NES or NLS may be at the N terminus of component. In some examples, the Cas protein and optionally said nucleotide deaminase protein or catalytic domain thereof comprise one or more heterologous nuclear export signal(s) (NES(s)) or nuclear localization signal(s) (NLS(s)), preferably an HIV Rev NES or MAPK NES, preferably C-terminal.

### Templates

In some embodiments, the composition for engineering cells comprises a template, e.g., a recombination template. A template may be a component of another vector as described herein, contained in a separate vector, or provided as a separate polynucleotide. In some embodiments, a recombination template is designed to serve as a template in homologous recombination, such as within or near a target sequence nicked or cleaved by a nucleic acid-targeting effector protein as a part of a nucleic acid-targeting complex.

In an embodiment, the template nucleic acid alters the sequence of the target position. In an embodiment, the template nucleic acid results in the incorporation of a modified, or non-naturally occurring base into the target nucleic acid.

The template sequence may undergo a breakage mediated or catalyzed recombination with the target sequence. In an embodiment, the template nucleic acid may include sequence that corresponds to a site on the target sequence that is cleaved by a Cas protein mediated cleavage event. In an embodiment, the template nucleic acid may include sequence that corresponds to both, a first site on the target sequence that is cleaved in a first Cas protein mediated event, and a second site on the target sequence that is cleaved in a second Cas protein mediated event.

In certain embodiments, the template nucleic acid can include sequence which results in an alteration in the coding sequence of a translated sequence, e.g., one which results in the substitution of one amino acid for another in a protein product, e.g., transforming a mutant allele into a wild type allele, transforming a wild type allele into a mutant allele, and/or introducing a stop codon, insertion of an amino acid residue, deletion of an amino acid residue, or a nonsense mutation. In certain embodiments, the template nucleic acid can include sequence which results in an alteration in a non-coding sequence, e.g., an alteration in an exon or in a 5′ or 3′ non-translated or non-transcribed region. Such alterations include an alteration in a control element, e.g., a promoter, enhancer, and an alteration in a cis-acting or trans-acting control element.

A template nucleic acid having homology with a target position in a target gene may be used to alter the structure of a target sequence. The template sequence may be used to alter an unwanted structure, e.g., an unwanted or mutant nucleotide. The template nucleic acid may include sequence which, when integrated, results in: decreasing the activity of a positive control element; increasing the activity of a positive control element; decreasing the activity of a negative control element; increasing the activity of a negative control element; decreasing the expression of a gene; increasing the expression of a gene; increasing resistance to a disorder or disease; increasing resistance to viral entry; correcting a mutation or altering an unwanted amino acid residue conferring, increasing, abolishing or decreasing a biological property of a gene product, e.g., increasing the enzymatic activity of an enzyme, or increasing the ability of a gene product to interact with another molecule.

The template nucleic acid may include sequence which results in: a change in sequence of 1, 2, 3, 4, 5, 6, 7, 8, 9, 10, 11, 12 or more nucleotides of the target sequence.

A template polynucleotide may be of any suitable length, such as about or more than about 10, 15, 20, 25, 50, 75, 100, 150, 200, 500, 1000, or more nucleotides in length. In an embodiment, the template nucleic acid may be 20+/−10, 30+/−10, 40+/−10, 50+/−10, 60+/−10, 70+/−10, 80+/−10, 90+/−10, 100+/−10, 110+/−10, 120+/−10, 130+/−10, 140+/−10, 150+/−10, 160+/−10, 170+/−10, 180+/−10, 190+/−10, 200+/−10, 210+/−10, of 220+/−10 nucleotides in length. In an embodiment, the template nucleic acid may be 30+/−20, 40+/−20, 50+/−20, 60+/−20, 70+/−20, 80+/−20, 90+/−20, 100+/−20, 110+/−20, 120+/−20, 130+/−20, 140+/−20, 150+/−20, 160+/−20, 170+/−20, 180+/−20, 190+/−20, 200+/−20, 210+/−20, of 220+/−20 nucleotides in length. In an embodiment, the template nucleic acid is 10 to 1,000, 20 to 900, 30 to 800, 40 to 700, 50 to 600, 50 to 500, 50 to 400, 50 to 300, 50 to 200, or 50 to 100 nucleotides in length.

In some embodiments, the template polynucleotide is complementary to a portion of a polynucleotide comprising the target sequence. When optimally aligned, a template polynucleotide might overlap with one or more nucleotides of a target sequences (e.g., about or more than about 1, 5, 10, 15, 20, 25, 30, 35, 40, 45, 50, 60, 70, 80, 90, 100 or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence.

The exogenous polynucleotide template comprises a sequence to be integrated (e.g., a mutated gene). The sequence for integration may be a sequence endogenous or exogenous to the cell. Examples of a sequence to be integrated include polynucleotides encoding a protein or a non-coding RNA (e.g., a microRNA). Thus, the sequence for integration may be operably linked to an appropriate control sequence or sequences. Alternatively, the sequence to be integrated may provide a regulatory function.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000.

In certain embodiments, one or both homology arms may be shortened to avoid including certain sequence repeat elements. For example, a 5′ homology arm may be shortened to avoid a sequence repeat element. In other embodiments, a 3′ homology arm may be shortened to avoid a sequence repeat element. In some embodiments, both the 5′ and the 3′ homology arms may be shortened to avoid including certain sequence repeat elements.

In some methods, the exogenous polynucleotide template may further comprise a marker. Such a marker may make it easy to screen for targeted integrations. Examples of suitable markers include restriction sites, fluorescent proteins, or selectable markers. The exogenous polynucleotide template of the disclosure can be constructed using recombinant techniques (see, for example, Sambrook et al., 2001 and Ausubel et al., 1996).

In certain embodiments, a template nucleic acid for correcting a mutation may be designed for use as a single-stranded oligonucleotide. When using a single-stranded oligonucleotide, 5′ and 3′ homology arms may range up to about 200 base pairs (bp) in length, e.g., at least 25, 50, 75, 100, 125, 150, 175, or 200 bp in length.

In certain embodiments, a template nucleic acid for correcting a mutation may be designed for use with a homology-independent targeted integration system. Suzuki et al. describe in vivo genome editing via CRISPR/Cas9 mediated homology-independent targeted integration (2016, Nature 540:144-149). Schmid-Burgk, et al. describe use of the CRISPR-Cas9 system to introduce a double-strand break (DSB) at a user-defined genomic location and insertion of a universal donor DNA (Nat Commun. 2016 Jul. 28; 7:12338). Gao, et al. describe “Plug-and-Play Protein Modification Using Homology-Independent Universal Genome Engineering” (Neuron. 2019 Aug. 21; 103(4):583-597).

### RNAi

In some embodiments, the genetic modulating agents may be interfering RNAs. In certain embodiments, diseases caused by a dominant mutation in a gene is targeted by silencing the mutated gene using RNAi. In some cases, the nucleotide sequence may comprise coding sequence for one or more interfering RNAs. In certain examples, the nucleotide sequence may be interfering RNA (RNAi). As used herein, the term “RNAi” refers to any type of interfering RNA, including but not limited to, siRNAi, shRNAi, endogenous microRNA and artificial microRNA. For instance, it includes sequences previously identified as siRNA, regardless of the mechanism of down-stream processing of the RNA (i.e., although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene.

In certain embodiments, a modulating agent may comprise silencing one or more endogenous genes. As used herein, “gene silencing” or “gene silenced” in reference to an activity of an RNAi molecule, for example a siRNA or miRNA refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA”, used interchangeably herein, are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 1 16:281-297), comprises a dsRNA molecule.

Further embodiments are illustrated in the following Examples which are given for illustrative purposes only and are not intended to limit the scope of the invention.

### EXAMPLES

### Example 1—Intact Hi-C Yields a Comprehensive Map of Looping Elements Across the Human Genome

The Applicants used the disclosed methods, termed intact Hi-C to construct comprehensive maps of looping elements across the human genome. Applicants discovered that intact Hi-C further allows generating fully phased diploid maps for any epigenetic assay, such as DNase hypersensitivity maps. Applicants use the methods to generate genome scale epigenetic maps (e.g., DNase sensitivity, DNA methylation and chromatin immunoprecipitation). A key feature of the methods disclosed herein is the fragmentation pattern generated by accessibility of intact chromatin can be used to confirm that the chromatin in an experiment is intact as defined herein.

FIG. 1A shows improved 3D genome mapping with intact Hi-C as compared to in situ Hi-C(Rao S S, Huntley M H, Durand N C, et al. A 3D map of the human genome at kilobase resolution reveals principles of chromatin looping [published correction appears in Cell. 2015 Jul. 30; 162(3):687-8]. Cell. 2014; 159(7):1665-1680). FIG. 1B shows that intact Hi-C can use any digestion strategy (MseI and Csp6I; MboI, MseI, NlaIII and Csp6I; MNase; and DNase). FIG. 2 shows that intact Hi-C allows further zooming in as compared to prior methods. FIG. 3 shows 1 bp resolution for intact Hi-C. FIG. 4 shows that intact Hi-C peaks line up precisely with ChIP-Seq peaks at 1 kb resolution down to 50 bp resolution.

FIG. 5 shows that intact Hi-C enables localization at 1-10 bp resolution purely from Hi-C data. Of 2681 uniquely localized convergent CTCF loops localized with ChIP-Seq data in 2014, 2479 (95%) localized to within 100 bp of both motifs, 1288 (48%) localized to within 30 bp of both motifs using intact Hi-C data alone.

FIG. 6 shows that intact Hi-C detects significantly more loops than in situ Hi-C (350,000 vs 9000) and that the same loops are identified. FIG. 6 also shows that ChIP peaks associated with active transcription line up with loops identified by intact Hi-C. Histone H3 lysine methylation is associated with active transcription (H3K4me3) and can recruit methyl-binding proteins to the loop anchor (see, e.g., Zhang T, Cooper S, Brockdorff N. The interplay of histone modifications—writers that read. EMBO Rep. 2015; 16(11):1467-1481). FIG. 6 also shows that in situ Hi-C loops were mostly at CTCF dependent loop anchors and new loops identified by intact-Hi-C include CTCF independent loops associated with transcription factors and chromatin marks associated with active transcription. Intact Hi-C detects promoter-enhancer (P-E) loops (10K loops with in situ Hi-C to 350K loops). Intact Hi-C localizes loops in the 2D contact matrix with ChIP-Seq resolution or better.

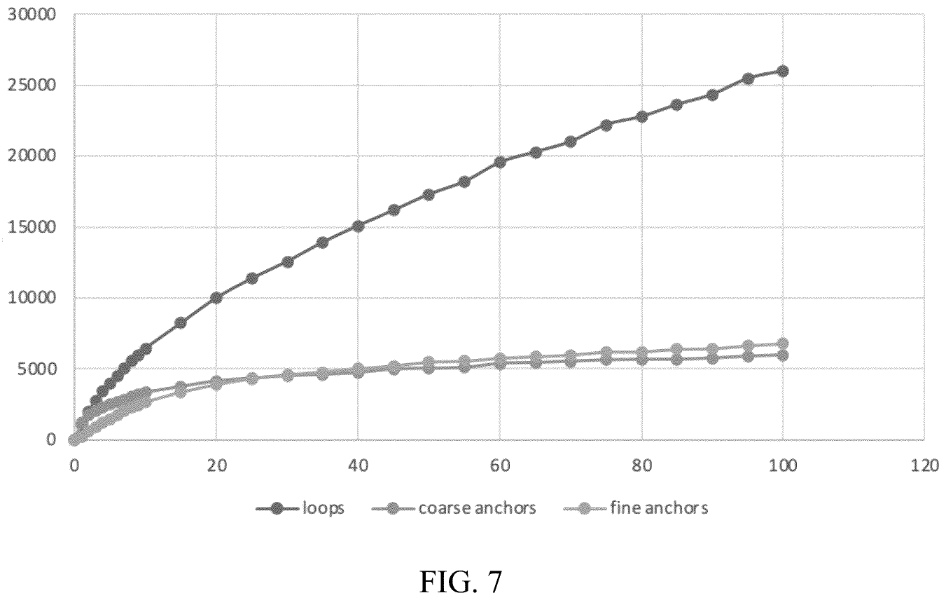

FIG. 7 shows that as sequencing depth increases more loops are identified, however, loop anchors become saturated as sequencing depth increases. The saturation of anchors indicates that intact-Hi-C identified every site capable of forming a loop, however, each loop anchor is capable of interacting with many other loop anchors. Thus, each loop anchor can form many loops.

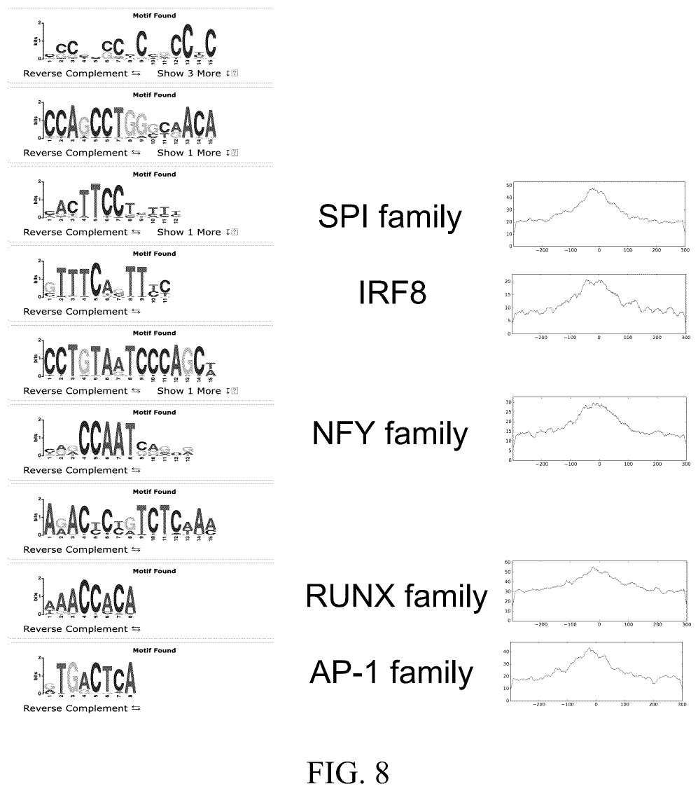

FIG. 8 shows motifs identified using de novo motif calling directly on 2D intact Hi-C localization. In situ Hi-C is poor at linking loops to the causal proteins because the exact sequence bound by a protein cannot be identified at 1 kb resolution. For example, a 15 kb loop anchor can be refined to about 200 bp resolution if combined with ChIP-seq data and further refined to about 1 bp resolution with known motif calling. Thus, in situ Hi-C requires knowledge of protein anchor and ChIP-seq data. Still only about 5000 of anchors are localized with in situ Hi-C. Table 1 shows all motifs identified as being associated with loop formation using the disclosed methods. Intact Hi-C can be used for motif finding to identify DNA motifs associated with loop formation, and thereby determining the protein at the anchor of each loop; or the use of such data to identify genetic variants that influence protein binding or DNA looping, which becomes apparent when homologs with genetic differences exhibit architectural differences at the corresponding loci.

TABLE 1

| | | | | | | | | | MOST_ | |
|---|---|---|---|---|---|---|---|---|---|---|
| | | | | | | | | | SIMILAR_ | MOST_ |
| MOTIF_ | MOTIF_ | MOTIF_ | ALT_ | | | | | E-VALUE_ | MOTIF_ | SIMILAR_ |
| INDEX | SOURCE | ID | ID | CONSENSUS | WIDTH | SITES | E-VALUE | SOURCE | SOURCE | MOTIF |
| 1 | JASPAR | MA0139.1 | MA0139.1. | YGRCCAS | 19 | 43545 | 1.1e−1442 | CENTRIMO | | |
| | 2022_ | | CTCF | YAGRKGG | | | | | | |
| | CORE_ | | | CRSYR | | | | | | |
| | non- | | | (SEQ | | | | | | |
| | redundant_ | | | ID | | | | | | |
| | pfms. | | | NO: | | | | | | |
| | meme | | | 20) | | | | | | |
| 2 | MEME | RSYGCCM | MEME-3 | RSYGCCM | 15 | 23928 | 1.7e−1194 | MEME | JASPAR | MA2025.1 |
| | | YCTRSTG | | YCTRSTG | | | | | 2022 | (MA2025.1. |
| | | G | | G | | | | | CORE_ | CTCF) |
| | | (SEQ | | (SEQ | | | | | non- | |
| | | ID | | ID | | | | | redundant_ | |
| | | NO: | | NO: | | | | | pfms. | |
| | | 21) | | 21) | | | | | meme | |
| 3 | STREME | 1-CCAC | STREME-1 | CCACTAG | 10 | 13962 | 1.3e−1057 | STREME | JASPAR | MA2026.1 |
| | | TAGRKG | | RKG | | | | | 2022 | (MA2026.1. |
| | | (SEQ | | (SEQ | | | | | CORE_ | CTCF) |
| | | ID | | ID | | | | | non- | |
| | | NO: | | NO: | | | | | redundant_ | |
| | | 22) | | 22) | | | | | pfms. | |
| | | | | | | | | | meme | |
| 4 | JASPAR | MA2026.1 | MA2026.1. | CTGCAGT | 35 | 29031 | 5.8e−535 | CENTRIMO | | |
| | 2022_ | | CTCF | KCCNVCH | | | | | | |
| | CORE_ | | | NNYRGCC | | | | | | |
| | non- | | | ASYAGRK | | | | | | |
| | redundant_ | | | GGCRSYN | | | | | | |
| | pfms. | | | (SEQ | | | | | | |
| | meme | | | ID | | | | | | |
| | | | | NO: | | | | | | |
| | | | | 23) | | | | | | |
| 5 | JASPAR | MA2025.1 | MA2025.1. | CTGCAGT | 34 | 42881 | 1.1e−516 | CENTRIMO | | |
| | 2022_ | | CTCF | KCCNNNN | | | | | | |
| | CORE_ | | | NYNRCCA | | | | | | |
| | non- | | | SYAGRKG | | | | | | |
| | redundant_ | | | GCRSYV | | | | | | |
| | pfms. | | | (SEQ | | | | | | |
| | meme | | | ID | | | | | | |
| | | | | NO: | | | | | | |
| | | | | 24) | | | | | | |
| 6 | JASPAR | MA0531.1 | MA0531.1. | CCRMYAG | 15 | 38260 | 3.8e−463 | CENTRIMO | | |
| | 2022_ | | CTCF | RTGGCGC | | | | | | |
| | CORE_ | | | Y | | | | | | |
| | non- | | | (SEQ | | | | | | |
| | redundant_ | | | ID | | | | | | |
| | pfms. | | | NO: | | | | | | |
| | meme | | | 25) | | | | | | |
| 7 | JASPAR | MA1102.2 | MA1102.2. | NSCAGGG | 12 | 58946 | 3.2e−425 | CENTRIMO | | |
| | 2022_ | | CTCFL | GGCGS | | | | | | |
| | CORE_ | | | (SEQ | | | | | | |
| | non- | | | ID | | | | | | |
| | redundant_ | | | NO: | | | | | | |
| | pfms. | | | 26) | | | | | | |
| | meme | | | | | | | | | |
| 8 | JASPAR | MA0373.1 | MA0373.1. | GGTGG | 7 | 37140 | 4.60E−225 | CENTRIMO | | |
| | 2022_ | | RPN4 | CG | | | | | | |
| | CORE_ | | | (SEQ | | | | | | |
| | non- | | | ID | | | | | | |
| | redundant_ | | | NO: | | | | | | |
| | pfms. | | | 27) | | | | | | |
| | meme | | | | | | | | | |
| 9 | MEME | TTTTTTT | MEME-1 | TTTTTTT | 15 | 20428 | 5.90E−181 | MEME | JASPAR | MA1274.1 |
| | | TTTTTTT | | TTTTTTT | | | | | 2022 | (MA1274.1. |
| | | T | | T | | | | | CORE_ | DOF3.6) |
| | | (SEQ | | (SEQ | | | | | non- | |
| | | ID | | ID | | | | | redundant_ | |
| | | NO: | | NO: | | | | | pfms. | |
| | | 28) | | 28) | | | | | meme | |
| 10 | JASPAR | MA0751.1 | MA0751.1. | GRCCCCC | 15 | 45299 | 4.10E−167 | CENTRIMO | | |
| | 2022_ | | ZIC4 | CGCKGYG | | | | | | |
| | CORE_ | | | H | | | | | | |
| | non- | | | (SEQ | | | | | | |
| | redundant_ | | | ID | | | | | | |
| | pfms. | | | NO: | | | | | | |
| | meme | | | 29) | | | | | | |
| 11 | STREME | 2-CCAGC | STREME-2 | CCAGCCT | 15 | 5530 | 1.00E−145 | STREME | | |
| | | CTGGGCR | | GGGCRAC | | | | | | |
| | | ACA | | A | | | | | | |
| | | (SEQ | | (SEQ | | | | | | |
| | | ID | | ID | | | | | | |
| | | NO: | | NO: | | | | | | |
| | | 30) | | 30) | | | | | | |
| 12 | STREME | 3-GCCTG | STREME-3 | GCCTGTA | 15 | 4917 | 1.30E−128 | STREME | | |
| | | TAATCCC | | ATCCCAG | | | | | | |
| | | AGC | | C | | | | | | |
| | | (SEQ | | (SEQ | | | | | | |
| | | ID | | ID | | | | | | |
| | | NO: | | NO: | | | | | | |
| | | 31) | | 31) | | | | | | |
| 13 | STREME | 4- | STREME-4 | RGYGCRG | 13 | 5138 | 5.70E−120 | STREME | | |
| | | RGYGCRG | | TGGCDC | | | | | | |
| | | TGGCDC | | (SEQ | | | | | | |
| | | (SEQ | | ID | | | | | | |
| | | ID | | NO: | | | | | | |
| | | NO: | | 32) | | | | | | |
| | | 32) | | | | | | | | |
| 14 | STREME | 5- | STREME-5 | GCCTCRG | 15 | 5034 | 5.50E−114 | STREME | JASPAR | MA1596.1 |
| | | GCCTCRG | | CCTCCCA | | | | | 2022 | (MA1596.1. |
| | | CCTCCCA | | A | | | | | CORE_ | ZNF460) |
| | | A | | (SEQ | | | | | non- | |
| | | (SEQ | | ID | | | | | redundant_ | |
| | | ID | | NO: | | | | | pfms. | |
| | | NO: | | 33) | | | | | meme | |
| | | 33) | | | | | | | | |
| 15 | MEME | GGAGGCB | MEME-2 | GGAGGCB | 15 | 19217 | 1.90E−112 | MEME | JASPAR | MA1977.1 |
| | | GRGGCRG | | GRGGCRG | | | | | 2022 | (MA1977.1. |
| | | G | | G | | | | | CORE_ | Zm00001 |
| | | (SEQ | | (SEQ | | | | | non- | d049364) |
| | | ID | | ID | | | | | redundant_ | |
| | | NO: | | NO: | | | | | pfms. | |
| | | 34) | | 34) | | | | | meme | |
| 16 | JASPAR | MA0696.1 | MA0696.1. | GACCCCC | 14 | 12102 | 3.40E−108 | CENTRIMO | | |
| | 2022_ | | ZIC1 | YGCTG | | | | | | |
| | CORE_ | | | TG | | | | | | |
| | non- | | | (SEQ | | | | | | |
| | redundant_ | | | ID | | | | | | |
| | pfms. | | | NO: | | | | | | |
| | meme | | | 35) | | | | | | |
| 17 | JASPAR | MA0334.1 | MA0334.1. | MGCCA | 7 | 94666 | 8.30E−104 | CENTRIMO | | |
| | 2022_ | | MET32 | CA | | | | | | |
| | CORE_ | | | (SEQ | | | | | | |
| | non- | | | ID | | | | | | |
| | redundant_ | | | NO: | | | | | | |
| | pfms. | | | 36) | | | | | | |
| | meme | | | | | | | | | |
| 18 | MEME | TGTYGCC | MEME-5 | TGTYGCC | 15 | 4824 | 2.50E−101 | MEME | | |
| | | CAGGCTG | | CAGGCTG | | | | | | |
| | | G | | G | | | | | | |
| | | (SEQ | | (SEQ | | | | | | |
| | | ID | | ID | | | | | | |
| | | NO: | | NO: | | | | | | |
| | | 37) | | 37) | | | | | | |
| 19 | MEME | GCCTGTA | MEME-4 | GCCTGTA | 15 | 3918 | 4.50E−99 | MEME | | |
| | | ATCCCAG | | ATCCCAG | | | | | | |
| | | C | | C | | | | | | |
| | | (SEQ | | (SEQ | | | | | | |
| | | ID | | ID | | | | | | |
| | | NO: | | NO: | | | | | | |
| | | 38) | | 38) | | | | | | |
| 20 | JASPAR | MA0697.2 | MA0697.2. | CNCAGCA | 13 | 73010 | 5.90E−99 | CENTRIMO | | |
| | 2022_ | | Zic3 | GGAGNN | | | | | | |
| | CORE_ | | | (SEQ | | | | | | |
| | non- | | | ID | | | | | | |
| | redundant_ | | | NO: | | | | | | |
| | pfms. | | | 39) | | | | | | |
| | meme | | | | | | | | | |
| 21 | STREME | 6- | STREME-6 | ARACYCY | 12 | 4119 | 1.40E−95 | STREME | | |
| | | ARACYCY | | GTCTC | | | | | | |
| | | GTCTC | | (SEQ | | | | | | |
| | | (SEQ | | ID | | | | | | |
| | | ID | | NO: | | | | | | |
| | | NO: | | 40) | | | | | | |
| | | 40) | | | | | | | | |
| 22 | STREME | 7- | STREME-7 | YTCAAGY | 15 | 3606 | 1.10E−94 | STREME | | |
| | | YTCAAGY | | GATYCTC | | | | | | |
| | | GATYCTC | | C | | | | | | |
| | | C | | (SEQ | | | | | | |
| | | (SEQ | | ID | | | | | | |
| | | ID | | NO: | | | | | | |
| | | NO: | | 41) | | | | | | |
| | | 41) | | | | | | | | |
| 23 | JASPAR | MA1628.1 | MA1628.1. | CVCAGCA | 11 | 61952 | 6.00E−94 | CENTRIMO | | |
| | 2022_ | | Zic1::Zic2 | GGNV | | | | | | |
| | CORE_ | | | (SEQ | | | | | | |
| | non- | | | ID | | | | | | |
| | redundant_ | | | NO: | | | | | | |
| | pfms. | | | 42) | | | | | | |
| | meme | | | | | | | | | |
| 24 | STREME | 8- | STREME-8 | AAAAAAA | 14 | 6619 | 3.90E−92 | STREME | JASPAR | MA1268.1 |
| | | AAAAAAA | | MAAAAAA | | | | | 2022_ | (MA1268.1. |
| | | MAAAAAA | | (SEQ | | | | | CORE_ | CDF5) |
| | | (SEQ | | ID | | | | | non- | |
| | | ID | | NO: | | | | | redundant_ | |
| | | NO: | | 43) | | | | | pfms. | |
| | | 43) | | | | | | | meme | |
| 25 | JASPAR | MA0118.1 | MA0118.1. | YGGGKGK | 9 | 102576 | 1.60E−90 | CENTRIMO | | |
| | 2022_ | | Mach0-1 | YV | | | | | | |
| | CORE_ | | | (SEQ | | | | | | |
| | non- | | | ID | | | | | | |
| | redundant_ | | | NO: | | | | | | |
| | pfms. | | | 44) | | | | | | |
| | meme | | | | | | | | | |
| 26 | STREME | 9.GCAGTGA | STREME-9 | GCAGTGA | 15 | 2929 | 1.90E−83 | STREME | JASPAR | MA1764.1 |
| | | GCYRAGA | | GCYRAGA | | | | | 2022_ | (MA1764.1. |
| | | T | | T | | | | | CORE_ | TREE1) |
| | | (SEQ | | (SEQ | | | | | non- | |
| | | ID | | ID | | | | | redundant_ | |
| | | NO: | | NO: | | | | | pfms. | |
| | | 45) | | 45) | | | | | meme | |
| 27 | JASPAR | MA1584.1 | MA1584.1. | VGACCCC | 16 | 10150 | 4.40E−82 | CENTRIMO | | |
| | 2022_ | | ZIC5 | CCGCTGH | | | | | | |
| | CORE_ | | | GM | | | | | | |
| | non- | | | (SEQ | | | | | | |
| | redundant_ | | | ID | | | | | | |
| | pfms. | | | NO: | | | | | | |
| | meme | | | 46) | | | | | | |
| 28 | JASPAR | MA1467.2 | MA1467.2. | RVCAGAT | 11 | 60821 | 2.50E−78 | CENTRIMO | | |
| | 2022_ | | Atoh1 | GGYN | | | | | | |
| | COREnon- | | | (SEQ | | | | | | |
| | redundant_ | | | ID | | | | | | |
| | pfms. | | | NO: | | | | | | |
| | meme | | | 47) | | | | | | |
| 29 | STREME | 10- | STREME-10 | 10-AGGA | 9 | 31958 | 4.10E−78 | STREME | JASPAR | MA0598.3 |
| | | AGGAAGT | | AGTGR | | | | | 2022 | (MA0598.3. |
| | | GR | | (SEQ | | | | | CORE_ | EHF) |
| | | (SEQ | | ID | | | | | non- | |
| | | ID | | NO: | | | | | redundant_ | |
| | | NO: | | 48) | | | | | pfms. | |
| | | 48) | | | | | | | meme | |
| 30 | JASPAR | MA0456.1 | MA0456.1. | GMCCCCC | 12 | 34526 | 1.30E−77 | CENTRIMO | | |
| | 2022_ | | opa | CGCTG | | | | | | |
| | CORE_ | | | (SEQ | | | | | | |
| | non- | | | ID | | | | | | |
| | redundant_ | | | NO: | | | | | | |
| | pfms. | | | 49 | | | | | | |
| | meme | | | | | | | | | |
| 31 | JASPAR | MA0333.1 | MA0333.1. | RNTGTGG | 9 | 37910 | 6.20E−76 | CENTRIMO | | |
| | 2022_ | | MET31 | CG | | | | | | |
| | CORE_ | | | (SEQ | | | | | | |
| | non- | | | ID | | | | | | |
| | redundant_ | | | NO: | | | | | | |
| | pfms. | | | 50) | | | | | | |
| | meme | | | | | | | | | |
| 32 | JASPAR | MA1629.1 | MA1629.1. | NDCACAG | 14 | 60293 | 1.70E−72 | CENTRIMO | | |
| | 2022_ | | Zic2 | CAGGD | | | | | | |
| | CORE_ | | | RG | | | | | | |
| | non- | | | (SEQ | | | | | | |
| | redundant_ | | | ID | | | | | | |
| | pfms. | | | NO: | | | | | | |
| | meme | | | 51) | | | | | | |
| 33 | JASPAR | MA0213.1 | MA0213.1. | SYGGCGC | 8 | 30817 | 1.90E−72 | CENTRIMO | | |
| | 2022_ | | brk | Y | | | | | | |
| | CORE_ | | | (SEQ | | | | | | |
| | non- | | | ID | | | | | | |
| | redundant_ | | | NO: | | | | | | |
| | pfms. | | | 52) | | | | | | |
| | meme | | | | | | | | | |
| 34 | JASPAR | MA1109.1 | MA1109.1. | NRACAGA | 13 | 61350 | 7.60E−70 | CENTRIMO | | |
| | 2022_ | | NEUROD1 | TGGYNN | | | | | | |
| | CORE_ | | | (SEQ | | | | | | |
| | non- | | | ID | | | | | | |
| | redundant_ | | | NO: | | | | | | |
| | pfms. | | | 53) | | | | | | |
| | meme | | | | | | | | | |
| 35 | JASPAR | MA0997.1 | MA0997.1. | NCGCCGB | 9 | 76698 | 5.30E−69 | CENTRIMO | | |
| | 2022_ | | ERFO69 | MN | | | | | | |
| | CORE_ | | | (SEQ | | | | | | |
| | non- | | | ID | | | | | | |
| | redundant_ | | | NO: | | | | | | |
| | pfms. | | | 54) | | | | | | |
| | meme | | | | | | | | | |
| 36 | JASPAR | MA1568.1 | MA1568.1. | CACCATA | 12 | 33532 | 2.70E−63 | CENTRIMO | | |
| | 2022_ | | TCF21 | TGKYR | | | | | | |
| | CORE_ | | | (SEQ | | | | | | |
| | non- | | | ID | | | | | | |
| | redundant_ | | | NO: | | | | | | |
| | pfms. | | | 55) | | | | | | |
| | meme | | | | | | | | | |
| 37 | JASPAR | MA0739.1 | MA0739.1. | RTGCCAA | 9 | 82810 | 2.50E−60 | CENTRIMO | | |
| | 2022_ | | Hic1 | CY | | | | | | |
| | CORE_ | | | (SEQ | | | | | | |
| | non- | | | ID | | | | | | |
| | redundant_ | | | NO: | | | | | | |
| | pfms. | | | 56) | | | | | | |
| | meme | | | | | | | | | |
| 38 | JASPAR | MA0104.4 | MA0104.4. | VVCCACG | 12 | 32225 | 6.90E−59 | CENTRIMO | | |
| | 2022_ | | MYCN | TGGBB | | | | | | |
| | CORE_ | | | (SEQ | | | | | | |
| | non- | | | ID | | | | | | |
| | redundant_ | | | NO: | | | | | | |
| | pfms. | | | 57) | | | | | | |
| | meme | | | | | | | | | |
| 39 | JASPAR | MA1414.1 | MA1414.1. | WVGCGCC | 10 | 48547 | 8.70E−59 | CENTRIMO | | |
| | 2022_ | | E2FA | AHN | | | | | | |
| | CORE_ | | | (SEQ | | | | | | |
| | non- | | | ID | | | | | | |
| | redundant_ | | | NO: | | | | | | |
| | pfms. | | | 58) | | | | | | |
| | meme | | | | | | | | | |
| 40 | JASPAR | MA0668.2 | MA0668.2. | NNGRACA | 15 | 59392 | 8.90E−58 | CENTRIMO | | |
| | 2022_ | | Neurod2 | GATGGYN | | | | | | |
| | CORE_ | | | N | | | | | | |
| | non- | | | (SEQ | | | | | | |
| | redundant_ | | | ID | | | | | | |
| | pfms. | | | NO: | | | | | | |
| | meme | | | 59) | | | | | | |
| 41 | JASPAR | MA1578.1 | MA1578.1. | CCCCCCM | 10 | 38771 | 1.30E−57 | CENTRIMO | | |
| | 2022_ | | VEZF1 | YDH | | | | | | |
| | CORE_ | | | (SEQ | | | | | | |
| | non- | | | ID | | | | | | |
| | redundant_ | | | NO: | | | | | | |
| | pfms. | | | 60) | | | | | | |
| | meme | | | | | | | | | |
| 42 | JASPAR | MA1986.1 | MA1986.1. | NNCCACG | 11 | 65822 | 1.80E−57 | CENTRIMO | | |
| | 2022_ | | Zm00001 | CGNN | | | | | | |
| | CORE_ | | d034298 | (SEQ | | | | | | |
| | non- | | | ID | | | | | | |
| | redundant_ | | | NO: | | | | | | |
| | pfms. | | | 61) | | | | | | |
| | meme | | | | | | | | | |
| 43 | JASPAR | MA1548.1 | MA1548.1. | NGGGCCC | 10 | 33583 | 2.40E−57 | CENTRIMO | | |
| | 2022_ | | PLAGL2 | CCN | | | | | | |
| | CORE_ | | | (SEQ | | | | | | |
| | non- | | | ID | | | | | | |
| | redundant_ | | | NO: | | | | | | |
| | pfms. | | | 62) | | | | | | |
| | meme | | | | | | | | | |
| 44 | JASPAR | MA1202.1 | MA1202.1. | TCACCA | 6 | 42239 | 3.40E−56 | CENTRIMO | | |
| | 2022_ | | AGL55 | (SEQ | | | | | | |
| | CORE_ | | | ID | | | | | | |
| | non- | | | NO: | | | | | | |
| | redundant_ | | | 63) | | | | | | |
| | pfms. | | | | | | | | | |
| | meme | | | | | | | | | |
| 45 | JASPAR | MA1968.1 | MA1968.1. | CACGTGG | 11 | 61994 | 9.20E−56 | CENTRIMO | | |
| | 2022_ | | GLYMA- | CANN | | | | | | |
| | CORE_ | | 06G314400 | (SEQ | | | | | | |
| | non- | | | ID | | | | | | |
| | redundant_ | | | NO: | | | | | | |
| | pfms. | | | 64) | | | | | | |
| | meme | | | | | | | | | |
| 46 | JASPAR | MA0748.2 | MA0748.2. | NVATGGC | 11 | 47647 | 2.10E−53 | CENTRIMO | | |
| | 2022_ | | YY2 | GGCS | | | | | | |
| | CORE_ | | | (SEQ | | | | | | |
| | non- | | | ID | | | | | | |
| | redundant_ | | | NO: | | | | | | |
| | pfms. | | | 65) | | | | | | |
| | meme | | | | | | | | | |
| 47 | JASPAR | MA0864.2 | MA0864.2. | RWTTTGG | 16 | 11251 | 1.20E−51 | CENTRIMO | | |
| | 2022_ | | E2F2 | CGCCAWW | | | | | | |
| | CORE_ | | | WY | | | | | | |
| | non- | | | (SEQ | | | | | | |
| | redundant_ | | | ID | | | | | | |
| | pfms. | | | NO: | | | | | | |
| | meme | | | 66) | | | | | | |
| 48 | JASPAR | MA1989.1 | MA1989.1. | CACGTGG | 11 | 55423 | 1.60E−51 | CENTRIMO | | |
| | 2022_ | | GLYMA- | CANN | | | | | | |
| | CORE_ | | 13G317000 | (SEQ | | | | | | |
| | non- | | | ID | | | | | | |
| | redundant_ | | | NO: | | | | | | |
| | pfms. | | | 67) | | | | | | |
| | meme | | | | | | | | | |
| 49 | JASPAR | MA1351.2 | MA1351.2. | SACGTGG | 11 | 58513 | 6.70E−51 | CENTRIMO | | |
| | 2022_ | | GBF3 | CANN | | | | | | |
| | CORE_ | | | (SEQ | | | | | | |
| | non- | | | ID | | | | | | |
| | redundant_ | | | NO: | | | | | | |
| | pfms. | | | 68) | | | | | | |
| | meme | | | | | | | | | |
| 50 | JASPAR | MA1468.1 | MA1468.1. | AVCATAT | 10 | 58316 | 9.50E−51 | CENTRIMO | | |
| | 2022_ | | ATOH7 | GBY | | | | | | |
| | CORE_ | | | (SEQ | | | | | | |
| | non- | | | ID | | | | | | |
| | redundant_ | | | NO: | | | | | | |
| | pfms. | | | 69) | | | | | | |
| | meme | | | | | | | | | |
| 51 | JASPAR | MA1642.1 | MA1642.1. | NNVACAG | 13 | 66727 | 5.40E−50 | CENTRIMO | | |
| | 2022_ | | NEUROG2 | ATGGNN | | | | | | |
| | CORE_ | | | (SEQ | | | | | | |
| | non- | | | ID | | | | | | |
| | redundant_ | | | NO: | | | | | | |
| | pfms. | | | 70) | | | | | | |
| | meme | | | | | | | | | |
| 52 | JASPAR | MA0872.1 | MA0872.1. | TGCCCYS | 13 | 18669 | 6.90E−49 | CENTRIMO | | |
| | 2022_ | | TFAP2A | RGGGCA | | | | | | |
| | CORE_ | | | (SEQ | | | | | | |
| | non- | | | ID | | | | | | |
| | redundant_ | | | NO: | | | | | | |
| | pfms. | | | 71) | | | | | | |
| | meme | | | | | | | | | |
| 53 | JASPAR | MA0820.1 | MA0820.1. | WMCACCT | 10 | 69658 | 3.00E−46 | CENTRIMO | | |
| | 2022_ | | FIGLA | GKW | | | | | | |
| | CORE_ | | | (SEQ | | | | | | |
| | non- | | | ID | | | | | | |
| | redundant_ | | | NO: | | | | | | |
| | pfms. | | | 72) | | | | | | |
| | meme | | | | | | | | | |
| 54 | JASPAR | MA0979.1 | MA0979.1. | CRCCG | 8 | 56194 | 3.40E−46 | CENTRIMO | | |
| | 2022_ | | ERFO08 | MCS | | | | | | |
| | CORE_ | | | (SEQ | | | | | | |
| | non- | | | ID | | | | | | |
| | redundant_ | | | NO: | | | | | | |
| | pfms. | | | 73) | | | | | | |
| | meme | | | | | | | | | |
| 55 | JASPAR | MA0366.1 | MA0366.1. | AGGGG | 5 | 90618 | 1.30E−45 | CENTRIMO | | |
| | 2022_ | | RGM1 | (SEQ | | | | | | |
| | CORE_ | | | ID | | | | | | |
| | non- | | | NO: | | | | | | |
| | redundant_ | | | 74) | | | | | | |
| | pfms. | | | | | | | | | |
| | meme | | | | | | | | | |
| 56 | MEME | GAGACRG | MEME-6 | GAGACRG | 15 | 4118 | 1.80E−45 | MEME | | |
| | | RGTYTCR | | RGTYTCR | | | | | | |
| | | C | | C | | | | | | |
| | | (SEQ | | (SEQ | | | | | | |
| | | ID | | ID | | | | | | |
| | | NO: | | NO: | | | | | | |
| | | 75) | | 75) | | | | | | |
| 57 | JASPAR | MA0830.2 | MA0830.2. | NNGCACC | 13 | 71787 | 3.30E−44 | CENTRIMO | | |
| | 2022_ | | TCF4 | TGCCNN | | | | | | |
| | CORE_ | | | (SEQ | | | | | | |
| | non- | | | ID | | | | | | |
| | redundant_ | | | NO: | | | | | | |
| | pfms. | | | 76) | | | | | | |
| | meme | | | | | | | | | |
| 58 | JASPAR | MA0193.1 | MA0193.1. | CYACYAA | 7 | 80536 | 3.70E−44 | CENTRIMO | | |
| | 2022_ | | schlank | (SEQ | | | | | | |
| | CORE_ | | | ID | | | | | | |
| | non- | | | NO: | | | | | | |
| | redundant_ | | | 77) | | | | | | |
| | pfms. | | | | | | | | | |
| | meme | | | | | | | | | |
| 59 | JASPAR | MA1648.1 | MA1648.1. | NNCACCT | 11 | 75972 | 5.00E−42 | CENTRIMO | | |
| | 2022_ | | TCF12 | GCNN | | | | | | |
| | CORE_ | | | (SEQ | | | | | | |
| | non- | | | ID | | | | | | |
| | redundant_ | | | NO: | | | | | | |
| | pfms. | | | 78) | | | | | | |
| | meme | | | | | | | | | |
| 60 | JASPAR | MA1767.1 | MA1767.1. | VCRCCGC | 10 | 76952 | 1.40E−41 | CENTRIMO | | |
| | 2022_ | | WIN1 | MRY | | | | | | |
| | CORE_ | | | (SEQ | | | | | | |
| | non- | | | ID | | | | | | |
| | redundant_ | | | NO: | | | | | | |
| | pfms. | | | 79) | | | | | | |
| | meme | | | | | | | | | |
| 61 | JASPAR | MA1053.1 | MA1053.1. | GCGCCGC | 8 | 27402 | 1.50E−41 | CENTRIMO | | |
| | 2022_ | | ERF109 | C | | | | | | |
| | CORE_ | | | (SEQ | | | | | | |
| | non- | | | ID | | | | | | |
| | redundant_ | | | NO: | | | | | | |
| | pfms. | | | 80) | | | | | | |
| | meme | | | | | | | | | |
| 62 | JASPAR | MA1410.1 | MA1410.1. | BGGGSCC | 10 | 53067 | 2.00E−41 | CENTRIMO | | |
| | 2022_ | | StBRC1 | MCC | | | | | | |
| | CORE_ | | | (SEQ | | | | | | |
| | non- | | | ID | | | | | | |
| | redundant_ | | | NO: | | | | | | |
| | pfms. | | | 81) | | | | | | |
| | meme | | | | | | | | | |
| 63 | JASPAR | MA0813.1 | MA0813.1. | TGCCCYB | 13 | 15739 | 2.20E−39 | CENTRIMO | | |
| | 2022_ | | TFAP2B | RGGGCA | | | | | | |
| | CORE_ | | | (SEQ | | | | | | |
| | non- | | | ID | | | | | | |
| | redundant_ | | | NO: | | | | | | |
| | pfms. | | | 82) | | | | | | |
| | meme | | | | | | | | | |
| 64 | JASPAR | MA0993.1 | MA0993.1. | MGCCGYC | 10 | 72855 | 2.40E−39 | CENTRIMO | | |
| | 2022_ | | ERF7 | RNN | | | | | | |
| | CORE_ | | | (SEQ | | | | | | |
| | non- | | | ID | | | | | | |
| | redundant_ | | | NO: | | | | | | |
| | pfms. | | | 83) | | | | | | |
| | meme | | | | | | | | | |
| 65 | JASPAR | MA0342.1 | MA0342.1. | AGGGG | 5 | 60244 | 1.30E−38 | CENTRIMO | | |
| | 2022_ | | MSN4 | (SEQ | | | | | | |
| | CORE_ | | | ID | | | | | | |
| | non- | | | NO: | | | | | | |
| | redundant_ | | | 84) | | | | | | |
| | pfms. | | | | | | | | | |
| | meme | | | | | | | | | |
| 66 | JASPAR | MA0738.1 | MA0738.1. | RTGCCCR | 9 | 96093 | 1.60E−38 | CENTRIMO | | |
| | 2022_ | | HIC2 | SB | | | | | | |
| | CORE_ | | | (SEQ | | | | | | |
| | non- | | | ID | | | | | | |
| | redundant_ | | | NO: | | | | | | |
| | pfms. | | | 85) | | | | | | |
| | meme | | | | | | | | | |
| 67 | JASPAR | MA1728.1 | MA1728.1. | NNTGCTG | 12 | 76634 | 7.80E−38 | CENTRIMO | | |
| | 2022_ | | ZNF549 | CCCWR | | | | | | |
| | CORE_ | | | (SEQ | | | | | | |
| | non- | | | ID | | | | | | |
| | redundant_ | | | NO: | | | | | | |
| | pfms. | | | 86) | | | | | | |
| | meme | | | | | | | | | |
| 68 | JASPAR | MA0470.2 | MA0470.2. | TTTTGGC | 14 | 7313 | 8.70E−38 | CENTRIMO | | |
| | 2022_ | | E2F4 | GCCAWW | | | | | | |
| | CORE_ | | | W | | | | | | |
| | non- | | | (SEQ | | | | | | |
| | redundant_ | | | ID | | | | | | |
| | pfms. | | | NO: | | | | | | |
| | meme | | | 87) | | | | | | |
| 69 | JASPAR | MA0147.3 | MA0147.3. | NNCCACG | 12 | 44997 | 9.00E−38 | CENTRIMO | | |
| | 2022_ | | MYC | TGCNB | | | | | | |
| | CORE_ | | | (SEQ | | | | | | |
| | non- | | | ID | | | | | | |
| | redundant_ | | | NO: | | | | | | |
| | pfms. | | | 88 | | | | | | |
| | meme | | | | | | | | | |
| 70 | JASPAR | MA0998.1 | MA0998.1. | NMGCCGC | 10 | 63711 | 2.70E−37 | CENTRIMO | | |
| | 2022_ | | ERFO96 | CDN | | | | | | |
| | CORE_ | | | (SEQ | | | | | | |
| | non- | | | ID | | | | | | |
| | redundant_ | | | NO: | | | | | | |
| | pfms. | | | 89) | | | | | | |
| | meme | | | | | | | | | |
| 71 | JASPAR | MA0815.1 | MA0815.1. | TGCCCYS | 13 | 15077 | 7.30E−37 | CENTRIMO | | |
| | 2022_ | | TFAP20 | RGGGCA | | | | | | |
| | CORE_ | | | (SEQ | | | | | | |
| | non- | | | ID | | | | | | |
| | redundant_ | | | NO: | | | | | | |
| | pfms. | | | 90) | | | | | | |
| | meme | | | | | | | | | |
| 72 | JASPAR | MA0024.3 | MA0024.3. | TTTGGCG | 12 | 11443 | 1.80E−36 | CENTRIMO | | |
| | 2022_ | | E2F1 | CCAAA | | | | | | |
| | CORE_ | | | (SEQ | | | | | | |
| | non- | | | ID | | | | | | |
| | redundant_ | | | NO: | | | | | | |
| | pfms. | | | 91) | | | | | | |
| | meme | | | | | | | | | |
| 73 | MEME | TGAGGYC | MEME-7 | TGAGGYC | 15 | 3306 | 1.90E−36 | MEME | JASPAR | MA0728.1 |
| | | AGGAGTT | | AGGAGTT | | | | | 2022_ | (MA0728.1. |
| | | Y | | Y | | | | | CORE_ | Nr2F6) |
| | | (SEQ | | (SEQ | | | | | non- | |
| | | ID | | ID | | | | | redundant_ | |
| | | NO: | | NO: | | | | | pfms. | |
| | | 92) | | 92) | | | | | meme | |
| 74 | JASPAR | MA1631.1 | MA1631.1. | NNGCACC | 13 | 65965 | 1.80E−35 | CENTRIMO | | |
| | 2022_ | | ASCL1 | TGCYNB | | | | | | |
| | CORE_ | | | (SEQ | | | | | | |
| | non- | | | ID | | | | | | |
| | redundant_ | | | NO: | | | | | | |
| | pfms. | | | 93 | | | | | | |
| | meme | | | | | | | | | |
| 75 | JASPAR | MA1727.1 | MA1727.1. | VRBVNTG | 15 | 19466 | 7.60E−35 | CENTRIMO | | |
| | 2022_ | | ZNF417 | GGCGCCA | | | | | | |
| | CORE_ | | | M | | | | | | |
| | non- | | | (SEQ | | | | | | |
| | redundant_ | | | ID | | | | | | |
| | pfms. | | | NO: | | | | | | |
| | meme | | | 94) | | | | | | |
| 76 | MEME | GCSGGGC | MEME-8 | GCSGGGC | 15 | 9125 | 1.10E−34 | MEME | JASPAR | MA1966.1 |
| | | GBGGTGG | | GBGGTGG | | | | | 2022 | (MA1966.1. |
| | | C | | C | | | | | CORE_ | Klf6-7- |
| | | (SEQ | | (SEQ | | | | | non- | like) |
| | | ID | | ID | | | | | redundant_ | |
| | | NO: | | NO: | | | | | pfms. | |
| | | 95) | | 95) | | | | | meme | |
| 77 | JASPAR | MA0341.1 | MA0341.1. | RGGGG | 5 | 65391 | 2.40E−34 | CENTRIMO | | |
| | 2022_ | | MSN2 | (SEQ | | | | | | |
| | CORE_ | | | ID | | | | | | |
| | non- | | | NO: | | | | | | |
| | redundant_ | | | 96) | | | | | | |
| | pfms. | | | | | | | | | |
| | meme | | | | | | | | | |
| 78 | JASPAR | MA0364.1 | MA0364.1. | CCCC | 7 | 57528 | 1.80E−33 | CENTRIMO | | |
| | 2022_ | | REI1 | TGA | | | | | | |
| | CORE_ | | | (SEQ | | | | | | |
| | non- | | | ID | | | | | | |
| | redundant_ | | | NO: | | | | | | |
| | pfms. | | | 97) | | | | | | |
| | meme | | | | | | | | | |
| 79 | JASPAR | MA0116.1 | MA0116.1. | GSMMCCY | 15 | 6813 | 2.90E−33 | CENTRIMO | | |
| | 2022_ | | Znf423 | ARGGKKB | | | | | | |
| | CORE_ | | | M | | | | | | |
| | non- | | | (SEQ | | | | | | |
| | redundant_ | | | ID | | | | | | |
| | pfms. | | | NO: | | | | | | |
| | meme | | | 98) | | | | | | |
| 80 | JASPAR | MA1685.1 | MA1685.1. | MHARNGG | 15 | 42281 | 4.60E−33 | CENTRIMO | | |
| | 2022_ | | ARF10 | GAGACAM | | | | | | |
| | CORE_ | | | B | | | | | | |
| | non- | | | (SEQ | | | | | | |
| | redundant_ | | | ID | | | | | | |
| | pfms. | | | NO: | | | | | | |
| | meme | | | 99) | | | | | | |
| 81 | JASPAR | MA0372.1 | MA0372.1. | ACCCCTA | 8 | 42137 | 2.60E−31 | CENTRIMO | | |
| | 2022_ | | RPH1 | A | | | | | | |
| | CORE_ | | | (SEQ | | | | | | |
| | non- | | | ID | | | | | | |
| | redundant_ | | | NO: | | | | | | |
| | pfms. | | | 100 | | | | | | |
| | meme | | | | | | | | | |
| 82 | JASPAR | MA0511.2 | MA0511.2. | WAACCGC | 9 | 47733 | 4.30E−31 | CENTRIMO | | |
| | 2022_ | | RUNX2 | AA | | | | | | |
| | CORE_ | | | (SEQ | | | | | | |
| | non- | | | ID | | | | | | |
| | redundant_ | | | NO: | | | | | | |
| | pfms. | | | 101) | | | | | | |
| | meme | | | | | | | | | |
| 83 | MEME | AGTGCAG | MEME-9 | AGTGCAG | 15 | 2727 | 4.70E−31 | MEME | | |
| | | TGGYRYR | | TGGYRYR | | | | | | |
| | | A | | A | | | | | | |
| | | | | (SEQ | | | | | | |
| | | | | ID | | | | | | |
| | | | | NO: | | | | | | |
| | | | | 102) | | | | | | |
| 84 | JASPAR | MA1892.1 | MA1892.1. | YDBNYNV | 20 | 79903 | 7.10E−31 | CENTRIMO | | |
| | 2022_ | | Tcf3-4-12 | CACCTGN | | | | | | |
| | CORE_ | | | MMVMHV | | | | | | |
| | non- | | | (SEQ | | | | | | |
| | redundant_ | | | ID | | | | | | |
| | pfms. | | | NO: | | | | | | |
| | meme | | | 103 | | | | | | |
| 85 | JASPAR | MA1051.1 | MA1051.1. | GCGCCGC | 8 | 34716 | 7.50E−31 | CENTRIMO | | |
| | 2022_ | | RAP2-3 | C | | | | | | |
| | CORE_ | | | (SEQ | | | | | | |
| | non- | | | ID | | | | | | |
| | redundant_ | | | NO: | | | | | | |
| | pfms. | | | 104) | | | | | | |
| | meme | | | | | | | | | |
| 86 | JASPAR | MA1535.1 | MA1535.1. | NRRGGTC | 9 | 62545 | 1.10E−30 | CENTRIMO | | |
| | 2022_ | | NR2C1 | AN | | | | | | |
| | CORE_ | | | (SEQ | | | | | | |
| | non- | | | ID | | | | | | |
| | redundant_ | | | NO: | | | | | | |
| | pfms. | | | 105) | | | | | | |
| | meme | | | | | | | | | |
| 87 | JASPAR | MA0522.3 | MA0522.3. | NVCACCT | 11 | 71643 | 1.10E−30 | CENTRIMO | | |
| | 2022_ | | TCF3 | GCNN | | | | | | |
| | CORE_ | | | (SEQ | | | | | | |
| | non- | | | ID | | | | | | |
| | redundant_ | | | NO: | | | | | | |
| | pfms. | | | 106) | | | | | | |
| | meme | | | | | | | | | |
| 88 | JASPAR | MA0615.1 | MA0615.1. | BHBBKKA | 17 | 27457 | 1.10E−30 | CENTRIMO | | |
| | 2022_ | | Gmeb1 | CGTMMNW | | | | | | |
| | CORE_ | | | NNN | | | | | | |
| | non- | | | (SEQ | | | | | | |
| | redundant_ | | | ID | | | | | | |
| | pfms. | | | NO: | | | | | | |
| | meme | | | 107) | | | | | | |
| 89 | JASPAR | MA1245.2 | MA1245.2. | DCCGCCG | 11 | 34168 | 5.50E−30 | CENTRIMO | | |
| | 2022_ | | ERF112 | CCRY | | | | | | |
| | CORE_ | | | (SEQ | | | | | | |
| | non- | | | ID | | | | | | |
| | redundant_ | | | NO: | | | | | | |
| | pfms. | | | 108) | | | | | | |
| | meme | | | | | | | | | |
| 90 | JASPAR | MA0744.2 | MA0744.2. | NNWGCAA | 16 | 51641 | 1.20E−29 | CENTRIMO | | |
| | 2022_ | | SCRT2 | CAGGTGD | | | | | | |
| | CORE_ | | | NN | | | | | | |
| | non- | | | (SEQ | | | | | | |
| | redundant_ | | | ID | | | | | | |
| | pfms. | | | NO: | | | | | | |
| | meme | | | 109) | | | | | | |
| 91 | JASPAR | MA0091.1 | MA0091.1. | NSAMCAT | 12 | 25806 | 4.80E−29 | CENTRIMO | | |
| | 2022_ | | TAL1:: | CTGKT | | | | | | |
| | CORE_ | | TCF3 | (SEQ | | | | | | |
| | non- | | | ID | | | | | | |
| | redundant_ | | | NO: | | | | | | |
| | pfms. | | | 110) | | | | | | |
| | meme | | | | | | | | | |
| 92 | JASPAR | MA1460.1 | MA1460.1. | NNATGGC | 11 | 57047 | 1.00E−28 | CENTRIMO | | |
| | 2022_ | | pho | CGNN | | | | | | |
| | CORE_ | | | (SEQ | | | | | | |
| | non- | | | ID | | | | | | |
| | redundant_ | | | NO: | | | | | | |
| | pfms. | | | 111) | | | | | | |
| | meme | | | | | | | | | |
| 93 | JASPAR | MA0582.1 | MA0582.1. | VNGCAAC | 12 | 79907 | 3.10E−28 | CENTRIMO | | |
| | 2022_ | | RAV1 | AKAWD | | | | | | |
| | CORE_ | | | (SEQ | | | | | | |
| | non- | | | ID | | | | | | |
| | redundant_ | | | NO: | | | | | | |
| | pfms. | | | 112) | | | | | | |
| | meme | | | | | | | | | |
| 94 | JASPAR | MA0695.1 | MA0695.1. | RCGACCA | 12 | 69792 | 3.20E−28 | CENTRIMO | | |
| | 2022_ | | ZBTB7C | CCGAN | | | | | | |
| | CORE_ | | | (SEQ | | | | | | |
| | non- | | | ID | | | | | | |
| | redundant_ | | | NO: | | | | | | |
| | pfms. | | | 113) | | | | | | |
| | meme | | | | | | | | | |
| 95 | JASPAR | MA1672.1 | MA1672.1. | NHSACGT | 13 | 51493 | 5.40E−28 | CENTRIMO | | |
| | 2022_ | | GBF2 | GGCANN | | | | | | |
| | CORE_ | | | (SEQ | | | | | | |
| | non- | | | ID | | | | | | |
| | redundant_ | | | NO: | | | | | | |
| | pfms. | | | 114) | | | | | | |
| | meme | | | | | | | | | |
| 96 | JASPAR | MA1570.1 | MA1570.1. | AHCATRT | 10 | 46657 | 5.60E−28 | CENTRIMO | | |
| | 2022_ | | TFAP4 | GDT | | | | | | |
| | CORE_ | | | (SEQ | | | | | | |
| | non- | | | ID | | | | | | |
| | redundant_ | | | NO: | | | | | | |
| | pfms. | | | 115) | | | | | | |
| | meme | | | | | | | | | |
| 97 | JASPAR | MA1005.2 | MA1005.2. | DCCGCCG | 11 | 32149 | 6.10E−28 | CENTRIMO | | |
| | 2022_ | | ERF3 | CCRY | | | | | | |
| | CORE_ | | | (SEQ | | | | | | |
| | non- | | | ID | | | | | | |
| | redundant_ | | | NO: | | | | | | |
| | pfms. | | | 116) | | | | | | |
| | meme | | | | | | | | | |
| 98 | JASPAR | MA0807.1 | MA0807.1. | AGGTGTK | 8 | 95821 | 1.00E−27 | CENTRIMO | | |
| | 2022_ | | TBX5 | A | | | | | | |
| | CORE_ | | | (SEQ | | | | | | |
| | non- | | | ID | | | | | | |
| | redundant_ | | | NO: | | | | | | |
| | pfms. | | | 117) | | | | | | |
| | meme | | | | | | | | | |
| 99 | JASPAR | MA1433.1 | MA1433.1. | VCCCCTD | 8 | 82525 | 7.70E−26 | CENTRIMO | | |
| | 2022_ | | msn-1 | A | | | | | | |
| | CORE_ | | | (SEQ | | | | | | |
| | non- | | | ID | | | | | | |
| | redundant_ | | | NO: | | | | | | |
| | pfms. | | | 118) | | | | | | |
| | meme | | | | | | | | | |
| 100 | JASPAR | MA0123.1 | MA0123.1. | CGSYGCC | 10 | 57863 | 3.50E−25 | CENTRIMO | | |
| | 2022_ | | abi4 | CCC | | | | | | |
| | COREnon- | | | (SEQ | | | | | | |
| | redundant_ | | | ID | | | | | | |
| | pfms. | | | NO: | | | | | | |
| | meme | | | 119) | | | | | | |
| 101 | JASPAR | MA0597.2 | MA0597.2. | VSGCAGG | 12 | 70290 | 4.10E−25 | CENTRIMO | | |
| | 2022_ | | THAP1 | GCASV | | | | | | |
| | COREnon- | | | (SEQ | | | | | | |
| | redundant_ | | | ID | | | | | | |
| | pfms. | | | NO: | | | | | | |
| | meme | | | 120) | | | | | | |
| 102 | JASPAR | MA1049.1 | MA1049.1. | MGCCGCC | 8 | 33683 | 4.30E−25 | CENTRIMO | | |
| | 2022_ | | ERFO94 | R | | | | | | |
| | CORE_ | | | (SEQ | | | | | | |
| | non- | | | ID | | | | | | |
| | redundant_ | | | NO: | | | | | | |
| | pfms. | | | 121) | | | | | | |
| | meme | | | | | | | | | |
| 103 | JASPAR | MA0743.2 | MA0743.2. | NDWKCAA | 16 | 43522 | 7.10E−25 | CENTRIMO | | |
| | 2022_ | | SCRT1 | CAGGTGK | | | | | | |
| | CORE_ | | | NN | | | | | | |
| | non- | | | (SEQ | | | | | | |
| | redundant_ | | | ID | | | | | | |
| | pfms. | | | NO: | | | | | | |
| | meme | | | 122) | | | | | | |
| 104 | JASPAR | MA0103.3 | MA0103.3. | SNCACCT | 11 | 61587 | 1.40E−24 | CENTRIMO | | |
| | 2022_ | | ZEB1 | GSVN | | | | | | |
| | CORE_ | | | (SEQ | | | | | | |
| | non- | | | ID | | | | | | |
| | redundant_ | | | NO: | | | | | | |
| | pfms. | | | 123) | | | | | | |
| | meme | | | | | | | | | |
| 105 | JASPAR | MA0917.1 | MA0917.1. | ATGCGGG | 8 | 72592 | 2.10E−24 | CENTRIMO | | |
| | 2022_ | | gcm2 | Y | | | | | | |
| | CORE_ | | | (SEQ | | | | | | |
| | non- | | | ID | | | | | | |
| | redundant_ | | | NO: | | | | | | |
| | pfms. | | | 124) | | | | | | |
| | meme | | | | | | | | | |
| 106 | JASPAR | MA1615.1 | MA1615.1. | NNCTGGG | 13 | 66385 | 3.00E−24 | CENTRIMO | | |
| | 2022_ | | Plagl1 | GCCABN | | | | | | |
| | CORE_ | | | (SEQ | | | | | | |
| | non- | | | ID | | | | | | |
| | redundant_ | | | NO: | | | | | | |
| | pfms. | | | 125) | | | | | | |
| | meme | | | | | | | | | |
| 107 | JASPAR | MA0545.1 | MA0545.1. | SAACAGC | 11 | 32643 | 3.50E−24 | CENTRIMO | | |
| | 2022_ | | hlh-1 | TGNC | | | | | | |
| | CORE_ | | | (SEQ | | | | | | |
| | non- | | | ID | | | | | | |
| | redundant_ | | | NO: | | | | | | |
| | pfms. | | | 126 | | | | | | |
| | meme | | | | | | | | | |
| 108 | JASPAR | MA1766.1 | MA1766.1. | CRCCGAC | 10 | 76338 | 7.60E−24 | CENTRIMO | | |
| | 2022_ | | RAP2-4 | CAN | | | | | | |
| | CORE_ | | | (SEQ | | | | | | |
| | non- | | | ID | | | | | | |
| | redundant_ | | | NO: | | | | | | |
| | pfms. | | | 127) | | | | | | |
| | meme | | | | | | | | | |
| 109 | JASPAR | MA0816.1 | MA0816.1. | ARCAGCT | 10 | 46494 | 3.50E−23 | CENTRIMO | | |
| | 2022_ | | Ascl2 | GCY | | | | | | |
| | CORE_ | | | (SEQ | | | | | | |
| | non- | | | ID | | | | | | |
| | redundant_ | | | NO: | | | | | | |
| | pfms. | | | 128 | | | | | | |
| | meme | | | | | | | | | |
| 110 | JASPAR | MA1100.2 | MA1100.2. | VGCAGCT | 10 | 73397 | 6.10E−23 | CENTRIMO | | |
| | 2022_ | | ASCL1 | GCN | | | | | | |
| | CORE_ | | | (SEQ | | | | | | |
| | non- | | | ID | | | | | | |
| | redundant_ | | | NO: | | | | | | |
| | pfms. | | | 129) | | | | | | |
| | meme | | | | | | | | | |
| 111 | JASPAR | MA0570.2 | MA0570.2. | ACACGTG | 12 | 26509 | 6.10E−23 | CENTRIMO | | |
| | 2022_ | | ABF1 | KCANN | | | | | | |
| | CORE_ | | | (SEQ | | | | | | |
| | non- | | | ID | | | | | | |
| | redundant_ | | | NO: | | | | | | |
| | pfms. | | | 130) | | | | | | |
| | meme | | | | | | | | | |
| 112 | JASPAR | MA0058.3 | MA0058.3. | AVCACGT | 10 | 29959 | 7.50E−23 | CENTRIMO | | |
| | 2022_ | | MAX | GNY | | | | | | |
| | CORE_ | | | (SEQ | | | | | | |
| | non- | | | ID | | | | | | |
| | redundant_ | | | NO: | | | | | | |
| | pfms. | | | 131) | | | | | | |
| | meme | | | | | | | | | |
| 113 | JASPAR | MA1034.1 | MA1034.1. | CGSCGCC | 8 | 20352 | 7.80E−23 | CENTRIMO | | |
| | 2022_ | | 0s05g | R | | | | | | |
| | CORE_ | | 0497200 | (SEQ | | | | | | |
| | non- | | | ID | | | | | | |
| | redundant_ | | | NO: | | | | | | |
| | pfms. | | | 132) | | | | | | |
| | meme | | | | | | | | | |
| 114 | JASPAR | MA0306.1 | MA0306.1. | HCCCCTW | 9 | 68605 | 5.80E−22 | CENTRIMO | | |
| | 2022_ | | GIS1 | WN | | | | | | |
| | CORE_ | | | (SEQ | | | | | | |
| | non- | | | ID | | | | | | |
| | redundant_ | | | NO: | | | | | | |
| | pfms. | | | 133) | | | | | | |
| | meme | | | | | | | | | |
| 115 | JASPAR | MA1004.1 | MA1004.1. | SGCCGCC | 8 | 31612 | 7.40E−22 | CENTRIMO | | |
| | 2022_ | | ERF13 | R | | | | | | |
| | CORE_ | | | (SEQ | | | | | | |
| | non- | | | ID | | | | | | |
| | redundant_ | | | NO: | | | | | | |
| | pfms. | | | 134) | | | | | | |
| | meme | | | | | | | | | |
| 116 | JASPAR | MA0760.1 | MA0760.1. | ACCGGAA | 10 | 35993 | 1.70E−21 | CENTRIMO | | |
| | 2022_ | | ERF | GTR | | | | | | |
| | CORE_ | | | (SEQ | | | | | | |
| | non- | | | ID | | | | | | |
| | redundant_ | | | NO: | | | | | | |
| | pfms. | | | 135) | | | | | | |
| | meme | | | | | | | | | |
| 117 | JASPAR | MA1990.1 | MA1990.1. | NWCTGAC | 11 | 85328 | 3.10E−21 | CENTRIMO | | |
| | 2022_ | | GLYMA- | ACNN | | | | | | |
| | CORE_ | | 07G038400 | (SEQ | | | | | | |
| | non- | | | ID | | | | | | |
| | redundant_ | | | NO: | | | | | | |
| | pfms. | | | 136) | | | | | | |
| | meme | | | | | | | | | |
| 118 | JASPAR | MA0825.1 | MA0825.1. | RVCACGT | 10 | 35209 | 4.30E−21 | CENTRIMO | | |
| | 2022_ | | MNT | GMH | | | | | | |
| | CORE_ | | | (SEQ | | | | | | |
| | non- | | | ID | | | | | | |
| | redundant_ | | | NO: | | | | | | |
| | pfms. | | | 137) | | | | | | |
| | meme | | | | | | | | | |
| 119 | JASPAR | MA0475.2 | MA0475.2. | ACCGGAA | 10 | 29604 | 4.60E−21 | CENTRIMO | | |
| | 2022_ | | FLI1 | RTR | | | | | | |
| | CORE_ | | | (SEQ | | | | | | |
| | non- | | | ID | | | | | | |
| | redundant_ | | | NO: | | | | | | |
| | pfms. | | | 138) | | | | | | |
| | meme | | | | | | | | | |
| 120 | JASPAR | MA1633.2 | MA1633.2. | ATGACTC | 9 | 21704 | 1.70E−20 | CENTRIMO | | |
| | 2022_ | | BACH1 | AT | | | | | | |
| | CORE_ | | | (SEQ | | | | | | |
| | non- | | | ID | | | | | | |
| | redundant_ | | | NO: | | | | | | |
| | pfms. | | | 139) | | | | | | |
| | meme | | | | | | | | | |
| 121 | JASPAR | MA1878.1 | MA1878.1. | HDGCAGC | 13 | 64266 | 1.80E−20 | CENTRIMO | | |
| | 2022_ | | GRF4 | AGCWDY | | | | | | |
| | CORE_ | | | (SEQ | | | | | | |
| | non- | | | ID | | | | | | |
| | redundant_ | | | NO: | | | | | | |
| | pfms. | | | 140) | | | | | | |
| | meme | | | | | | | | | |
| 122 | JASPAR | MA0521.2 | MA0521.2. | NNACAGC | 12 | 54154 | 2.80E−20 | CENTRIMO | | |
| | 2022_ | | Tcf12 | TGTNN | | | | | | |
| | CORE_ | | | (SEQ | | | | | | |
| | non- | | | ID | | | | | | |
| | redundant_ | | | NO: | | | | | | |
| | pfms. | | | 141) | | | | | | |
| | meme | | | | | | | | | |
| 123 | JASPAR | MA1233.2 | MA1233.2. | HHDCCGC | 15 | 27637 | 5.00E−20 | CENTRIMO | | |
| | 2022_ | | ERFO21 | CGACAHN | | | | | | |
| | COREnon- | | | D | | | | | | |
| | redundant_ | | | (SEQ | | | | | | |
| | pfms. | | | ID | | | | | | |
| | meme | | | NO: | | | | | | |
| | | | | 142) | | | | | | |
| 124 | JASPAR | MA0002.2 | MA0002.2. | BBYTGTG | 11 | 91553 | 6.10E−20 | CENTRIMO | | |
| | 2022_ | | Runx1 | GTTT | | | | | | |
| | CORE_ | | | (SEQ | | | | | | |
| | non- | | | ID | | | | | | |
| | redundant_ | | | NO: | | | | | | |
| | pfms. | | | 143) | | | | | | |
| | meme | | | | | | | | | |
| 125 | JASPAR | MA1484.1 | MA1484.1. | DACCGGA | 10 | 26413 | 1.10E−19 | CENTRIMO | | |
| | 2022_ | | ETS2 | AGY | | | | | | |
| | CORE_ | | | (SEQ | | | | | | |
| | non- | | | ID | | | | | | |
| | redundant_ | | | NO: | | | | | | |
| | pfms. | | | 144) | | | | | | |
| | meme | | | | | | | | | |
| 126 | JASPAR | MA0764.3 | MA0764.3. | ACCGGAA | 10 | 40991 | 2.00E−19 | CENTRIMO | | |
| | 2022_ | | ETV4 | GTR | | | | | | |
| | CORE_ | | | (SEQ | | | | | | |
| | non- | | | ID | | | | | | |
| | redundant_ | | | NO: | | | | | | |
| | pfms. | | | 145} | | | | | | |
| | meme | | | | | | | | | |
| 127 | JASPAR | MA1426.1 | MA1426.1. | NNACGCG | 10 | 52353 | 2.30E−19 | CENTRIMO | | |
| | 2022_ | | MYB124 | CCN | | | | | | |
| | CORE_ | | | (SEQ | | | | | | |
| | non- | | | ID | | | | | | |
| | redundant_ | | | NO: | | | | | | |
| | pfms. | | | 146) | | | | | | |
| | meme | | | | | | | | | |
| 128 | JASPAR | MA1690.1 | MA1690.1. | MARMGGG | 15 | 36453 | 2.50E−19 | CENTRIMO | | |
| | 2022_ | | ARF25 | RGACAMK | | | | | | |
| | CORE_ | | | K | | | | | | |
| | non- | | | (SEQ | | | | | | |
| | redundant_ | | | ID | | | | | | |
| | pfms. | | | NO: | | | | | | |
| | meme | | | 147) | | | | | | |
| 129 | JASPAR | MA2034.1 | MA2034.1. | NNAAACC | 14 | 83326 | 3.50E−19 | CENTRIMO | | |
| | 2022_ | | Bcl11B | ACAARNN | | | | | | |
| | CORE_ | | | | | | | | | |
| | non- | | | (SEQ | | | | | | |
| | redundant_ | | | ID | | | | | | |
| | pfms. | | | NO: | | | | | | |
| | meme | | | 148) | | | | | | |
| 130 | JASPAR | MA0098.3 | MA0098.3. | ACCGGAA | 10 | 43579 | 4.00E−19 | CENTRIMO | | |
| | 2022_ | | ETS1 | RTR | | | | | | |
| | CORE_ | | | (SEQ | | | | | | |
| | non- | | | ID | | | | | | |
| | redundant_ | | | NO: | | | | | | |
| | pfms. | | | 149) | | | | | | |
| | meme | | | | | | | | | |
| 131 | JASPAR | MA1671.1 | MA1671.1. | CDCCGCC | 11 | 26334 | 5.20E−19 | CENTRIMO | | |
| | 2022_ | | ERF118 | GCCR | | | | | | |
| | CORE_ | | | (SEQ | | | | | | |
| | non- | | | ID | | | | | | |
| | redundant_ | | | NO: | | | | | | |
| | pfms. | | | 150) | | | | | | |
| | meme | | | | | | | | | |
| 132 | JASPAR | MA1054.1 | MA1054.1. | YKGGGAC | 10 | 44665 | 6.90E−19 | CENTRIMO | | |
| | 2022_ | | ARALYDR | CAC | | | | | | |
| | CORE_ | | AFT_ | (SEQ | | | | | | |
| | non- | | 897773 | ID | | | | | | |
| | redundant_ | | | NO: | | | | | | |
| | pfms. | | | 151) | | | | | | |
| | meme | | | | | | | | | |
| 133 | JASPAR | MA0130.1 | MA0130.1. | MTCCAC | 6 | 90380 | 1.30E−18 | CENTRIMO | | |
| | 2022_ | | ZNF354C | (SEQ | | | | | | |
| | CORE_ | | | ID | | | | | | |
| | non- | | | NO: | | | | | | |
| | redundant_ | | | 152) | | | | | | |
| | pfms. | | | | | | | | | |
| | meme | | | | | | | | | |
| 134 | JASPAR | MA1619.1 | MA1619.1. | NNACAGC | 12 | 47455 | 1.50E−18 | CENTRIMO | | |
| | 2022_ | | Ptf1A | TGTNN | | | | | | |
| | CORE_ | | | (SEQ | | | | | | |
| | non- | | | ID | | | | | | |
| | redundant_ | | | NO: | | | | | | |
| | pfms. | | | 153) | | | | | | |
| | meme | | | | | | | | | |
| 135 | JASPAR | MA0242.1 | MA0242.1. | WAACCGC | 9 | 24760 | 7.10E−17 | CENTRIMO | | |
| | 2022_ | | Bgb::rur | AA | | | | | | |
| | CORE_ | | | (SEQ | | | | | | |
| | non- | | | ID | | | | | | |
| | redundant_ | | | NO: | | | | | | |
| | pfms. | | | 154) | | | | | | |
| | meme | | | | | | | | | |
| 136 | JASPAR | MA0653.1 | MA0653.1. | AACGAAA | 15 | 2386 | 1.70E−16 | CENTRIMO | | |
| | 2022_ | | IRF9 | CCGAAAC | | | | | | |
| | CORE_ | | | T | | | | | | |
| | non- | | | (SEQ | | | | | | |
| | redundant_ | | | ID | | | | | | |
| | pfms. | | | NO: | | | | | | |
| | meme | | | 155) | | | | | | |
| 137 | JASPAR | MA1483.2 | MA1483.2. | AAMCCGG | 12 | 37695 | 2.60E−16 | CENTRIMO | | |
| | 2022_ | | ELF2 | AAGTR | | | | | | |
| | CORE_ | | | (SEQ | | | | | | |
| | non- | | | ID | | | | | | |
| | redundant_ | | | NO: | | | | | | |
| | pfms. | | | 156) | | | | | | |
| | meme | | | | | | | | | |
| 138 | JASPAR | MA0156.3 | MA0156.3. | VACCGGA | 12 | 16468 | 3.60E−16 | CENTRIMO | | |
| | 2022_ | | FEV | AGTVV | | | | | | |
| | CORE_ | | | (SEQ | | | | | | |
| | non- | | | ID | | | | | | |
| | redundant_ | | | NO: | | | | | | |
| | pfms. | | | 157) | | | | | | |
| | meme | | | | | | | | | |
| 139 | JASPAR | MA0476.1 | MA0476.1. | DVTGAST | 11 | 16714 | 4.30E−16 | CENTRIMO | | |
| | 2022_ | | FOS | CATB | | | | | | |
| | CORE_ | | | (SEQ | | | | | | |
| | non- | | | ID | | | | | | |
| | redundant_ | | | NO: | | | | | | |
| | pfms. | | | 158) | | | | | | |
| | meme | | | | | | | | | |
| 140 | JASPAR | MA1141.1 | MA1141.1. | NKATGAG | 13 | 24318 | 6.70E−16 | CENTRIMO | | |
| | 2022_ | | FOS::JUND | TCATNN | | | | | | |
| | CORE_ | | | (SEQ | | | | | | |
| | non- | | | ID | | | | | | |
| | redundant_ | | | NO: | | | | | | |
| | pfms. | | | 159) | | | | | | |
| | meme | | | | | | | | | |
| 141 | JASPAR | MA0266.1 | MA0266.1. | STCTA | 7 | 31829 | 1.10E−15 | CENTRIMO | | |
| | 2022_ | | ABF2 | GA | | | | | | |
| | CORE_ | | | (SEQ | | | | | | |
| | non- | | | ID | | | | | | |
| | redundant_ | | | NO: | | | | | | |
| | pfms. | | | 160) | | | | | | |
| | meme | | | | | | | | | |
| 142 | JASPAR | MA1001.3 | MA1001.3. | CCGCCGC | 12 | 31852 | 1.40E−15 | CENTRIMO | | |
| | 2022_ | | ERF11 | CRCCD | | | | | | |
| | CORE_ | | | (SEQ | | | | | | |
| | non- | | | ID | | | | | | |
| | redundant_ | | | NO: | | | | | | |
| | pfms. | | | 161) | | | | | | |
| | meme | | | | | | | | | |
| 143 | JASPAR | MA0649.1 | MA0649.1. | GRCACGT | 10 | 30359 | 1.60E−15 | CENTRIMO | | |
| | 2022_ | | HEY2 | GYC | | | | | | |
| | CORE_ | | | (SEQ | | | | | | |
| | non- | | | ID | | | | | | |
| | redundant_ | | | NO: | | | | | | |
| | pfms. | | | 162) | | | | | | |
| | meme | | | | | | | | | |
| 144 | JASPAR | MA0652.1 | MA0652.1. | HCGAAAC | 14 | 2199 | 2.70E−15 | CENTRIMO | | |
| | 2022_ | | IRF8 | CGAAACT | | | | | | |
| | CORE_ | | | (SEQ | | | | | | |
| | non- | | | ID | | | | | | |
| | redundant_ | | | NO: | | | | | | |
| | pfms. | | | 163) | | | | | | |
| | meme | | | | | | | | | |
| 145 | JASPAR | MA0665.1 | MA0665.1. | AACAGCT | 10 | 28247 | 3.20E−15 | CENTRIMO | | |
| | 2022_ | | MSC | GTT | | | | | | |
| | CORE_ | | | (SEQ | | | | | | |
| | non- | | | ID | | | | | | |
| | redundant_ | | | NO: | | | | | | |
| | pfms. | | | 164) | | | | | | |
| | meme | | | | | | | | | |
| 146 | JASPAR | MA1358.1 | MA1358.1. | DKCMACT | 11 | 16773 | 3.80E−15 | CENTRIMO | | |
| | 2022_ | | bHLH130 | TGCM | | | | | | |
| | CORE_ | | | (SEQ | | | | | | |
| | non- | | | ID | | | | | | |
| | redundant_ | | | NO: | | | | | | |
| | pfms. | | | 165) | | | | | | |
| | meme | | | | | | | | | |
| 147 | JASPAR | MA1419.1 | MA1419.1. | HCGAAAC | 15 | 2347 | 4.90E−15 | CENTRIMO | | |
| | 2022_ | | IRF4 | CGAAACY | | | | | | |
| | CORE_ | | | A | | | | | | |
| | non- | | | (SEQ | | | | | | |
| | redundant_ | | | ID | | | | | | |
| | pfms. | | | NO: | | | | | | |
| | meme | | | 166) | | | | | | |
| 148 | JASPAR | MA0692.1 | MA0692.1. | RYCACGT | 10 | 40695 | 6.40E−15 | CENTRIMO | | |
| | 2022_ | | TFEB | GAC | | | | | | |
| | CORE_ | | | (SEQ | | | | | | |
| | non- | | | ID | | | | | | |
| | redundant_ | | | NO: | | | | | | |
| | pfms. | | | 167) | | | | | | |
| | meme | | | | | | | | | |
| 149 | JASPAR | MA0821.2 | MA0821.2. | GRCACGT | 10 | 33670 | 1.60E−14 | CENTRIMO | | |
| | 2022_ | | HES5 | GYC | | | | | | |
| | CORE_ | | | (SEQ | | | | | | |
| | non- | | | ID | | | | | | |
| | redundant_ | | | NO: | | | | | | |
| | pfms. | | | 168) | | | | | | |
| | meme | | | | | | | | | |
| 150 | JASPAR | MA1250.1 | MA1250.1. | CCDCCDC | 15 | 26563 | 1.70E−14 | CENTRIMO | | |
| | 2022_ | | DREB2D | CACCGCC | | | | | | |
| | CORE_ | | | D | | | | | | |
| | non- | | | (SEQ | | | | | | |
| | redundant_ | | | ID | | | | | | |
| | pfms. | | | NO: | | | | | | |
| | meme | | | 169) | | | | | | |
| 151 | JASPAR | MA1972.1 | MA1972.1. | SSCGCCG | 12 | 28561 | 5.30E−14 | CENTRIMO | | |
| | 2022_ | | Zm00001 | CCGCC | | | | | | |
| | CORE_ | | d005892 | (SEQ | | | | | | |
| | non- | | | ID | | | | | | |
| | redundant_ | | | NO: | | | | | | |
| | pfms. | | | 170) | | | | | | |
| | meme | | | | | | | | | |
| 152 | JASPAR | MA1883.1 | MA1883.1. | BKNNNNV | 20 | 37160 | 5.50E−14 | CENTRIMO | | |
| | 2022_ | | Max | CACGTGB | | | | | | |
| | CORE_ | | | NNNNMV | | | | | | |
| | non- | | | (SEQ | | | | | | |
| | redundant_ | | | ID | | | | | | |
| | pfms. | | | NO: | | | | | | |
| | meme | | | 171 | | | | | | |
| 153 | JASPAR | MA0641.1 | MA0641.1. | AACCCGG | 12 | 16647 | 6.20E−14 | CENTRIMO | | |
| | 2022_ | | ELF4 | AAGTR | | | | | | |
| | CORE_ | | | (SEQ | | | | | | |
| | non- | | | ID | | | | | | |
| | redundant_ | | | NO: | | | | | | |
| | pfms. | | | 172 | | | | | | |
| | meme | | | | | | | | | |
| 154 | JASPAR | MA0765.3 | MA0765.3. | ACCGGAA | 10 | 14363 | 9.10E−14 | CENTRIMO | | |
| | 2022_ | | ETV5 | GTR | | | | | | |
| | CORE_ | | | (SEQ | | | | | | |
| | non- | | | ID | | | | | | |
| | redundant_ | | | NO: | | | | | | |
| | pfms. | | | 173 | | | | | | |
| | meme | | | | | | | | | |
| 155 | JASPAR | MA0750.2 | MA0750.2. | NVCCGGA | 13 | 62914 | 9.30E−14 | CENTRIMO | | |
| | 2022_ | | ZBTB7A | AGTGSV | | | | | | |
| | CORE_ | | | (SEQ | | | | | | |
| | non- | | | ID | | | | | | |
| | redundant_ | | | NO: | | | | | | |
| | pfms. | | | 174) | | | | | | |
| | meme | | | | | | | | | |
| 156 | JASPAR | MA1472.2 | MA1472.2. | NVACAGC | 12 | 46672 | 1.00E−13 | CENTRIMO | | |
| | 2022_ | | Bhlha15 | TGTBN | | | | | | |
| | CORE_ | | | (SEQ | | | | | | |
| | non- | | | ID | | | | | | |
| | redundant_ | | | NO: | | | | | | |
| | pfms. | | | 175) | | | | | | |
| | meme | | | | | | | | | |
| 157 | JASPAR | MA0567.1 | MA0567.1. | MGCCGCC | 8 | 36139 | 1.20E−13 | CENTRIMO | | |
| | 2022_ | | ERF1B | A | | | | | | |
| | CORE_ | | | (SEQ | | | | | | |
| | non- | | | ID | | | | | | |
| | redundant_ | | | NO: | | | | | | |
| | pfms. | | | 176) | | | | | | |
| | meme | | | | | | | | | |
| 158 | JASPAR | MA1895.1 | MA1895.1. | NNNNNND | 20 | 54168 | 1.80E−13 | CENTRIMO | | |
| | 2022_ | | Fli-Erg-a | CCGGAAR | | | | | | |
| | CORE_ | | | YNVNNN | | | | | | |
| | non- | | | (SEQ | | | | | | |
| | redundant_ | | | ID | | | | | | |
| | pfms. | | | NO: | | | | | | |
| | meme | | | 177) | | | | | | |
| 159 | JASPAR | MA1134.1 | MA1134.1. | KATGAST | 12 | 23089 | 1.80E−13 | CENTRIMO | | |
| | 2022_ | | FOS::JUNB | CATHN | | | | | | |
| | CORE_ | | | (SEQ | | | | | | |
| | non- | | | ID | | | | | | |
| | redundant_ | | | NO: | | | | | | |
| | pfms. | | | 178) | | | | | | |
| | meme | | | | | | | | | |
| 160 | JASPAR | MA1896.1 | MA1896.1. | NNNNNBR | 22 | 57161 | 1.90E−13 | CENTRIMO | | |
| | 2022_ | | Fli-Erg-b | YTTCCGG | | | | | | |
| | CORE_ | | | TNNNNNN | | | | | | |
| | non- | | | N | | | | | | |
| | redundant_ | | | (SEQ | | | | | | |
| | pfms. | | | ID | | | | | | |
| | meme | | | NO: | | | | | | |
| | | | | 179) | | | | | | |
| 161 | JASPAR | MA1101.2 | MA1101.2. | DWANCAT | 19 | 5291 | 3.60E−13 | CENTRIMO | | |
| | 2022_ | | BACH2 | GASTCAT | | | | | | |
| | CORE_ | | | SNTWH | | | | | | |
| | non- | | | (SEQ | | | | | | |
| | redundant_ | | | ID | | | | | | |
| | pfms. | | | NO: | | | | | | |
| | meme | | | 180) | | | | | | |
| 162 | JASPAR | MA0762.1 | MA0762.1. | AACCGGA | 11 | 22671 | 3.60E−13 | CENTRIMO | | |
| | 2022_ | | ETV2 | AATR | | | | | | |
| | CORE_ | | | (SEQ | | | | | | |
| | non- | | | ID | | | | | | |
| | redundant_ | | | NO: | | | | | | |
| | pfms. | | | 181) | | | | | | |
| | meme | | | | | | | | | |
| 163 | JASPAR | MA0499.2 | MA0499.2. | NNGCACC | 13 | 64360 | 4.70E−13 | CENTRIMO | | |
| | 2022_ | | MYOD1 | TGTCNB | | | | | | |
| | CORE_ | | | (SEQ | | | | | | |
| | non- | | | ID | | | | | | |
| | redundant_ | | | NO: | | | | | | |
| | pfms. | | | 182) | | | | | | |
| | meme | | | | | | | | | |
| 164 | JASPAR | MA1816.1 | MA1816.1. | CCDCCDC | 15 | 28542 | 5.80E−13 | CENTRIMO | | |
| | 2022_ | | ERFO57 | CRCCGCC | | | | | | |
| | CORE_ | | | A | | | | | | |
| | non- | | | (SEQ | | | | | | |
| | redundant_ | | | ID | | | | | | |
| | pfms. | | | NO: | | | | | | |
| | meme | | | 183) | | | | | | |
| 165 | JASPAR | MA0494.1 | MA0494.1. | TGACCTN | 19 | 42262 | 6.50E−13 | CENTRIMO | | |
| | 2022_ | | Nr1h3::Rxra | NAGTRAC | | | | | | |
| | CORE_ | | | CYYDN | | | | | | |
| | non- | | | (SEQ | | | | | | |
| | redundant_ | | | ID | | | | | | |
| | pfms. | | | NO: | | | | | | |
| | meme | | | 184 | | | | | | |
| 166 | JASPAR | MA0986.1 | MA0986.1. | CACCGAC | 8 | 27916 | 7.70E−13 | CENTRIMO | | |
| | 2022_ | | DREB20 | A | | | | | | |
| | CORE_ | | | (SEQ | | | | | | |
| | non- | | | ID | | | | | | |
| | redundant_ | | | NO: | | | | | | |
| | pfms. | | | 185 | | | | | | |
| | meme | | | | | | | | | |
| 167 | JASPAR | MA0608.1 | MA0608.1. | GCCACGT | 9 | 9588 | 1.00E−12 | CENTRIMO | | |
| | 2022_ | | Creb312 | GD | | | | | | |
| | CORE_ | | | (SEQ | | | | | | |
| | non- | | | ID | | | | | | |
| | redundant_ | | | NO: | | | | | | |
| | pfms. | | | 186) | | | | | | |
| | meme | | | | | | | | | |
| 168 | JASPAR | MA0285.1 | MA0285.1. | CNVMGCC | 9 | 94943 | 1.90E−12 | CENTRIMO | | |
| | 2022_ | | CRZ1 | HC | | | | | | |
| | CORE_ | | | (SEQ | | | | | | |
| | non- | | | ID | | | | | | |
| | redundant_ | | | NO: | | | | | | |
| | pfms. | | | 187 | | | | | | |
| | meme | | | | | | | | | |
| 169 | JASPAR | MA0028.2 | MA0028.2. | ACCGGAA | 10 | 15422 | 2.50E−12 | CENTRIMO | | |
| | 2022_ | | ELK1 | GTR | | | | | | |
| | CORE_ | | | (SEQ | | | | | | |
| | non- | | | ID | | | | | | |
| | redundant_ | | | NO: | | | | | | |
| | pfms. | | | 188) | | | | | | |
| | meme | | | | | | | | | |
| 170 | JASPAR | MA0806.1 | MA0806.1. | AGGTGTG | 8 | 76093 | 2.50E−12 | CENTRIMO | | |
| | 2022_ | | TBX4 | A | | | | | | |
| | CORE_ | | | (SEQ | | | | | | |
| | non- | | | ID | | | | | | |
| | redundant_ | | | NO: | | | | | | |
| | pfms. | | | 189) | | | | | | |
| | meme | | | | | | | | | |
| 171 | JASPAR | MA0976.2 | MA0976.2. | CCGCCGC | 12 | 31169 | 2.50E−12 | CENTRIMO | | |
| | 2022_ | | CRF4 | CRCCR | | | | | | |
| | CORE_ | | | (SEQ | | | | | | |
| | non- | | | ID | | | | | | |
| | redundant_ | | | NO: | | | | | | |
| | pfms. | | | 190) | | | | | | |
| | meme | | | | | | | | | |
| 172 | JASPAR | MA1516.1 | MA1516.1. | GRCCRCG | 11 | 31320 | 2.70E−12 | CENTRIMO | | |
| | 2022_ | | KLF3 | CCCH | | | | | | |
| | CORE_ | | | (SEQ | | | | | | |
| | non- | | | ID | | | | | | |
| | redundant_ | | | NO: | | | | | | |
| | pfms. | | | 191) | | | | | | |
| | meme | | | | | | | | | |
| 173 | JASPAR | MA0473.3 | MA0473.3. | RDVCAGG | 14 | 72508 | 3.20E−12 | CENTRIMO | | |
| | 2022_ | | ELF1 | AAGTG | | | | | | |
| | CORE_ | | | VN | | | | | | |
| | non- | | | (SEQ | | | | | | |
| | redundant_ | | | ID | | | | | | |
| | pfms. | | | NO: | | | | | | |
| | meme | | | 192) | | | | | | |
| 174 | JASPAR | MA0655.1 | MA0655.1. | ATGACTC | 9 | 13249 | 3.80E−12 | CENTRIMO | | |
| | 2022_ | | JDP2 | AT | | | | | | |
| | CORE_ | | | (SEQ | | | | | | |
| | non- | | | ID | | | | | | |
| | redundant_ | | | NO: | | | | | | |
| | pfms. | | | 193) | | | | | | |
| | meme | | | | | | | | | |
| 175 | JASPAR | MA1770.1 | MA1770.1. | YGMCAGC | 10 | 78311 | 4.40E−12 | CENTRIMO | | |
| | 2022_ | | BZIP30 | TGK | | | | | | |
| | CORE_ | | | (SEQ | | | | | | |
| | non- | | | ID | | | | | | |
| | redundant_ | | | NO: | | | | | | |
| | pfms. | | | 194 | | | | | | |
| | meme | | | | | | | | | |
| 176 | JASPAR | MA1515.1 | MA1515.1. | NRCCACR | 11 | 66316 | 5.20E−12 | CENTRIMO | | |
| | 2022_ | | KLF2 | CCCH | | | | | | |
| | CORE_ | | | (SEQ | | | | | | |
| | non- | | | ID | | | | | | |
| | redundant_ | | | NO: | | | | | | |
| | pfms. | | | 195) | | | | | | |
| | meme | | | | | | | | | |
| 177 | JASPAR | MA0076.2 | MA0076.2. | BCRCTTC | 11 | 36259 | 5.70E−12 | CENTRIMO | | |
| | 2022_ | | ELK4 | CGGB | | | | | | |
| | CORE_ | | | (SEQ | | | | | | |
| | non- | | | ID | | | | | | |
| | redundant_ | | | NO: | | | | | | |
| | pfms. | | | 196) | | | | | | |
| | meme | | | | | | | | | |
| 178 | JASPAR | MA1659.1 | MA1659.1. | NKCCACG | 12 | 55833 | 9.00E−12 | CENTRIMO | | |
| | 2022_ | | ABF4 | TSDHH | | | | | | |
| | CORE_ | | | (SEQ | | | | | | |
| | non- | | | ID | | | | | | |
| | redundant_ | | | NO: | | | | | | |
| | pfms. | | | 197) | | | | | | |
| | meme | | | | | | | | | |
| 179 | JASPAR | MA1138.1 | MA1138.1. | KRTGAST | 10 | 23003 | 1.40E−11 | CENTRIMO | | |
| | 2022_ | | FOSL2:: | CAT | | | | | | |
| | CORE_ | | JUNB | (SEQ | | | | | | |
| | non- | | | ID | | | | | | |
| | redundant_ | | | NO: | | | | | | |
| | pfms. | | | 198 | | | | | | |
| | meme | | | | | | | | | |
| 180 | JASPAR | MA0995.2 | MA0995.2. | YCRCCGA | 11 | 33596 | 2.50E−11 | CENTRIMO | | |
| | 2022_ | | ERFO39 | CAHN | | | | | | |
| | CORE_ | | | (SEQ | | | | | | |
| | non- | | | ID | | | | | | |
| | redundant_ | | | NO: | | | | | | |
| | pfms. | | | 199) | | | | | | |
| | meme | | | | | | | | | |
| 181 | JASPAR | MA0841.1 | MA0841.1. | VATGACT | 11 | 4456 | 3.20E−11 | CENTRIMO | | |
| | 2022_ | | NFE2 | CATS | | | | | | |
| | CORE_ | | | (SEQ | | | | | | |
| | non- | | | ID | | | | | | |
| | redundant_ | | | NO: | | | | | | |
| | pfms. | | | 200) | | | | | | |
| | meme | | | | | | | | | |
| 182 | JASPAR | MA1721.1 | MA1721.1. | GGYAGCR | 16 | 27220 | 5.70E−11 | CENTRIMO | | |
| | 2022_ | | ZNF93 | GCAGCGG | | | | | | |
| | CORE_ | | | YG | | | | | | |
| | non- | | | (SEQ | | | | | | |
| | redundant_ | | | ID | | | | | | |
| | pfms. | | | NO: | | | | | | |
| | meme | | | 201) | | | | | | |
| 183 | JASPAR | MA1123.2 | MA1123.2. | NNDCCAG | 13 | 69945 | 6.50E−11 | CENTRIMO | | |
| | 2022_ | | TWIST1 | ATGTBN | | | | | | |
| | CORE_ | | | (SEQ | | | | | | |
| | non- | | | ID | | | | | | |
| | redundant_ | | | NO: | | | | | | |
| | pfms. | | | 202) | | | | | | |
| | meme | | | | | | | | | |
| 184 | JASPAR | MA0646.1 | MA0646.1. | BATGCGG | 11 | 35178 | 6.70E−11 | CENTRIMO | | |
| | 2022_ | | GCM1 | GTAC | | | | | | |
| | COREnon- | | | (SEQ | | | | | | |
| | redundant_ | | | ID | | | | | | |
| | pfms. | | | NO: | | | | | | |
| | meme | | | 203) | | | | | | |
| 185 | JASPAR | MA2020.1 | MA2020.1. | NNMMCGA | 14 | 49578 | 1.30E−10 | CENTRIMO | | |
| | 2022_ | | ZBED2 | AACCNNV | | | | | | |
| | CORE_ | | | (SEQ | | | | | | |
| | non- | | | ID | | | | | | |
| | redundant_ | | | NO: | | | | | | |
| | pfms. | | | 204) | | | | | | |
| | meme | | | | | | | | | |
| 186 | JASPAR | MA0645.1 | MA0645.1. | MSCGGAA | 10 | 53426 | 1.30E−10 | CENTRIMO | | |
| | 2022_ | | ETV6 | GTR | | | | | | |
| | CORE_ | | | (SEQ | | | | | | |
| | non- | | | ID | | | | | | |
| | redundant_ | | | NO: | | | | | | |
| | pfms. | | | 205) | | | | | | |
| | meme | | | | | | | | | |
| 187 | JASPAR | MA0500.2 | MA0500.2. | NDRCAGC | 12 | 40714 | 1.60E−10 | CENTRIMO | | |
| | 2022_ | | MYOG | TGYHN | | | | | | |
| | CORE_ | | | (SEQ | | | | | | |
| | non- | | | ID | | | | | | |
| | redundant_ | | | NO: | | | | | | |
| | pfms. | | | 206) | | | | | | |
| | meme | | | | | | | | | |
| 188 | JASPAR | MA0423.1 | MA0423.1. | VCCCCTW | 9 | 49472 | 1.60E−10 | CENTRIMO | | |
| | 2022_ | | YER130C | TH | | | | | | |
| | CORE_ | | | (SEQ | | | | | | |
| | non- | | | ID | | | | | | |
| | redundant_ | | | NO: | | | | | | |
| | pfms. | | | 207 | | | | | | |
| | meme | | | | | | | | | |
| 189 | JASPAR | MA1886.1 | MA1886.1. | NNNNVTC | 20 | 45831 | 1.60E−10 | CENTRIMO | | |
| | 2022_ | | Mitf | ACGTGAY | | | | | | |
| | CORE_ | | | NNNNNN | | | | | | |
| | non- | | | (SEQ | | | | | | |
| | redundant_ | | | ID | | | | | | |
| | pfms. | | | NO: | | | | | | |
| | meme | | | 208) | | | | | | |
| 190 | JASPAR | MA1033.1 | MA1033.1. | MCACGTG | 8 | 21085 | 3.00E−10 | CENTRIMO | | |
| | 2022_ | | OJ1058_ | K | | | | | | |
| | CORE_ | | F05.8 | (SEQ | | | | | | |
| | non- | | | ID | | | | | | |
| | redundant_ | | | NO: | | | | | | |
| | pfms. | | | 209 | | | | | | |
| | meme | | | | | | | | | |
| 191 | JASPAR | MA1686.1 | MA1686.1. | ARCGGGG | 14 | 17070 | 3.10E−10 | CENTRIMO | | |
| | 2022_ | | ARF13 | GACAYGT | | | | | | |
| | CORE_ | | | (SEQ | | | | | | |
| | non- | | | ID | | | | | | |
| | redundant_ | | | NO: | | | | | | |
| | pfms. | | | 210) | | | | | | |
| | meme | | | | | | | | | |
| 192 | JASPAR | MA1144.1 | MA1144.1. | KATGACT | 10 | 27251 | 4.20E−10 | CENTRIMO | | |
| | 2022_ | | FOSL2:: | CAT | | | | | | |
| | CORE_ | | JUND | (SEQ | | | | | | |
| | non- | | | ID | | | | | | |
| | redundant_ | | | NO: | | | | | | |
| | pfms. | | | 211) | | | | | | |
| | meme | | | | | | | | | |
| 193 | JASPAR | MA0258.2 | MA0258.2. | AGGTCAS | 15 | 48304 | 4.30E−10 | CENTRIMO | | |
| | 2022_ | | ESR2 | VNTGMCC | | | | | | |
| | CORE_ | | | Y | | | | | | |
| | non- | | | (SEQ | | | | | | |
| | redundant_ | | | ID | | | | | | |
| | pfms. | | | NO: | | | | | | |
| | meme | | | 212) | | | | | | |
| 194 | JASPAR | MA1558.1 | MA1558.1. | DRCAGGT | 10 | 65055 | 6.70E−10 | CENTRIMO | | |
| | 2022_ | | SNAI1 | GYD | | | | | | |
| | CORE_ | | | (SEQ | | | | | | |
| | non- | | | ID | | | | | | |
| | redundant_ | | | NO: | | | | | | |
| | pfms. | | | 213) | | | | | | |
| | meme | | | | | | | | | |
| 195 | JASPAR | MA0409.1 | MA0409.1. | CACGTGA | 7 | 37816 | 8.70E−10 | CENTRIMO | | |
| | 2022_ | | TYE7 | (SEQ | | | | | | |
| | CORE_ | | | ID | | | | | | |
| | non- | | | NO: | | | | | | |
| | redundant_ | | | 214) | | | | | | |
| | pfms. | | | | | | | | | |
| | meme | | | | | | | | | |
| 196 | JASPAR | MA2001.1 | MA2001.1. | YMTCCAC | 13 | 50204 | 9.70E−10 | CENTRIMO | | |
| | 2022_ | | LBD13 | CGTHDH | | | | | | |
| | CORE_ | | | (SEQ | | | | | | |
| | non- | | | ID | | | | | | |
| | redundant_ | | | NO: | | | | | | |
| | pfms. | | | 215) | | | | | | |
| | meme | | | | | | | | | |
| 197 | JASPAR | MA2059.1 | MA2059.1. | YMTCCAC | 13 | 50204 | 9.70E−10 | CENTRIMO | | |
| | 2022_ | | LBD13 | CGTHDH | | | | | | |
| | CORE_ | | | (SEQ | | | | | | |
| | non- | | | ID | | | | | | |
| | redundant_ | | | NO: | | | | | | |
| | pfms. | | | 216) | | | | | | |
| | meme | | | | | | | | | |
| 198 | JASPAR | MA0332.1 | MA0332.1. | CTGTGG | 6 | 21935 | 1.00E−09 | CENTRIMO | | |
| | 2022_ | | MET28 | (SEQ | | | | | | |
| | CORE_ | | | ID | | | | | | |
| | non- | | | NO: | | | | | | |
| | redundant_ | | | 217) | | | | | | |
| | pfms. | | | | | | | | | |
| | meme | | | | | | | | | |
| 199 | JASPAR | MA0818.2 | MA0818.2. | AMCATAT | 10 | 12093 | 1.00E−09 | CENTRIMO | | |
| | 2022_ | | BHLHE22 | GKY | | | | | | |
| | CORE_ | | | (SEQ | | | | | | |
| | non- | | | ID | | | | | | |
| | redundant_ | | | NO: | | | | | | |
| | pfms. | | | 218) | | | | | | |
| | meme | | | | | | | | | |
| 200 | JASPAR | MA0736.1 | MA0736.1. | GACCCCC | 14 | 14975 | 1.20E−09 | CENTRIMO | | |
| | 2022_ | | GLIS2 | CGCRAMG | | | | | | |
| | CORE_ | | | (SEQ | | | | | | |
| | non- | | | ID | | | | | | |
| | redundant_ | | | NO: | | | | | | |
| | pfms. | | | 219) | | | | | | |
| | meme | | | | | | | | | |
| 201 | JASPAR | MA0551.1 | MA0551.1. | NNTGMCA | 16 | 7764 | 1.20E−09 | CENTRIMO | | |
| | 2022_ | | HY5 | CGTGKCA | | | | | | |
| | CORE_ | | | NN | | | | | | |
| | non- | | | (SEQ | | | | | | |
| | redundant_ | | | ID | | | | | | |
| | pfms. | | | NO: | | | | | | |
| | meme | | | 220) | | | | | | |
| 202 | JASPAR | MA1554.1 | MA1554.1. | CGTTGCY | 9 | 70601 | 1.40E−09 | CENTRIMO | | |
| | 2022_ | | RFX7 | AY | | | | | | |
| | CORE_ | | | (SEQ | | | | | | |
| | non- | | | ID | | | | | | |
| | redundant_ | | | NO: | | | | | | |
| | pfms. | | | 221) | | | | | | |
| | meme | | | | | | | | | |
| 203 | JASPAR | MA1932.1 | MA1932.1. | NNNNNHR | 20 | 77739 | 1.40E−09 | CENTRIMO | | |
| | 2022_ | | Snail | CACCTGY | | | | | | |
| | CORE_ | | | HNNNNN | | | | | | |
| | non- | | | (SEQ | | | | | | |
| | redundant_ | | | ID | | | | | | |
| | pfms. | | | NO: | | | | | | |
| | meme | | | 222) | | | | | | |
| 204 | JASPAR | MA1593.1 | MA1593.1. | WVACAGC | 12 | 71614 | 1.70E−09 | CENTRIMO | | |
| | 2022_ | | ZNF317 | AGAYW | | | | | | |
| | CORE_ | | | (SEQ | | | | | | |
| | non- | | | ID | | | | | | |
| | redundant_ | | | NO: | | | | | | |
| | pfms. | | | 223) | | | | | | |
| | meme | | | | | | | | | |
| 205 | JASPAR | MA0449.1 | MA0449.1.h | GGCACGT | 10 | 36396 | 2.60E−09 | CENTRIMO | | |
| | 2022_ | | | GCC | | | | | | |
| | CORE_ | | | (SEQ | | | | | | |
| | non- | | | ID | | | | | | |
| | redundant_ | | | NO: | | | | | | |
| | pfms. | | | 224) | | | | | | |
| | meme | | | | | | | | | |
| 206 | JASPAR | MA1564.1 | MA1564.1. | RCCACGC | 12 | 57126 | 2.80E−09 | CENTRIMO | | |
| | 2022_ | | SP9 | CCMCY | | | | | | |
| | CORE_ | | | (SEQ | | | | | | |
| | non- | | | ID | | | | | | |
| | redundant_ | | | NO: | | | | | | |
| | pfms. | | | 225) | | | | | | |
| | meme | | | | | | | | | |
| 207 | JASPAR | MA1641.1 | MA1641.1. | NVACAGC | 12 | 46584 | 3.30E−09 | CENTRIMO | | |
| | 2022_ | | MYF5 | TGTBN | | | | | | |
| | CORE_ | | | (SEQ | | | | | | |
| | non- | | | ID | | | | | | |
| | redundant_ | | | NO: | | | | | | |
| | pfms. | | | 226) | | | | | | |
| | meme | | | | | | | | | |
| 208 | JASPAR | MA0759.2 | MA0759.2. | ACCGGAA | 11 | 13130 | 3.70E−09 | CENTRIMO | | |
| | 2022_ | | ELK3 | GTRV | | | | | | |
| | CORE_ | | | (SEQ | | | | | | |
| | non- | | | ID | | | | | | |
| | redundant_ | | | NO: | | | | | | |
| | pfms. | | | 227) | | | | | | |
| | meme | | | | | | | | | |
| 209 | JASPAR | MA0803.1 | MA0803.1. | AGGTGTG | 8 | 41361 | 4.00E−09 | CENTRIMO | | |
| | 2022_ | | TBX15 | A | | | | | | |
| | CORE_ | | | (SEQ | | | | | | |
| | non- | | | ID | | | | | | |
| | redundant_ | | | NO: | | | | | | |
| | pfms. | | | 228) | | | | | | |
| | meme | | | | | | | | | |
| 210 | JASPAR | MA1517.1 | MA1517.1. | NRCCACG | 11 | 51358 | 5.30E−09 | CENTRIMO | | |
| | 2022_ | | KLF6 | CCCH | | | | | | |
| | CORE_ | | | (SEQ | | | | | | |
| | non- | | | ID | | | | | | |
| | redundant_ | | | NO: | | | | | | |
| | pfms. | | | 229) | | | | | | |
| | meme | | | | | | | | | |
| 211 | JASPAR | MA1618.1 | MA1618.1. | NNACAGA | 13 | 70708 | 5.60E−09 | CENTRIMO | | |
| | 2022_ | | Ptf1a | TGTTNN | | | | | | |
| | CORE_ | | | (SEQ | | | | | | |
| | non- | | | ID | | | | | | |
| | redundant_ | | | NO: | | | | | | |
| | pfms. | | | 230) | | | | | | |
| | meme | | | | | | | | | |
| 212 | JASPAR | MA0381.1 | MA0381.1. | GGCCRN | 6 | 67499 | 5.60E−09 | CENTRIMO | | |
| | 2022_ | | SKN7 | (SEQ | | | | | | |
| | CORE_ | | | ID | | | | | | |
| | non- | | | NO: | | | | | | |
| | redundant_ | | | 231) | | | | | | |
| | pfms. | | | | | | | | | |
| | meme | | | | | | | | | |
| 213 | JASPAR | MA0686.1 | MA0686.1. | AMCCGGA | 11 | 14132 | 6.10E−09 | CENTRIMO | | |
| | 2022_ | | SPDEF | TGTR | | | | | | |
| | CORE_ | | | (SEQ | | | | | | |
| | non- | | | ID | | | | | | |
| | redundant_ | | | NO: | | | | | | |
| | pfms. | | | 232) | | | | | | |
| | meme | | | | | | | | | |
| 214 | JASPAR | MA1474.1 | MA1474.1. | YGCCACG | 12 | 43612 | 7.10E−09 | CENTRIMO | | |
| | 2022_ | | CREB3L4 | TCAYC | | | | | | |
| | CORE_ | | | (SEQ | | | | | | |
| | non- | | | ID | | | | | | |
| | redundant_ | | | NO: | | | | | | |
| | pfms. | | | 233) | | | | | | |
| | meme | | | | | | | | | |
| 215 | JASPAR | MA0664.1 | MA0664.1. | RTCACGT | 10 | 25631 | 7.90E−09 | CENTRIMO | | |
| | 2022_ | | MLXIPL | GAT | | | | | | |
| | CORE_ | | | (SEQ | | | | | | |
| | non- | | | ID | | | | | | |
| | redundant_ | | | NO: | | | | | | |
| | pfms. | | | 234) | | | | | | |
| | meme | | | | | | | | | |
| 216 | JASPAR | MA0640.2 | MA0640.2. | NNCCACT | 14 | 83934 | 1.00E−08 | CENTRIMO | | |
| | 2022_ | | ELF3 | TCCTGNT | | | | | | |
| | CORE_ | | | (SEQ | | | | | | |
| | non- | | | ID | | | | | | |
| | redundant_ | | | NO: | | | | | | |
| | pfms. | | | 235) | | | | | | |
| | meme | | | | | | | | | |
| 217 | JASPAR | MA1973.1 | MA1973.1. | CCGCCGC | 13 | 30422 | 1.40E−08 | CENTRIMO | | |
| | 2022_ | | Zm00001 | CGCCGC | | | | | | |
| | COREnon- | | d020267 | (SEQ | | | | | | |
| | redundant_ | | | ID | | | | | | |
| | pfms. | | | NO: | | | | | | |
| | meme | | | 236) | | | | | | |
| 218 | JASPAR | MA0267.1 | MA0267.1. | MCCAGCA | 7 | 78570 | 1.90E−08 | CENTRIMO | | |
| | 2022_ | | ACE2 | (SEQ | | | | | | |
| | CORE_ | | | ID | | | | | | |
| | non- | | | NO: | | | | | | |
| | redundant_ | | | 237) | | | | | | |
| | pfms. | | | | | | | | | |
| | meme | | | | | | | | | |
| 219 | JASPAR | MA1977.1 | MA1977.1. | CSCCGCC | 16 | 31173 | 2.30E−08 | CENTRIMO | | |
| | 2022_ | | Zm00001 | GCCGCCR | | | | | | |
| | CORE_ | | d049364 | CC | | | | | | |
| | non- | | | (SEQ | | | | | | |
| | redundant_ | | | ID | | | | | | |
| | pfms. | | | NO: | | | | | | |
| | meme | | | 238) | | | | | | |
| 220 | JASPAR | MA1485.1 | MA1485.1. | GCRMCAG | 14 | 8769 | 2.40E−08 | CENTRIMO | | |
| | 2022_ | | FERD3L | CTGTYAC | | | | | | |
| | CORE_ | | | (SEQ | | | | | | |
| | non- | | | ID | | | | | | |
| | redundant_ | | | NO: | | | | | | |
| | pfms. | | | 239) | | | | | | |
| | meme | | | | | | | | | |
| 221 | JASPAR | MA0062.3 | MA0062.3. | NNCACTT | 14 | 84572 | 2.50E−08 | CENTRIMO | | |
| | 2022_ | | GABPA | CCTGTNN | | | | | | |
| | CORE_ | | | (SEQ | | | | | | |
| | non- | | | ID | | | | | | |
| | redundant_ | | | NO: | | | | | | |
| | pfms. | | | 240) | | | | | | |
| | meme | | | | | | | | | |
| 222 | JASPAR | MA1475.1 | MA1475.1. | GRTGACG | 12 | 22955 | 3.30E−08 | CENTRIMO | | |
| | 2022_ | | CREB3L4 | TCAYC | | | | | | |
| | CORE_ | | | (SEQ | | | | | | |
| | non- | | | ID | | | | | | |
| | redundant_ | | | NO: | | | | | | |
| | pfms. | | | 241) | | | | | | |
| | meme | | | | | | | | | |
| 223 | JASPAR | MA1418.1 | MA1418.1. | NSRRAAM | 21 | 6790 | 3.80E−08 | CENTRIMO | | |
| | 2022_ | | IRF3 | GGAAACC | | | | | | |
| | CORE_ | | | GAAACYR | | | | | | |
| | non- | | | (SEQ | | | | | | |
| | redundant_ | | | ID | | | | | | |
| | pfms. | | | NO: | | | | | | |
| | meme | | | 242) | | | | | | |
| 224 | JASPAR | MA0474.3 | MA0474.3. | NNACAGG | 14 | 76517 | 4.30E−08 | CENTRIMO | | |
| | 2022_ | | Erg | AAGTGVN | | | | | | |
| | CORE_ | | | (SEQ | | | | | | |
| | non- | | | ID | | | | | | |
| | redundant_ | | | NO: | | | | | | |
| | pfms. | | | 243) | | | | | | |
| | meme | | | | | | | | | |
| 225 | JASPAR | MA1726.1 | MA1726.1. | NMYTGCA | 14 | 50646 | 4.60E−08 | CENTRIMO | | |
| | 2022_ | | ZNF331 | GAGCCCH | | | | | | |
| | CORE_ | | | (SEQ | | | | | | |
| | non- | | | ID | | | | | | |
| | redundant_ | | | NO: | | | | | | |
| | pfms. | | | 244) | | | | | | |
| | meme | | | | | | | | | |
| 226 | JASPAR | MA1865.1 | MA1865.1. | VGSCTAG | 15 | 27474 | 5.10E−08 | CENTRIMO | | |
| | 2022_ | | ZNF574 | AGMGGCC | | | | | | |
| | CORE_ | | | S | | | | | | |
| | non- | | | (SEQ | | | | | | |
| | redundant_ | | | ID | | | | | | |
| | pfms. | | | NO: | | | | | | |
| | meme | | | 245) | | | | | | |
| 227 | JASPAR | MA0734.3 | MA0734.3. | NRGACCA | 13 | 47726 | 6.20E−08 | CENTRIMO | | |
| | 2022_ | | Gli2 | CCCASV | | | | | | |
| | CORE_ | | | (SEQ | | | | | | |
| | non- | | | ID | | | | | | |
| | redundant_ | | | NO: | | | | | | |
| | pfms. | | | 246) | | | | | | |
| | meme | | | | | | | | | |
| 228 | JASPAR | MA0775.1 | MA0775.1. | DTGACAG | 8 | 82127 | 6.30E−08 | CENTRIMO | | |
| | 2022_ | | MEIS3 | S | | | | | | |
| | CORE_ | | | (SEQ | | | | | | |
| | non- | | | ID | | | | | | |
| | redundant_ | | | NO: | | | | | | |
| | pfms. | | | 247) | | | | | | |
| | meme | | | | | | | | | |
| 229 | JASPAR | MA1135.1 | MA1135.1. | KRTGAST | 10 | 27501 | 7.10E−08 | CENTRIMO | | |
| | 2022_ | | FOSB::JUNB | CAT | | | | | | |
| | CORE_ | | | (SEQ | | | | | | |
| | non- | | | ID | | | | | | |
| | redundant_ | | | NO: | | | | | | |
| | pfms. | | | 248 | | | | | | |
| | meme | | | | | | | | | |
| 230 | JASPAR | MA2042.1 | MA2042.1. | NNTCGTG | 11 | 64093 | 7.80E−08 | CENTRIMO | | |
| | 2022_ | | Npas4 | ACHN | | | | | | |
| | CORE_ | | | (SEQ | | | | | | |
| | non- | | | ID | | | | | | |
| | redundant_ | | | NO: | | | | | | |
| | pfms. | | | 249) | | | | | | |
| | meme | | | | | | | | | |
| 231 | JASPAR | MA0747.1 | MA0747.1. | RCCACGC | 12 | 61372 | 8.20E−08 | CENTRIMO | | |
| | 2022_ | | SP8 | CCMCY | | | | | | |
| | CORE_ | | | (SEQ | | | | | | |
| | non- | | | ID | | | | | | |
| | redundant_ | | | NO: | | | | | | |
| | pfms. | | | 250) | | | | | | |
| | meme | | | | | | | | | |
| 232 | JASPAR | MA1231.2 | MA1231.2. | YHTYMGC | 14 | 32785 | 8.30E−08 | CENTRIMO | | |
| | 2022_ | | ERF15 | CGCCDYN | | | | | | |
| | CORE_ | | | | | | | | | |
| | non- | | | (SEQ | | | | | | |
| | redundant_ | | | ID | | | | | | |
| | pfms. | | | NO: | | | | | | |
| | meme | | | 251) | | | | | | |
| 233 | JASPAR | MA0607.2 | MA0607.2. | ACCATAT | 10 | 14336 | 9.90E−08 | CENTRIMO | | |
| | 2022_ | | BHLHA15 | GGT | | | | | | |
| | CORE_ | | | (SEQ | | | | | | |
| | non- | | | ID | | | | | | |
| | redundant_ | | | NO: | | | | | | |
| | pfms. | | | 252 | | | | | | |
| | meme | | | | | | | | | |
| 234 | JASPAR | MA1842.1 | MA1842.1. | YCACCAA | 11 | 72806 | 1.00E−07 | CENTRIMO | | |
| | 2022_ | | MYB83 | CMNC | | | | | | |
| | CORE_ | | | (SEQ | | | | | | |
| | non- | | | ID | | | | | | |
| | redundant_ | | | NO: | | | | | | |
| | pfms. | | | 253) | | | | | | |
| | meme | | | | | | | | | |
| 235 | JASPAR | MA0395.1 | MA0395.1. | YNANYGG | 20 | 26220 | 1.50E−07 | CENTRIMO | | |
| | 2022_ | | STP2 | CGCCGYR | | | | | | |
| | CORE_ | | | YVNMBH | | | | | | |
| | non- | | | (SEQ | | | | | | |
| | redundant_ | | | ID | | | | | | |
| | pfms. | | | NO: | | | | | | |
| | meme | | | 254) | | | | | | |
| 236 | JASPAR | MA1803.1 | MA1803.1. | RWMAACA | 14 | 41898 | 1.80E−07 | CENTRIMO | | |
| | 2022_ | | FOXO1:: | GGAAGTD | | | | | | |
| | CORE_ | | ELK1 | (SEQ | | | | | | |
| | non- | | | ID | | | | | | |
| | redundant_ | | | NO: | | | | | | |
| | pfms. | | | 255) | | | | | | |
| | meme | | | | | | | | | |
| 237 | JASPAR | MA0048.2 | MA0048.2. | CGCAGCT | 10 | 34260 | 1.80E−07 | CENTRIMO | | |
| | 2022_ | | NHLH1 | GCK | | | | | | |
| | CORE_ | | | (SEQ | | | | | | |
| | non- | | | ID | | | | | | |
| | redundant_ | | | NO: | | | | | | |
| | pfms. | | | 256) | | | | | | |
| | meme | | | | | | | | | |
| 238 | JASPAR | MA1958.1 | MA1958.1. | NNNNRRC | 20 | 77164 | 2.20E−07 | CENTRIMO | | |
| | 2022_ | | Atoh7 | AGCTGTY | | | | | | |
| | CORE_ | | | NNNNNN | | | | | | |
| | non- | | | (SEQ | | | | | | |
| | redundant_ | | | ID | | | | | | |
| | pfms. | | | NO: | | | | | | |
| | meme | | | 257) | | | | | | |
| 239 | JASPAR | MA1916.1 | MA1916.1. | NNNNNGR | 22 | 42047 | 2.20E−07 | CENTRIMO | | |
| | 2022_ | | Hey | CACGTGC | | | | | | |
| | CORE_ | | | CNNNNNN | | | | | | |
| | non- | | | N | | | | | | |
| | redundant_ | | | (SEQ | | | | | | |
| | pfms. | | | ID | | | | | | |
| | meme | | | NO: | | | | | | |
| | | | | 258) | | | | | | |
| 240 | JASPAR | MA1349.1 | MA1349.1. | DDWKSHS | 15 | 6487 | 2.30E−07 | CENTRIMO | | |
| | 2022_ | | BZIP16 | ACGTGGC | | | | | | |
| | CORE_ | | | A | | | | | | |
| | non- | | | (SEQ | | | | | | |
| | redundant_ | | | ID | | | | | | |
| | pfms. | | | NO: | | | | | | |
| | meme | | | 259) | | | | | | |
| 241 | JASPAR | MA1420.1 | MA1420.1. | CCGAAAC | 14 | 25311 | 2.40E−07 | CENTRIMO | | |
| | 2022_ | | IRF5 | CGAAACY | | | | | | |
| | COREnon- | | | (SEQ | | | | | | |
| | redundant_ | | | ID | | | | | | |
| | pfms. | | | NO: | | | | | | |
| | meme | | | 260) | | | | | | |
| 242 | JASPAR | MA0763.1 | MA0763.1. | ACCGGAA | 10 | 49343 | 2.40E−07 | CENTRIMO | | |
| | 2022_ | | ETV3 | GTR | | | | | | |
| | CORE_ | | | (SEQ | | | | | | |
| | non- | | | ID | | | | | | |
| | redundant_ | | | NO: | | | | | | |
| | pfms. | | | 261) | | | | | | |
| | meme | | | | | | | | | |
| 243 | JASPAR | MA0669.1 | MA0669.1. | RACATAT | 10 | 13681 | 2.40E−07 | CENTRIMO | | |
| | 2022_ | | NEUROG2 | GTC | | | | | | |
| | CORE_ | | | (SEQ | | | | | | |
| | non- | | | ID | | | | | | |
| | redundant_ | | | NO: | | | | | | |
| | pfms. | | | 262 | | | | | | |
| | meme | | | | | | | | | |
| 244 | MEME | TTCACAT | MEME-10 | TTCACAT | 15 | 430 | 2.60E−07 | MEME | | |
| | | AAAAACT | | AAAAACT | | | | | | |
| | | A | | A | | | | | | |
| | | (SEQ | | (SEQ | | | | | | |
| | | ID | | ID | | | | | | |
| | | NO: | | NO: | | | | | | |
| | | 263) | | 263) | | | | | | |
| 245 | JASPAR | MA0303.2 | MA0303.2. | NATGACT | 11 | 48470 | 2.80E−07 | CENTRIMO | | |
| | 2022_ | | GCN4 | CATH | | | | | | |
| | CORE_ | | | (SEQ | | | | | | |
| | non- | | | ID | | | | | | |
| | redundant_ | | | NO: | | | | | | |
| | pfms. | | | 264) | | | | | | |
| | meme | | | | | | | | | |
| 246 | JASPAR | MA0034.1 | MA0034.1. | SVYAACC | 10 | 70007 | 3.00E−07 | CENTRIMO | | |
| | 2022_ | | Gam1 | GMC | | | | | | |
| | CORE_ | | | (SEQ | | | | | | |
| | non- | | | ID | | | | | | |
| | redundant_ | | | NO: | | | | | | |
| | pfms. | | | 265) | | | | | | |
| | meme | | | | | | | | | |
| 247 | JASPAR | MA0374.1 | MA0374.1. | CGCGCVN | 7 | 20244 | 3.40E−07 | CENTRIMO | | |
| | 2022_ | | RSC3 | (SEQ | | | | | | |
| | CORE_ | | | ID | | | | | | |
| | non- | | | NO: | | | | | | |
| | redundant_ | | | 266) | | | | | | |
| | pfms. | | | | | | | | | |
| | meme | | | | | | | | | |
| 248 | JASPAR | MA0941.1 | MA0941.1. | NNNDACA | 13 | 43939 | 3.70E−07 | CENTRIMO | | |
| | 2022_ | | ABF2 | CGTGDN | | | | | | |
| | CORE_ | | | (SEQ | | | | | | |
| | non- | | | ID | | | | | | |
| | redundant_ | | | NO: | | | | | | |
| | pfms. | | | 267) | | | | | | |
| | meme | | | | | | | | | |
| 249 | JASPAR | MA0832.1 | MA0832.1. | RYAACAG | 14 | 6506 | 4.30E−07 | CENTRIMO | | |
| | 2022_ | | Tcf21 | CTGTTRN | | | | | | |
| | CORE_ | | | (SEQ | | | | | | |
| | non- | | | ID | | | | | | |
| | redundant_ | | | NO: | | | | | | |
| | pfms. | | | 268) | | | | | | |
| | meme | | | | | | | | | |
| 250 | JASPAR | MA1222.1 | MA1222.1. | CCDCCDC | 15 | 15902 | 6.40E−07 | CENTRIMO | | |
| | 2022_ | | ERFO14 | CACCGMC | | | | | | |
| | CORE_ | | | A | | | | | | |
| | non- | | | (SEQ | | | | | | |
| | redundant_ | | | ID | | | | | | |
| | pfms. | | | NO: | | | | | | |
| | meme | | | 269) | | | | | | |
| 251 | JASPAR | MA1638.1 | MA1638.1. | NVCAGAT | 10 | 27700 | 6.50E−07 | CENTRIMO | | |
| | 2022_ | | HAND2 | GNN | | | | | | |
| | CORE_ | | | (SEQ | | | | | | |
| | non- | | | ID | | | | | | |
| | redundant_ | | | NO: | | | | | | |
| | pfms. | | | 270} | | | | | | |
| | meme | | | | | | | | | |
| 252 | JASPAR | MA0394.1 | MA0394.1. | YGCGGCK | 8 | 25905 | 6.60E−07 | CENTRIMO | | |
| | 2022_ | | STP1 | B | | | | | | |
| | CORE_ | | | (SEQ | | | | | | |
| | non- | | | ID | | | | | | |
| | redundant_ | | | NO: | | | | | | |
| | pfms. | | | 271} | | | | | | |
| | meme | | | | | | | | | |
| 253 | JASPAR | MA0865.2 | MA0865.2. | TTCCCGC | 12 | 40782 | 6.70E−07 | CENTRIMO | | |
| | 2022_ | | E2F8 | CAHWA | | | | | | |
| | CORE_ | | | (SEQ | | | | | | |
| | non- | | | ID | | | | | | |
| | redundant_ | | | NO: | | | | | | |
| | pfms. | | | 272) | | | | | | |
| | meme | | | | | | | | | |
| 254 | JASPAR | MA0975.1 | MA0975.1. | SCGCCGC | 8 | 21119 | 7.20E−07 | CENTRIMO | | |
| | 2022_ | | CRF2 | C | | | | | | |
| | COREnon- | | | (SEQ | | | | | | |
| | redundant_ | | | ID | | | | | | |
| | pfms. | | | NO: | | | | | | |
| | meme | | | 273) | | | | | | |
| 255 | JASPAR | MA1405.1 | MA1405.1. | BACTGAC | 10 | 43190 | 8.20E−07 | CENTRIMO | | |
| | 2022_ | | SIZF2 | AGT | | | | | | |
| | CORE_ | | | (SEQ | | | | | | |
| | non- | | | ID | | | | | | |
| | redundant_ | | | NO: | | | | | | |
| | pfms. | | | 274) | | | | | | |
| | meme | | | | | | | | | |
| 256 | JASPAR | MA1428.1 | MA1428.1. | BGGSCCC | 9 | 88643 | 8.50E−07 | CENTRIMO | | |
| | 2022_ | | TCP8 | AC | | | | | | |
| | CORE_ | | | (SEQ | | | | | | |
| | non- | | | ID | | | | | | |
| | redundant_ | | | NO: | | | | | | |
| | pfms. | | | 275) | | | | | | |
| | meme | | | | | | | | | |
| 257 | JASPAR | MA1225.1 | MA1225.1. | CCDCCGC | 15 | 24831 | 9.50E−07 | CENTRIMO | | |
| | 2022_ | | ERF5 | CGCCGCC | | | | | | |
| | CORE_ | | | R | | | | | | |
| | non- | | | (SEQ | | | | | | |
| | redundant_ | | | ID | | | | | | |
| | pfms. | | | NO: | | | | | | |
| | meme | | | 276) | | | | | | |
| 258 | JASPAR | MA1228.1 | MA1228.1. | RYGGCGG | 17 | 14123 | 1.00E−06 | CENTRIMO | | |
| | 2022_ | | ERFO91 | CGGHGGH | | | | | | |
| | CORE_ | | | GGH | | | | | | |
| | non- | | | (SEQ | | | | | | |
| | redundant_ | | | ID | | | | | | |
| | pfms. | | | NO: | | | | | | |
| | meme | | | 277) | | | | | | |
| 259 | JASPAR | MA0089.2 | MA0089.2. | NVNATGA | 16 | 15829 | 1.00E−06 | CENTRIMO | | |
| | 2022_ | | MAFG:: | CTCAGCA | | | | | | |
| | COREnon- | | NFE2L1 | DW | | | | | | |
| | redundant_ | | | (SEQ | | | | | | |
| | pfms. | | | ID | | | | | | |
| | meme | | | NO: | | | | | | |
| | | | | 278) | | | | | | |
| 260 | JASPAR | MA0079.5 | MA0079.5. | GGGGGGG | 9 | 33669 | 1.10E−06 | CENTRIMO | | |
| | 2022_ | | SP1 | G | | | | | | |
| | CORE_ | | | (SEQ | | | | | | |
| | non- | | | ID | | | | | | |
| | redundant_ | | | NO: | | | | | | |
| | pfms. | | | 279) | | | | | | |
| | meme | | | | | | | | | |
| 261 | JASPAR | MA1698.1 | MA1698.1. | MCWGCCG | 14 | 34146 | 1.10E−06 | CENTRIMO | | |
| | 2022_ | | ARF7 | ACAAGSH | | | | | | |
| | CORE_ | | | (SEQ | | | | | | |
| | non- | | | ID | | | | | | |
| | redundant_ | | | NO: | | | | | | |
| | pfms. | | | 280) | | | | | | |
| | meme | | | | | | | | | |
| 262 | JASPAR | MA0145.2 | MA0145.2. | CCAGYYY | 14 | 60361 | 1.20E−06 | CENTRIMO | | |
| | 2022_ | | Tfcp211 | VADCCRG | | | | | | |
| | CORE_ | | | (SEQ | | | | | | |
| | non- | | | ID | | | | | | |
| | redundant_ | | | NO: | | | | | | |
| | pfms. | | | 281) | | | | | | |
| | meme | | | | | | | | | |
| 263 | JASPAR | MA1914.1 | MA1914.1. | NNNNNNN | 22 | 55501 | 1.40E−06 | CENTRIMO | | |
| | 2022_ | | Hes-b | GGCACGT | | | | | | |
| | CORE_ | | | GBBNNNN | | | | | | |
| | non- | | | N | | | | | | |
| | redundant_ | | | (SEQ | | | | | | |
| | pfms. | | | ID | | | | | | |
| | meme | | | NO: | | | | | | |
| | | | | 282) | | | | | | |
| 264 | JASPAR | MA0477.2 | MA0477.2. | NNATGAC | 13 | 35637 | 1.50E−06 | CENTRIMO | | |
| | 2022_ | | FOSL1 | TCATNN | | | | | | |
| | CORE_ | | | (SEQ | | | | | | |
| | non- | | | ID | | | | | | |
| | redundant_ | | | NO: | | | | | | |
| | pfms. | | | 283) | | | | | | |
| | meme | | | | | | | | | |
| 265 | JASPAR | MA2046.1 | MA2046.1. | NNRCAGG | 15 | 80407 | 1.70E−06 | CENTRIMO | | |
| | 2022_ | | Ikzf3 | AAGTGGV | | | | | | |
| | CORE_ | | | N | | | | | | |
| | non- | | | (SEQ | | | | | | |
| | redundant_ | | | ID | | | | | | |
| | pfms. | | | NO: | | | | | | |
| | meme | | | 284) | | | | | | |
| 266 | JASPAR | MA1031.1 | MA1031.1. | KKGGGCC | 10 | 51696 | 2.00E−06 | CENTRIMO | | |
| | 2022_ | | 0J1581_ | CMM | | | | | | |
| | CORE_ | | H09.2 | (SEQ | | | | | | |
| | non- | | | ID | | | | | | |
| | redundant_ | | | NO: | | | | | | |
| | pfms. | | | 285) | | | | | | |
| | meme | | | | | | | | | |
| 267 | JASPAR | MA0086.2 | MA0086.2. | NBRACAG | 13 | 44714 | 2.30E−06 | CENTRIMO | | |
| | 2022_ | | sna | GTGYAN | | | | | | |
| | CORE_ | | | (SEQ | | | | | | |
| | non- | | | ID | | | | | | |
| | redundant_ | | | NO: | | | | | | |
| | pfms. | | | 286) | | | | | | |
| | meme | | | | | | | | | |
| 268 | JASPAR | MA1620.1 | MA1620.1. | NVACACC | 12 | 69191 | 2.50E−06 | CENTRIMO | | |
| | 2022_ | | Ptf1A | TGTNN | | | | | | |
| | CORE_ | | | (SEQ | | | | | | |
| | non- | | | ID | | | | | | |
| | redundant_ | | | NO: | | | | | | |
| | pfms. | | | 287) | | | | | | |
| | meme | | | | | | | | | |
| 269 | JASPAR | MA1897.1 | MA1897.1. | NNNNNND | 20 | 77993 | 4.30E−06 | CENTRIMO | | |
| | 2022_ | | Fli-Erg-c | CCGGAAR | | | | | | |
| | CORE_ | | | HNNNNN | | | | | | |
| | non- | | | (SEQ | | | | | | |
| | redundant_ | | | ID | | | | | | |
| | pfms. | | | NO: | | | | | | |
| | meme | | | 288 | | | | | | |
| 270 | JASPAR | MA0443.1 | MA0443.1. | RRGGGGC | 10 | 34858 | 5.00E−06 | CENTRIMO | | |
| | 2022_ | | btd | GKR | | | | | | |
| | CORE_ | | | (SEQ | | | | | | |
| | non- | | | ID | | | | | | |
| | redundant_ | | | NO: | | | | | | |
| | pfms. | | | 289) | | | | | | |
| | meme | | | | | | | | | |
| 271 | JASPAR | MA0478.1 | MA0478.1. | KRRTGAS | 11 | 19087 | 5.10E−06 | CENTRIMO | | |
| | 2022_ | | FOSL2 | TCAB | | | | | | |
| | CORE_ | | | (SEQ | | | | | | |
| | non- | | | ID | | | | | | |
| | redundant_ | | | NO: | | | | | | |
| | pfms. | | | 290) | | | | | | |
| | meme | | | | | | | | | |
| 272 | JASPAR | MA0338.1 | MA0338.1. | CCCCRCV | 7 | 72021 | 5.40E−06 | CENTRIMO | | |
| | 2022_ | | MIG2 | (SEQ | | | | | | |
| | CORE_ | | | ID | | | | | | |
| | non- | | | NO: | | | | | | |
| | redundant_ | | | 291) | | | | | | |
| | pfms. | | | | | | | | | |
| | meme | | | | | | | | | |
| 273 | JASPAR | MA0778.1 | MA0778.1. | AGGGGAW | 13 | 9977 | 6.00E−06 | CENTRIMO | | |
| | 2022_ | | NFKB2 | TCCCCY | | | | | | |
| | CORE_ | | | (SEQ | | | | | | |
| | non- | | | ID | | | | | | |
| | redundant_ | | | NO: | | | | | | |
| | pfms. | | | 292) | | | | | | |
| | meme | | | | | | | | | |
| 274 | JASPAR | MA0761.2 | MA0761.2. | NNACAGG | 14 | 78087 | 6.40E−06 | CENTRIMO | | |
| | 2022_ | | ETV1 | AAGTGNN | | | | | | |
| | CORE_ | | | (SEQ | | | | | | |
| | non- | | | ID | | | | | | |
| | redundant_ | | | NO: | | | | | | |
| | pfms. | | | 293) | | | | | | |
| | meme | | | | | | | | | |
| 275 | JASPAR | MA1976.1 | MA1976.1. | SGACGGC | 12 | 24147 | 6.90E−06 | CENTRIMO | | |
| | 2022_ | | Zm00001 | GACGV | | | | | | |
| | CORE_ | | d031796 | (SEQ | | | | | | |
| | non- | | | ID | | | | | | |
| | redundant_ | | | NO: | | | | | | |
| | pfms. | | | 294) | | | | | | |
| | meme | | | | | | | | | |
| 276 | JASPAR | MA1621.1 | MA1621.1. | NNVACAC | 14 | 71592 | 7.00E−06 | CENTRIMO | | |
| | 2022_ | | Rbpjl | CTGTBNN | | | | | | |
| | CORE_ | | | (SEQ | | | | | | |
| | non- | | | ID | | | | | | |
| | redundant_ | | | NO: | | | | | | |
| | pfms. | | | 295) | | | | | | |
| | meme | | | | | | | | | |
| 277 | JASPAR | MA1679.1 | MA1679.1. | HDYCACC | 15 | 20652 | 7.20E−06 | CENTRIMO | | |
| | 2022_ | | RAP2-1 | GACAHHN | | | | | | |
| | CORE_ | | | N | | | | | | |
| | non- | | | (SEQ | | | | | | |
| | redundant_ | | | ID | | | | | | |
| | pfms. | | | NO: | | | | | | |
| | meme | | | 296) | | | | | | |
| 278 | JASPAR | MA0491.2 | MA0491.2. | NNATGAC | 13 | 33174 | 7.40E−06 | CENTRIMO | | |
| | 2022_ | | JUND | TCATNN | | | | | | |
| | CORE_ | | | (SEQ | | | | | | |
| | non- | | | ID | | | | | | |
| | redundant_ | | | NO: | | | | | | |
| | pfms. | | | 297) | | | | | | |
| | meme | | | | | | | | | |
| 279 | JASPAR | MA2038.1 | MA2038.1. | NNRGACC | 14 | 58731 | 8.20E−06 | CENTRIMO | | |
| | 2022_ | | Gli1 | ACCCASV | | | | | | |
| | CORE_ | | | (SEQ | | | | | | |
| | non- | | | ID | | | | | | |
| | redundant_ | | | NO: | | | | | | |
| | pfms. | | | 298) | | | | | | |
| | meme | | | | | | | | | |
| 280 | JASPAR | MA1130.1 | MA1130.1. | NNRTGAG | 12 | 37234 | 8.70E−06 | CENTRIMO | | |
| | 2022_ | | FOSL2::JUN | TCAYN | | | | | | |
| | CORE_ | | | (SEQ | | | | | | |
| | non- | | | ID | | | | | | |
| | redundant_ | | | NO: | | | | | | |
| | pfms. | | | 299 | | | | | | |
| | meme | | | | | | | | | |
| 281 | JASPAR | MA1513.1 | MA1513.1. | SCCCCGC | 11 | 18052 | 1.20E−05 | CENTRIMO | | |
| | 2022_ | | KLF15 | CCCS | | | | | | |
| | CORE_ | | | (SEQ | | | | | | |
| | non- | | | ID | | | | | | |
| | redundant_ | | | NO: | | | | | | |
| | pfms. | | | 300) | | | | | | |
| | meme | | | | | | | | | |
| 282 | JASPAR | MA1063.1 | MA1063.1. | TGGGSCC | 10 | 78100 | 1.20E−05 | CENTRIMO | | |
| | 2022_ | | TCP19 | CAC | | | | | | |
| | CORE_ | | | (SEQ | | | | | | |
| | non- | | | ID | | | | | | |
| | redundant_ | | | NO: | | | | | | |
| | pfms. | | | 301) | | | | | | |
| | meme | | | | | | | | | |
| 283 | JASPAR | MA1651.1 | MA1651.1. | NNNHCAA | 21 | 27618 | 1.30E−05 | CENTRIMO | | |
| | 2022_ | | ZFP42 | RATGGCT | | | | | | |
| | CORE_ | | | GCCNBNN | | | | | | |
| | non- | | | (SEQ | | | | | | |
| | redundant_ | | | ID | | | | | | |
| | pfms. | | | NO: | | | | | | |
| | meme | | | 302) | | | | | | |
| 284 | JASPAR | MA1512.1 | MA1512.1. | SCCACGC | 11 | 43941 | 1.50E−05 | CENTRIMO | | |
| | 2022_ | | KLF11 | CCMC | | | | | | |
| | CORE_ | | | (SEQ | | | | | | |
| | non- | | | ID | | | | | | |
| | redundant_ | | | NO: | | | | | | |
| | pfms. | | | 303) | | | | | | |
| | meme | | | | | | | | | |
| 285 | JASPAR | MA1097.1 | MA1097.1. | GGSMCCA | 8 | 39705 | 1.50E−05 | CENTRIMO | | |
| | 2022_ | | ARALYDR | C | | | | | | |
| | CORE_ | | AFT_ | (SEQ | | | | | | |
| | non- | | 493022 | ID | | | | | | |
| | redundant_ | | | NO: | | | | | | |
| | pfms. | | | 304) | | | | | | |
| | meme | | | | | | | | | |
| 286 | JASPAR | MA0823.1 | MA0823.1. | GRCACGT | 10 | 17561 | 1.50E−05 | CENTRIMO | | |
| | 2022_ | | HEY1 | GCC | | | | | | |
| | CORE_ | | | (SEQ | | | | | | |
| | non- | | | ID | | | | | | |
| | redundant_ | | | NO: | | | | | | |
| | pfms. | | | 305} | | | | | | |
| | meme | | | | | | | | | |
| 287 | JASPAR | MA0397.1 | MA0397.1. | GVTAGCG | 9 | 5772 | 1.70E−05 | CENTRIMO | | |
| | 2022_ | | STP4 | CA | | | | | | |
| | CORE_ | | | (SEQ | | | | | | |
| | non- | | | ID | | | | | | |
| | redundant_ | | | NO: | | | | | | |
| | pfms. | | | 306) | | | | | | |
| | meme | | | | | | | | | |
| 288 | JASPAR | MA1875.1 | MA1875.1. | GGGGYGA | 15 | 15246 | 1.70E−05 | CENTRIMO | | |
| | 2022_ | | ZNF669 | YGACCRC | | | | | | |
| | CORE_ | | | T | | | | | | |
| | non- | | | (SEQ | | | | | | |
| | redundant_ | | | ID | | | | | | |
| | pfms. | | | NO: | | | | | | |
| | meme | | | 307) | | | | | | |
| 289 | JASPAR | MA1635.1 | MA1635.1. | NVCAGCT | 10 | 17285 | 2.20E−05 | CENTRIMO | | |
| | 2022_ | | BHLHE22 | GBN | | | | | | |
| | CORE_ | | | (SEQ | | | | | | |
| | non- | | | ID | | | | | | |
| | redundant_ | | | NO: | | | | | | |
| | pfms. | | | 308) | | | | | | |
| | meme | | | | | | | | | |
| 290 | JASPAR | MA1894.1 | MA1894.1. | NNNNNRY | 20 | 63429 | 2.40E−05 | CENTRIMO | | |
| | 2022_ | | Etv1/4/5 | TTCCGGN | | | | | | |
| | CORE_ | | | NNNNNN | | | | | | |
| | non- | | | (SEQ | | | | | | |
| | redundant_ | | | ID | | | | | | |
| | pfms. | | | NO: | | | | | | |
| | meme | | | 309) | | | | | | |
| 291 | JASPAR | MA0598.3 | MA0598.3. | NNCACTT | 15 | 77456 | 2.40E−05 | CENTRIMO | | |
| | 2022_ | | EHF | CCTGTTN | | | | | | |
| | CORE_ | | | N | | | | | | |
| | non- | | | (SEQ | | | | | | |
| | redundant_ | | | ID | | | | | | |
| | pfms. | | | NO: | | | | | | |
| | meme | | | 310) | | | | | | |
| 292 | JASPAR | MA1789.1 | MA1789.1. | ACCGGAA | 14 | 10349 | 2.50E−05 | CENTRIMO | | |
| | 2022_ | | ELK1:: | GTAATTA | | | | | | |
| | CORE_ | | HOXA1 | (SEQ | | | | | | |
| | non- | | | ID | | | | | | |
| | redundant_ | | | NO: | | | | | | |
| | pfms. | | | 311) | | | | | | |
| | meme | | | | | | | | | |
| 293 | JASPAR | MA0396.1 | MA0396.1. | RSTAGCG | 9 | 5811 | 2.70E−05 | CENTRIMO | | |
| | 2022_ | | STP3 | CA | | | | | | |
| | CORE_ | | | (SEQ | | | | | | |
| | non- | | | ID | | | | | | |
| | redundant_ | | | NO: | | | | | | |
| | pfms. | | | 312) | | | | | | |
| | meme | | | | | | | | | |
| 294 | JASPAR | MA1143.1 | MA1143.1. | RTGACGT | 10 | 72639 | 3.00E−05 | CENTRIMO | | |
| | 2022_ | | FOSL1:: | MAY | | | | | | |
| | CORE_ | | JUND | (SEQ | | | | | | |
| | non- | | | ID | | | | | | |
| | redundant_ | | | NO: | | | | | | |
| | pfms. | | | 313) | | | | | | |
| | meme | | | | | | | | | |
| 295 | JASPAR | MA1262.1 | MA1262.1. | YCDCCDC | 21 | 20784 | 3.50E−05 | CENTRIMO | | |
| | 2022_ | | ERF2 | CDCCGCC | | | | | | |
| | CORE_ | | | GCCRYY | | | | | | |
| | non- | | | D | | | | | | |
| | redundant_ | | | (SEQ | | | | | | |
| | pfms. | | | ID | | | | | | |
| | meme | | | NO: | | | | | | |
| | | | | 314) | | | | | | |
| 296 | JASPAR | MA1542.1 | MA1542.1. | HGCTACY | 10 | 39976 | 3.80E−05 | CENTRIMO | | |
| | 2022_ | | OSR1 | GTD | | | | | | |
| | CORE_ | | | (SEQ | | | | | | |
| | non- | | | ID | | | | | | |
| | redundant_ | | | NO: | | | | | | |
| | pfms. | | | 315) | | | | | | |
| | meme | | | | | | | | | |
| 297 | JASPAR | MA0826.1 | MA0826.1. | AMCATAT | 10 | 10512 | 4.20E−05 | CENTRIMO | | |
| | 2022_ | | OLIG1 | GKT | | | | | | |
| | CORE_ | | | (SEQ | | | | | | |
| | non- | | | ID | | | | | | |
| | redundant_ | | | NO: | | | | | | |
| | pfms. | | | 316) | | | | | | |
| | meme | | | | | | | | | |
| 298 | JASPAR | MA0745.2 | MA0745.2. | NBGCACC | 13 | 46609 | 4.50E−05 | CENTRIMO | | |
| | 2022_ | | SNAI2 | TGTMNY | | | | | | |
| | CORE_ | | | (SEQ | | | | | | |
| | non- | | | ID | | | | | | |
| | redundant_ | | | NO: | | | | | | |
| | pfms. | | | 317) | | | | | | |
| | meme | | | | | | | | | |
| 299 | JASPAR | MA1128.1 | MA1128.1. | NKATGAC | 13 | 36860 | 6.70E−05 | CENTRIMO | | |
| | 2022_ | | FOSL1::JUN | TCATNN | | | | | | |
| | CORE_ | | | (SEQ | | | | | | |
| | non- | | | ID | | | | | | |
| | redundant_ | | | NO: | | | | | | |
| | pfms. | | | 318) | | | | | | |
| | meme | | | | | | | | | |
| 300 | JASPAR | MA0657.1 | MA0657.1. | RTGMCAC | 18 | 3567 | 7.60E−05 | CENTRIMO | | |
| | 2022_ | | KLF13 | GCCCCTT | | | | | | |
| | CORE_ | | | TTTG | | | | | | |
| | non- | | | (SEQ | | | | | | |
| | redundant_ | | | ID | | | | | | |
| | pfms. | | | NO: | | | | | | |
| | meme | | | 319) | | | | | | |
| 301 | JASPAR | MA0099.3 | MA0099.3. | ATGAGTC | 10 | 43795 | 8.10E−05 | CENTRIMO | | |
| | 2022_ | | FOS::JUN | AYM | | | | | | |
| | CORE_ | | | (SEQ | | | | | | |
| | non- | | | ID | | | | | | |
| | redundant_ | | | NO: | | | | | | |
| | pfms. | | | 320) | | | | | | |
| | meme | | | | | | | | | |
| 302 | JASPAR | MA1019.1 | MA1019.1. | GGGSCCC | 9 | 59761 | 8.70E−05 | CENTRIMO | | |
| | 2022_ | | Glyma19g | AC | | | | | | |
| | CORE_ | | 26560.1 | (SEQ | | | | | | |
| | non- | | | ID | | | | | | |
| | redundant_ | | | NO: | | | | | | |
| | pfms. | | | 321) | | | | | | |
| | meme | | | | | | | | | |
| 303 | JASPAR | MA1536.1 | MA1536.1. | RRGGTCA | 8 | 102705 | 8.70E−05 | CENTRIMO | | |
| | 2022_ | | NR2C2 | N | | | | | | |
| | CORE_ | | | (SEQ | | | | | | |
| | non- | | | ID | | | | | | |
| | redundant_ | | | NO: | | | | | | |
| | pfms. | | | 322) | | | | | | |
| | meme | | | | | | | | | |
| 304 | JASPAR | MA0583.1 | MA0583.1. | HYCACCT | 12 | 100671 | 9.20E−05 | CENTRIMO | | |
| | 2022_ | | RAV1 | GRNNY | | | | | | |
| | CORE_ | | | (SEQ | | | | | | |
| | non- | | | ID | | | | | | |
| | redundant_ | | | NO: | | | | | | |
| | pfms. | | | 323) | | | | | | |
| | meme | | | | | | | | | |
| 305 | JASPAR | MA0260.1 | MA0260.1. | GAARCC | 6 | 36498 | 1.10E−04 | CENTRIMO | | |
| | 2022_ | | che−1 | (SEQ | | | | | | |
| | CORE_ | | | ID | | | | | | |
| | non- | | | NO: | | | | | | |
| | redundant_ | | | 324) | | | | | | |
| | pfms. | | | | | | | | | |
| | meme | | | | | | | | | |
| 306 | JASPAR | MA1785.1 | MA1785.1. | BGTAAAC | 15 | 54610 | 1.20E−04 | CENTRIMO | | |
| | 2022_ | | ETV2::FOXI1 | AGGAAGY | | | | | | |
| | CORE_ | | | R | | | | | | |
| | non- | | | (SEQ | | | | | | |
| | redundant_ | | | ID | | | | | | |
| | pfms. | | | NO: | | | | | | |
| | meme | | | 325) | | | | | | |
| 307 | JASPAR | MA1565.1 | MA1565.1. | DRAGGTG | 12 | 70900 | 1.20E−04 | CENTRIMO | | |
| | 2022_ | | TBX18 | TGAAR | | | | | | |
| | CORE_ | | | (SEQ | | | | | | |
| | non- | | | ID | | | | | | |
| | redundant_ | | | NO: | | | | | | |
| | pfms. | | | 326) | | | | | | |
| | meme | | | | | | | | | |
| 308 | JASPAR | MA0541.1 | MA0541.1. | HDHKSGC | 15 | 15120 | 1.30E−04 | CENTRIMO | | |
| | 2022_ | | efl-1 | GSGAAAW | | | | | | |
| | CORE_ | | | T | | | | | | |
| | non- | | | (SEQ | | | | | | |
| | redundant_ | | | ID | | | | | | |
| | pfms. | | | NO: | | | | | | |
| | meme | | | 327) | | | | | | |
| 309 | JASPAR | MA1524.2 | MA1524.2. | VRRRACA | 16 | 30585 | 1.30E−04 | CENTRIMO | | |
| | 2022_ | | Msgn1 | AATGGTN | | | | | | |
| | CORE_ | | | NN | | | | | | |
| | non- | | | (SEQ | | | | | | |
| | redundant_ | | | ID | | | | | | |
| | pfms. | | | NO: | | | | | | |
| | meme | | | 328) | | | | | | |
| 310 | JASPAR | MA0384.1 | MA0384.1. | TGRTAGC | 11 | 1307 | 1.40E−04 | CENTRIMO | | |
| | 2022_ | | SNT2 | GCCR | | | | | | |
| | COREnon- | | | (SEQ | | | | | | |
| | redundant_ | | | ID | | | | | | |
| | pfms. | | | NO: | | | | | | |
| | meme | | | 329) | | | | | | |
| 311 | JASPAR | MA1746.1 | MA1746.1. | YYCACCT | 10 | 25035 | 1.40E−04 | CENTRIMO | | |
| | 2022_ | | MYB99 | AMY | | | | | | |
| | CORE_ | | | (SEQ | | | | | | |
| | non- | | | ID | | | | | | |
| | redundant_ | | | NO: | | | | | | |
| | pfms. | | | 330) | | | | | | |
| | meme | | | | | | | | | |
| 312 | JASPAR | MA2082.1 | MA2082.1. | YYCACCT | 10 | 25035 | 1.40E−04 | CENTRIMO | | |
| | 2022_ | | MYB99 | AMY | | | | | | |
| | CORE_ | | | (SEQ | | | | | | |
| | non- | | | ID | | | | | | |
| | redundant_ | | | NO: | | | | | | |
| | pfms. | | | 331) | | | | | | |
| | meme | | | | | | | | | |
| 313 | JASPAR | MA0059.1 | MA0059.1. | RASCACG | 11 | 18359 | 1.40E−04 | CENTRIMO | | |
| | 2022_ | | MAX::MYC | TGGT | | | | | | |
| | CORE_ | | | (SEQ | | | | | | |
| | non- | | | ID | | | | | | |
| | redundant_ | | | NO: | | | | | | |
| | pfms. | | | 332) | | | | | | |
| | meme | | | | | | | | | |
| 314 | JASPAR | MA1786.1 | MA1786.1. | GTAAACA | 13 | 40924 | 1.60E−04 | CENTRIMO | | |
| | 2022_ | | ETV5:: | GGAWGY | | | | | | |
| | CORE_ | | FOXI1 | (SEQ | | | | | | |
| | non- | | | ID | | | | | | |
| | redundant_ | | | NO: | | | | | | |
| | pfms. | | | 333) | | | | | | |
| | meme | | | | | | | | | |
| 315 | JASPAR | MA0694.1 | MA0694.1. | RCGACCA | 12 | 23517 | 1.70E−04 | CENTRIMO | | |
| | 2022_ | | ZBTB7B | CCGAA | | | | | | |
| | CORE_ | | | (SEQ | | | | | | |
| | non- | | | ID | | | | | | |
| | redundant_ | | | NO: | | | | | | |
| | pfms. | | | 334) | | | | | | |
| | meme | | | | | | | | | |
| 316 | JASPAR | MA1637.1 | MA1637.1. | NYCCCAA | 13 | 51943 | 1.90E−04 | CENTRIMO | | |
| | 2022_ | | EBF3 | GGGANN | | | | | | |
| | CORE_ | | | (SEQ | | | | | | |
| | non- | | | ID | | | | | | |
| | redundant_ | | | NO: | | | | | | |
| | pfms. | | | 335) | | | | | | |
| | meme | | | | | | | | | |
| 317 | JASPAR | MA0587.1 | MA0587.1. | GTGGACC | 10 | 23642 | 2.40E−04 | CENTRIMO | | |
| | 2022_ | | TCP16 | CRS | | | | | | |
| | CORE_ | | | (SEQ | | | | | | |
| | non- | | | ID | | | | | | |
| | redundant_ | | | NO: | | | | | | |
| | pfms. | | | 336) | | | | | | |
| | meme | | | | | | | | | |
| 318 | JASPAR | MA1779.1 | MA1779.1. | RSCGGAA | 16 | 39284 | 2.50E−04 | CENTRIMO | | |
| | 2022_ | | TFAP4:: | GCAGSTG | | | | | | |
| | CORE_ | | ETV1 | KN | | | | | | |
| | non- | | | (SEQ | | | | | | |
| | redundant_ | | | ID | | | | | | |
| | pfms. | | | NO: | | | | | | |
| | meme | | | 337) | | | | | | |
| 319 | JASPAR | MA0535.1 | MA0535.1. | SHGRCGC | 15 | 14224 | 2.50E−04 | CENTRIMO | | |
| | 2022_ | | Mad | CGVCGSH | | | | | | |
| | CORE_ | | | G | | | | | | |
| | non- | | | (SEQ | | | | | | |
| | redundant_ | | | ID | | | | | | |
| | pfms. | | | NO: | | | | | | |
| | meme | | | 338) | | | | | | |
| 320 | JASPAR | MA0671.1 | MA0671.1. | NNTGCCA | 9 | 102407 | 3.30E−04 | CENTRIMO | | |
| | 2022_ | | NFIX | AN | | | | | | |
| | CORE_ | | | (SEQ | | | | | | |
| | non- | | | ID | | | | | | |
| | redundant_ | | | NO: | | | | | | |
| | pfms. | | | 339) | | | | | | |
| | meme | | | | | | | | | |
| 321 | JASPAR | MA0811.1 | MA0811.1. | YGCCCBV | 12 | 49606 | 3.50E−04 | CENTRIMO | | |
| | 2022_ | | TFAP2B | RGGCA | | | | | | |
| | CORE_ | | | (SEQ | | | | | | |
| | non- | | | ID | | | | | | |
| | redundant_ | | | NO: | | | | | | |
| | pfms. | | | 340) | | | | | | |
| | meme | | | | | | | | | |
| 322 | JASPAR | MA1011.1 | MA1011.1. | NNCACGT | 10 | 48778 | 4.00E−04 | CENTRIMO | | |
| | 2022_ | | PHYPADR | GNN | | | | | | |
| | CORE_ | | AFT_ | (SEQ | | | | | | |
| | non- | | 72483 | ID | | | | | | |
| | redundant_ | | | NO: | | | | | | |
| | pfms. | | | 341) | | | | | | |
| | meme | | | | | | | | | |
| 323 | JASPAR | MA2044.1 | MA2044.1. | VVCAGCT | 10 | 19952 | 4.70E−04 | CENTRIMO | | |
| | 2022_ | | Neurod2 | GBB | | | | | | |
| | CORE_ | | | (SEQ | | | | | | |
| | non- | | | ID | | | | | | |
| | redundant_ | | | NO: | | | | | | |
| | pfms. | | | 342 | | | | | | |
| | meme | | | | | | | | | |
| 324 | JASPAR | MA0502.2 | MA0502.2. | CYCATTG | 12 | 45592 | 5.10E−04 | CENTRIMO | | |
| | 2022_ | | NFYB | GCCVV | | | | | | |
| | COREnon- | | | (SEQ | | | | | | |
| | redundant_ | | | ID | | | | | | |
| | pfms. | | | NO: | | | | | | |
| | meme | | | 343) | | | | | | |
| 325 | JASPAR | MA0269.1 | MA0269.1. | KBNBMTA | 21 | 33472 | 5.50E−04 | CENTRIMO | | |
| | 2022_ | | AFT1 | KTGCACC | | | | | | |
| | CORE_ | | | CSNWW | | | | | | |
| | non- | | | BS | | | | | | |
| | redundant_ | | | (SEQ | | | | | | |
| | pfms. | | | ID | | | | | | |
| | meme | | | NO: | | | | | | |
| | | | | 344) | | | | | | |
| 326 | JASPAR | MA0609.2 | MA0609.2. | NNDGTGA | 16 | 29249 | 6.00E−04 | CENTRIMO | | |
| | 2022_ | | CREM | CGTCACH | | | | | | |
| | CORE_ | | | NN | | | | | | |
| | non- | | | (SEQ | | | | | | |
| | redundant_ | | | ID | | | | | | |
| | pfms. | | | NO: | | | | | | |
| | meme | | | 345) | | | | | | |
| 327 | JASPAR | MA0810.1 | MA0810.1. | YGCCCBV | 12 | 52151 | 6.60E−04 | CENTRIMO | | |
| | 2022_ | | TFAP2A | RGGCR | | | | | | |
| | CORE_ | | | (SEQ | | | | | | |
| | non- | | | ID | | | | | | |
| | redundant_ | | | NO: | | | | | | |
| | pfms. | | | 346) | | | | | | |
| | meme | | | | | | | | | |
| 328 | JASPAR | MA0162.4 | MA0162.4. | VCMCGCC | 14 | 49922 | 8.50E−04 | CENTRIMO | | |
| | 2022_ | | EGR1 | CACGC | | | | | | |
| | CORE_ | | | VS | | | | | | |
| | non- | | | (SEQ | | | | | | |
| | redundant_ | | | ID | | | | | | |
| | pfms. | | | NO: | | | | | | |
| | meme | | | 347) | | | | | | |
| 329 | JASPAR | MA1693.1 | MA1693.1. | NNCAGAC | 13 | 74733 | 9.70E−04 | CENTRIMO | | |
| | 2022_ | | ARF34 | AGCMNN | | | | | | |
| | CORE_ | | | (SEQ | | | | | | |
| | non- | | | ID | | | | | | |
| | redundant_ | | | NO: | | | | | | |
| | pfms. | | | 348) | | | | | | |
| | meme | | | | | | | | | |
| 330 | JASPAR | MA0774.1 | MA0774.1. | TTGACAG | 8 | 62536 | 9.80E−04 | CENTRIMO | | |
| | 2022_ | | MEIS2 | S | | | | | | |
| | CORE_ | | | (SEQ | | | | | | |
| | non- | | | ID | | | | | | |
| | redundant_ | | | NO: | | | | | | |
| | pfms. | | | 349) | | | | | | |
| | meme | | | | | | | | | |
| 331 | JASPAR | MA0557.1 | MA0557.1. | HHCACGC | 12 | 25277 | 1.00E−03 | CENTRIMO | | |
| | 2022_ | | FHY3 | GCTNN | | | | | | |
| | CORE_ | | | (SEQ | | | | | | |
| | non- | | | ID | | | | | | |
| | redundant_ | | | NO: | | | | | | |
| | pfms. | | | 350) | | | | | | |
| | meme | | | | | | | | | |
| 332 | JASPAR | MA1010.1 | MA1010.1. | NTGTCGG | 13 | 32136 | 1.00E−03 | CENTRIMO | | |
| | 2022_ | | PHYPADR | TANNNN | | | | | | |
| | CORE_ | | AFT_ | (SEQ | | | | | | |
| | non- | | 64121 | ID | | | | | | |
| | redundant_ | | | NO: | | | | | | |
| | pfms. | | | 351) | | | | | | |
| | meme | | | | | | | | | |
| 333 | JASPAR | MA1863.1 | MA1863.1. | WWWTGVC | 15 | 64323 | 1.10E−03 | CENTRIMO | | |
| | 2022_ | | NLP7 | YYTTSRD | | | | | | |
| | CORE_ | | | D | | | | | | |
| | non- | | | (SEQ | | | | | | |
| | redundant_ | | | ID | | | | | | |
| | pfms. | | | NO: | | | | | | |
| | meme | | | 352) | | | | | | |
| 334 | JASPAR | MA1870.1 | MA1870.1. | DGGGGGG | 9 | 36167 | 1.20E−03 | CENTRIMO | | |
| | 2022_ | | KLF7 | GG | | | | | | |
| | CORE_ | | | (SEQ | | | | | | |
| | non- | | | ID | | | | | | |
| | redundant_ | | | NO: | | | | | | |
| | pfms. | | | 353) | | | | | | |
| | meme | | | | | | | | | |
| 335 | JASPAR | MA1969.1 | MA1969.1. | BNCGCAC | 14 | 23796 | 1.40E−03 | CENTRIMO | | |
| | 2022_ | | bHLH145 | GTGCG | | | | | | |
| | CORE_ | | | NV | | | | | | |
| | non- | | | (SEQ | | | | | | |
| | redundant_ | | | ID | | | | | | |
| | pfms. | | | NO: | | | | | | |
| | meme | | | 354) | | | | | | |
| 336 | JASPAR | MA1713.1 | MA1713.1. | SSCGCCG | 14 | 30717 | 1.60E−03 | CENTRIMO | | |
| | 2022_ | | ZNF610 | CTCCSS | | | | | | |
| | CORE_ | | | S | | | | | | |
| | non- | | | (SEQ | | | | | | |
| | redundant_ | | | ID | | | | | | |
| | pfms. | | | NO: | | | | | | |
| | meme | | | 355) | | | | | | |
| 337 | JASPAR | MA0490.2 | MA0490.2. | NNATGAC | 13 | 37080 | 1.60E−03 | CENTRIMO | | |
| | 2022_ | | JUNB | TCATNN | | | | | | |
| | CORE_ | | | (SEQ | | | | | | |
| | non- | | | ID | | | | | | |
| | redundant_ | | | NO: | | | | | | |
| | pfms. | | | 356) | | | | | | |
| | meme | | | | | | | | | |
| 338 | JASPAR | MA1264.1 | MA1264.1. | HGRYGGC | 15 | 17921 | 1.70E−03 | CENTRIMO | | |
| | 2022_ | | ERFO95 | GGCGGHG | | | | | | |
| | CORE_ | | | G | | | | | | |
| | non- | | | (SEQ | | | | | | |
| | redundant_ | | | ID | | | | | | |
| | pfms. | | | NO: | | | | | | |
| | meme | | | 357) | | | | | | |
| 339 | JASPAR | MA0633.2 | MA0633.2. | NVCAGCT | 10 | 20668 | 2.30E−03 | CENTRIMO | | |
| | 2022_ | | Twist2 | GBN | | | | | | |
| | CORE_ | | | (SEQ | | | | | | |
| | non- | | | ID | | | | | | |
| | redundant_ | | | NO: | | | | | | |
| | pfms. | | | 358 | | | | | | |
| | meme | | | | | | | | | |
| 340 | JASPAR | MA1132.1 | MA1132.1. | KATGACK | 10 | 66465 | 2.50E−03 | CENTRIMO | | |
| | 2022_ | | JUN::JUNB | CAT | | | | | | |
| | CORE_ | | | (SEQ | | | | | | |
| | non- | | | ID | | | | | | |
| | redundant_ | | | NO: | | | | | | |
| | pfms. | | | 3591 | | | | | | |
| | meme | | | | | | | | | |
| 341 | JASPAR | MA0163.1 | MA0163.1. | GGGGCCC | 14 | 13615 | 2.70E−03 | CENTRIMO | | |
| | 2022_ | | PLAG1 | WAGGGGG | | | | | | |
| | CORE_ | | | (SEQ | | | | | | |
| | non- | | | ID | | | | | | |
| | redundant_ | | | NO: | | | | | | |
| | pfms. | | | 360) | | | | | | |
| | meme | | | | | | | | | |
| 342 | JASPAR | MA0691.1 | MA0691.1. | AWCAGCT | 10 | 20433 | 2.80E−03 | CENTRIMO | | |
| | 2022_ | | TFAP4 | GWT | | | | | | |
| | COREnon- | | | (SEQ | | | | | | |
| | redundant_ | | | ID | | | | | | |
| | pfms. | | | NO: | | | | | | |
| | meme | | | 361) | | | | | | |
| 343 | JASPAR | MA0967.1 | MA0967.1. | TGACGTC | 8 | 30299 | 2.90E−03 | CENTRIMO | | |
| | 2022_ | | BZIP60 | A | | | | | | |
| | CORE_ | | | (SEQ | | | | | | |
| | non- | | | ID | | | | | | |
| | redundant_ | | | NO: | | | | | | |
| | pfms. | | | 362 | | | | | | |
| | meme | | | | | | | | | |
| 344 | JASPAR | MA1221.1 | MA1221.1. | TKGCGGC | 15 | 17466 | 3.00E−03 | CENTRIMO | | |
| | 2022_ | | RAP2-6 | GGMGGHG | | | | | | |
| | CORE_ | | | G | | | | | | |
| | non- | | | (SEQ | | | | | | |
| | redundant_ | | | ID | | | | | | |
| | pfms. | | | NO: | | | | | | |
| | meme | | | 363) | | | | | | |
| 345 | JASPAR | MA1781.1 | MA1781.1. | DCCGGAA | 16 | 8825 | 3.10E−03 | CENTRIMO | | |
| | 2022_ | | ELK1::SREBF2 | GTSRCGT | | | | | | |
| | CORE_ | | | GA | | | | | | |
| | non- | | | (SEQ | | | | | | |
| | redundant_ | | | ID | | | | | | |
| | pfms. | | | NO: | | | | | | |
| | meme | | | 364) | | | | | | |
| 346 | JASPAR | MA1715.1 | MA1715.1. | CCCCACT | 15 | 14897 | 3.30E−03 | CENTRIMO | | |
| | 2022_ | | ZNF707 | CCTGGTA | | | | | | |
| | CORE_ | | | C | | | | | | |
| | non- | | | (SEQ | | | | | | |
| | redundant_ | | | ID | | | | | | |
| | pfms. | | | NO: | | | | | | |
| | meme | | | 365) | | | | | | |
| 347 | JASPAR | MA1959.1 | MA1959.1. | NNNNNNR | 22 | 81599 | 3.50E−03 | CENTRIMO | | |
| | 2022_ | | Tbox-a | GGTGTGA | | | | | | |
| | CORE_ | | | ANDNNNN | | | | | | |
| | non- | | | N | | | | | | |
| | redundant_ | | | (SEQ | | | | | | |
| | pfms. | | | ID | | | | | | |
| | meme | | | NO: | | | | | | |
| | | | | 366) | | | | | | |
| 348 | JASPAR | MA1559.1 | MA1559.1. | RRCAGGT | 10 | 33543 | 3.50E−03 | CENTRIMO | | |
| | 2022_ | | SNAI3 | GYA | | | | | | |
| | CORE_ | | | (SEQ | | | | | | |
| | non- | | | ID | | | | | | |
| | redundant_ | | | NO: | | | | | | |
| | pfms. | | | 367) | | | | | | |
| | meme | | | | | | | | | |
| 349 | JASPAR | MA0283.1 | MA0283.1. | GGCGGAG | 8 | 24572 | 4.00E−03 | CENTRIMO | | |
| | 2022_ | | CHA4 | W | | | | | | |
| | CORE_ | | | (SEQ | | | | | | |
| | non- | | | ID | | | | | | |
| | redundant_ | | | NO: | | | | | | |
| | pfms. | | | 368 | | | | | | |
| | meme | | | | | | | | | |
| 350 | JASPAR | MA0741.1 | MA0741.1. | GMCACGC | 11 | 49151 | 4.30E−03 | CENTRIMO | | |
| | 2022_ | | KLF16 | CCCC | | | | | | |
| | CORE_ | | | (SEQ | | | | | | |
| | non- | | | ID | | | | | | |
| | redundant_ | | | NO: | | | | | | |
| | pfms. | | | 369) | | | | | | |
| | meme | | | | | | | | | |
| 351 | JASPAR | MA1338.2 | MA1338.2. | DDNTGMC | 17 | 11233 | 4.50E−03 | CENTRIMO | | |
| | 2022_ | | DPBF3 | ACGTGTC | | | | | | |
| | CORE_ | | | MHH | | | | | | |
| | non- | | | (SEQ | | | | | | |
| | redundant_ | | | ID | | | | | | |
| | pfms. | | | NO: | | | | | | |
| | meme | | | 370 | | | | | | |
| 352 | JASPAR | MA0957.1 | MA0957.1. | GCACGTG | 8 | 29739 | 4.60E−03 | CENTRIMO | | |
| | 2022_ | | BHLH3 | C | | | | | | |
| | CORE_ | | | (SEQ | | | | | | |
| | non- | | | ID | | | | | | |
| | redundant_ | | | NO: | | | | | | |
| | pfms. | | | 371) | | | | | | |
| | meme | | | | | | | | | |
| 353 | JASPAR | MA1149.1 | MA1149.1. | RRGGTCA | 18 | 45630 | 4.80E−03 | CENTRIMO | | |
| | 2022_ | | RARA::RXRG | HNNNRRG | | | | | | |
| | CORE_ | | | GTCA | | | | | | |
| | non- | | | (SEQ | | | | | | |
| | redundant_ | | | ID | | | | | | |
| | pfms. | | | NO: | | | | | | |
| | meme | | | 372) | | | | | | |
| 354 | JASPAR | MA0916.1 | MA0916.1. | CCGGAAR | 8 | 6450 | 5.30E−03 | CENTRIMO | | |
| | 2022_ | | Ets21C | T | | | | | | |
| | CORE_ | | | (SEQ | | | | | | |
| | non- | | | ID | | | | | | |
| | redundant_ | | | NO: | | | | | | |
| | pfms. | | | 373) | | | | | | |
| | meme | | | | | | | | | |
| 355 | JASPAR | MA2033.1 | MA2033.1. | NYTGTGT | 24 | 13559 | 5.90E−03 | CENTRIMO | | |
| | 2022_ | | THRA | CCTCABR | | | | | | |
| | CORE_ | | | TGACCTY | | | | | | |
| | non- | | | WBB | | | | | | |
| | redundant_ | | | (SEQ | | | | | | |
| | pfms. | | | ID | | | | | | |
| | meme | | | NO: | | | | | | |
| | | | | 374) | | | | | | |
| 356 | JASPAR | MA1511.2 | MA1511.2. | GGGGCGG | 9 | 38081 | 6.00E−03 | CENTRIMO | | |
| | 2022_ | | KLF10 | GG | | | | | | |
| | CORE_ | | | (SEQ | | | | | | |
| | non- | | | ID | | | | | | |
| | redundant_ | | | NO: | | | | | | |
| | pfms. | | | 375) | | | | | | |
| | meme | | | | | | | | | |
| 357 | JASPAR | MA1866.1 | MA1866.1. | SSGGGGM | 12 | 35890 | 6.00E−03 | CENTRIMO | | |
| | 2022_ | | PATZ1 | GGGGS | | | | | | |
| | CORE_ | | | (SEQ | | | | | | |
| | non- | | | ID | | | | | | |
| | redundant_ | | | NO: | | | | | | |
| | pfms. | | | 376) | | | | | | |
| | meme | | | | | | | | | |
| 358 | JASPAR | MA1006.1 | MA1006.1. | NTGCCGG | 10 | 11947 | 6.00E−03 | CENTRIMO | | |
| | 2022_ | | ERF6 | (SEQ | | | | | | |
| | CORE_ | | | ID | | | | | | |
| | non- | | | NO: | | | | | | |
| | redundant_ | | | 377) | | | | | | |
| | pfms. | | | | | | | | | |
| | meme | | | | | | | | | |
| 359 | JASPAR | MA2036.1 | MA2036.1. | NRTGACT | 11 | 58349 | 6.40E−03 | CENTRIMO | | |
| | 2022_ | | Atf3 | CABN | | | | | | |
| | CORE_ | | | (SEQ | | | | | | |
| | non- | | | ID | | | | | | |
| | redundant_ | | | NO: | | | | | | |
| | pfms. | | | 378) | | | | | | |
| | meme | | | | | | | | | |
| 360 | JASPAR | MA2045.1 | MA2045.1. | NVCAGCT | 10 | 21965 | 7.70E−03 | CENTRIMO | | |
| | 2022_ | | Olig2 | GBN | | | | | | |
| | CORE_ | | | (SEQ | | | | | | |
| | non- | | | ID | | | | | | |
| | redundant_ | | | NO: | | | | | | |
| | pfms. | | | 379) | | | | | | |
| | meme | | | | | | | | | |
| 361 | JASPAR | MA0524.2 | MA0524.2. | YGCCYBV | 12 | 53106 | 7.80E−03 | CENTRIMO | | |
| | 2022_ | | TFAP2C | RGGCA | | | | | | |
| | CORE_ | | | (SEQ | | | | | | |
| | non- | | | ID | | | | | | |
| | redundant_ | | | NO: | | | | | | |
| | pfms. | | | 380) | | | | | | |
| | meme | | | | | | | | | |
| 362 | JASPAR | MA1975.1 | MA1975.1. | SSCGCCG | 13 | 24975 | 7.90E−03 | CENTRIMO | | |
| | 2022_ | | Zm00001 | CCGCCG | | | | | | |
| | CORE_ | | d024324 | (SEQ | | | | | | |
| | non- | | | ID | | | | | | |
| | redundant_ | | | NO: | | | | | | |
| | pfms. | | | 381) | | | | | | |
| | meme | | | | | | | | | |
| 363 | JASPAR | MA0270.1 | MA0270.1. | SACACCC | 8 | 20663 | 8.80E−03 | CENTRIMO | | |
| | 2022_ | | AFT2 | B | | | | | | |
| | CORE_ | | | (SEQ | | | | | | |
| | non- | | | ID | | | | | | |
| | redundant_ | | | NO: | | | | | | |
| | pfms. | | | 382) | | | | | | |
| | meme | | | | | | | | | |
| 364 | JASPAR | MA0014.3 | MA0014.3. | RRGCGTG | 12 | 51679 | 8.90E−03 | CENTRIMO | | |
| | 2022_ | | PAX5 | ACCNN | | | | | | |
| | CORE_ | | | (SEQ | | | | | | |
| | non- | | | ID | | | | | | |
| | redundant_ | | | NO: | | | | | | |
| | pfms. | | | 383) | | | | | | |
| | meme | | | | | | | | | |
| 365 | JASPAR | MA0410.1 | MA0410.1. | SGGCGGG | 8 | 26087 | 9.00E−03 | CENTRIMO | | |
| | 2022_ | | UGA3 | A | | | | | | |
| | CORE_ | | | (SEQ | | | | | | |
| | non- | | | ID | | | | | | |
| | redundant_ | | | NO: | | | | | | |
| | pfms. | | | 384) | | | | | | |
| | meme | | | | | | | | | |
| 366 | JASPAR | MA0051.1 | MA0051.1. | SGAAAGY | 18 | 6781 | 9.30E−03 | CENTRIMO | | |
| | 2022_ | | IRF2 | GAAASCR | | | | | | |
| | CORE_ | | | WWWM | | | | | | |
| | non- | | | (SEQ | | | | | | |
| | redundant_ | | | ID | | | | | | |
| | pfms. | | | NO: | | | | | | |
| | meme | | | 385) | | | | | | |
| 367 | JASPAR | MA1646.1 | MA1646.1. | NNACAGA | 12 | 87181 | 9.70E−03 | CENTRIMO | | |
| | 2022_ | | OSR2 | AGCNN | | | | | | |
| | CORE_ | | | (SEQ | | | | | | |
| | non- | | | ID | | | | | | |
| | redundant_ | | | NO: | | | | | | |
| | pfms. | | | 386) | | | | | | |
| | meme | | | | | | | | | |
| 368 | JASPAR | MA1627.1 | MA1627.1. | YBCCTCC | 14 | 57229 | 9.70E−03 | CENTRIMO | | |
| | 2022_ | | Wt1 | CCCACV | | | | | | |
| | CORE_ | | | B | | | | | | |
| | non- | | | (SEQ | | | | | | |
| | redundant_ | | | ID | | | | | | |
| | pfms. | | | NO: | | | | | | |
| | meme | | | 387) | | | | | | |
| 369 | JASPAR | MA1604.1 | MA1604.1. | NYCCCAA | 13 | 51534 | 1.00E−02 | CENTRIMO | | |
| | 2022_ | | Ebf2 | GGGANN | | | | | | |
| | COREnon- | | | (SEQ | | | | | | |
| | redundant_ | | | ID | | | | | | |
| | pfms. | | | NO: | | | | | | |
| | meme | | | 388) | | | | | | |
| 370 | JASPAR | MA1242.1 | MA1242.1. | CCDCCAC | 11 | 18784 | 1.10E−02 | CENTRIMO | | |
| | 2022_ | | DREB2F | CGCC | | | | | | |
| | CORE_ | | | (SEQ | | | | | | |
| | non- | | | ID | | | | | | |
| | redundant_ | | | NO: | | | | | | |
| | pfms. | | | 389) | | | | | | |
| | meme | | | | | | | | | |
| 371 | JASPAR | MA1219.2 | MA1219.2. | HDYCACC | 14 | 22757 | 1.10E−02 | CENTRIMO | | |
| | 2022_ | | ERFO11 | GACMAN | | | | | | |
| | CORE_ | | | N | | | | | | |
| | non- | | | (SEQ | | | | | | |
| | redundant_ | | | ID | | | | | | |
| | pfms. | | | NO: | | | | | | |
| | meme | | | 390) | | | | | | |
| 372 | JASPAR | MA0684.2 | MA0684.2. | NHAACCT | 12 | 77892 | 1.10E−02 | CENTRIMO | | |
| | 2022_ | | RUNX3 | CAANN | | | | | | |
| | CORE_ | | | (SEQ | | | | | | |
| | non- | | | ID | | | | | | |
| | redundant_ | | | NO: | | | | | | |
| | pfms. | | | 391) | | | | | | |
| | meme | | | | | | | | | |
| 373 | JASPAR | MA0772.1 | MA0772.1. | HCGAAAR | 14 | 23587 | 1.20E−02 | CENTRIMO | | |
| | 2022_ | | IRF7 | YGAAAV | | | | | | |
| | CORE_ | | | T | | | | | | |
| | non- | | | (SEQ | | | | | | |
| | redundant_ | | | ID | | | | | | |
| | pfms. | | | NO: | | | | | | |
| | meme | | | 392) | | | | | | |
| 374 | JASPAR | MA2009.1 | MA2009.1. | HSACGCT | 13 | 27588 | 1.20E−02 | CENTRIMO | | |
| | 2022_ | | MYB88 | CCTCHN | | | | | | |
| | CORE_ | | | (SEQ | | | | | | |
| | non- | | | ID | | | | | | |
| | redundant_ | | | NO: | | | | | | |
| | pfms. | | | 393) | | | | | | |
| | meme | | | | | | | | | |
| 375 | JASPAR | MA2067.1 | MA2067.1. | HSACGCT | 13 | 27588 | 1.20E−02 | CENTRIMO | | |
| | 2022_ | | MYB88 | CCTCHN | | | | | | |
| | CORE_ | | | (SEQ | | | | | | |
| | non- | | | ID | | | | | | |
| | redundant_ | | | NO: | | | | | | |
| | pfms. | | | 394) | | | | | | |
| | meme | | | | | | | | | |
| 376 | JASPAR | MA1774.1 | MA1774.1. | YHHYWTC | 11 | 89297 | 1.20E−02 | CENTRIMO | | |
| | 2022_ | | AT5G04390 | ACTN | | | | | | |
| | CORE_ | | | (SEQ | | | | | | |
| | non- | | | ID | | | | | | |
| | redundant_ | | | NO: | | | | | | |
| | pfms. | | | 395 | | | | | | |
| | meme | | | | | | | | | |
| 377 | JASPAR | MA1140.2 | MA1140.2. | GATGACG | 12 | 3127 | 1.30E−02 | CENTRIMO | | |
| | 2022_ | | JUNB | TCAYC | | | | | | |
| | CORE_ | | | (SEQ | | | | | | |
| | non- | | | ID | | | | | | |
| | redundant_ | | | NO: | | | | | | |
| | pfms. | | | 396) | | | | | | |
| | meme | | | | | | | | | |
| 378 | JASPAR | MA1466.1 | MA1466.1. | TGRTGAC | 14 | 1642 | 1.30E−02 | CENTRIMO | | |
| | 2022_ | | ATF6 | GTGGCA | | | | | | |
| | CORE_ | | | N | | | | | | |
| | non- | | | (SEQ | | | | | | |
| | redundant_ | | | ID | | | | | | |
| | pfms. | | | NO: | | | | | | |
| | meme | | | 397) | | | | | | |
| 379 | JASPAR | MA1893.1 | MA1893.1. | NNNNRNC | 20 | 90329 | 1.70E−02 | CENTRIMO | | |
| | 2022_ | | Erf-a | GGAAGTN | | | | | | |
| | CORE_ | | | NNNNNN | | | | | | |
| | non- | | | (SEQ | | | | | | |
| | redundant_ | | | ID | | | | | | |
| | pfms. | | | NO: | | | | | | |
| | meme | | | 398) | | | | | | |
| 380 | JASPAR | MA0150.2 | MA0150.2. | CASNATG | 15 | 24098 | 1.80E−02 | CENTRIMO | | |
| | 2022_ | | Nfe212 | ACTCAGC | | | | | | |
| | CORE_ | | | A | | | | | | |
| | non- | | | (SEQ | | | | | | |
| | redundant_ | | | ID | | | | | | |
| | pfms. | | | NO: | | | | | | |
| | meme | | | 399) | | | | | | |
| 381 | JASPAR | MA1095.1 | MA1095.1. | GGSCCCA | 8 | 30665 | 1.90E−02 | CENTRIMO | | |
| | 2022_ | | ARALYDR | C | | | | | | |
| | CORE_ | | AFT_ | (SEQ | | | | | | |
| | non- | | 495258 | ID | | | | | | |
| | redundant_ | | | NO: | | | | | | |
| | pfms. | | | 400) | | | | | | |
| | meme | | | | | | | | | |
| 382 | JASPAR | MA1098.1 | MA1098.1. | GGSCCCA | 8 | 30665 | 1.90E−02 | CENTRIMO | | |
| | 2022_ | | ARALYDR | C | | | | | | |
| | CORE_ | | AFT_ | (SEQ | | | | | | |
| | non- | | 484486 | ID | | | | | | |
| | redundant_ | | | NO: | | | | | | |
| | pfms. | | | 401) | | | | | | |
| | meme | | | | | | | | | |
| 383 | JASPAR | MA1265.2 | MA1265.2. | DYCACCG | 12 | 19703 | 1.90E−02 | CENTRIMO | | |
| | 2022_ | | ERFO15 | ACAHH | | | | | | |
| | CORE_ | | | (SEQ | | | | | | |
| | non- | | | ID | | | | | | |
| | redundant_ | | | NO: | | | | | | |
| | pfms. | | | 402) | | | | | | |
| | meme | | | | | | | | | |
| 384 | JASPAR | MA1655.1 | MA1655.1. | NRGAACA | 12 | 73159 | 2.00E−02 | CENTRIMO | | |
| | 2022_ | | ZNF341 | GCCNN | | | | | | |
| | CORE_ | | | (SEQ | | | | | | |
| | non- | | | ID | | | | | | |
| | redundant_ | | | NO: | | | | | | |
| | pfms. | | | 403} | | | | | | |
| | meme | | | | | | | | | |
| 385 | JASPAR | MA1696.1 | MA1696.1. | CGGGGRA | 12 | 64819 | 2.20E−02 | CENTRIMO | | |
| | 2022_ | | ARF39 | CACGT | | | | | | |
| | CORE_ | | | (SEQ | | | | | | |
| | non- | | | ID | | | | | | |
| | redundant_ | | | NO: | | | | | | |
| | pfms. | | | 404) | | | | | | |
| | meme | | | | | | | | | |
| 386 | JASPAR | MA1960.1 | MA1960.1. | CYNNNNN | 22 | 71866 | 2.30E−02 | CENTRIMO | | |
| | 2022_ | | Tbox-b | AGGTGTG | | | | | | |
| | CORE_ | | | AAWHNYM | | | | | | |
| | non- | | | N | | | | | | |
| | redundant_ | | | (SEQ | | | | | | |
| | pfms. | | | ID | | | | | | |
| | meme | | | NO: | | | | | | |
| | | | | 405) | | | | | | |
| 387 | JASPAR | MA1887.1 | MA1887.1. | NDCRNNN | 22 | 81755 | 2.30E−02 | CENTRIMO | | |
| | 2022_ | | Brachyury | AGGTGTG | | | | | | |
| | CORE_ | | | AWWWNNN | | | | | | |
| | non- | | | N | | | | | | |
| | redundant_ | | | (SEQ | | | | | | |
| | pfms. | | | ID | | | | | | |
| | meme | | | NO: | | | | | | |
| | | | | 406) | | | | | | |
| 388 | JASPAR | MA0093.3 | MA0093.3. | NDGTCAT | 14 | 37175 | 2.40E−02 | CENTRIMO | | |
| | 2022_ | | USF1 | GTGACH | | | | | | |
| | CORE_ | | | N | | | | | | |
| | non- | | | (SEQ | | | | | | |
| | redundant_ | | | ID | | | | | | |
| | pfms. | | | NO: | | | | | | |
| | meme | | | 407) | | | | | | |
| 389 | JASPAR | MA1731.1 | MA1731.1. | YBVCYBR | 18 | 50124 | 2.40E−02 | CENTRIMO | | |
| | 2022_ | | ZNF768 | SCCTCTC | | | | | | |
| | COREnon- | | | TGDG | | | | | | |
| | redundant_ | | | (SEQ | | | | | | |
| | pfms. | | | ID | | | | | | |
| | meme | | | NO: | | | | | | |
| | | | | 408) | | | | | | |
| 390 | JASPAR | MA1585.1 | MA1585.1. | AYAGTAG | 10 | 14346 | 2.60E−02 | CENTRIMO | | |
| | 2022_ | | ZKSCAN1 | GTS | | | | | | |
| | CORE_ | | | (SEQ | | | | | | |
| | non- | | | ID | | | | | | |
| | redundant_ | | | NO: | | | | | | |
| | pfms. | | | 409) | | | | | | |
| | meme | | | | | | | | | |
| 391 | JASPAR | MA1787.1 | MA1787.1. | GTMAACA | 13 | 60046 | 2.70E−02 | CENTRIMO | | |
| | 2022_ | | ETV5:: | GGAWRY | | | | | | |
| | CORE_ | | FOX01 | (SEQ | | | | | | |
| | non- | | | ID | | | | | | |
| | redundant_ | | | NO: | | | | | | |
| | pfms. | | | 410) | | | | | | |
| | meme | | | | | | | | | |
| 392 | JASPAR | MA0375.1 | MA0375.1. | CSCGCGC | 8 | 26047 | 3.30E−02 | CENTRIMO | | |
| | 2022_ | | RSC30 | G | | | | | | |
| | CORE_ | | | (SEQ | | | | | | |
| | non- | | | ID | | | | | | |
| | redundant_ | | | NO: | | | | | | |
| | pfms. | | | 411) | | | | | | |
| | meme | | | | | | | | | |
| 393 | JASPAR | MA1048.1 | MA1048.1. | RCCGACC | 8 | 16645 | 3.50E−02 | CENTRIMO | | |
| | 2022_ | | ERFO18 | A | | | | | | |
| | CORE_ | | | (SEQ | | | | | | |
| | non- | | | ID | | | | | | |
| | redundant_ | | | NO: | | | | | | |
| | pfms. | | | 412) | | | | | | |
| | meme | | | | | | | | | |
| 394 | JASPAR | MA1064.1 | MA1064.1. | RTGGKMC | 10 | 62543 | 3.60E−02 | CENTRIMO | | |
| | 2022_ | | TCP2 | CAY | | | | | | |
| | CORE_ | | | (SEQ | | | | | | |
| | non- | | | ID | | | | | | |
| | redundant_ | | | NO: | | | | | | |
| | pfms. | | | 413) | | | | | | |
| | meme | | | | | | | | | |
| 395 | JASPAR | MA0585.1 | MA0585.1. | NTTDCCW | 18 | 50205 | 3.60E−02 | CENTRIMO | | |
| | 2022_ | | AGL1 | WWWHDGG | | | | | | |
| | CORE_ | | | WAAN | | | | | | |
| | non- | | | (SEQ | | | | | | |
| | redundant_ | | | ID | | | | | | |
| | pfms. | | | NO: | | | | | | |
| | meme | | | 414) | | | | | | |
| 396 | JASPAR | MA1965.1 | MA1965.1. | CCVNNCC | 20 | 67795 | 4.10E−02 | CENTRIMO | | |
| | 2022_ | | Klf5-like | ACGCCCH | | | | | | |
| | CORE_ | | | NNVVCV | | | | | | |
| | non- | | | (SEQ | | | | | | |
| | redundant_ | | | ID | | | | | | |
| | pfms. | | | NO: | | | | | | |
| | meme | | | 415) | | | | | | |
| 397 | JASPAR | MA0801.1 | MA0801.1. | AGGTGTG | 8 | 61687 | 4.10E−02 | CENTRIMO | | |
| | 2022_ | | MGA | A | | | | | | |
| | CORE_ | | | (SEQ | | | | | | |
| | non- | | | ID | | | | | | |
| | redundant_ | | | NO: | | | | | | |
| | pfms. | | | 416) | | | | | | |
| | meme | | | | | | | | | |
| 398 | JASPAR | MA0288.1 | MA0288.1. | TGACACA | 9 | 56285 | 4.20E−02 | CENTRIMO | | |
| | 2022_ | | CUP9 | WW | | | | | | |
| | CORE_ | | | (SEQ | | | | | | |
| | non- | | | ID | | | | | | |
| | redundant_ | | | NO: | | | | | | |
| | pfms. | | | 417) | | | | | | |
| | meme | | | | | | | | | |
| 399 | JASPAR | MA0659.3 | MA0659.3. | NWGMTGA | 15 | 36891 | 4.30E−02 | CENTRIMO | | |
| | 2022_ | | Mafg | CTCAGCA | | | | | | |
| | CORE_ | | | N | | | | | | |
| | non- | | | (SEQ | | | | | | |
| | redundant_ | | | ID | | | | | | |
| | pfms. | | | NO: | | | | | | |
| | meme | | | 418) | | | | | | |
| 400 | JASPAR | MA0462.2 | MA0462.2. | DATGACT | 11 | 52964 | 5.00E−02 | CENTRIMO | | |
| | 2022_ | | BATF::JUN | CATH | | | | | | |
| | CORE_ | | | (SEQ | | | | | | |
| | non- | | | ID | | | | | | |
| | redundant_ | | | NO: | | | | | | |
| | pfms. | | | 419) | | | | | | |
| | meme | | | | | | | | | |
| 401 | JASPAR | MA1695.1 | MA1695.1. | RCGGGGG | 14 | 39450 | 5.00E−02 | CENTRIMO | | |
| | 2022_ | | ARF36 | ACAHGTC | | | | | | |
| | CORE_ | | | (SEQ | | | | | | |
| | non- | | | ID | | | | | | |
| | redundant_ | | | NO: | | | | | | |
| | pfms. | | | 420) | | | | | | |
| | meme | | | | | | | | | |

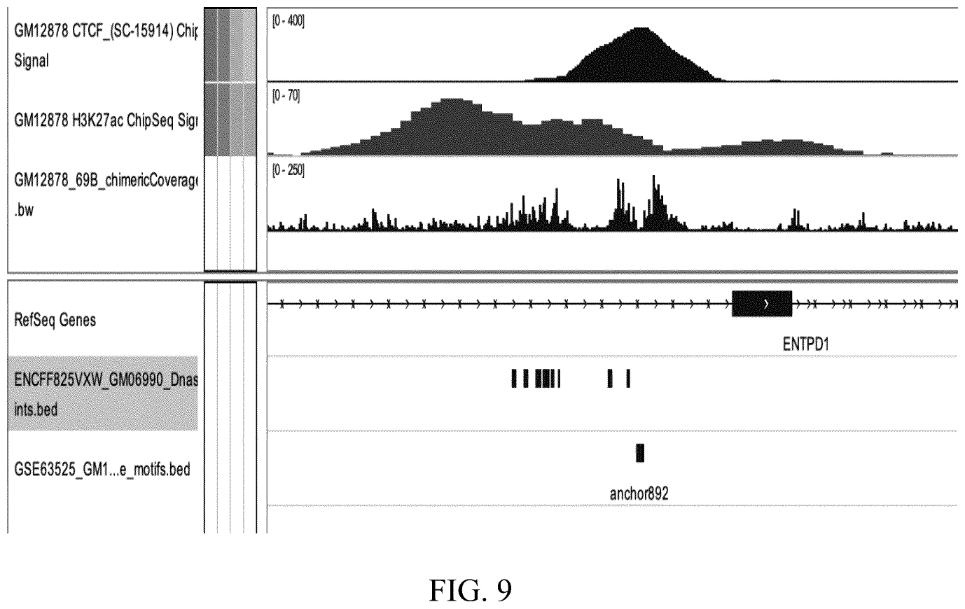

FIG. 9 shows that intact Hi-C can be used similarly to ultra-deep DNase-Seq to identify protected areas of DNA in addition to DNA contacts and phasing. The cut sites identified with intact Hi-C correspond to the DNA hypersensitivity sites surrounding the CTCF motif and correspond to the peak of ChIP-seq for CTCF. The CTCF motif also forms a boundary for H3K27ac.

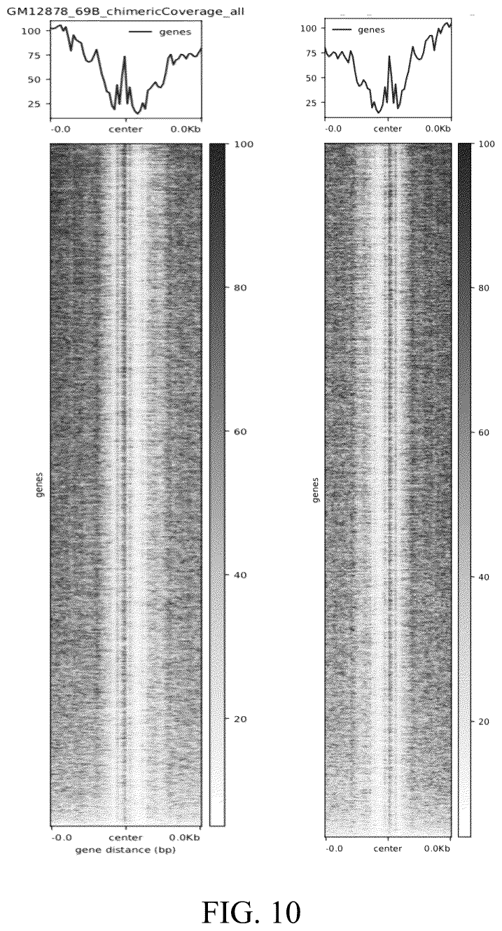

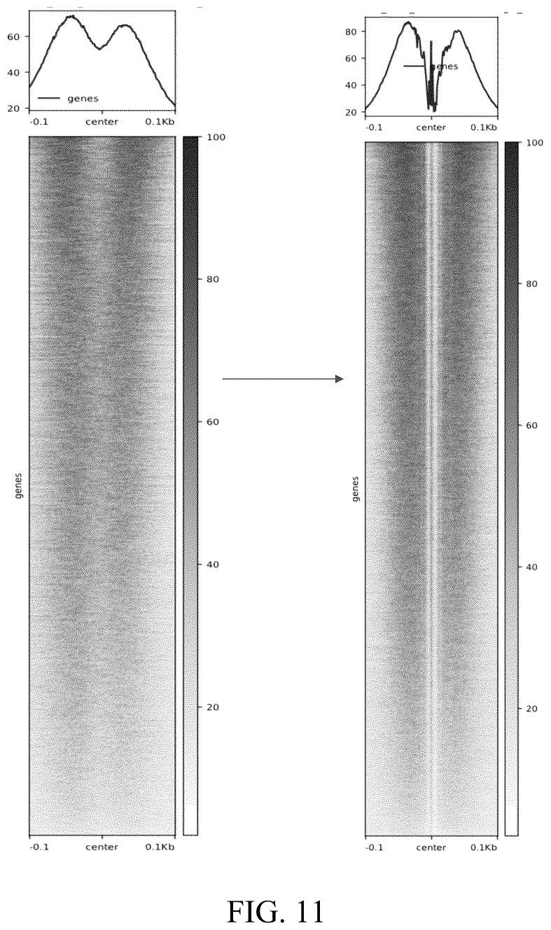

FIG. 10 shows that intact Hi-C can show exact footprints of CTCF binding to convergent CTCF motifs as shown by the area where there are no cut sites. The pattern shows the exact contact sites and the patterns are in a convergent orientation as the fragmentation pattern is reversed for the forward and reverse CTCF anchors. The footprinting also shows that the native conformation of CTCF and chromatin binding is maintained in all nuclei analyzed. The pattern of cut sites is consistent in all sequenced ligation junctions. In methods where intact chromatin is not maintained CTCF can fall off and it would not be possible to generate a sharp footprint as shown with intact Hi-C. FIG. 11 further shows that loop anchor localization can be improved by using the DNase footprint that can be obtained with intact Hi-C. Intact Hi-C can produce deep, 1 bp resolution chromatin accessibility tracks. DNase footprints reveal the specific protein motif for each loop anchor. Intact Hi-C can identify proteins associated with each loop.

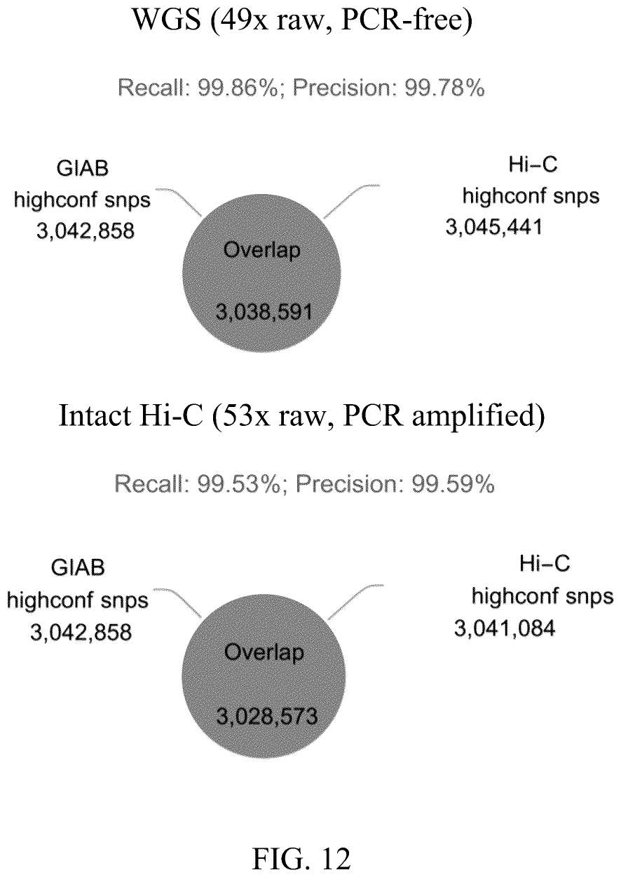

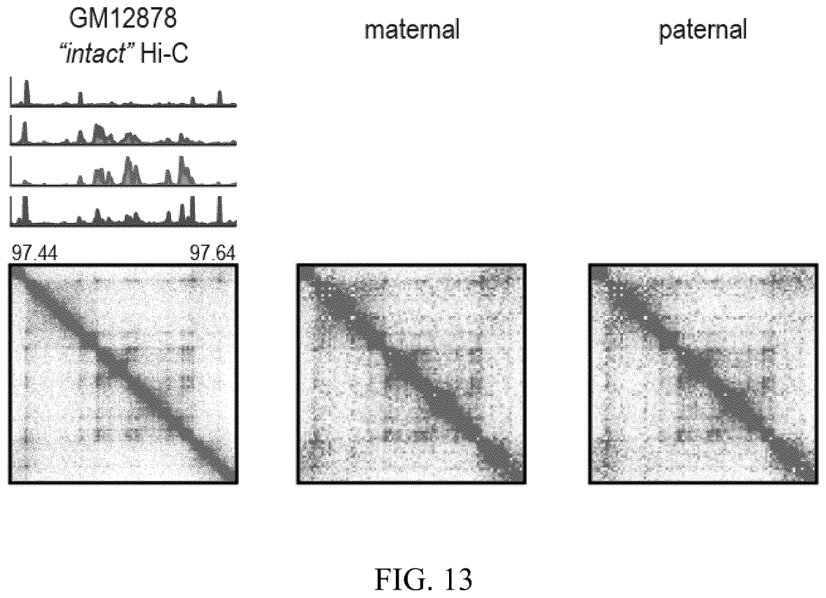

Using external SNP data, in situ Hi-C maps can be phased to generate allelic contact maps, but previous attempts poorly resolved features at the scale of loops (Rao and Huntly et al., Cell 2014). Intact Hi-C can be used to call SNPs with high precision (FIG. 12). The Hi-C resequencing pipeline can be used to call SNPs and phase them onto chromosome length haploblocks. This enables loop resolution diploid Hi-C contact maps for every experiment (FIG. 13).

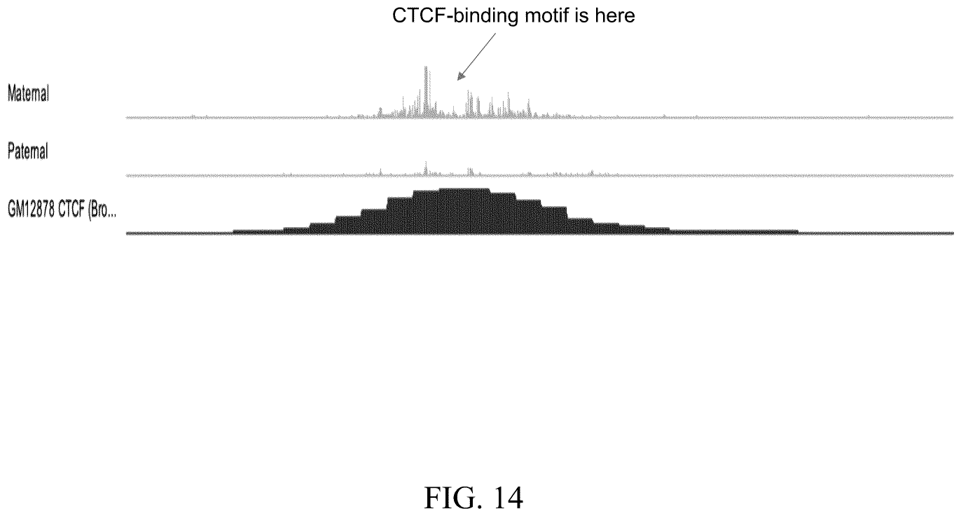

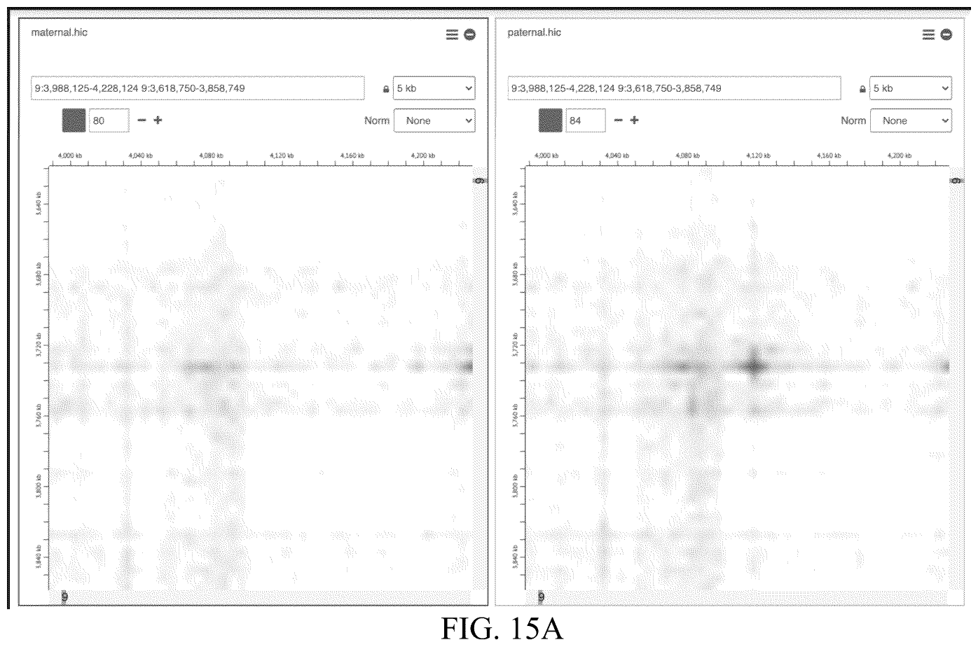

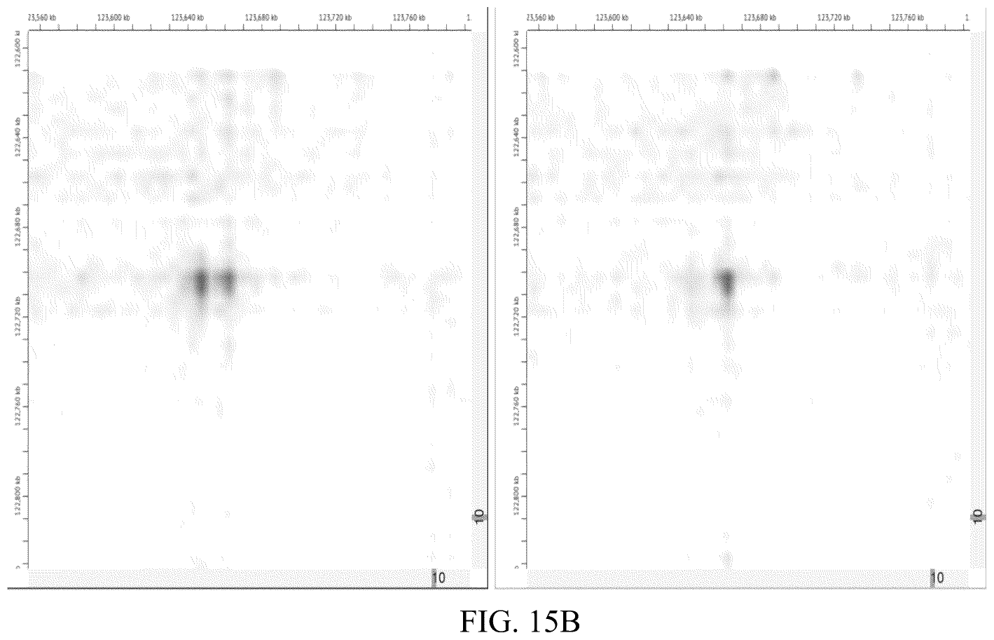

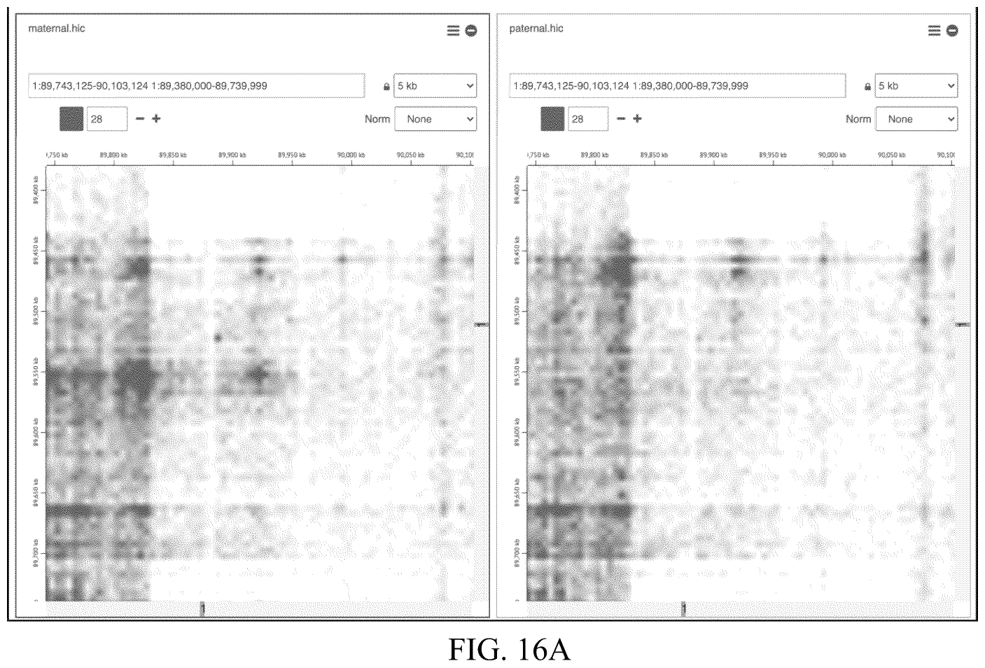

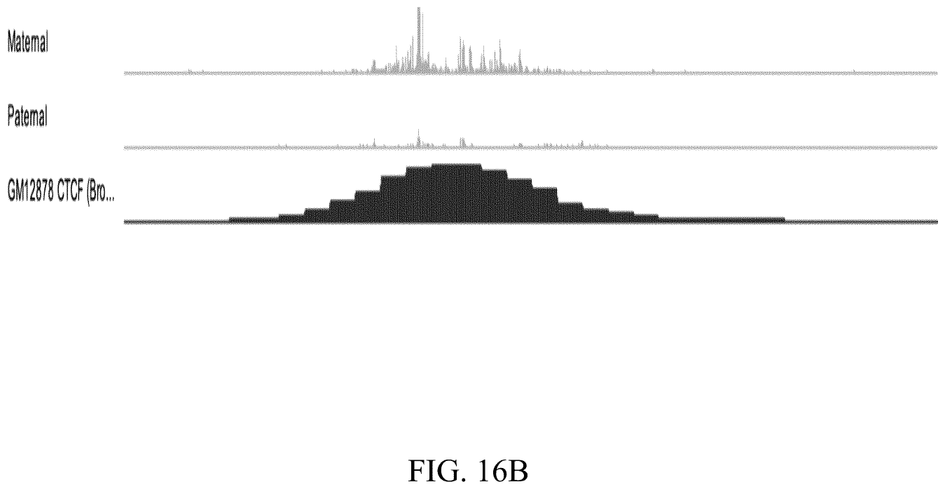

FIG. 14 shows that intact Hi-C can be used to phase the paternal and maternal chromosomes by using DNA contacts to indicate fragments on the same chromosome. In this example, CTCF binding is localized to the maternal chromosome, indicating a loop on the maternal chromosome. FIG. 15 shows SNPs in CTCF motifs on one chromosome causes no loop to be formed on that chromosome. FIG. 16 shows loops in the maternal chromosome that are not present on the paternal chromosome. The DNase sensitivity map of the maternal chromosome shows CTCF binding that is consistent with unphased ChIP-seq data. The DNase sensitivity of the paternal chromosome shows no CTCF binding. Thus, intact Hi-C can predict the effect of every single variant on protein binding, loop formation, and gene expression.

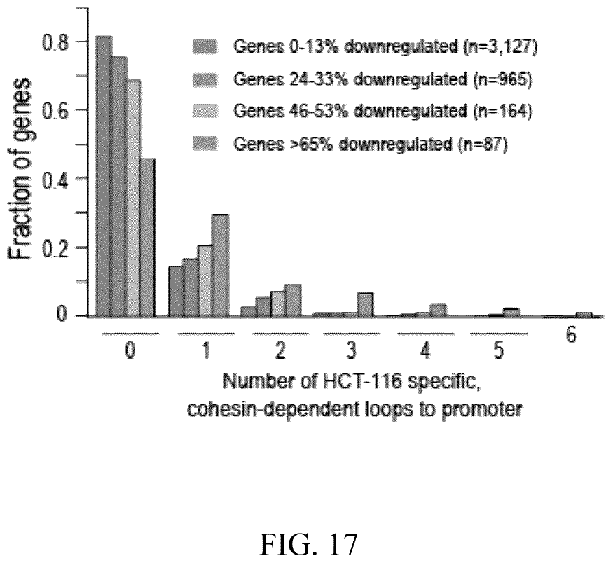

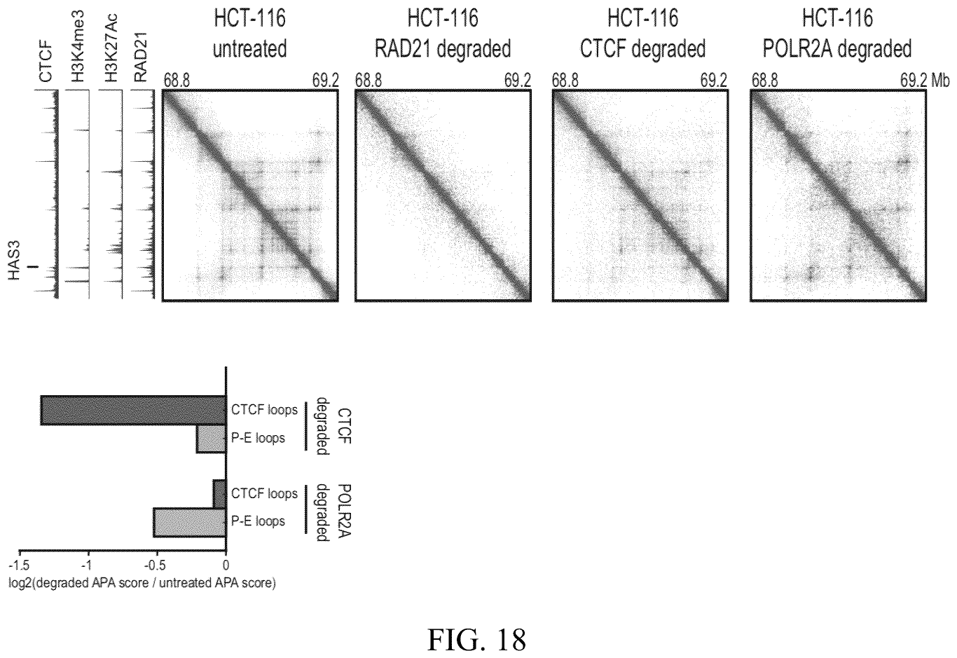

FIG. 17 shows that promoter-enhancer loop loss results in downregulation of genes. FIG. 18 shows that intact Hi-C makes degron-mediated experiments much more informative. FIG. 18 shows that all loops are cohesin dependent (RAD21). P-E loops form when RNA polymerase II blocks cohesin at a promoter sequence. CTCF loops form when CTCF blocks cohesin at a CTCF motif. ChIP indicates the location of CTCF, cohesin complex, and histone modifications associated with active transcription. This is consistent with data showing that deletion of CTCF does not eliminate all loops, but deletion of cohesin does eliminate all loops (see, e.g., Rao S S P, Huang S C, Glenn St Hilaire B, et al. Cohesin Loss Eliminates All Loop Domains. Cell. 2017; 171(2):305-320.e24).

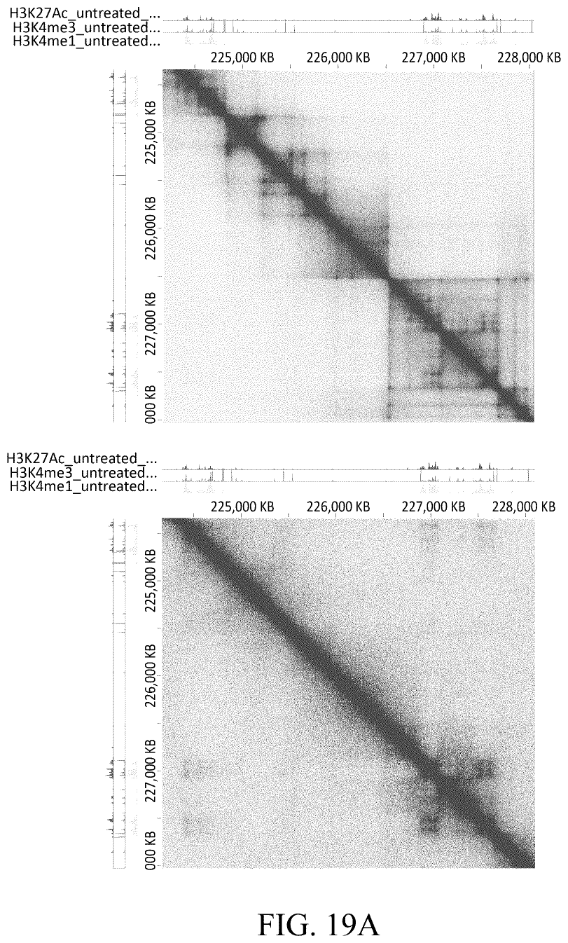

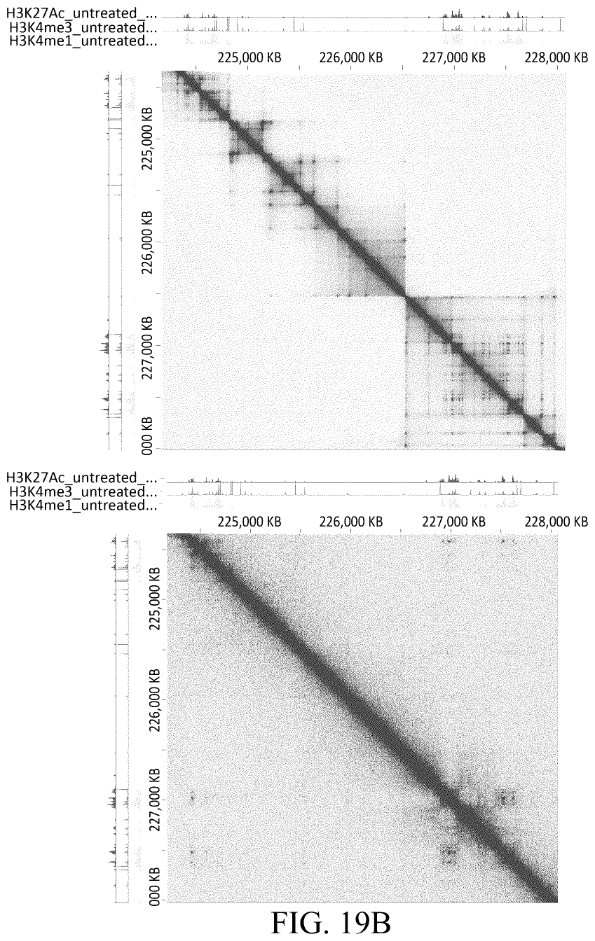

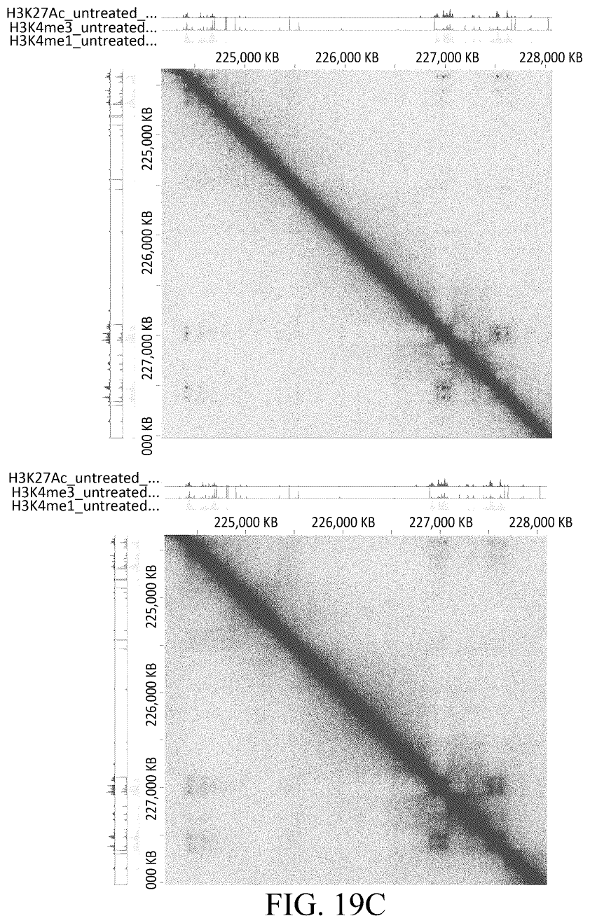

In the absence of cohesin, superenhancers colocalize (see, e.g., Rao S S P, Huang S C, Glenn St Hilaire B, et al. Cohesin Loss Eliminates All Loop Domains. Cell. 2017; 171(2):305-320.e24). FIG. 19 shows superenhancers using intact Hi-C as compared to in situ Hi-C. Superenhancer links show increasingly punctate signal in intact Hi-C data.

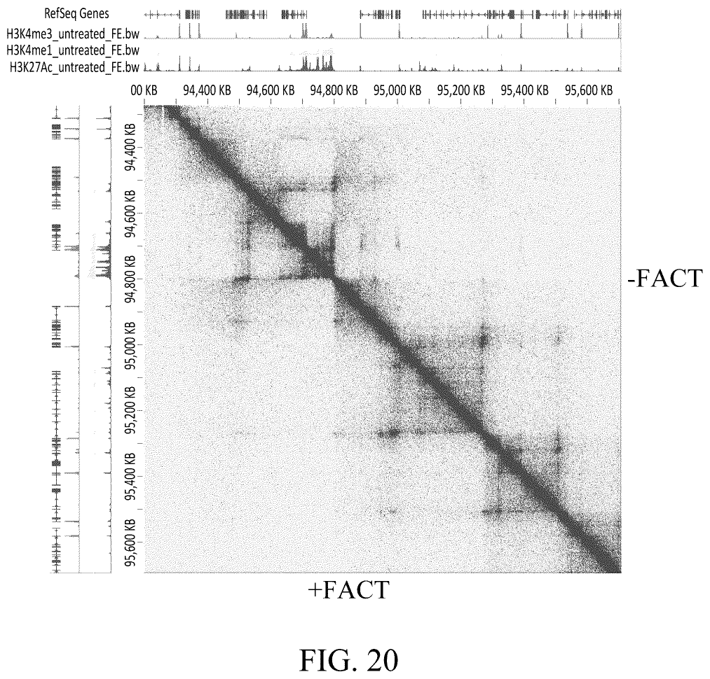

FAcilitates Chromatin Transcription (FACT), a histone chaperone complex, is involved in nucleosome remodeling via eviction or assembly of histones during transcription, replication, and DNA repair (see, e.g., Bhakat K K, Ray S. The Facilitates Chromatin Transcription (FACT) complex: Its roles in DNA repair and implications for cancer therapy. DNA Repair (Amst). 2022; 109:103246; and Belotserkovskaya R, Reinberg D. Facts about FACT and transcript elongation through chromatin. Curr Opin Genet Dev. 2004; 14(2):139-146). FIG. 20 shows that in the absence of FACT promoters colocalize.

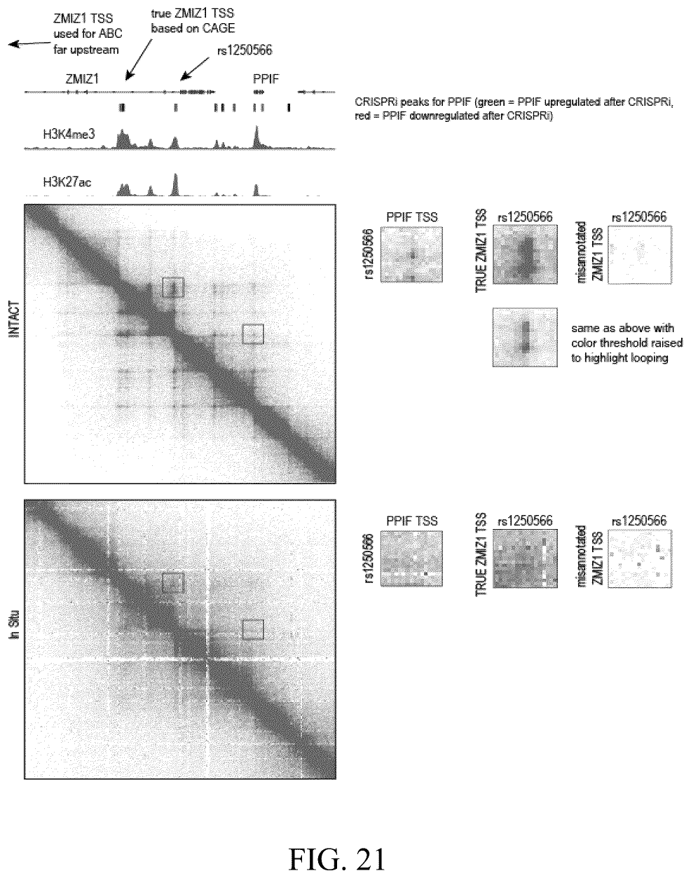

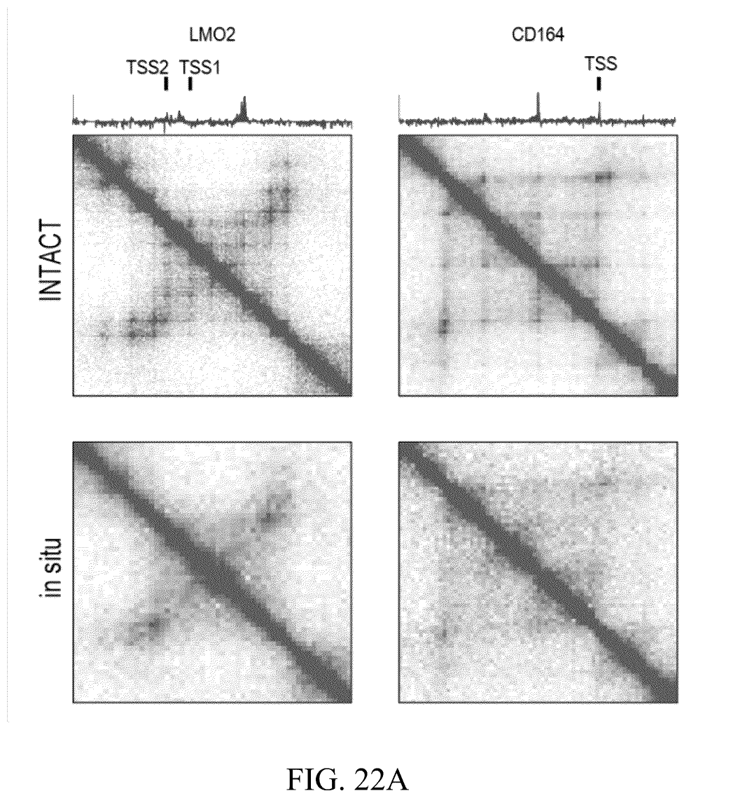

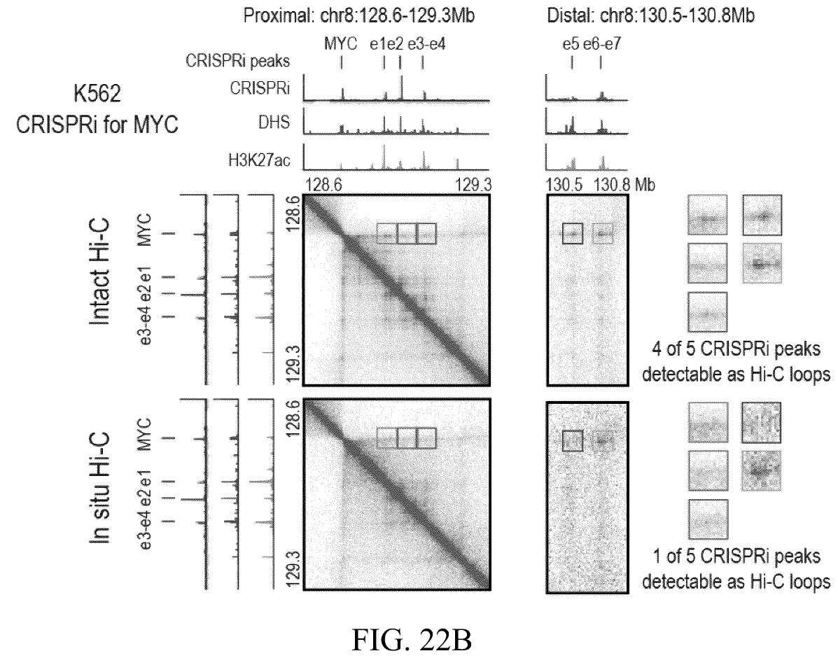

FIG. 21 demonstrates determining function from looping. Nasser et al, predict regulation of PPIF by an intronic enhancer in ZMIZ1 containing an IBD associated SNP in immune cells using the ABC model and validated the prediction with CRISPRi in several immune cell lines, including GM12878 (Nasser J, Bergman D T, Fulco C P, et al. Genome-wide enhancer maps link risk variants to disease genes. Nature. 2021; 593(7858):238-243). Intact Hi-C detects a more complicated network of loops between the regulatory elements at this locus, including a strong loop between the IBD associated SNP and an alternate intronic transcript supported by CAGE data. FIG. 22 shows that lower depth intact Hi-C still efficiently detects functional promoter-enhancer loops validated by CRISPRi.

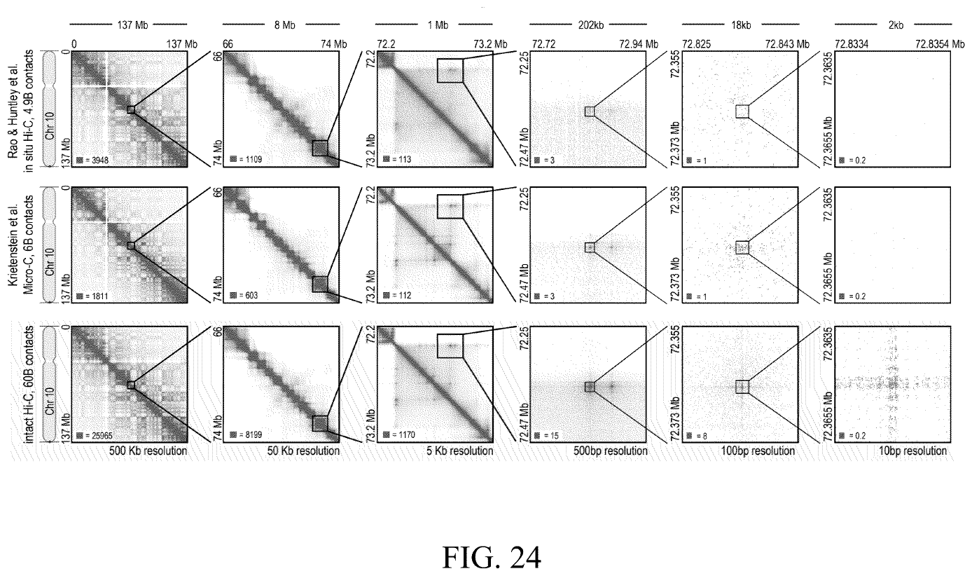

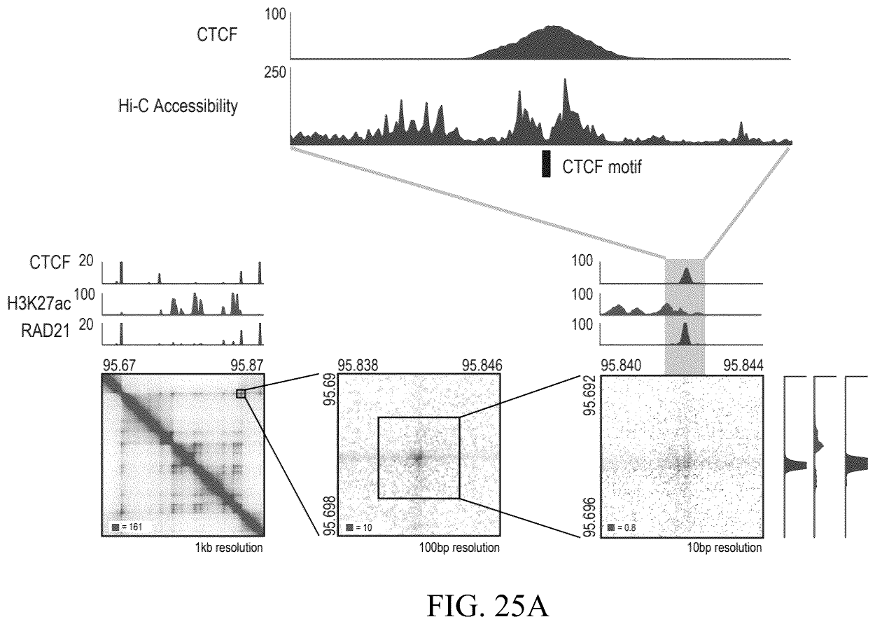

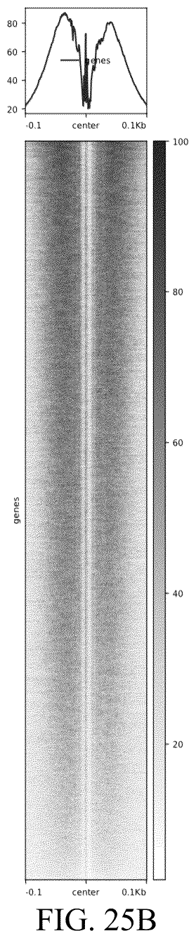

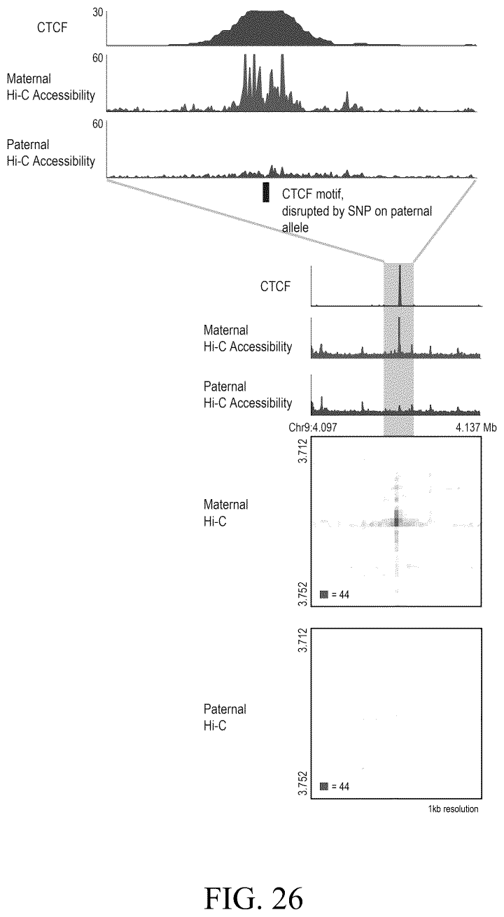

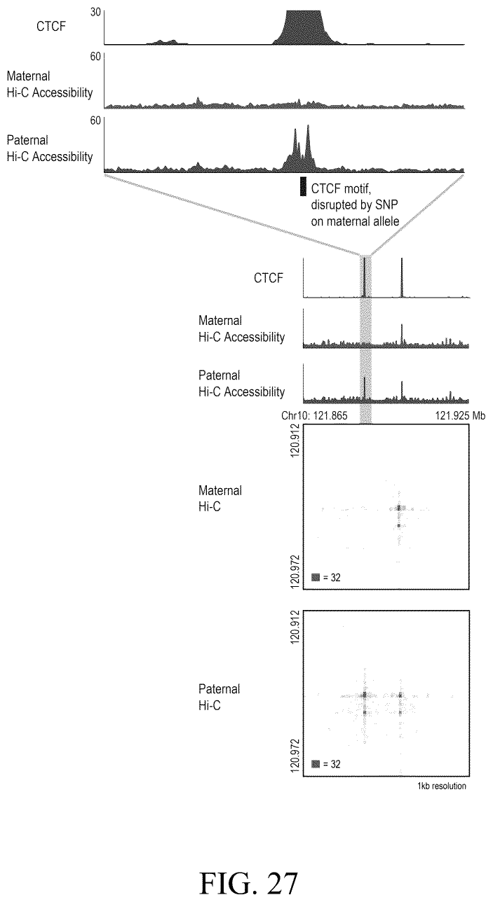

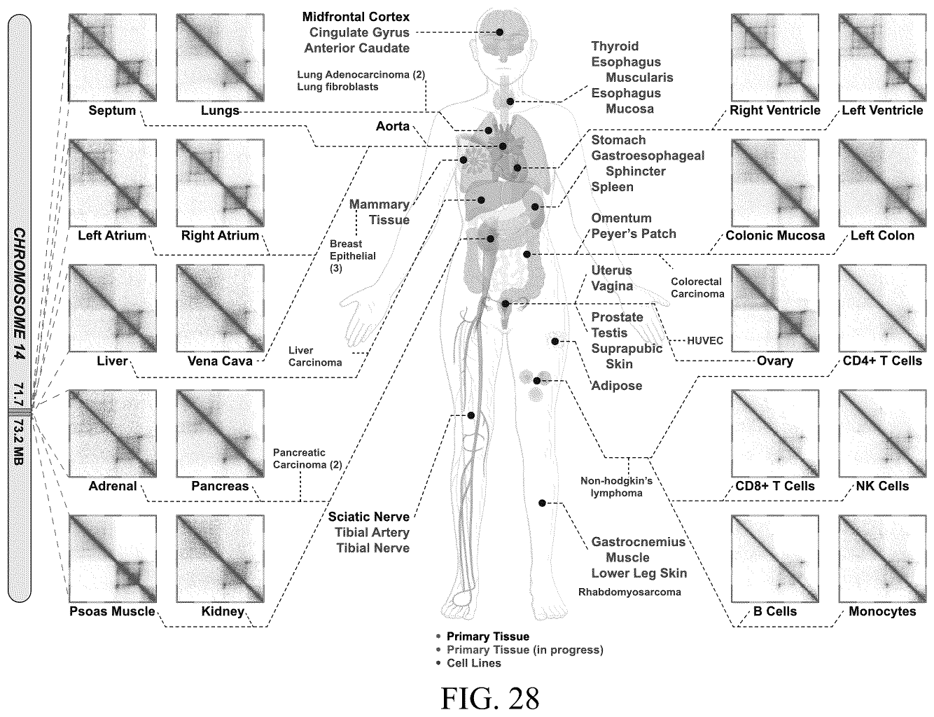

FIG. 24 shows that intact Hi-C has base pair resolution. FIG. 25 shows that intact Hi-C can be used to determine protein binding on the genome. FIGS. 26 and 27 show that intact Hi-C can be used to phase protein binding to chromosomes. FIG. 28 shows that intact Hi-C can be used to build an atlas of the loops in every human tissue.

### Example 2—Exemplary Protocols for Intact Hi-C

Intact Hi-C is a method for probing the three-dimensional architecture of a genome using DNA-to-DNA contact mapping. The core step of intact Hi-C uses the enzyme T4 DNA ligase to preferentially ligate genomic DNA fragments that are in close physical proximity within the cell nucleus. The resulting ligation junctions are then characterized by means of DNA sequencing.

Intact Hi-C is a modular protocol, which means that at several steps, the experimenter can choose between multiple robust, interchangeable options. The options should be chosen to best fit the experimental needs. The choice of modules makes it possible to process a wide variety of samples and to create multi-omics assays that simultaneously measure contact frequency and, for example, DNase accessibility or DNA methylation.

For the protocols described below, the input is a population of mammalian cells with intact nuclei, and the output is a library of double-stranded DNA fragments ready for next-generation sequencing. The fastest iteration of this modular protocol can be done in ˜2 days, but depending on specific modules chosen as well as the number of samples, the workflow may be better accommodated over 3-5 days and contains many natural pause points to facilitate this.

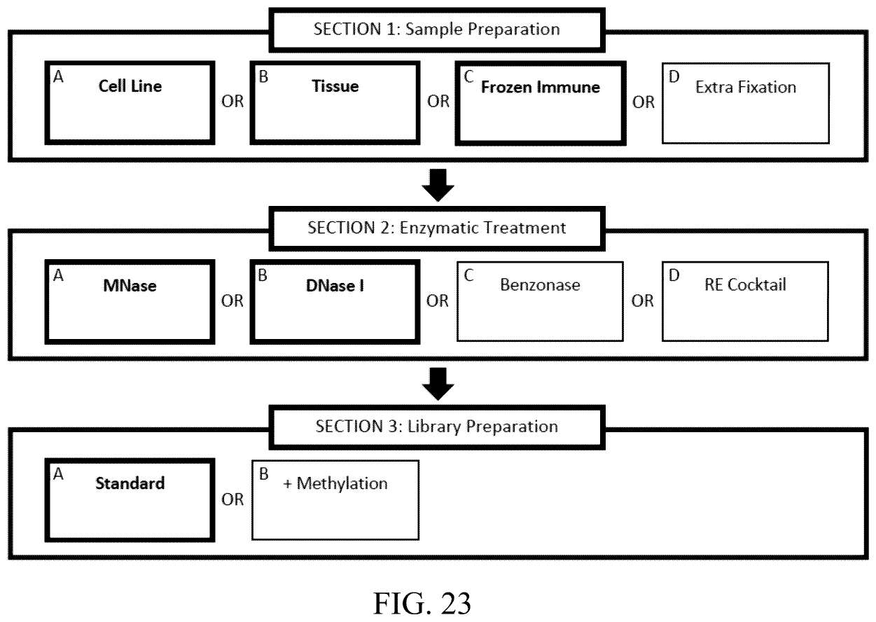

FIG. 23 provides the Intact Hi-C protocol in a flowchart. The protocol consists of 3 sections: (1) sample preparation, (2) enzymatic treatment, and (3) library preparation. Each section can be completed in one or two workdays. When planning a new intact Hi-C experiment, the first step is to decide which modules to use. Exactly one module is chosen from each section. Then the flowchart or the table of contents is used to locate, print out, and follow only the steps from the three modules chosen, ignoring all of the remaining modules.

There are three specific combinations of modules that are used for large-scale ENCODE (Encyclopedia of DNA Elements) production efforts. The modules used in these combinations are shown in bold font in the flowchart and the table of contents.

ENCODE Standard Protocol #1: Cell lines

### Module 1A+Module 2A+Module 3A

ENCODE Standard Protocol #2: Solid tissues

### Module 1B+Module 2B+Module 3A

ENCODE Standard Protocol #3: Cryopreserved immune cells

### Module 1C+Module 2A+Module 3A

### Table of Contents

### Flowchart

### General Notes Before Beginning

### Stock Solutions

### Section 1: Sample Preparation

Module 1A: Fixation of Liquid Culture with Formaldehyde
Module 1B: Fixation of Solid Tissue with Formaldehyde
Module 1C: Fixation of Cryopreserved Immune Cells with Formaldehyde
Module 1D: Fixation with Additional Crosslinking

### Section 2: Enzymatic Treatment

Module 2A: Digestion with Micrococcal Nuclease
Module 2B: Digestion with DNase I
Module 2C: Digestion with Benzonase
Module 2D: Digestion with Restriction Enzyme Cocktail

### Section 3: Library Preparation

Module 3A: Illumina Library Preparation (without Methylation Detection)
Module 3B: Illumina Library Preparation with Methylation Detection

### General Notes Before Beginning

1) Throughput: This protocol is written with the assumption that you are handling one sample at a time, using single-channel pipettes. However, several samples can be comfortably processed in parallel. To further increase throughput, Sections 2 and 3 are fully compatible with multichannel pipetting. The volumes will fit comfortably in 0.2 ml PCR tubes without needing to be scaled down. When processing multiple samples in parallel, add an extra 10% volume to each master mix to account for pipetting error.
2) Centrifugation: All centrifuge speeds are given in RCF (for example, 300×g) and not in RPM because RPM depends on the specifications of each particular centrifuge rotor, whereas RCF is universal.
3) Sequencing Platforms: The library preparation instructions in Section 3 are described for the Illumina paired-end sequencing platform, but the Ultima Genomics single-end sequencing platform may be used instead. Either amplify the genomic library directly with Ultima adaptors or convert a finished Illumina library to be compatible with the Ultima platform following the manufacturer's recommendations. Regardless of the sequencing platform, it is extremely important to obtain reads that are long enough to span the entire length of the insert, capturing the ligation junction. Creating a high-resolution contact map with precise localization of each interacting piece of DNA depends on sequencing through the ligation junction. If using the Illumina platform, 150PE reads are strongly recommended.

### Stock Solutions

The following four stock solutions are used across all of the modules of intact Hi-C:

### Lysis Buffer

Combine the following ingredients in a 50 ml conical tube:
i. 19.36 ml of water (ThermoFisher #10977-023)
ii. 200 μl of 1M Tris-HCl pH 8.0 [final: 10 mM] (ThermoFisher, AM9855G or VWR #97062-674)
iii. 40 μl of 5M NaCl [final: 10 mM] (ThermoFisher #AM9759)
iv. 400 μl of 10% (v/v) IGEPAL CA-630 [final: 0.2%] (ThermoFisher #J61055-AE)

Mix by inverting and store at 4° C. for up to 1 month. This buffer is used in Sections 1 and 2.

### 10 mM Tris Buffer

Combine the following ingredients in a 50 ml conical tube:
i. 39.6 ml of water
ii. 400 μl of 1M Tris-HCl pH 8.0 [final: 10 mM]

Mix by vortexing and store at room temperature for up to 1 year. This buffer is used in Sections 2 and 3.

### 3× Tween Wash Buffer (3×TWB)

Combine the following ingredients in a 50 ml conical tube:
i. 14.68 ml of water
ii. 24 ml of 5M NaCl [final: 3M]
iii. 600 μl of 1M Tris-HCl pH 8.0 [final: 15 mM]
iv. 120 μl of 500 mM EDTA pH 8.0 [final: 1.5 mM] (ThermoFisher, AM9260G or Corning #46-034-CI)
v. 600 μl of 10% (w/v) Tween 20 [final: 0.15%] (ThermoFisher #28320)

Mix by inverting and store at 4° C. for up to 1 month. This buffer is used in Section 3.

### 1× Tween Wash Buffer (1×TWB)

Combine the following ingredients in a 50 ml conical tube:
i. 20 ml of water
ii. 10 ml of 3×TWB

Mix by inverting and store at 4° C. for up to 1 month. This buffer is used in Section 3.

### Section 1: Sample Preparation

Module 1A: Fixation of Liquid Culture with Formaldehyde

Use this module when starting with a live immortalized or primary cell line.

### Module 1A Step 1 of 5: Cell Culture

Grow mammalian cells in vitro to ˜80% confluence following the manufacturer's recommended culturing protocol. Use proper aseptic technique to limit contamination.

If the cells are adherent, trypsinize or scrape to detach them from the inner surface of the flask. Working quickly, transfer the cells in their growth medium to one or more 50 ml conical tubes. Pool together flasks or plates as needed. Mix by gentle pipetting, then take a small aliquot from each tube for counting and mycoplasma testing.

Centrifuge at 300×g for 5 minutes. Meanwhile, count the cells in each aliquot to estimate the total number of cells in each tube. Use these estimates to calculate the required volumes of formaldehyde and glycine in Steps 2 and 3.

Immediately discard the supernatant and resuspend the cell pellet in fresh growth medium at a concentration of 1 million cells per 1 ml of medium. Plan ahead so that the volumes of formaldehyde and glycine added in Steps 2 and 3 do not exceed the capacity of the tube. Split the sample volume into multiple tubes if necessary.

### Module 1A Step 2 of 5: Fixation

In a chemical fume hood, add freshly opened formaldehyde solution (ThermoFisher, 28908) to a final concentration of 1% (w/v). Close the tube cap securely. Incubate at room temperature with constant rocking or nutation for exactly 10 minutes to crosslink proteins and fix chromatin in place. [Meanwhile, pre-chill centrifuges to 4° C. for Steps 4 and 5, and fill an ice bucket.]

### Module 1A Step 3 of 5: Quenching

In a chemical fume hood, add a glycine (Sigma, G7403-1KG) stock solution to a final concentration of 200 mM. Close the tube cap securely. Incubate at room temperature with constant rocking or nutation for 5 minutes to quench the formaldehyde and prevent over-crosslinking. [Meanwhile, prepare the cold bath for Step 5.]

### Module 1A Step 4 of 5: Post-Fixation Wash

Centrifuge at 300×g for 5 minutes in a pre-chilled 4° C. centrifuge (Eppendorf, 5804 R). In a chemical fume hood, immediately discard the supernatant into a hazardous waste container, following your institution's guidelines.

Optional: You may wash the cell pellet to more thoroughly remove any traces of formaldehyde and glycine. Resuspend the cell pellet in ice-cold 1×PBS (ThermoFisher, 10010-023) at a concentration of 1 million cells per 1 ml of buffer. Centrifuge at 300×g for 5 minutes in a pre-chilled 4° C. centrifuge. In a chemical fume hood, immediately discard the supernatant into a hazardous waste container, following your institution's guidelines.

Resuspend the cell pellet in ice-cold 1×PBS (ThermoFisher, 10010-023) such that the sample volume (in ml, rounded down to the nearest ml) corresponds to the number of flash-frozen pellets you intend to make. For example, to make flash-frozen pellets of 8 million cells each, resuspend the cell pellet in one-eighth of the volume used in Step 1.

On ice, mix well by pipetting, and aliquot the sample into meticulously labeled 1.5 ml microcentrifuge tubes (VWR, 80077-230) at 1 ml per tube.

### Module 1A Step 5 of 5: Flash-Freezing

Centrifuge at 300×g for 5 minutes in a pre-chilled 4° C. centrifuge (Eppendorf, 5424 R). Immediately discard the supernatant, close the tube securely, and flash-freeze the cell pellet in a liquid nitrogen bath or in a dry ice and 100% (v/v) ethanol bath.

Store the flash-frozen cell pellets at −80° C. indefinitely.

### Section 1: Sample Preparation

Module 1B: Fixation of Solid Tissue with Formaldehyde

Use this module when starting with a solid piece of tissue.

### Module 1B Step 1 of 9: Buffer Preparation

The following six stock solutions can be prepared in advance:
i. 60% (w/v) sucrose: Dissolve 300 g of sucrose (Sigma, S8501-10KG) in deionized water up to a volume of 500 ml. Sterilize by filtering through a 0.2 μm filter. Store at 4° C.
ii. 500 mM CaCl₂): Dissolve 3.675 g of calcium chloride dihydrate (Sigma, C3881-500G) in deionized water up to a volume of 50 ml. Sterilize by filtering through a 0.2 μm filter. Store at room temperature for up to 6 months.
iii. 300 mM Mg(OAc)₂: Dissolve 3.217 g of magnesium acetate tetrahydrate (Sigma, M5661-50G) in deionized water up to a volume of 50 ml. Sterilize by filtering through a 0.2 μm filter. Store at room temperature for up to 6 months.
iv. 1.25M glycine: Dissolve 46.919 g of glycine (Sigma, G7403-1KG) in deionized water up to a volume of 500 ml. Sterilize by filtering through a 0.2 μm filter. Store at 4° C.
v. 10% (v/v) IGEPAL CA-630: Combine 9 ml of water with 1 ml of IGEPAL CA-630 (Sigma, I8896-100ML) in a 50 ml conical tube. Vortex to homogenize. Store at room temperature for up to 2 weeks, but preferably freshly prepare every week.

Freshly prepare the following dilutions on the day of sample preparation and store them on ice until they are needed:
i. 1% (w/v) formaldehyde: Working in a chemical fume hood, combine 13.4 ml of water, 1.6 ml of 10×PBS pH 7.4 (ThermoFisher, 70011-044), and 1 ml of freshly opened 16% (w/v) formaldehyde (ThermoFisher, 28906) in a 50 ml conical tube.
ii. 200 mM glycine: Combine 37 ml of water, 8 ml of 1.25M glycine, and 5 ml of 10×PBS pH 7.4 in a 50 ml conical tube.

Freshly prepare the following working solutions on the day of sample preparation and store them on ice until they are needed. If processing multiple samples in parallel (recommended for experiment replication and to facilitate centrifuge balancing), multiply each volume below by the number of tissue samples plus an extra one in order to guarantee a sufficient volume of each solution. To maintain sample integrity, plan to process no more than six samples at a time.

### Homogenization Buffer:

i. 3.2 ml of water (ThermoFisher, 10977-023)
ii. 1.6 ml of 60% (w/v) sucrose
iii. 50 μl of 1M Tris pH 8.0 (ThermoFisher, AM9855G)
iv. 50 μl of 10% (v/v) IGEPAL CA-630
v. 50 μl of 500 mM CaCl₂)
vi. 50 μl of 300 mM Mg(OAc)₂

### 83% OptiPrep Solution:

i. 4.15 ml of OptiPrep Density Gradient Medium (Sigma, D1556-250ML)
ii. 700 μl of water
iii. 50 μl of 1M Tris pH 8.0
iv. 50 μl of 500 mM CaCl₂)
v. 50 μl of 300 mM Mg(OAc)₂

### 48% OptiPrep Solution:

i. 4.8 ml of OptiPrep Density Gradient Medium
ii. 3.05 ml of water
iii. 1.8 ml of 60% (w/v) sucrose
iv. 100 μl of 1M Tris pH 8.0
v. 50 μl of 10% (v/v) IGEPAL CA-630
vi. 100 μl of 500 mM CaCl₂)
vii. 100 μl of 300 mM Mg(OAc)₂

### Module 1B Step 2 of 9: Mincing

Fill an ice bucket and place a fresh Petri dish (VWR, 25384-342) directly on top of the ice. Place the solid tissue sample in the Petri dish.

Using a fresh razor blade (VWR, 55411-050) and clean forceps, quickly cut and weigh 20-30 mg of the tissue in a fresh weigh boat. Put the rest of the tissue away, and place the 20-30 mg sample back into the Petri dish on ice. Note that approximately 2-3 mg of tissue is the appropriate amount for one intact Hi-C library. A 20-30 mg sample is a comfortable amount to process at one time and will yield cell pellets sufficient to make 10 intact Hi-C libraries. Handling more than 30 mg is not recommended because it may be too much material for the subsequent steps to work effectively. If you have much less starting material, you may still attempt the protocol, but be aware that it may be lossy and your yield may be very low.

To ensure homogeneous crosslinking, mince the sample with a fresh razor blade into the smallest possible pieces, ideally less than 1 mm³ in size. Transfer the tissue pieces into a fresh 1.5 ml microcentrifuge tube (VWR, 80077-230) on ice.

Alternative Options: When working with exceptionally fragile and delicate tissues, it is vital to handle them as gently as possible and to minimize the amount of time between removing the tissue from the freezer and crosslinking it. Instead of a simple ice bucket, you may use a Cooling Workstation Core (Azenta, BCS-511) pre-chilled at −80° C. as a stable platform for the Petri dish. Before taking out the tissue sample, fill afresh 1.5 ml tube with a 1 ml aliquot of ice-cold 1% (w v) formaldehyde and place this tube on a balance in a chemical fume hood. Then place the tissue sample in the ice-cold Petri dish and immediately cut very thin slices of the tissue, putting each slice directly in the 1.5 ml tube with formaldehyde instead of in a weigh boat. Keep adding slices of tissue to the 1.5 ml tube until you reach a total of 20-30 mg. Do not spend any time mincing the tissue pieces and instead proceed directly to Step 3.

### Module 1B Step 3 of 9: Fixation

In a chemical fume hood, add 1 ml of ice-cold 1% (w/v) formaldehyde. Close the tube cap securely. Incubate at room temperature with gentle, continuous inverting by hand for exactly 10 minutes to crosslink proteins and fix chromatin in place. [Meanwhile, pre-chill a centrifuge to 4° C.]

Centrifuge at 6000×g for 2 minutes in a pre-chilled 4° C. centrifuge (Eppendorf, 5424 R). In a chemical fume hood, immediately place on ice and discard the supernatant into a hazardous waste container, following your institution's guidelines.

### Module 1B Step 4 of 9: Quenching

In a chemical fume hood, add 1 ml of ice-cold 200 mM glycine. Close the tube cap securely. Incubate at room temperature with gentle, continuous inverting by hand for exactly 5 minutes to quench the formaldehyde.

Centrifuge at 6000×g for 2 minutes in a pre-chilled 4° C. centrifuge. In a chemical fume hood, immediately place on ice and discard the supernatant into a hazardous waste container, following your institution's guidelines.

Repeat this step once more to fully quench the formaldehyde and prevent over-crosslinking.

### Module 1B Step 5 of 9: Post-Fixation Washes

Add 1 ml of ice-cold 1×PBS (ThermoFisher, 10010-023). Mix by inverting and centrifuge at 6000×g for 2 minutes in a pre-chilled 4° C. centrifuge. Place on ice and discard the supernatant. Repeat this step once more to thoroughly wash the tissue sample.

### Module 1B Step 6 of 9: Homogenization

Add 1 ml of ice-cold Homogenization Buffer. Mix by inverting and incubate on ice for 10 minutes. [Meanwhile, pre-chill a clean Dounce tissue grinder on ice.]

Transfer the entire sample volume to a clean 7 ml Dounce tissue grinder tube (DWK, 885303-0007) on ice. Using a clean large-clearance pestle A (DWK, 885301-0007), apply 15-20 strokes to crush the tissue. Fibrous tissues, such as muscle, may require up to 25 strokes. Apply forceful pressure and rotate the pestle to fully dissociate the cells. Keeping the pestle within the Douncer, carefully rinse the pestle with 1 ml of Homogenization Buffer, collecting the rinse volume in the Douncer.

Using a clean small-clearance pestle B (DWK, 885302-0007), apply 10-15 strokes to fully homogenize the tissue. Keeping the pestle within the Douncer, carefully rinse the pestle with 1 ml of Homogenization Buffer, collecting the rinse volume in the Douncer.

### Module 1B Step 7 of 9: Filtering

Place a fresh 50 ml conical tube on ice and remove the cap. Place a 100 μm cell strainer (Fisher, 22-363-549) or a 70 μm cell strainer (Fisher, 22-363-548) in the tube.

Transfer the entire sample volume through the cell strainer into the tube. Large pieces, especially fibers from fibrous tissues, will be retained on the filter, while the filtrate will contain nuclei and smaller cell debris. Discard the cell strainer.

Measure the volume of the filtrate. Add Homogenization Buffer to bring the total sample volume to exactly 5 ml. Then add exactly 5 ml of 83% OptiPrep Solution. Mix by gently pipetting the entire volume twice, and place on ice.

### Module 1B Step 8 of 9: Density Gradient Centrifugation

Pre-chill a centrifuge to 4° C. (Eppendorf, 5804 R). Place a fresh 45 ml round-bottom centrifuge tube (Crystalgen, 23-2589) on ice. Add 10 ml of 48% OptiPrep Solution to the bottom of the 45 ml tube.

Extremely slowly and carefully layer the 10 ml sample volume on top of the 48% OptiPrep Solution by tilting the 45 ml tube at an angle and pipetting a thin stream down the inner wall of the tube, so as not to mix the two layers together. The interface between the two layers should be clearly visible.

Close the cap securely and carefully place the sample into the pre-chilled centrifuge, without disturbing the two layers. Set the centrifuge acceleration rate to 5/9 (i.e., half of the maximum acceleration rate) and the deceleration rate to 0/9 (i.e., no brake). Centrifuge at 3200×g for 30 minutes at 4° C. to separate the nuclei from miscellaneous cell debris (including membranes and cytoplasmic organelles).

Immediately pour off the supernatant and discard it, gradually so as not to dislodge the nuclear pellet.

Optional: To more thoroughly remove the supernatant, place 2-3 layers of fresh paper towels on a clean area of the bench and put the 45 ml tube upside down on the paper towels, without the cap. Blot away the excess supernatant, then let the remaining liquid drain away for 5 minutes.

### Module 1B Step 9 of 9: Pelleting

Place the sample tube on ice and gently resuspend the nuclear pellet in 1 ml of Lysis Buffer (recipe on page 4). Incubate on ice for 15 minutes. [Meanwhile, pre-chill a centrifuge to 4° C.]

Mix by gentle pipetting and aliquot the lysate into one or more fresh, meticulously labeled 1.5 ml tubes. Note that 100 μl of lysate corresponds to an estimated 1 million cells (2-3 mg of starting material), which is sufficient to produce one intact Hi-C library.

Centrifuge at 300×g for 5 minutes in a pre-chilled 4° C. centrifuge. Immediately discard the supernatant, close the tube securely, and freeze the cell pellet.

Store the frozen cell pellets at −80° C. indefinitely.

### Section 1: Sample Preparation

Module 1C: Fixation of Cryopreserved Immune Cells with Formaldehyde

Use this module when starting directly from a cryopreserved sample of live cells. This module is identical to Module 1A, except for Step 1 and the centrifugation speeds. This is the ENCODE standard protocol for all intact Hi-C libraries produced from cryopreserved immune cells.

### Module 1C Step 1 of 5: Thawing

Warm a water bath to 37° C., and warm a bottle of fresh growth medium appropriate for the cell type to 37° C. Retrieve a frozen cryovial of cells and quickly carry it in a −20° C. carrier to the water bath. Thaw the cryovial on a float in the 37° C. water bath until it is almost completely thawed.

Transfer the cell suspension from the cryovial to a fresh 15 ml conical tube. Gently, one drop at a time, add 1 ml of warm growth medium. Then steadily add more warm growth medium up to a total volume of 10 ml.

Centrifuge at 1000×g for 5 minutes. Immediately discard the supernatant and resuspend the cell pellet in 1×PBS (ThermoFisher, 10010-023) at a concentration of 1 million cells per 1 ml of buffer. Plan ahead so that the volumes of formaldehyde and glycine added in Steps 2 and 3 do not exceed the capacity of the tube. Split the sample volume into multiple tubes if necessary.

### Module 1C Step 2 of 5: Fixation

In a chemical fume hood, add freshly opened formaldehyde solution (ThermoFisher, 28908) to a final concentration of 1% (w/v). Close the tube cap securely. Incubate at room temperature with constant rocking or nutation for exactly 10 minutes to crosslink proteins and fix chromatin in place. [Meanwhile, pre-chill centrifuges to 4° C. for Steps 4 and 5, and fill an ice bucket.]

### Module 1C Step 3 of 5: Quenching

In a chemical fume hood, add a glycine (Sigma, G7403-1KG) stock solution to a final concentration of 200 mM. Close the tube cap securely. Incubate at room temperature with constant rocking or nutation for 5 minutes to quench the formaldehyde and prevent over-crosslinking. [Meanwhile, prepare the cold bath for Step 5.]

### Module 1C Step 4 of 5: Post-Fixation Wash

Centrifuge at 1000×g for 5 minutes in a pre-chilled 4° C. centrifuge (Eppendorf, 5804 R). In a chemical fume hood, immediately discard the supernatant into a hazardous waste container, following your institution's guidelines.

Optional: You may wash the cell pellet to more thoroughly remove any traces of formaldehyde and glycine. Resuspend the cell pellet in ice-cold 1×PBS at a concentration of 1 million cells per 1 ml of buffer. Centrifuge at 1000×g for 5 minutes in a pre-chilled 4° C. centrifuge. In a chemical fume hood, immediately discard the supernatant into a hazardous waste container, following your institution's guidelines.

Resuspend the cell pellet in ice-cold 1×PBS such that the sample volume (in ml, rounded down to the nearest ml) corresponds to the number of flash-frozen pellets you intend to make. For example, to make flash-frozen pellets of 8 million cells each, resuspend the cell pellet in one-eighth of the buffer volume used in Step 1.

On ice, mix well by pipetting, and aliquot the sample into meticulously labeled 1.5 ml microcentrifuge tubes (VWR, 80077-230) at 1 ml per tube.

### Module 1C Step 5 of 5: Flash-Freezing

Centrifuge at 2500×g for 5 minutes in a pre-chilled 4° C. centrifuge (Eppendorf, 5424 R). Immediately discard the supernatant, close the tube securely, and flash-freeze the cell pellet in a liquid nitrogen bath or in a dry ice and 100% (v/v) ethanol bath.

Store the flash-frozen cell pellets at −80° C. indefinitely.

### Section 1: Sample Preparation

Module 1D: Fixation with Additional Crosslinking

The quality of intact Hi-C libraries in a given cell line or tissue type-whether assessed by the detection and precise localization of architectural features at high resolution or by the achievement of other experimental goals-benefits greatly from optimization of the fixation step. A variety of crosslinking agents-applied individually, sequentially, or simultaneously—can produce good results. Formaldehyde on its own may be added for 10 minutes, as in the ENCODE standard protocols, or for a longer time (such as 30 minutes) to achieve a firmer level of fixation. Other crosslinking agents, such as disuccinimidyl glutarate (DSG) and ethylene glycol bis(succinimidylsuccinate) (EGS), may be used in combination with formaldehyde. When combining multiple crosslinkers, you may add them simultaneously in a single crosslinking reaction or sequentially in multiple fixation steps separated by quenching and wash steps. The variant crosslinking methods can be applied to any starting sample types: cell lines in liquid culture, solid tissues, or cryopreserved cells.

The module presented here is a combination of formaldehyde and DSG, added simultaneously in a single 30-minute fixation step. This is one representative example of stronger crosslinking, but it is not necessarily the optimal method for every sample type and experimental goal. Apart from the fixation step, the rest of the module is identical to Module 1A.

### Module 1D Step 1 of 5: Cell Culture

DSG (ThermoFisher, 20593) is stored at 4° C. in powder form. Warm a bottle of DSG to room temperature to avoid condensation, as DSG is moisture sensitive, but do not put it into solution yet. A 300 mM stock solution in dimethyl sulfoxide (DMSO) (VWR, 97063-136) must be freshly prepared right before adding it to the cells because DSG loses efficacy very quickly in solution.

Grow mammalian cells in vitro to ˜₈₀% confluence following the manufacturer's recommended culturing protocol. Use proper aseptic technique to limit contamination.

If the cells are adherent, trypsinize or scrape to detach them from the inner surface of the flask. Working quickly, transfer the cells in their growth medium to one or more 50 ml conical tubes. Pool together flasks or plates as needed. Mix by gentle pipetting, then take a small aliquot from each tube for counting and mycoplasma testing.

Centrifuge at 300×g for 5 minutes. Meanwhile, count the cells in each aliquot to estimate the total number of cells in each tube. Use these estimates to calculate the required volumes of formaldehyde, DSG, and glycine in Steps 2 and 3.

Immediately discard the supernatant and resuspend the cell pellet in fresh growth medium at a concentration of 1 million cells per 1 ml of medium. Plan ahead so that the volumes of formaldehyde, DSG, and glycine added in Steps 2 and 3 do not exceed the capacity of the tube. Split the sample volume into multiple tubes if necessary.

### Module 1D Step 2 of 5: Fixation

In a 1.5 ml microcentrifuge tube (VWR, 80077-230), prepare an aliquot of 300 mM DSG in DMSO by weighing 98 mg of DSG and adding 1 ml of DMSO.

In a chemical fume hood, add freshly opened formaldehyde solution (ThermoFisher, 28908) to the sample to a final concentration of 1% (w/v). Then add the freshly prepared DSG to a final concentration of 3 mM. Close the tube cap securely. Incubate at room temperature with constant rocking or nutation for exactly 30 minutes to crosslink proteins and fix chromatin in place. [Meanwhile, pre-chill centrifuges to 4° C. for Steps 4 and 5, and fill an ice bucket.]

Alternative Option: EGS (ThermoFisher, 21565) may be directly substituted for DSG. If using EGS, handle it in exactly the same way as DSG, except you will need to add 137 mg of EGS to 1 ml of DMSO for a 300 mM stock solution.

### Module 1D Step 3 of 5: Quenching

In a chemical fume hood, add a glycine (Sigma, G7403-1KG) stock solution to a final concentration of 200 mM. Close the tube cap securely. Incubate at room temperature with constant rocking or nutation for 5 minutes to quench the formaldehyde and prevent over-crosslinking. [Meanwhile, prepare the cold bath for Step 5.]

### Module 1D Step 4 of 5: Post-Fixation Wash

Centrifuge at 300×g for 5 minutes in a pre-chilled 4° C. centrifuge (Eppendorf, 5804 R). In a chemical fume hood, immediately discard the supernatant into a hazardous waste container, following your institution's guidelines.

Optional: You may wash the cell pellet to more thoroughly remove any traces of formaldehyde and glycine. Resuspend the cell pellet in ice-cold 1×PBS (ThermoFisher, 10010-023) at a concentration of 1 million cells per 1 ml of buffer. Centrifuge at 300×g for 5 minutes in a pre-chilled 4° C. centrifuge. In a chemical fume hood, immediately discard the supernatant into a hazardous waste container, following your institution's guidelines.

Resuspend the cell pellet in ice-cold 1×PBS (ThermoFisher, 10010-023) such that the sample volume (in ml, rounded down to the nearest ml) corresponds to the number of flash-frozen pellets you intend to make. For example, to make flash-frozen pellets of 8 million cells each, resuspend the cell pellet in one-eighth of the volume used in Step 1.

On ice, mix well by pipetting, and aliquot the sample into meticulously labeled 1.5 ml microcentrifuge tubes (VWR, 80077-230) at 1 ml per tube.

### Module 1D Step 5 of 5: Flash-Freezing

Centrifuge at 300×g for 5 minutes in a pre-chilled 4° C. centrifuge (Eppendorf, 5424 R). Immediately discard the supernatant, close the tube securely, and flash-freeze the cell pellet in a liquid nitrogen bath or in a dry ice and 100% (v/v) ethanol bath.

Store the flash-frozen cell pellets at −80° C. indefinitely.

### Section 2: Enzymatic Treatment

Module 2A: Digestion with Micrococcal Nuclease

Use this module when digesting chromatin with micrococcal nuclease (MNase), which preferentially cleaves the linker regions between nucleosomes genome-wide. Note that in addition to the digestion step, some of the other enzymatic reactions differ between this module and the other modules in Section 2.

### Module 2A Step 1 of 9: Cell Lysis

Fill an ice bucket. Very gently and slowly resuspend a frozen cell pellet (the output of Section 1) in ice-cold Lysis Buffer (recipe on page 4) at a concentration of 1 million cells per 100 μl of buffer. On ice, mix well by gently pipetting and transfer 100 μl of the sample (1 million cells) to a fresh 1.5 ml tube or a fresh 0.2 ml PCR microcentrifuge tube. Incubate on ice for 5 minutes to rupture the plasma membranes of the cells, releasing their intact nuclei into solution. [Meanwhile, begin thawing the buffer for Step 2.]

Optional: Multiple technical replicates of 1 million cells each may be processed in parallel starting from the same cell pellet, using either single-channel pipettes or multichannel pipettes. When processing multiple samples in parallel, to account for pipetting error, add an extra 10% volume to each component in each master mix.

Optional: Any excess nuclei in Lysis Buffer may be pulse centrifuged and stored at −80° C. indefinitely, to be thawed and processed at a later time. If you choose to do this, you may first centrifuge the excess nuclei at 2000×g for 5 minutes and discard the supernatant, freezing only the nuclear pellet; or you may freeze the excess nuclei suspended in Lysis Buffer.

Centrifuge at 2000×g for 5 minutes in a tabletop centrifuge or minifuge. [Meanwhile, prepare the master mix for Step 2.] Discard the supernatant conservatively. It is fine to leave behind a small amount of supernatant in order to avoid aspirating part of the pellet. Work quickly because the nuclear pellets tend to be very loose; if a pellet comes loose, it is fine to repeat the centrifugation for another 5 minutes at 2000×g.

### Module 2A Step 2 of 9: MNase Digestion

Very gently resuspend the nuclear pellet in 50 μl of MNase Master Mix:
i. 43.75 μl of water
ii. 5 μl of 10× Micrococcal Nuclease Reaction Buffer (NEB, B0247S)
iii. 0.5 μl of 10 mg/ml Purified BSA (NEB, B9001S)
iv. 0.75 μl of 20 U/μl Micrococcal Nuclease, diluted in 1× Micrococcal Nuclease Reaction Buffer from 2000 U/μl stock solution (NEB, M0247S)

Pulse centrifuge and incubate at 37° C. for 10 minutes to digest chromatin.

### Module 2A Step 3 of 9: MNase Inactivation

Pulse centrifuge and add 2 μl of 500 mM EGTA pH 8.0 (Fisher, 50-255-956) to stop the digestion reaction. Mix by gently pipetting with a P200 or P300 pipette. Pulse centrifuge and incubate at 62° C. for 10 minutes.

Centrifuge at 2000×g for 5 minutes. [Meanwhile, prepare the buffer for Step 4, and begin thawing the buffer for Step 5.] Discard the supernatant conservatively.

### Module 2A Step 4 of 9: Post-Digestion Wash

Prepare a stock solution of Hi-C Wash Buffer by combining the following ingredients in a 50 ml conical tube (mix by inverting and store at room temperature for up to 1 year):
i. 19.76 ml of water
ii. 200 μl of 1M Tris pH 8.0 [final: 10 mM]
iii. 40 μl of 5M NaCl [final: 10 mM]

Resuspend the nuclear pellet in 100 μl of Hi-C Wash Buffer. Centrifuge at 2000×g for 5 minutes. [Meanwhile, prepare the master mix for Step 5.] Discard the supernatant conservatively.

### Module 2A Step 5 of 9: MNase End Repair

Resuspend the nuclear pellet in 40 μl of MNase Repair Master Mix:
i. 33.5 μl of water
ii. 4 μl of 10×T4 DNA Ligase Reaction Buffer (NEB, B0202S)
iii. 2.5 μl of 10 U/μl T4 Polynucleotide Kinase (NEB, M0201L)

Pulse centrifuge and incubate at 37° C. for 30 minutes to repair MNase-digested DNA ends. [Meanwhile, begin thawing the buffer and nucleotides for Step 6.]

Centrifuge at 2000×g for 5 minutes. [Meanwhile, prepare the master mix for Step 6.] Discard the supernatant conservatively.

### Module 2A Step 6 of 9: Biotinylation and Proximity Ligation

Resuspend the nuclear pellet in 50 μl of Ligase Master Mix:
i. 18 μl of water
ii. 5 μl of 1 mM Biotin-11-dUTP (Jena Biosciences, NU-803-BIOX-S)
iii. 5 μl of 1 mM dATP, diluted in water from 100 mM stock solution (NEB, N0440S)
iv. 5 μl of 1 mM dCTP, diluted in water from 100 mM stock solution (NEB, N0441S)
v. 5 μl of 1 mM dGTP, diluted in water from 100 mM stock solution (NEB, N0442S)
vi. 5 μl of 10×T4 DNA Ligase Reaction Buffer
vii. 2 μl of 5 U/μl DNA Polymerase I, Large (Klenow) Fragment (NEB, M0210L)
viii. 5 μl of 400 U/μl T4 DNA Ligase (NEB, M0202L)

Pulse centrifuge and incubate at 25° C. for 1.5 hours to simultaneously biotinylate and ligate colocalized DNA fragments.

Alternative Option: Instead of combining the biotinylation and proximity ligation in one simultaneous reaction, you may do them as separate reactions. If you choose to do this, replace this step with Steps 4 and 5 of Module 2B.

Centrifuge at 2000×g for 5 minutes. [Meanwhile, prepare the master mix for Step 7. The SDS may precipitate, which is fine unless it interferes with pipetting. Mix by vigorously pipetting and incubate the master mix at 37° C. to help it solubilize.] Discard the supernatant conservatively.

### Module 2A Step 7 of 9: Crosslink Reversal

Resuspend the nuclear pellet in 100 μl of Proteinase Master Mix:
i. 74 μl of water
ii. 1 μl of 1M Tris pH 8.0 [final: 10 mM]
iii. 10 μl of 10% (w/v) SDS [final: 1%] (ThermoFisher, AM9822)
iv. 10 μl of 5M NaCl [final: 500 mM]
v. 5 μl of 0.8 U/μl Proteinase K [final: 4 U] (NEB, P8107S)

Vortex, pulse centrifuge, and incubate at 55° C. for 10 minutes to digest proteins. Then incubate at 75° C. for 1 hour to remove crosslinks. [Meanwhile, prepare the magnetic beads for Step 8.]

The protocol may be briefly paused here. Keep the sample at 4° C.

### Module 2A Step 8 of 9: DNA Purification

Warm an aliquot of sparQ PureMag solid-phase reversible immobilization (SPRI) beads (Quantabio, 95196-450) to room temperature. Vortex to resuspend the beads.

Pulse centrifuge the sample and add 100 μl of SPRI beads to bind DNA fragments longer than ˜100 bp. Vortex, pulse centrifuge, and incubate at room temperature for 10 minutes. Separate the supernatant from the beads on a magnet. Carefully discard the supernatant without disturbing the beads. Keeping the beads on the magnet, wash twice for 30 seconds with 200 μl of freshly prepared 70% (v/v) ethanol (VWR, 71002-508) without mixing. Do not pipet the ethanol directly onto the beads, instead targeting the opposite side of the tube. Remove the ethanol completely, and leave the beads on the magnet for a few minutes with open cap to allow trace ethanol to evaporate (but do not over-dry; the beads should look glossy and not cracked).

Resuspend the beads in 130 μl of Tris Buffer (recipe on page 4). Vortex, pulse centrifuge, and incubate at room temperature for 5 minutes to elute DNA. Separate on a magnet. Transfer the supernatant to a fresh 1.5 ml or 0.2 ml tube. Discard the beads.

This is a safe long-term pause point. Keep the sample at room temperature or at 4° C.

### Module 2A Step 9 of 9: Shearing

Transfer the entire sample volume to a Pre-Slit Snap-Cap 6×16 mm glass microTUBE vial (Covaris, 520045). To make the biotinylated DNA suitable for high-throughput sequencing, shear to a size of 250-300 bp using the following parameters:
i. Instrument=Covaris M220 Focused-ultrasonicator
ii. Temperature Setpoint=20.0° C., Minimum=18.0° C., Maximum=22.0° C.
iii. Peak Power=75.0, Duty Factor=26.0, Cycles/Burst=500
iv. Duration=60 seconds

Pulse centrifuge and remove the Covaris vial cap. Transfer the sample to a fresh 0.2 ml tube.

This is a safe long-term pause point. Keep the sample at room temperature or at 4° C.

Optional: To verify successful DNA purification and shearing, you may load 1 μl of the sample on an agarose gel or a Bioanalyzer instrument. Combine 1 μl of the sample with 4 μl of water and 1 μl of 6×DNA Loading Dye (ThermoFisher, R0611), then load this mixture on a FlashGel cassette (VWR, 95015-618) alongside 1 μl of the GeneRuler 1 kb Plus DNA Ladder (ThermoFisher, SM1333). Run the gel at 130V for 12 minutes. Alternatively, load 1 μl of the sample on a Bioanalyzer DNA 1000 chip (Agilent, 5067-1504) and run the DNA 1000 Assay. You should see a smear of DNA with a peak at approximately 250-300 bp. If the DNA is undersheared or oversheared, titrate the duration of shearing in 15-second intervals.

### Section 2: Enzymatic Treatment

Module 2B: Digestion with DNase I

Use this module when digesting chromatin with DNase I, which preferentially cleaves accessible DNA loci genome-wide. Note that in addition to the digestion step, some of the other enzymatic reactions differ between this module and the other modules in Section 2.

### Module 2B Step 1 of 9: Cell Lysis

Fill an ice bucket. Very gently and slowly resuspend a frozen cell pellet (the output of Section 1) in ice-cold Lysis Buffer (recipe on page 4) at a concentration of 1 million cells per 100 μl of buffer. On ice, mix well by gently pipetting and transfer 100 μl of the sample (1 million cells) to a fresh 1.5 ml tube or a fresh 0.2 ml PCR microcentrifuge tube. Incubate on ice for 5 minutes to rupture the plasma membranes of the cells, releasing their intact nuclei into solution. [Meanwhile, begin thawing the buffer for Step 2.]

Optional: Multiple technical replicates of 1 million cells each may be processed in parallel starting from the same cell pellet, using either single-channel pipettes or multichannel pipettes. When processing multiple samples in parallel, to account for pipetting error, add an extra 10% volume to each component in each master mix.

Optional: Any excess nuclei in Lysis Buffer may be pulse centrifuged and stored at −80° C. indefinitely, to be thawed and processed at a later time. If you choose to do this, you may first centrifuge the excess nuclei at 2000×g for 5 minutes and discard the supernatant, freezing only the nuclear pellet; or you may freeze the excess nuclei suspended in Lysis Buffer.

Centrifuge at 2000×g for 5 minutes in a tabletop centrifuge or minifuge. [Meanwhile, prepare the master mix for Step 2.] Discard the supernatant conservatively. It is fine to leave behind a small amount of supernatant in order to avoid aspirating part of the pellet. Work quickly because the nuclear pellets tend to be very loose; if a pellet comes loose, it is fine to repeat the centrifugation for another 5 minutes at 2000×g.

### Module 2B Step 2 of 9: DNase Digestion

Very gently resuspend the nuclear pellet in 100 μl of DNase Master Mix:

### EITHER

i. 85 μl of water
ii. 10 μl of 10× DNase I Reaction Buffer (NEB, B0303S)
iii. 5 μl of 2 U/μl DNase I (RNase-free) (NEB, M0303L)

### OR

i. 80 μl of water
ii. 10 μl of 10× Reaction Buffer with MgCl₂ (ThermoFisher, B43)
iii. 10 μl of 1 U/μl DNase I (ThermoFisher, EN0525)

Avoid vigorous pipetting and vortexing because DNase I is sensitive to physical denaturation. Pulse centrifuge and incubate at 37° C. for 25 minutes to digest chromatin. [Meanwhile, begin thawing the buffer and nucleotides for Step 4.]

Note that there are two alternative options for the DNase I enzyme. NEB DNase I tends to digest more gently and is suitable for fragile cell lines and tissues, whereas ThermoFisher DNase I tends to digest more aggressively and is best suited for robust cell lines. To find the optimal level of digestion for each given sample type, test both options and titrate the amount of enzyme in factors of 2.

### Module 2B Step 3 of 9: DNase Inactivation

Pulse centrifuge and add 2 μl of 500 mM EDTA pH 8.0 (ThermoFisher, AM9260G) to stop the digestion reaction. Mix by gently pipetting with a P200 or P300 pipette.

Pulse centrifuge and incubate at 65° C. for 10 minutes to inactivate the DNase I enzyme without reversing crosslinks. [Meanwhile, prepare the master mix for Step 4.]

Centrifuge at 2000×g for 5 minutes. Discard the supernatant conservatively.

### Module 2B Step 4 of 9: Biotinylation

Resuspend the nuclear pellet in 50 μl of Biotin Master Mix:
i. 20 μl of water
ii. 5 μl of 10×NEBuffer 2 (NEB, B7002S)
iii. 5 μl of 1 mM Biotin-11-dUTP (Jena Biosciences, NU-803-BIOX-S)
iv. 5 μl of 1 mM dATP, diluted in water from 100 mM stock solution (NEB, N0440S)
v. 5 μl of 1 mM dCTP, diluted in water from 100 mM stock solution (NEB, N0441S)
vi. 5 μl of 1 mM dGTP, diluted in water from 100 mM stock solution (NEB, N0442S)
vii. 5 μl of 5 U/μl DNA Polymerase I, Large (Klenow) Fragment (NEB, M0210L)

Pulse centrifuge and incubate at 37° C. for 15 minutes to create 3′ recessed DNA ends using the exonuclease activity of the enzyme. Then incubate at 25° C. for 15 minutes to fill in the recessed ends and tag them with biotin. [Meanwhile, begin thawing the buffer for Step 5.]

The protocol may be briefly paused here. Keep the sample at 4° C.

Centrifuge at 2000×g for 5 minutes. [Meanwhile, prepare the master mix for Step 5.] Discard the supernatant conservatively.

### Module 2B Step 5 of 9: Proximity Ligation

Resuspend the nuclear pellet in 50 μl of Ligase Master Mix:
i. 40 μl of water
ii. 5 μl of 10×T4 DNA Ligase Reaction Buffer (NEB, B0202S)
iii. 5 μl of 400 U/μl T4 DNA Ligase (NEB, M0202L)

Pulse centrifuge and incubate at 16° C. for 2 hours to ligate colocalized DNA fragments. [Meanwhile, begin thawing the buffer for Step 6.]

The protocol may be briefly paused here. Keep the sample at 4° C.

Centrifuge at 2000×g for 5 minutes. [Meanwhile, prepare the master mix for Step 6.] Discard the supernatant conservatively.

### Module 2B Step 6 of 9: Exonuclease III Digestion

Resuspend the nuclear pellet in 50 μl of ExoIII Master Mix:
i. 40 μl of water
ii. 5 μl of 10×NEBuffer I (NEB, B7001S)
iii. 5 μl of 100 U/μl Exonuclease III (NEB, M0206L)

Pulse centrifuge and incubate at 37° C. for 30 minutes to remove biotinylated but unligated DNA ends (“dangling ends”).

Centrifuge at 2000×g for 5 minutes. [Meanwhile, prepare the master mix for Step 7. The SDS may precipitate, which is fine unless it interferes with pipetting. Mix by vigorously pipetting and incubate the master mix at 37° C. to help it solubilize.] Discard the supernatant conservatively.

### Module 2B Step 7 of 9: Crosslink Reversal

Resuspend the nuclear pellet in 100 μl of Proteinase Master Mix:
i. 74 μl of water
ii. 1 μl of 1M Tris pH 8.0 [final: 10 mM]
iii. 10 μl of 10% (w/v) SDS [final: 1%] (ThermoFisher, AM9822)
iv. 10 μl of 5M NaCl [final: 500 mM]
v. 5 μl of 0.8 U/μl Proteinase K [final: 4 U] (NEB, P8107S)

Vortex, pulse centrifuge, and incubate at 55° C. for 10 minutes to digest proteins. Then incubate at 75° C. for 1 hour to remove crosslinks. [Meanwhile, prepare the magnetic beads for Step 8.]

The protocol may be briefly paused here. Keep the sample at 4° C.

### Module 2B Step 8 of 9: DNA Purification

Warm an aliquot of sparQ PureMag solid-phase reversible immobilization (SPRI) beads (Quantabio, 95196-450) to room temperature. Vortex to resuspend the beads.

Pulse centrifuge the sample and add 100 μl of SPRI beads to bind DNA fragments longer than ˜100 bp. Vortex, pulse centrifuge, and incubate at room temperature for 10 minutes.

Separate the supernatant from the beads on a magnet. Carefully discard the supernatant without disturbing the beads. Keeping the beads on the magnet, wash twice for 30 seconds with 200 μl of freshly prepared 70% (v/v) ethanol (VWR, 71002-508) without mixing. Do not pipet the ethanol directly onto the beads, instead targeting the opposite side of the tube. Remove the ethanol completely, and leave the beads on the magnet for a few minutes with open cap to allow trace ethanol to evaporate (but do not over-dry; the beads should look glossy and not cracked).

Resuspend the beads in 130 μl of Tris Buffer (recipe on page 4). Vortex, pulse centrifuge, and incubate at room temperature for 5 minutes to elute DNA. Separate on a magnet. Transfer the supernatant to a fresh 1.5 ml or 0.2 ml tube. Discard the beads.

This is a safe long-term pause point. Keep the sample at room temperature or at 4° C.

### Module 2B Step 9 of 9: Shearing

Transfer the entire sample volume to a Pre-Slit Snap-Cap 6×16 mm glass microTUBE vial (Covaris, 520045). To make the biotinylated DNA suitable for high-throughput sequencing, shear to a size of 250-300 bp using the following parameters:
i. Instrument=Covaris M220 Focused-ultrasonicator
ii. Temperature Setpoint=20.0° C., Minimum=18.0° C., Maximum=22.0° C.
iii. Peak Power=75.0, Duty Factor=26.0, Cycles/Burst=500
iv. Duration=60 seconds

Pulse centrifuge and remove the Covaris vial cap. Transfer the sample to a fresh 0.2 ml tube.

This is a safe long-term pause point. Keep the sample at room temperature or at 4° C.

Optional: To verify successful DNA purification and shearing, you may load 1 μl of the sample on an agarose gel or a Bioanalyzer instrument. Combine 1 μl of the sample with 4 μl of water and 1 μl of 6×DNA Loading Dye (ThermoFisher, R0611), then load this mixture on a FlashGel cassette (VWR, 95015-618) alongside 1 μl of the GeneRuler 1 kb Plus DNA Ladder (ThermoFisher, SM1333). Run the gel at 130V for 12 minutes. Alternatively, load 1 μl of the sample on a Bioanalyzer DNA 1000 chip (Agilent, 5067-1504) and run the DNA 1000 Assay. You should see a smear of DNA with a peak at approximately 250-300 bp. If the DNA is undersheared or oversheared, titrate the duration of shearing in 15-second intervals.

### Section 2: Enzymatic Treatment

Module 2C: Digestion with Benzonase

Use this module when digesting chromatin with a small amount (such as 0.5 units or 1 unit) of Benzonase Nuclease, which is a very powerful endonuclease that can completely degrade all forms of DNA and RNA. It is important to dilute the stock solution of the enzyme and to titrate the amount of enzyme in factors of 2 to find the optimal level of digestion that yields post-digestion fragments with an average length of 350-1000 bp. Apart from the digestion step, the enzymatic reactions in this module are identical to those of Module 2B.

### Module 2C Step 1 of 9: Cell Lysis

Fill an ice bucket. Very gently and slowly resuspend a frozen cell pellet (the output of Section 1) in ice-cold Lysis Buffer (recipe on page 4) at a concentration of 1 million cells per 100 μl of buffer. On ice, mix well by gently pipetting and transfer 100 μl of the sample (1 million cells) to a fresh 1.5 ml tube or a fresh 0.2 ml PCR microcentrifuge tube. Incubate on ice for 5 minutes to rupture the plasma membranes of the cells, releasing their intact nuclei into solution. [Meanwhile, begin thawing the buffer for Step 2.]

Optional: Multiple technical replicates of 1 million cells each may be processed in parallel starting from the same cell pellet, using either single-channel pipettes or multichannel pipettes. When processing multiple samples in parallel, to account for pipetting error, add an extra 10% volume to each component in each master mix.

Optional: Any excess nuclei in Lysis Buffer may be pulse centrifuged and stored at −80° C. indefinitely, to be thawed and processed at a later time. If you choose to do this, you may first centrifuge the excess nuclei at 2000×g for 5 minutes and discard the supernatant, freezing only the nuclear pellet; or you may freeze the excess nuclei suspended in Lysis Buffer.

Centrifuge at 2000×g for 5 minutes in a tabletop centrifuge or minifuge. [Meanwhile, prepare the master mix for Step 2.] Discard the supernatant conservatively. It is fine to leave behind a small amount of supernatant in order to avoid aspirating part of the pellet. Work quickly because the nuclear pellets tend to be very loose; if a pellet comes loose, it is fine to repeat the centrifugation for another 5 minutes at 2000×g.

### Module 2C Step 2 of 9: Benzonase Digestion

Very gently resuspend the nuclear pellet in 50 μl of Benzonase Master Mix:
i. 44 μl OR 43.5 μl of water
ii. 5 μl of 10× Benzonase Reaction Buffer (Sigma, E8263-5KU)
iii. 0.5 μl of 10 mg/ml Purified BSA (NEB, B9001S)
iv. 0.5 μl OR 1 μl of 1 U/μl Benzonase Nuclease, diluted in 1× Benzonase Reaction Buffer from 250 U/μl ultrapure stock solution (Sigma, E8263-5KU)

Pulse centrifuge and incubate at 37° C. for 30 minutes to digest chromatin. [Meanwhile, begin thawing the buffer and nucleotides for Step 4.]

### Module 2C Step 3 of 9: Benzonase Inactivation

Pulse centrifuge and add 2 μl of 500 mM EDTA pH 8.0 (ThermoFisher, AM9260G) to stop the digestion reaction. Mix by gently pipetting with a P200 or P300 pipette. Pulse centrifuge and incubate at 65° C. for 10 minutes. [Meanwhile, prepare the master mix for Step 4.]

Centrifuge at 2000×g for 5 minutes. Discard the supernatant conservatively.

### Module 2C Step 4 of 9: Biotinylation

Resuspend the nuclear pellet in 50 μl of Biotin Master Mix:
i. 20 μl of water
ii. 5 μl of 10×NEBuffer 2 (NEB, B7002S)
iii. 5 μl of 1 mM Biotin-11-dUTP (Jena Biosciences, NU-803-BIOX-S)
iv. 5 μl of 1 mM dATP, diluted in water from 100 mM stock solution (NEB, N0440S)
v. 5 μl of 1 mM dCTP, diluted in water from 100 mM stock solution (NEB, N0441S)
vi. 5 μl of 1 mM dGTP, diluted in water from 100 mM stock solution (NEB, N0442S)
vii. 5 μl of 5 U/μl DNA Polymerase I, Large (Klenow) Fragment (NEB, M0210L)

Pulse centrifuge and incubate at 37° C. for 15 minutes to create 3′ recessed DNA ends using the exonuclease activity of the enzyme. Then incubate at 25° C. for 15 minutes to fill in the recessed ends and tag them with biotin. [Meanwhile, begin thawing the buffer for Step 5.]

The protocol may be briefly paused here. Keep the sample at 4° C.

Centrifuge at 2000×g for 5 minutes. [Meanwhile, prepare the master mix for Step 5.] Discard the supernatant conservatively.

### Module 2C Step 5 of 9: Proximity Ligation

Resuspend the nuclear pellet in 50 μl of Ligase Master Mix:
i. 40 μl of water
ii. 5 μl of 10×T4 DNA Ligase Reaction Buffer (NEB, B0202S)
iii. 5 μl of 400 U/μl T4 DNA Ligase (NEB, M0202L)

Pulse centrifuge and incubate at 16° C. for 2 hours to ligate colocalized DNA fragments. [Meanwhile, begin thawing the buffer for Step 6.]

The protocol may be briefly paused here. Keep the sample at 4° C.

Centrifuge at 2000×g for 5 minutes. [Meanwhile, prepare the master mix for Step 6.] Discard the supernatant conservatively.

### Module 2C Step 6 of 9: Exonuclease III Digestion

Resuspend the nuclear pellet in 50 μl of ExoIII Master Mix:
i. 40 μl of water
ii. 5 μl of 10×NEBuffer I (NEB, B7001S)
iii. 5 μl of 100 U/μl Exonuclease III (NEB, M0206L)

Pulse centrifuge and incubate at 37° C. for 30 minutes to remove biotinylated but unligated DNA ends (“dangling ends”).

Centrifuge at 2000×g for 5 minutes. [Meanwhile, prepare the master mix for Step 7. The SDS may precipitate, which is fine unless it interferes with pipetting. Mix by vigorously pipetting and incubate the master mix at 37° C. to help it solubilize.] Discard the supernatant conservatively.

### Module 2C Step 7 of 9: Crosslink Reversal

Resuspend the nuclear pellet in 100 μl of Proteinase Master Mix:
i. 74 μl of water
ii. 1 μl of 1M Tris pH 8.0 [final: 10 mM]
iii. 10 μl of 10% (w/v) SDS [final: 1%] (ThermoFisher, AM9822)
iv. 10 μl of 5M NaCl [final: 500 mM]
v. 5 μl of 0.8 U/μl Proteinase K [final: 4 U] (NEB, P8107S)

Vortex, pulse centrifuge, and incubate at 55° C. for 10 minutes to digest proteins. Then incubate at 75° C. for 1 hour to remove crosslinks. [Meanwhile, prepare the magnetic beads for Step 8.]

The protocol may be briefly paused here. Keep the sample at 4° C.

### Module 2C Step 8 of 9: DNA Purification

Warm an aliquot of sparQ PureMag solid-phase reversible immobilization (SPRI) beads (Quantabio, 95196-450) to room temperature. Vortex to resuspend the beads.

Pulse centrifuge the sample and add 100 μl of SPRI beads to bind DNA fragments longer than ˜100 bp. Vortex, pulse centrifuge, and incubate at room temperature for 10 minutes.

Separate the supernatant from the beads on a magnet. Carefully discard the supernatant without disturbing the beads. Keeping the beads on the magnet, wash twice for 30 seconds with 200 μl of freshly prepared 70% (v/v) ethanol (VWR, 71002-508) without mixing. Do not pipet the ethanol directly onto the beads, instead targeting the opposite side of the tube. Remove the ethanol completely, and leave the beads on the magnet for a few minutes with open cap to allow trace ethanol to evaporate (but do not over-dry; the beads should look glossy and not cracked).

Resuspend the beads in 130 μl of Tris Buffer (recipe on page 4). Vortex, pulse centrifuge, and incubate at room temperature for 5 minutes to elute DNA. Separate on a magnet. Transfer the supernatant to a fresh 1.5 ml or 0.2 ml tube. Discard the beads.

This is a safe long-term pause point. Keep the sample at room temperature or at 4° C.

### Module 2C Step 9 of 9: Shearing

Transfer the entire sample volume to a Pre-Slit Snap-Cap 6×16 mm glass microTUBE vial (Covaris, 520045). To make the biotinylated DNA suitable for high-throughput sequencing, shear to a size of 250-300 bp using the following parameters:
i. Instrument=Covaris M220 Focused-ultrasonicator
ii. Temperature Setpoint=20.0° C., Minimum=18.0° C., Maximum=22.0° C.
iii. Peak Power=75.0, Duty Factor=26.0, Cycles/Burst=500
iv. Duration=60 seconds

Pulse centrifuge and remove the Covaris vial cap. Transfer the sample to a fresh 0.2 ml tube.

This is a safe long-term pause point. Keep the sample at room temperature or at 4° C.

Optional: To verify successful DNA purification and shearing, you may load 1 μl of the sample on an agarose gel or a Bioanalyzer instrument. Combine 1 μl of the sample with 4 μl of water and 1 μl of 6×DNA Loading Dye (ThermoFisher, R0611), then load this mixture on a FlashGel cassette (VWR, 95015-618) alongside 1 μl of the GeneRuler 1 kb Plus DNA Ladder (ThermoFisher, SM1333). Run the gel at 130V for 12 minutes. Alternatively, load 1 μl of the sample on a Bioanalyzer DNA 1000 chip (Agilent, 5067-1504) and run the DNA 1000 Assay. You should see a smear of DNA with a peak at approximately 250-300 bp. If the DNA is undersheared or oversheared, titrate the duration of shearing in 15-second intervals.

### Section 2: Enzymatic Treatment

Module 2D: Digestion with Restriction Enzyme Cocktail

Use this module when digesting chromatin with a cocktail of several different restriction endonucleases. By combining four restriction enzymes that each recognize a different restriction site, the genome is cut at a finer resolution than what is possible with a single restriction enzyme. Note that in addition to the digestion step, some of the other enzymatic reactions differ between this module and the other modules in Section 2.

### Module 2D Step 1 of 8: Cell Lysis

Fill an ice bucket. Very gently and slowly resuspend a frozen cell pellet (the output of Section 1) in ice-cold Lysis Buffer (recipe on page 4) at a concentration of 1 million cells per 200 μl of buffer. On ice, mix well by gently pipetting and transfer 200 μl of the sample (1 million cells) to a fresh 1.5 ml tube or a fresh 0.2 ml PCR microcentrifuge tube. Incubate on ice for 5 minutes to rupture the plasma membranes of the cells, releasing their intact nuclei into solution. [Meanwhile, begin thawing the buffer for Step 2.]

Optional: Multiple technical replicates of 1 million cells each may be processed in parallel starting from the same cell pellet, using either single-channel pipettes or multichannel pipettes. When processing multiple samples in parallel, to account for pipetting error, add an extra 10% volume to each component in each master mix.

Optional: Any excess nuclei in Lysis Buffer may be pulse centrifuged and stored at −80° C. indefinitely, to be thawed and processed at a later time. If you choose to do this, you may first centrifuge the excess nuclei at 2000×g for 5 minutes and discard the supernatant, freezing only the nuclear pellet; or you may freeze the excess nuclei suspended in Lysis Buffer.

Centrifuge at 2000×g for 5 minutes in a tabletop centrifuge or minifuge. [Meanwhile, prepare the master mix for Step 2.] Discard the supernatant conservatively. It is fine to leave behind a small amount of supernatant in order to avoid aspirating part of the pellet. Work quickly because the nuclear pellets tend to be very loose; if a pellet comes loose, it is fine to repeat the centrifugation for another 5 minutes at 2000×g.

### Module 2D Step 2 of 8: Digestion

Very gently resuspend the nuclear pellet in 50 μl of 1× rCutSmart Buffer, diluted in water from 10× stock solution (NEB, B6004S). Centrifuge at 2000×g for 5 minutes. Discard the supernatant conservatively.

Very gently resuspend the nuclear pellet in 75 μl of Digestion Master Mix:
i. 55.5 μl of water
ii. 7.5 μl of 10× rCutSmart Buffer (NEB, B6004S)
iii. 2 μl of 25 U/μl MboI (NEB, R0147M)
iv. 1 μl of 50 U/μl MseI (NEB, R0525M)
v. 5 μl of 10 U/μl NlaIII (NEB, R0125L)
vi. 4 μl of FastDigest Csp6I (ThermoFisher, FD0214)

Mix by pipetting once and gently flicking the tube. Pulse centrifuge and incubate at 37° C. for 1.5 hours to digest chromatin.

### Module 2D Step 3 of 8: Restriction Enzyme Inactivation

Pulse centrifuge and add 3 μl of 500 mM EDTA pH 8.0 (ThermoFisher, AM9260G) to stop the digestion reaction. Mix by gently pipetting with a P200 or P300 pipette.

Centrifuge at 2000×g for 5 minutes. [Meanwhile, begin thawing the buffer and nucleotides for Step 5.] Discard the supernatant conservatively.

### Module 2D Step 4 of 8: Post-Digestion Wash

Resuspend the nuclear pellet in 200 μl of Lysis Buffer.

Centrifuge at 2000×g for 5 minutes. [Meanwhile, prepare the master mix for Step 5.] Discard the supernatant conservatively.

### Module 2D Step 5 of 8: Biotinylation and Proximity Ligation

Resuspend the nuclear pellet in 75 μl of Ligase Master Mix:
i. 37 μl of water
ii. 7.5 μl of 10×T4 DNA Ligase Reaction Buffer (NEB, B0202S)
iii. 3.5 μl of 10% (w/v) Triton X-100 (ThermoFisher, 28314)
iv. 5 μl of 1 mM Biotin-11-dUTP (Jena Biosciences, NU-803-BIOX-S)
v. 5 μl of 1 mM dATP, diluted in water from 100 mM stock solution (NEB, N0440S)
vi. 5 μl of 1 mM dCTP, diluted in water from 100 mM stock solution (NEB, N0441S)
vii. 5 μl of 1 mM dGTP, diluted in water from 100 mM stock solution (NEB, N0442S)
viii. 2 μl of 5 U/μl DNA Polymerase I, Large (Klenow) Fragment (NEB, M0210L)
ix. 5 μl of 400 U/μl T4 DNA Ligase (NEB, M0202L)

Pulse centrifuge and incubate at 37° C. for 1.5 hours to simultaneously biotinylate and ligate colocalized DNA fragments.

Alternative Option: Instead of combining the biotinylation and proximity ligation in one simultaneous reaction, you may do them as separate reactions. If you choose to do this, replace this step with Steps 4 and 5 of Module 2B.

Centrifuge at 2000×g for 5 minutes. [Meanwhile, prepare the master mix for Step 6. The SDS may precipitate, which is fine unless it interferes with pipetting. Mix by vigorously pipetting and incubate the master mix at 37° C. to help it solubilize.] Discard the supernatant conservatively.

### Module 2D Step 6 of 8: Crosslink Reversal

Resuspend the nuclear pellet in 100 μl of Proteinase Master Mix:
i. 74 μl of water
ii. 1 μl of 1M Tris pH 8.0 [final: 10 mM]
iii. 10 μl of 10% (w/v) SDS [final: 1%] (ThermoFisher, AM9822)
iv. 10 μl of 5M NaCl [final: 500 mM]
v. 5 μl of 0.8 U/μl Proteinase K [final: 4 U] (NEB, P8107S)

Vortex, pulse centrifuge, and incubate at 55° C. for 10 minutes to digest proteins. Then incubate at 75° C. for 1 hour to remove crosslinks. [Meanwhile, prepare the magnetic beads for Step 7.]

The protocol may be briefly paused here. Keep the sample at 4° C.

### Module 2D Step 7 of 8: DNA Purification

Warm an aliquot of sparQ PureMag solid-phase reversible immobilization (SPRI) beads (Quantabio, 95196-450) to room temperature. Vortex to resuspend the beads.

Pulse centrifuge the sample and add 100 μl of SPRI beads to bind DNA fragments longer than ˜100 bp. Vortex, pulse centrifuge, and incubate at room temperature for 10 minutes.

Separate the supernatant from the beads on a magnet. Carefully discard the supernatant without disturbing the beads. Keeping the beads on the magnet, wash twice for 30 seconds with 200 μl of freshly prepared 70% (v/v) ethanol (VWR, 71002-508) without mixing. Do not pipet the ethanol directly onto the beads, instead targeting the opposite side of the tube. Remove the ethanol completely, and leave the beads on the magnet for a few minutes with open cap to allow trace ethanol to evaporate (but do not over-dry; the beads should look glossy and not cracked).

Resuspend the beads in 130 μl of Tris Buffer (recipe on page 4). Vortex, pulse centrifuge, and incubate at room temperature for 5 minutes to elute DNA. Separate on a magnet. Transfer the supernatant to a fresh 1.5 ml or 0.2 ml tube. Discard the beads.

This is a safe long-term pause point. Keep the sample at room temperature or at 4° C.

### Module 2D Step 8 of 8: Shearing

Transfer the entire sample volume to a Pre-Slit Snap-Cap 6×16 mm glass microTUBE vial (Covaris, 520045). To make the biotinylated DNA suitable for high-throughput sequencing, shear to a size of 250-300 bp using the following parameters:
i. Instrument=Covaris M220 Focused-ultrasonicator
ii. Temperature Setpoint=20.0° C., Minimum=18.0° C., Maximum=22.0° C.
iii. Peak Power=75.0, Duty Factor=26.0, Cycles/Burst=500, Duration=60 seconds

Pulse centrifuge and remove the Covaris vial cap. Transfer the sample to a fresh 0.2 ml tube.

This is a safe long-term pause point. Keep the sample at room temperature or at 4° C.

Optional: To verify successful DNA purification and shearing, you may load 1 μl of the sample on an agarose gel or a Bioanalyzer instrument. Combine 1 μl of the sample with 4 μl of water and 1 μl of 6×DNA Loading Dye (ThermoFisher, R0611), then load this mixture on a FlashGel cassette (VWR, 95015-618) alongside 1 μl of the GeneRuler 1 kb Plus DNA Ladder (ThermoFisher, SM1333). Run the gel at 130V for 12 minutes. Alternatively, load 1 μl of the sample on a Bioanalyzer DNA 1000 chip (Agilent, 5067-1504) and run the DNA 1000 Assay. You should see a smear of DNA with a peak at approximately 250-300 bp. If the DNA is undersheared or oversheared, titrate the duration of shearing in 15-second intervals.

### Section 3: Library Preparation

Module 3A: Illumina Library Preparation (without Methylation Detection)

Following the intact Hi-C enzymatic reactions and purification of DNA, use this module to select and sequence chimeric DNA fragments in which the ligation junctions are labeled with biotinylated nucleotides. The ENCODE standard protocol creates a DNA library with indexed Illumina adaptors, whose quality can be assessed using shallow paired-end sequencing (˜4 million reads) on an Illumina NextSeq instrument. A successful library can then be sequenced more deeply with paired-end reads on an Illumina NextSeq, HiSeq, or NovaSeq instrument; or it may be converted to an Ultima-compatible library for deep single-end sequencing on an Ultima Genomics instrument.

### Module 3A Step 1 of 8: Biotin Pulldown

Warm a tube of 3×TWB (recipe on page 4) to room temperature and preheat a tube of 1×TWB to 55° C.

Vortex a bottle of 10 mg/ml Dynabeads MyOne Streptavidin T1 (ThermoFisher, 65604D) and, for each sample that will be processed in parallel, aliquot 25 μl of T1 beads to a fresh 0.2 ml tube. Pulse centrifuge each aliquot, separate on a magnet, and discard the supernatant to remove the T1 storage buffer. Add 100 μl of 3×TWB to the T1 beads to wash them. Vortex, pulse centrifuge, separate on a magnet, and discard the supernatant.

Resuspend the T1 beads again in 65 μl of 3×TWB and add them to a sample of purified, sheared DNA (the output of Section 2). Vortex, pulse centrifuge, and incubate at room temperature for 30 minutes to bind biotinylated DNA to the streptavidin-coated beads.

### Module 3A Step 2 of 8: Post-Pulldown Washes

Separate on a magnet and discard the supernatant, then wash the beads as follows:
i. Add 160 μl of preheated 1×TWB. Vortex, pulse centrifuge, separate on a magnet, and discard the supernatant. [Meanwhile, begin thawing the buffer for Step 3.]
ii. Add 100 μl of Tris Buffer. Vortex, pulse centrifuge, separate on a magnet, and discard the supernatant. Repeat this wash once more to thoroughly remove nonbiotinylated fragments. [Meanwhile, prepare the master mix for Step 3.]

Resuspend the beads in 25 μl of Tris Buffer. Note that the volumes specified for the NEBNext Ultra II kit reagents in Steps 3 and 4 are half of the manufacturer's recommended volumes and work well for low-yield samples (less than 1 ng of biotinylated DNA). For high-yield samples, instead resuspend the beads in 50 μl of Tris Buffer and double all of the volumes in Steps 3 and 4, as per the manufacturer's recommendations.

This is a safe long-term pause point. Keep the sample at room temperature or at 4° C.

### Module 3A Step 3 of 8: End Repair

Add 5 μl of End Repair Master Mix:
i. 3.5 μl of NEBNext Ultra II End Prep Reaction Buffer (NEB, E7647AA)
ii. 1.5 μl of NEBNext Ultra II End Prep Enzyme Mix (NEB, E7646AA)

Mix by pipetting. Pulse centrifuge and incubate at 20° C. for 30 minutes to repair sheared DNA ends. Then incubate at 65° C. for 30 minutes. [Meanwhile, begin thawing adaptors for Step 4.]

### Module 3A Step 4 of 8: Adaptor Ligation

Pulse centrifuge and add 15.5 μl of Adaptor Ligation Master Mix:
i. 15 μl of NEBNext Ultra II Ligation Master Mix (NEB, E7648AA)
ii. 0.5 μl of NEBNext Ligation Enhancer (NEB, E7374AA)

Add 2.5 μl of a sample-specific 15 μM Illumina Dual Index TruSeq adaptor (Illumina, 20023784). Record each sample-index combination. Mix thoroughly by pipetting, pulse centrifuge, and incubate at 20° C. for 15 minutes to ligate the individually barcoded adaptors to the DNA library. If using a thermal cycler, keep the heated lid turned off.

Alternative Option: Instead of using Illumina adaptors and primers, it is possible to use Ultima Genomics adaptors and primers to directly create an Ultima-compatible library, following the manufacturer's recommendations.

### Module 3A Step 5 of 8: Unbound Adaptor Removal

Separate on a magnet and discard the supernatant, then wash the beads as follows:
i. Add 160 μl of preheated 1×TWB. Vortex, pulse centrifuge, separate on a magnet, and discard the supernatant. [Meanwhile, begin thawing reagents for Step 6.]
ii. Add 100 μl of Tris Buffer. Vortex, pulse centrifuge, separate on a magnet, and discard the supernatant. [Meanwhile, prepare the master mix for Step 6.]

### Module 3A Step 6 of 8: Polymerase Chain Reaction

Resuspend the beads in 100 μl of PCR Master Mix:
i. 40 μl of water
ii. 50 μl of 2× Kapa HiFi HotStart ReadyMix (KAPA Biosystems, KK2602)
iii. 10 μl of 25 μM Illumina forward and reverse primer mix (IDT, custom order)

Alternative Option: Instead of using Illumina adaptors and primers, it is possible to use Ultima Genomics adaptors and primers to directly create an Ultima-compatible library, following the manufacturer's recommendations.

Vortex, pulse centrifuge, and run the following PCR amplification program:
i. 98° C. for 45 seconds
ii. Cycle 6-16 times (8 or 9 cycles is a good default):
   98° C. for 15 seconds
   55° C. for 30 seconds
   72° C. for 30 seconds
iii. 72° C. for 1 minute
iv. Hold at 4° C.

This is a safe pause point. Keep the sample at room temperature or at 4° C.

Optional: To verify successful library amplification, combine 2 μl of the sample with 3 μl of water and 1 μl of 6×DNA Loading Dye (ThermoFisher, R0611). Load 5 μl of this mixture on a FlashGel cassette (VWR, 95015-618) alongside 1 μl of the GeneRuler 1 kb Plus DNA Ladder (ThermoFisher, SM1333). Run the gel at 130V for 12 minutes. A band of amplified DNA should be visible on the gel. Rerun the PCR with additional cycles if necessary.

### Module 3A Step 7 of 8: Size Selection

Warm an aliquot of sparQ PureMag solid-phase reversible immobilization (SPRI) beads (Quantabio, 95196-450) to room temperature. Vortex to resuspend the beads.

Pulse centrifuge the sample, separate on a magnet, and transfer the supernatant to a fresh 0.2 ml tube. Add 60 μl of SPRI beads (SPRI:sample ratio 0.6:1) to remove overly long DNA molecules. Vortex, pulse centrifuge, and incubate at room temperature for 10 minutes.

Separate on a magnet. Transfer the supernatant to a fresh 0.2 ml tube. Discard the beads. Add another 30 μl of SPRI beads (SPRI:sample final ratio 0.9:1) to remove short DNA pieces, PCR primers, any remaining unbound adaptors, and adaptor dimers. Vortex, pulse centrifuge, and incubate at room temperature for 5 minutes.

### Module 3A Step 8 of 8: Final Library Clean-Up

Separate on a magnet. Discard the supernatant. Keeping the beads on the magnet, wash twice for 30 seconds with 200 μl of freshly prepared 70% (v/v) ethanol without mixing. Do not pipet the ethanol directly onto the beads, instead targeting the opposite side of the tube. Remove the ethanol completely, and leave the beads on the magnet for a few minutes with open cap to allow trace ethanol to evaporate (but do not over-dry the beads).

Resuspend the beads in 20-30 μl of Tris Buffer to elute DNA. Vortex, pulse centrifuge, and incubate at room temperature for 5 minutes. Separate on a magnet. Transfer the supernatant to a fresh 1.5 ml tube meticulously labeled for long-term storage. Discard the beads. Store the final intact Hi-C library at −20° C. or −30° C.

Measure the DNA concentration and fragment size distribution of the completed intact Hi-C library using the Qubit dsDNA High Sensitivity Assay (ThermoFisher, Q32854) and Agilent Bioanalyzer. Sequence the library with the longest available paired-end reads on an Illumina NextSeq, HiSeq, or NovaSeq instrument (150PE reads are strongly recommended). You may also convert all or part of the final library into an Ultima Genomics-compatible library by following the latest version of the Ultima Genomics Library Amplification Kit User Guide, allowing for single-end sequencing on the Ultima Genomics platform. (This was done for the majority of ENCODE intact Hi-C experiments.) Regardless of the sequencing platform, the reads must be long enough to span any ligation junctions on each library fragment.

### Section 3: Library Preparation

Module 3B: Illumina Library Preparation with Methylation Detection

In addition to the Hi-C signal of the intact Hi-C protocol, the library can be modified to simultaneously provide information about the cytosine methylation state of the chimeric reads by adding the Enzymatic Methyl-seq (EM-seq) method during library preparation. Note that it is vitally important to shake the T1 beads during all incubations in Steps 6-10 fast enough to keep the beads suspended in solution and prevent them from settling on the bottom of the tube. Failure to do so may result in incomplete conversion of unmethylated cytosine to uracil.

### Module 3B Step 1 of 13: Biotin Pulldown

Warm a tube of 3×TWB (recipe on page 4) to room temperature and preheat a tube of 1×TWB to 55° C. As an additional stock solution for this module, prepare a tube of TET2 Buffer: Pulse centrifuge one tube of TET2 Reaction Buffer Supplement (NEB, E7127AA) from the NEBNext Enzymatic Methyl-seq Kit (NEB, E7120L). Add 400 μl of TET2 Reaction Buffer (NEB, E7126AA) from the same kit. Mix by pipetting and store at −20° C. for up to 4 months.

Vortex a bottle of 10 mg/ml Dynabeads MyOne Streptavidin T1 (ThermoFisher, 65604D) and, for each sample that will be processed in parallel, aliquot 25 μl of T1 beads to a fresh 0.2 ml tube. Pulse centrifuge each aliquot, separate on a magnet, and discard the supernatant to remove the T1 storage buffer. Add 100 μl of 3×TWB to the T1 beads to wash them. Vortex, pulse centrifuge, separate on a magnet, and discard the supernatant.

Resuspend the T1 beads again in 65 μl of 3×TWB and add them to a sample of purified, sheared DNA (the output of Section 2). Vortex, pulse centrifuge, and incubate at room temperature for 30 minutes to bind biotinylated DNA to the streptavidin-coated beads.

### Module 3B Step 2 of 13: Post-Pulldown Washes

Separate on a magnet and discard the supernatant, then wash the beads as follows:
i. Add 160 μl of preheated 1×TWB. Vortex, pulse centrifuge, separate on a magnet, and discard the supernatant. [Meanwhile, begin thawing the buffer for Step 3.]
ii. Add 100 μl of Tris Buffer. Vortex, pulse centrifuge, separate on a magnet, and discard the supernatant. Repeat this wash once more to thoroughly remove nonbiotinylated fragments. [Meanwhile, prepare the master mix for Step 3.]

Resuspend the beads in 50 μl of Tris Buffer.

This is a safe long-term pause point. Keep the sample at room temperature or at 4° C.

### Module 3B Step 3 of 13: End Repair

Add 10 μl of End Repair Master Mix:
i. 7 μl of NEBNext Ultra II End Prep Reaction Buffer (NEB, E7647AA)
ii. 3 μl of NEBNext Ultra II End Prep Enzyme Mix (NEB, E7646AA)

Mix by pipetting. Pulse centrifuge and incubate at 20° C. for 30 minutes to repair sheared DNA ends. Then incubate at 65° C. for 30 minutes. [Meanwhile, prepare reagents for Step 4.]

### Module 3B Step 4 of 13: Adaptor Ligation

Pulse centrifuge and add 2.5 μl of NEBNext EM-seq Adaptor (NEB, E7165AA). Then add 31 μl of Adaptor Ligation Master Mix:
i. 30 μl of NEBNext Ultra II Ligation Master Mix (NEB, E7648AA)
ii. 1 μl of NEBNext Ligation Enhancer (NEB, E7374AA)

Mix thoroughly by pipetting, pulse centrifuge, and incubate at 20° C. for 15 minutes to ligate the EM-seq adaptor to the DNA library. [Meanwhile, begin thawing the buffer for Step 5.]

### Module 3B Step 5 of 13: Post-Ligation Washes

Separate on a magnet and discard the supernatant, then wash the beads as follows:
i. Add 160 μl of 1×TWB heated to 55° C. Vortex, pulse centrifuge, separate on a magnet, and discard the supernatant. [Meanwhile, begin thawing reagents for Step 6.]
ii. Add 100 μl of Tris Buffer. Vortex, pulse centrifuge, separate on a magnet, and discard the supernatant. [Meanwhile, prepare the master mix for Step 6 and fill an ice bucket.]

Resuspend the beads in 28 μl of Elution Buffer (NEB, E7124AA).

This is a safe pause point. Keep the sample at room temperature or at 4° C.

### Module 3B Step 6 of 13: Oxidation of 5 mC and 5 hmC

On ice, add 17 μl of ice-cold TET2 Master Mix:
i. 10 μl of TET2 Buffer
ii. 1 μl of Oxidation Supplement (NEB, E7128AA)
iii. 1l of DTT (NEB, E7139AA)
iv. 1 μl of Oxidation Enhancer (NEB, E7129AA)
v. 4 μl of TET2 (NEB, E7130AA)

Vortex and pulse centrifuge. At room temperature, make a fresh dilute aliquot of Fe(II) Solution by adding 1 μl of 500 mM Fe(II) Solution (NEB, E7131AA) to 1249 μl of water. Add 5 μl of this aliquot to the sample.

Vortex, pulse centrifuge, and incubate in a heated shaker (Eppendorf, 5382000023) at 37° C. with 2000 rpm shaking for 1 hour to convert 5-methylcytosine and 5-hydroxymethylcytosine into deamination-resistant 5-carboxylcytosine and 5-glucosylmethylcytosine.

### Module 3B Step 7 of 13: Oxidation Enzyme Inactivation

Pulse centrifuge, place on ice, and add 1 μl of Stop Reagent (NEB, E7132AA). Vortex, pulse centrifuge, and incubate in a heated shaker at 37° C. with 2000 rpm shaking for 30 minutes.

This is a safe pause point. Keep the sample at 4° C.

### Module 3B Step 8 of 13: Post-Oxidation Washes

Pulse centrifuge, separate on a magnet and discard the supernatant, then wash the beads exactly as in Step 5. Resuspend in 28 μl of Elution Buffer and repeat Steps 6 and 7 once more to fully oxidize methylated cytosines that were missed during the first reaction.

Again pulse centrifuge, separate on a magnet and discard the supernatant, then wash the beads exactly as in Step 5. [Meanwhile, prepare the master mix for Step 9.] This time, resuspend in 16 μl of Elution Buffer.

This is a safe pause point. Keep the sample at room temperature or at 4° C.

### Module 3B Step 9 of 13: Cytosine Deamination

Preheat a heated shaker to 85° C. In a chemical fume hood, add 4 μl of formamide (Millipore, 344206) to the sample. Vortex, pulse centrifuge, and incubate in the preheated shaker at 85° C. with 2000 rpm shaking for 5 minutes to denature DNA.

Pulse centrifuge, place on ice, and add 80 μl of ice-cold APOBEC Master Mix:
i. 68 μl of water
ii. 10 μl of APOBEC Reaction Buffer (NEB, E7134AA)
iii. 1l of BSA (NEB, E7135AA)
iv. 1 μl of APOBEC (NEB, E7133AA)

Immediately vortex, pulse centrifuge, and incubate in a heated shaker at 37° C. with 2000 rpm shaking for 3 hours to deaminate unmodified cytosines.

This is a safe pause point. Keep the sample at 4° C.

### Module 3B Step 10 of 13: Post-Deamination Washes

Pulse centrifuge, separate on a magnet and discard the supernatant, then wash the beads exactly as in Step 5. Resuspend in 16 μl of Elution Buffer and repeat Step 9 once more to fully deaminate cytosines that were missed during the first reaction.

Again pulse centrifuge, separate on a magnet and discard the supernatant, then wash the beads exactly as in Step 5. [Meanwhile, thaw and pulse centrifuge the primer plate and thaw the master mix for Step 11.] This time, resuspend in 20 μl of Elution Buffer.

This is a safe pause point. Keep the sample at room temperature or at 4° C.

### Module 3B Step 11 of 13: Polymerase Chain Reaction

Add 5 μl of a sample-specific EM-seq primer pair from the NEBNext 96 Unique Dual Index Primer Pairs Plate (NEB, E7166A). Record each sample-index combination. Then add 25 μl of NEBNext Q5 U Master Mix (NEB, E7136AA). Vortex, pulse centrifuge, and run the following PCR amplification program:
i. 98° C. for 30 seconds
ii. Cycle 6-16 times (8 cycles is a good default):
   98° C. for 10 seconds
   62° C. for 30 seconds
   65° C. for 1 minute
iii. 65° C. for 5 minutes
iv. Hold at 4° C.

This is a safe pause point. Keep the sample at room temperature or at 4° C.

Optional: To verify successful library amplification, combine 1 μl of the sample with 4 μl of water and 1 μl of 6×DNA Loading Dye (ThermoFisher, R0611). Load 5 μl of this mixture on a FlashGel cassette (VWR, 95015-618) alongside 1 μl of the GeneRuler 1 kb Plus DNA Ladder (ThermoFisher, SM1333). Run the gel at 130V for 12 minutes. A band of amplified DNA should be visible on the gel. Rerun the PCR with additional cycles if necessary.

### Module 3B Step 12 of 13: Size Selection

Warm an aliquot of sparQ PureMag solid-phase reversible immobilization (SPRI) beads (Quantabio, 95196-450) to room temperature. Vortex to resuspend the beads.

Pulse centrifuge the sample, separate on a magnet, transfer the supernatant to a fresh 0.2 ml tube, and add 50 μl of water. Then add 60 μl of SPRI beads (SPRI:sample ratio 0.6:1) to remove overly long DNA molecules. Vortex, pulse centrifuge, and incubate at room temperature for 10 minutes.

Separate on a magnet. Transfer the supernatant to a fresh 0.2 ml tube. Discard the beads. Add another 30 μl of SPRI beads (SPRI:sample final ratio 0.9:1) to remove overly short DNA pieces. Vortex, pulse centrifuge, and incubate at room temperature for 5 minutes.

### Module 3B Step 13 of 13: Final Library Clean-Up

Separate on a magnet. Discard the supernatant. Keeping the beads on the magnet, wash twice for 30 seconds with 200 μl of freshly prepared 70% (v/v) ethanol without mixing. Do not pipet the ethanol directly onto the beads, instead targeting the opposite side of the tube. Remove the ethanol completely, and leave the beads on the magnet for a few minutes with open cap to allow trace ethanol to evaporate (but do not over-dry the beads).

Resuspend the beads in 20-30 μl of Tris Buffer to elute DNA. Vortex, pulse centrifuge, and incubate at room temperature for 5 minutes. Separate on a magnet. Transfer the supernatant to a fresh 1.5 ml tube meticulously labeled for long-term storage. Discard the beads. Store the final intact Hi-C library at −20° C. or −30° C.

Measure the DNA concentration and fragment size distribution of the completed intact Hi-C library using the Qubit dsDNA High Sensitivity Assay (ThermoFisher, Q32854) and Agilent Bioanalyzer. Sequence the library with the longest available paired-end reads on an Illumina NextSeq, HiSeq, or NovaSeq instrument (150PE reads are strongly recommended). You may also convert all or part of the final library into an Ultima Genomics-compatible library by following the latest version of the Ultima Genomics Library Amplification Kit User Guide, allowing for single-end sequencing on the Ultima Genomics platform. Regardless of the sequencing platform, the reads must be long enough to span any ligation junctions on each library fragment.

### Alternative Intact DNase Hi-C Protocol

### Protocol Notes:

1. This protocol is optimized for 1M cells. For more than 1M cells, all reagents and reactions need to be scaled up accordingly. Use this protocol cautiously when working with >1M cells.
2. The library preparation for Next-Generation Sequencing in this protocol provides adapter instructions for Illumina-based sequencing, as well as Ultima Genomics sequencing. Follow the appropriate adaptor ligation and PCR priming steps according to sequencing platform.
3. This protocol is written for multi-channel-based sample processing, but can be scaled down for single channel use as well.

### Stock Solutions

### Lysis Buffer

Combine the following ingredients in a 50 ml conical tube:
v. 19.36 ml of water (ThermoFisher #10977-023)
vi. 200 μl of 1M Tris-HCl pH 8.0 [final: 10 mM] (VWR #97062-674)
vii. 40 μl of 5M NaCl [final: 10 mM] (ThermoFisher #AM9759)
viii. 400 μl of 10% (v/v) IGEPAL CA-630 [final: 0.2%] (ThermoFisher #J61055-AE)

Mix by inverting and store at 4° C. for up to 1 month.

### 10 mM Tris Buffer

Combine the following ingredients in a 50 ml conical tube:
iii. 39.6 ml of water
iv. 400 μl of 1M Tris-HCl pH 8.0 [final: 10 mM]

Mix by vortexing and store at room temperature for up to 1 year.

### 3×Tween Wash Buffer (3×TWB)

Combine the following ingredients in a 50 ml conical tube:
vi. 14.68 ml of water
vii. 24 ml of 5M NaCl [final: 3M]
viii. 600 μl of 1M Tris-HCl pH 8.0 [final: 15 mM]
ix. 120 μl of 500 mM EDTA [final: 1.5 mM] (Corning #46-034-CI)
x. 600 μl of 10% (w/v) Tween 20 [final: 0.15%] (ThermoFisher #28320)

Mix by inverting and store at 4° C. for up to 1 month.

### 1× Tween Wash Buffer (1×TWB)

Combine the following ingredients in a 50 ml conical tube:
iii. 20 ml of water
iv. 10 ml of 3×TWB

Mix by inverting and store at 4° C. for up to 1 month

### Procedure

### Step 1: Cell Lysis

Fill an ice bucket. [Meanwhile, begin thawing the buffer for Step 2.] Very gently and slowly resuspend ˜1 million cross-linked mammalian cells in 100 μl of ice-cold Lysis Buffer to rupture their plasma membranes, releasing their intact nuclei into solution. Transfer the entire sample to a fresh tube on ice.

Optional Quality Checkpoint: Save ˜2.5% of the sample volume as a pre-digestion aliquot by transferring 2.5 μl of the suspension to a fresh PCR tube. Set aside at 4° C. until Step 7.

Centrifuge at 2000×g for 5 minutes in a tabletop minifuge. Discard the supernatant conservatively. It is fine to leave behind a small amount of supernatant to avoid aspirating part of the pellet.

### Step 2: DNase Digestion

Very gently resuspend the nuclear pellet in 50 μl of DNase Master Mix:
i. 44 μl of water
ii. 5.5 μl of 10× DNase I Reaction Buffer (NEB #B0303S)
iii. 5.5 μl of 2 U/μl DNase I (NEB #M0303L)

Avoid vigorous pipetting and vortexing because DNase I is sensitive to physical denaturation. Pulse centrifuge and incubate at 37° C. for 25 minutes to digest chromatin.

### Step 3: DNase Inactivation

Pulse centrifuge and add 1 μl of 500 mM EDTA to stop the digestion reaction. Mix by gently pipetting with a P200 or P300 pipette.

Pulse centrifuge and incubate at 65° C. for 10 minutes to inactivate the DNase I enzyme without reversing cross-links. [Meanwhile, begin thawing the buffer and nucleotides for Step 4.]

Centrifuge at 2000×g for 5 minutes. [Meanwhile, prepare the master mix for Step 4.] Discard the supernatant conservatively.

### Step 4: Biotinylation

Resuspend the nuclear pellet in 50 μl of Biotin Master Mix:
i. 22 μl of water
ii. 5.5 μl of 10×NEBuffer 2 (NEB #B7002S)
iii. 5.5 μl of 1 mM Biotin-11-dUTP (Jena Biosciences #NU-803-BIOX-S)
iv. 5.5 μl of 1 mM dATP, diluted in water from 100 mM stock solution (NEB #N0440S)
v. 5.5 μl of 1 mM dCTP, diluted in water from 100 mM stock solution (NEB #N0441S)
vi. 5.5 μl of 1 mM dGTP, diluted in water from 100 mM stock solution (NEB #N0442S)
vii. 5.5 μl of 5 U/μl DNA Polymerase I, Large (Klenow) Fragment (NEB #M0210L)

Pulse centrifuge and incubate at 37° C. for 15 minutes to create 3′ recessed DNA ends using the exonuclease activity of the enzyme. Then incubate at 25° C. for 15 minutes to fill in the recessed ends and tag them with biotin. [Meanwhile, begin thawing the buffer for Step 5.]

The protocol may be briefly paused here. Keep the sample at 4° C.

Optional Quality Checkpoint: Save ˜5% of the sample volume as a post-digestion aliquot by transferring 2.5 μl of the suspension to a fresh PCR tube. Set aside at 4° C. until Step 7.

Centrifuge at 2000×g for 5 minutes. [Meanwhile, prepare the master mix for Step 5.] Discard the supernatant conservatively.

### Step 5: Proximity Ligation

Resuspend the nuclear pellet in 50 μl of Ligase Master Mix:
i. 44 μl of water
ii. 5.5 μl of 10×T4 DNA Ligase Reaction Buffer (NEB #B0202S)
iii. 5.5 μl of 400 U/μl T4 DNA Ligase (NEB #M0202L)

Pulse centrifuge and incubate at 16° C. for 2 hours to ligate colocalized DNA fragments. [Meanwhile, begin thawing the buffer for Step 6.]

The protocol may be briefly paused here. Keep the sample at 4° C.

Optional Quality Checkpoint: Save ˜5% of the sample volume as a post-ligation aliquot by transferring 2.5 μl of the suspension to a fresh PCR tube. Set aside at 4° C. until Step 7.

Centrifuge at 2000×g for 5 minutes. [Meanwhile, prepare the master mix for Step 6.] Discard the supernatant conservatively.

### Step 6: Exonuclease III Digestion

Resuspend the nuclear pellet in 50 μl of ExoIII Master Mix:
i. 44 μl of water
ii. 5.5 μl of 10×NEBuffer I (NEB #B7001S)
iii. 5.5 μl of 100 U/μl Exonuclease III (NEB #M0206L)

Pulse centrifuge and incubate at 37° C. for 30 minutes to remove biotinylated but unligated DNA ends (“dangling ends”).

Optional Quality Checkpoint: Save ˜5% of the sample volume as a post-exonuclease aliquot by transferring 2.5 μl of the suspension to a fresh PCR tube. Set aside at 4° C. until Step 7.

Centrifuge at 2000×g for 5 minutes. [Meanwhile, prepare the master mix for Step 7.] Discard the supernatant conservatively.

### Step 7: Cross-Link Reversal

Prepare 300 μl of Proteinase Master Mix:
i. 222 μl of water
ii. 3 μl of 1M Tris-HCl pH 8.0
iii. 30 μl of 10% (w/v) SDS (ThermoFisher #AM9822)
iv. 30 μl of 5M NaCl
v. 15 μl of 0.8 U/μl Proteinase K (NEB #P8107S)

If the SDS precipitates, incubate the master mix at 37° C. until it solubilizes. Resuspend the nuclear pellet in 100 μl of Proteinase Master Mix. Add 37.5 μl of Proteinase Master Mix to each quality control (QC) aliquot. Vortex every tube, pulse centrifuge, and incubate at 55° C. for 10 minutes to digest proteins. Then incubate at 75° C. for 1 hour to remove cross-links. [Meanwhile, prepare the magnetic beads for Step 8.]

### Step 8: DNA Purification

Warm an aliquot of sparQ PureMag solid-phase reversible immobilization (SPRI) beads (Quantabio #95196-450) to room temperature. Vortex to resuspend the beads. Pulse centrifuge the sample and all QC aliquots. Add 100 μl of SPRI beads to the sample (SPRI:sample ratio 1:1) to bind DNA fragments longer than −100 bp. Add 60 μl of SPRI beads to each QC aliquot (SPRI:aliquot ratio 1.5:1) to bind all DNA. Mix each tube by pipetting at least 10 times, pulse centrifuge, and incubate at room temperature for 10 minutes.

Separate the supernatant from the beads on a magnet. Carefully discard the supernatant without disturbing the beads. Keeping the beads on the magnet, wash each tube twice for 30 seconds with 200 μl of freshly prepared 70% (v/v) ethanol (VWR #71002-508) without mixing. Do not pipet the ethanol directly onto the beads, instead targeting the opposite side of the tube. Remove the ethanol completely, and leave the beads on the magnet for a few minutes with open caps to allow trace ethanol to evaporate (but do not over-dry; the beads should look glossy and not cracked).

Resuspend the beads containing the sample in 130 μl of Tris Buffer, and resuspend the beads containing each QC aliquot in 15 μl of Tris Buffer. Mix each tube by pipetting at least 10 times, pulse centrifuge, and incubate at room temperature for 5 minutes to elute DNA.

Separate on a magnet. Transfer the supernatant to fresh PCR tubes. Discard the beads.

For each purified QC aliquot, combine 5 μl with 1 μl of 6×DNA Loading Dye (ThermoFisher #R0611) and load this mixture on a FlashGel cassette (VWR #95015-618) alongside 1 μl of the GeneRuler 1 kb Plus DNA Ladder (ThermoFisher #SM1333). Run the QC gel at 130V for 12 minutes. The pre-digestion aliquot should have a bright band of high-molecular-weight DNA and possibly a smear of RNA. The other aliquots should show wide smears of digested DNA.

This is a good long-term pause point. Keep the sample at room temperature or at 4° C.

### Step 9: Shearing

Transfer the entire sample volume to a Pre-Slit Snap-Cap 6×16 mm glass microTUBE vial (Covaris #520045). To make the biotinylated DNA suitable for high-throughput sequencing using Illumina sequencers, shear to a size of 250-300 bp using the following parameters:
i. Instrument=Covaris M220 Focused-ultrasonicator
ii. Temperature Setpoint=20.0° C., Minimum=18.0° C., Maximum=22.0° C.
iii. Peak Power=75.0, Duty Factor=26.0, Cycles/Burst=500, Duration=60 seconds

Pulse centrifuge and remove the Covaris vial cap. Transfer the sample to a fresh PCR tube.

This is a good long-term pause point. Keep the sample at room temperature or at 4° C.

Optional Quality Checkpoint: Load 1 μl of the sample on a Bioanalyzer DNA 1000 chip (Agilent #5067-1504) and run the DNA 1000 Assay to verify successful shearing. [Meanwhile, prepare the buffers for Step 10.]

### Step 10: Biotin Pulldown

Warm a tube of 3×TWB to room temperature and preheat a tube of 1×TWB to 55° C. Vortex a bottle of 10 mg/ml Dynabeads MyOne Streptavidin T1 (ThermoFisher #65604D) and aliquot 25 μl to a fresh PCR tube. Pulse centrifuge, separate on a magnet, and discard the supernatant. Add 100 μl of 3×TWB to the T1 beads to wash them. Vortex, pulse centrifuge, separate on a magnet, and discard the supernatant.

Resuspend the T1 beads again in 65 μl of 3×TWB and add them to the sample. Vortex, pulse centrifuge, and incubate at room temperature for 30 minutes to bind biotinylated DNA to the streptavidin-coated beads.

### Step 11: Post-Pulldown Washes

Separate on a magnet and discard the supernatant, then wash the beads as follows:
i. Add 160 μl of preheated 1×TWB. Vortex, pulse centrifuge, separate on a magnet, and discard the supernatant. [Meanwhile, begin thawing the buffer for Step 12.]
ii. Add 100 μl of Tris Buffer. Vortex, pulse centrifuge, separate on a magnet, and discard the supernatant. [Meanwhile, prepare the master mix for Step 12.]

Resuspend the beads in 20 μl of Tris Buffer.

This is a good long-term pause point. Keep the sample at room temperature or at 4° C.

### Step 12: End Repair

Add 10 μl of End Repair Master Mix:
i. 5.5 μl of water
ii. 3.85 μl of NEBNext Ultra II End Prep Reaction Buffer (NEB #E7647AA)
iii. 1.65 μl of NEBNext Ultra II End Prep Enzyme Mix (NEB #E7646AA)

Mix by pipetting. Pulse centrifuge and incubate at 20° C. for 30 minutes to repair sheared DNA ends. Then incubate at 65° C. for 30 minutes. [Meanwhile, begin thawing adaptors for Step 13.]

### Step 13: Adaptor Ligation

Pulse centrifuge and add 15.5 μl of Adaptor Ligation Master Mix:
i. 16.5 μl of NEBNext Ultra II Ligation Master Mix
ii. 0.55 μl of NEBNext Ligation Enhancer

To the ligation mix, add sequencing-platform appropriate adaptors and record sample index.
i. 2.5 μl of 15 μM Illumina dual index TruSeq adaptors (Illumina #20023784) OR for Ultima Sequencing
ii. 3 μl Ultima Genomics Adaptors with barcodes (BCxxx)+3 μl Ultima Genomics Universal Adaptors (UC-P1).

Mix thoroughly by pipetting, pulse centrifuge, and incubate the sample at 20° C. for 15 minutes to ligate the individually barcoded adaptors to the DNA library. If using a thermocycler for this step, keep the heated lid off.

### Step 14: Unbound Adaptor Removal

Separate on a magnet and discard the supernatant, then wash the beads as follows:
i. Add 160 μl of 1×TWB heated to 55° C. Vortex, pulse centrifuge, separate on a magnet, and discard the supernatant. [Meanwhile, begin thawing reagents for Step 15.]
ii. Add 100 μl of Tris Buffer. Vortex, pulse centrifuge, separate on a magnet, and discard the supernatant. [Meanwhile, prepare the master mix for Step 15.]

### Step 15: Polymerase Chain Reaction

Resuspend the beads in 100 μl of PCR Master Mix:
i. 55 μl of 2× Kapa HiFi HotStart ReadyMix (KAPA Biosystems #KK2602)
ii. 44 μl of water
iii. 11 μl of 25 μl M Illumina forward and reverse primer mix (IDT)
   OR
iv. 5.5 μl of 10 μM Ultima Genomics forward primer (PA30)+5.5 μl of 10 μM Ultima Genomics reverse primer (trP1).

Vortex, pulse centrifuge, and run the following PCR amplification program:
i. 98° C. for 45 seconds
ii. Cycle 6-16 times (8 cycles is standard):
   98° C. for 15 seconds
   55° C. for 30 seconds
   72° C. for 30 seconds
iii. 72° C. for 1 minute
iv. Hold at 4° C.

This is a safe pause point. Keep the sample at room temperature or at 4° C.

Optional Quality Checkpoint: Combine 2 μl of the sample with 3 μl of water and 1 μl of 6×DNA Loading Dye. Load 5 μl of this mixture on a FlashGel cassette alongside 1 μl of the GeneRuler 1 kb Plus DNA Ladder. Run the QC gel at 130V for 12 minutes to verify successful library amplification. Rerun the PCR with additional cycles if necessary.

### Step 16: Final Library Clean-Up

Warm an aliquot of SPRI beads to room temperature. Vortex to resuspend the beads.

Pulse centrifuge the sample, separate on a magnet, and transfer the supernatant to a fresh PCR tube. Add 60 μl of SPRI beads (SPRI:sample ratio 0.6:1) to remove overly long DNA molecules. Vortex, pulse centrifuge, and incubate at room temperature for 10 minutes.

Separate on a magnet. Transfer the supernatant to a fresh PCR tube. Discard the beads. Add another 30 μl of SPRI beads (SPRI:sample final ratio 0.9:1) to remove short DNA pieces, PCR primers, any remaining unbound adaptors, and adaptor dimers. Vortex, pulse centrifuge, and incubate at room temperature for 5 minutes.

Separate on a magnet. Discard the supernatant. Keeping the beads on the magnet, wash twice for 30 seconds with 200 μl of freshly prepared 70% (v/v) ethanol without mixing. Do not pipet the ethanol directly onto the beads, instead targeting the opposite side of the tube. Remove the ethanol completely and leave the beads on the magnet for a few minutes with open cap to allow trace ethanol to evaporate (but do not over-dry the beads).

Resuspend the beads in 20 μl of Tris Buffer to elute DNA. Vortex, pulse centrifuge, and incubate at room temperature for 5 minutes. Separate on a magnet. Transfer the supernatant to a fresh 1.5 ml microcentrifuge tube labeled appropriately for long-term storage. Discard the beads. Store the library at −20° C. or −30° C.

Measure the DNA concentration and fragment size distribution of the Hi-C library using the Qubit dsDNA High Sensitivity Assay and Agilent Bioanalyzer. Use an Illumina NextSeq 550 instrument for QC sequencing and a HiSeq or NovaSeq instrument for deeper sequencing.

### Alternative Intact MNase Hi-C Protocol

### Protocol Notes:

1. This protocol is optimized for 1M cells. For more than 1M cells, all reagents and reactions need to be scaled up accordingly. Use this protocol cautiously when working with >1M cells.
2. The library preparation for Next-Generation Sequencing in this protocol provides steps for Illumina-based sequencing, as well as Ultima Genomics sequencing. Follow the appropriate Adaptor Ligation and PCR primer steps according to sequencing platform.

### Stock Solutions:

### Lysis Buffer

Combine the following ingredients in a 50 ml conical tube:
i. 38.72 ml of water (ThermoFisher #10977-023)
ii. 400 μl of 1M Tris-HCl pH 8.0 [final: 10 mM] (VWR #97062-674)
iii. 80 μl of 5M NaCl [final: 10 mM] (ThermoFisher #AM9759)
iv. 800 μl of 10% (v/v) IGEPAL CA-630 [final: 0.2%] (ThermoFisher #J61055-AE)

Mix by inverting and store at 4° C. for up to 1 month.

### Wash Buffer

Combine the following ingredients in a 50 ml conical tube:
39.52 ml of water (ThermoFisher #10977-023)
400 μl of 1M Tris-HCl pH 8.0 [final: 10 mM] (VWR #97062-674)
80 μl of 5M NaCl [final: 10 mM] (ThermoFisher #AM9759)

Mix by inverting and store at 4° C. for up to 1 month.

### 10 mM Tris Buffer

Combine the following ingredients in a 50 ml conical tube:
i. 39.6 ml of water
ii. 400 μl of 1M Tris-HCl pH 8.0 [final: 10 mM]

Mix by vortexing and store at room temperature for up to 1 year.

### 2× Tween Wash Buffer (2×TWB)

Combine the following ingredients in a 50 ml conical tube:
i. 23.13 ml of water
ii. 16 ml of 5M NaCl [final: 3M]
iii. 400 μl of 1M Tris-HCl pH 8.0 [final: 15 mM]
iv. 80 μl of 500 mM EDTA [final: 1.5 mM] (Corning #46-034-CI)
v. 400 μl of 10% (w/v) Tween 20 [final: 0.15%] (ThermoFisher #28320)

Mix by inverting and store at 4° C. for up to 1 month.

### 1× Tween Wash Buffer (1×TWB)

Combine the following ingredients in a 50 ml conical tube:
i. 20 ml of water
ii. 20 ml of 2×TWB

Mix by inverting and store at 4° C. for up to 1 month.

### Procedure

### Step 1: Cell Lysis

Fill an ice bucket. [Meanwhile, begin thawing the buffer for Step 2.] Very gently and slowly resuspend ˜1 million cross-linked mammalian cells in 100 μl of ice-cold Lysis Buffer to rupture their plasma membranes, releasing their intact nuclei into solution. Transfer to a fresh tube and incubate on ice for 5 minutes.

Centrifuge at 2000×g for 5 minutes. Discard the supernatant conservatively. It is fine to leave behind a small amount of supernatant to avoid aspirating part of the pellet.

### Step 2: MNase Digestion

Very gently resuspend the nuclear pellet in 50 μl of DNase Master Mix:
i. 43.75 μl of water
ii. 5 μl of 10× Micrococcal nuclease buffer (NEB, B0247S)
iii. 0.5 μl of 10 mg/ml Bovine Serum Albumin (NEB, B9001S)
iv. 0.75 μl of 20 Gel U/μl Micrococcal nuclease, diluted from 2000 Gel U/μl (NEB, M0247S)

Pulse centrifuge and incubate at 37° C. for 10 minutes to digest chromatin.

### Step 3: MNase Inactivation

Pulse centrifuge and add 2 μl of 500 mM EGTA to stop the digestion reaction. Mix by gently pipetting with a P200 or P300 pipette.

Pulse centrifuge and incubate at 62° C. for 10 minutes to inactivate the MNase enzyme without reversing cross-links.

Centrifuge at 2000×g for 5 minutes. Discard the supernatant conservatively. Resuspend the nuclear pellet in 100 uL of wash buffer. Centrifuge at 2000×g for 5 minutes and discard the supernatant.

Optional Quality Checkpoint: Save ˜10% of the sample volume as a post-digestion aliquot by transferring 10 μl of wash buffer solution. Set aside at 4° C. until Step 7

### Step 4: End-Repair

Resuspend the nuclear pellet in 40 μl of End-Repair Master Mix:
i. 33.5 μl of water
i. 4 μl of 10×T4 DNA Ligase Reaction Buffer (NEB #B0202S)
ii. 2.5 μl of 10 U/μl T4 polynucleotide kinase (NEB, M0201L)

Pulse centrifuge and incubate at 37° C. for 30 minutes.

Centrifuge at 2000×g for 5 minutes. [Meanwhile, prepare the master mix for Step 5.] Discard the supernatant conservatively.

### Step 5: Proximity Ligation

Resuspend the nuclear pellet in 50 μl of Ligase Master Mix:
iii. 14 μl of water
ii. 8 μl of 1 mM Biotin-11-dUTP (Jena Biosciences #NU-803-BIOX-S)
iii. 8 μl of 1 mM dATP, diluted in water from 100 mM stock solution (NEB #N0440S)
iv. 8 μl of 1 mM dCTP, diluted in water from 100 mM stock solution (NEB #N0440S)
v. 8 μl of 1 mM dGTP, diluted in water from 100 mM stock solution (NEB #N0440S)
iv. 5 μl of 10×T4 DNA Ligase Reaction Buffer (NEB #B0202S)
v. 2 μl of 5 U/μl DNA polymerase I, large (Klenow) fragment (NEB, M0210L)
vi. 5 μl of 400 U/μl T4 DNA Ligase (NEB #M0202L)

Pulse centrifuge and incubate at 25° C. (room temperature) for 1.5 hours to ligate colocalized DNA fragments. [Meanwhile, begin thawing the buffer for Step 6.]

Add 2 ul of 500 mM EDTA. Centrifuge at 2000×g for 5 minutes. Discard the supernatant conservatively.

### Step 7: Cross-link Reversal

Prepare 30 μl of Proteinase Master Mix per sample:
i. 23 μl of 10 mM Tris-HCl pH 8.0
ii. 1l of 10% (w/v) SDS (ThermoFisher #AM9822)
iii. 1 μl of 5M NaCl
iv. 5 μl of 0.8 U/μl Proteinase K (NEB #P8107S)

If the SDS precipitates, incubate the master mix at 37° C. until it solubilizes. Resuspend the nuclear pellet in 30 μl of Proteinase Master Mix. Vortex every tube, pulse centrifuge, and incubate at 55° C. for 10 minutes to digest proteins. Then incubate at 75° C. for 1 hour to remove cross-links. [Meanwhile, prepare the magnetic beads for Step 8.]

Optional Quality Checkpoint: Reverse crosslink the post-digestion aliquot from Step 3 using the above mix and steps. Combine 2 μl of the de-crosslinked sample with 3 μl of water and 1 μl of 6×DNA Loading Dye. Load 5 μl of this mixture on a FlashGel cassette alongside 1 μl of the GeneRuler 1 kb Plus DNA Ladder and verify MNase digestion of DNA. Discard quality-control aliquots after this step and only proceed with sample.

The protocol may be briefly paused here. Keep the sample at 4° C. after cross-link reversal.

### Step 8: Shearing

Add 100 μl of 10 mM Tris-HCl (pH 8.0) to de-crosslinked sample, bringing up sample volume to 130 μl.

Transfer the entire sample volume to a Pre-Slit Snap-Cap 6×16 mm glass microTUBE vial (Covaris #520045). To make the biotinylated DNA suitable for high-throughput sequencing using Illumina sequencers, shear to a size of 250-400 bp using the following parameters:
i. Instrument=Covaris S220 Focused-ultrasonicator
ii. Temperature Setpoint=20.0° C., Minimum=4.0° C., Maximum=22.0° C.
iii. Peak Power=300, Duty Factor=30.0, Cycles/Burst=500, Duration=110 seconds

Pulse centrifuge and remove the Covaris vial cap. Transfer the sample to a fresh tube.

This is a good long-term pause point. Keep the sample at room temperature or at 4° C.

### Step 9: First Size Selection

Warm an aliquot of sparQ PureMag solid-phase reversible immobilization (SPRI) beads (Quantabio #95196-450) to room temperature. Vortex to resuspend the beads.

Pulse centrifuge the 130 μl sample in the new tube. If the volume is not exactly 130 μl, bring it up with 10 mM Tris-HCl (pH 8.0). To avoid loss in yield, size selection must be precise and according to proper volumes and ratios.

Add 78 μl of SPRI beads to the sample (SPRI:sample ratio 0.6:1) to remove longer DNA fragments. Mix each tube by pipetting at least 10 times, pulse centrifuge, and incubate at room temperature for 10 minutes.

Transfer the supernatant from the beads on a magnet into a new tube while avoiding any transfer of beads. The beads can be discarded.

Add 52 μl of SPRI beads (SPRI:sample 1:1) to the collected supernatant from the previous step. Mix tube, pulse centrifuge, and incubate at room temperature for 5 minutes. Separate on a magnet

Carefully discard the supernatant without disturbing the beads. Keeping the beads on the magnet, wash each tube twice for 30 seconds with 200 μl of freshly prepared 70% (v/v) ethanol (VWR #71002-508) without mixing. Do not pipet the ethanol directly onto the beads, instead targeting the opposite side of the tube. Remove the ethanol completely and leave the beads on the magnet for a few minutes with open caps to allow trace ethanol to evaporate (but do not over-dry; the beads should look glossy and not cracked).

Resuspend the beads containing the sample in 100 μl of Tris Buffer. Mix each tube by pipetting at least 10 times, pulse centrifuge, and incubate at room temperature for 5 minutes to elute DNA.

Separate on a magnet. Transfer the supernatant to fresh tubes. Discard the beads.

This is a good long-term pause point. Keep the sample at room temperature or at 4° C.

### Step 10: Biotin Pulldown

Warm a tube of 2×TWB to room temperature and preheat a tube of 1×TWB to 55° C. Vortex a bottle of 10 mg/ml Dynabeads MyOne Streptavidin T1 (ThermoFisher #65604D) and take out 25 μl per sample into a new tube. Pulse centrifuge, separate on a magnet, and discard the supernatant. Add 100 μl of 2×TWB to the T1 beads to wash them. Vortex, pulse centrifuge, separate on a magnet, and discard the supernatant.

Resuspend the T1 beads again in 100 μl of 2×TWB per sample, and 100 μl to each sample (making final buffer concentration 1×). Vortex, pulse centrifuge, and incubate at room temperature for 10 minutes to bind biotinylated DNA to the streptavidin-coated beads.

### Step 11: Post-Pulldown Washes

Separate on a magnet and discard the supernatant, then wash the beads as follows:
i. Add 160 μl of preheated 1×TWB. Vortex, pulse centrifuge, separate on a magnet, and discard the supernatant.
ii. Add 100 μl of Tris Buffer. Vortex, pulse centrifuge, separate on a magnet, and discard the supernatant.
iii.

Resuspend the beads in 25 μl of Tris Buffer.

This is a good long-term pause point. Keep the sample at room temperature or at 4° C.

### Note:

This protocol uses T1 beads throughout the library preparation, for any purposes, T1 beads can be removed by heating samples to 98° C. for 10 mins. Cool to room temperature and reclaim bead with magnets, transfer supernatant to a new 1.5 ml tube (Now DNA is dissolved in water phase, people can quantify DNA concentration by Qubit or other devices). If working with free DNA with no beads attached, use SPRI beads when transit from one reaction to another.

The reaction volumes given below for the NEBNext Ultra II are half of manufacturer recommendation and work well for lower-yield samples (<1 ng). If sample concentration is high, double the reaction volumes for End-Repair and Ligation, and use according to manufacturer recommendation.

### Step 12: End Repair

Add 5 μl of End Repair Master Mix:
i. 3.5 μl of NEBNext Ultra II End Prep Reaction Buffer (NEB #E7647AA)
ii. 1.5 μl of NEBNext Ultra II End Prep Enzyme Mix (NEB #E7646AA)

Mix by pipetting. Pulse centrifuge and incubate at 20° C. for 30 minutes to repair sheared DNA ends. Then incubate at 65° C. for 30 minutes.

### Step 13: Adaptor Ligation

Pulse centrifuge sample with End-Repair mix and add 15.5 μl of Adaptor Ligation mix.
iii. 15 μl of NEBNext Ultra II Ligation Master Mix
iv. 0.5 μl of NEBNext Ligation Enhancer

To the ligation mix, add sequencing-platform appropriate adaptors and record sample index.
v. 2.5 μl of 15 μM Illumina dual index TruSeq adaptors (Illumina #20023784) OR for Ultima Sequencing
vi. 3 μl Ultima Genomics Adaptors with barcodes (BCxxx)+3 μl Ultima Genomics Universal Adaptors (UC-P1).

Mix thoroughly by pipetting, pulse centrifuge, and incubate the sample at 20° C. for 15 minutes to ligate the individually barcoded adaptors to the DNA library. If using a thermocycler for this step, keep the heated lid off.

### Step 14: Unbound Adaptor Removal

Separate on a magnet and discard the supernatant, then wash the beads as follows:
i. Add 160 μl of 1×TWB heated to 55° C. Vortex, pulse centrifuge, separate on a magnet, and discard the supernatant.
ii. Add 100 μl of Tris Buffer. Vortex, pulse centrifuge, separate on a magnet, and discard the supernatant.

### Step 15: Polymerase Chain Reaction

Resuspend the beads in 100 μl of PCR Master Mix:
i. 50 μl of 2× Kapa HiFi HotStart ReadyMix (KAPA Biosystems #KK2602)
ii. 40 μl of water
iii. 10 μl of 25 μM Illumina forward and reverse primer mix (IDT)
   OR
   5 μl of 10 μM Ultima Genomics forward primer (PA30)+5 μl of 10 μM Ultima Genomics reverse primer (trP1).

Vortex, pulse centrifuge, and run the following PCR amplification program (8-9 cycles is standard):
i. 98° C. for 45 seconds
ii. Cycle 6-16 times (8 cycles is standard):
   98° C. for 15 seconds
   55° C. for 30 seconds
   72° C. for 30 seconds
iii. 72° C. for 1 minute
iv. Hold at 4° C.

This is a safe pause point. Keep the sample at room temperature or at 4° C.

Optional Quality Checkpoint: Combine 2 μl of the sample with 3 μl of water and 1 μl of 6×DNA Loading Dye. Load 5 μl of this mixture on a FlashGel cassette alongside 1 μl of the GeneRuler 1 kb Plus DNA Ladder. Run the QC gel at 130V for 12 minutes to verify successful library amplification. Rerun the PCR with additional cycles if necessary.

### Step 16: Final Library Clean-Up

Warm an aliquot of SPRI beads to room temperature. Vortex to resuspend the beads.

Pulse centrifuge the sample, separate on a magnet, and transfer the supernatant to a fresh PCR tube. Add 60 μl of SPRI beads (SPRI:sample ratio 0.6:1) to remove overly long DNA molecules. Vortex, pulse centrifuge, and incubate at room temperature for 10 minutes.

Separate on a magnet. Transfer the supernatant to a fresh tube. Discard the beads.

Add another 30 μl of SPRI beads (SPRI:sample final ratio 0.9:1) to remove short DNA pieces, PCR primers, any remaining unbound adaptors, and adaptor dimers. Vortex, pulse centrifuge, and incubate at room temperature for 5 minutes.

Separate on a magnet. Discard the supernatant. Keeping the beads on the magnet, wash twice for 30 seconds with 200 μl of freshly prepared 70% (v/v) ethanol without mixing. Do not pipet the ethanol directly onto the beads, instead targeting the opposite side of the tube. Remove the ethanol completely and leave the beads on the magnet for a few minutes with open cap to allow trace ethanol to evaporate (but do not over-dry the beads).

Resuspend the beads in 20 μl of Tris Buffer to elute DNA. Vortex, pulse centrifuge, and incubate at room temperature for 5 minutes. Separate on a magnet. Transfer the supernatant to a fresh 1.5 ml microcentrifuge tube labeled appropriately for long-term storage. Discard the beads. Store the library at −20° C. or −30° C.

Measure the DNA concentration and fragment size distribution of the Hi-C library using the Qubit dsDNA High Sensitivity Assay and Agilent Bioanalyzer. Use the appropriate sequencing platform for QC and deeper sequencing.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. A phased genome scale genomics map selected from the group consisting of:
a nuclease sensitivity or chromatin accessibility map for a cell, wherein the nuclease cut sites are determined with 1000, 500, 200, 100, 50, 10 or 1 base pair resolution, or any values in between;
a DNA methylation map for a cell, wherein the DNA methylation sites are determined with 1000, 500, 200, 100, 50, 10 or 1 base pair resolution, or any values in between; and
a DNA protein-binding map for a cell, wherein the sequence bound by a chromatin protein or chromatin modification is determined with 1000, 500, 200, 100, 50, 10 or 1 base pair resolution, or any values in between.

2-3. (canceled)

4. The phased genome scale nuclease sensitivity or chromatin accessibility map for a cell of claim 1, wherein the map is obtained by a method comprising:
enzymatically fragmenting intact chromatin in a cell;
performing proximity ligation of the fragmented chromatin;
sequencing ligation junctions of the ligated chromatin fragments obtained by proximity ligation to determine DNA contacts in the cell and chromatin cut sites;
phasing the sequenced chromatin fragments onto individual homologs in the cell based on DNA contacts; and
phasing the cut sites from the fragmenting step onto the individual homologs to generate a phased genome scale nuclease sensitivity map.

5. The phased genome scale DNA methylation map for a cell of claim 1, wherein the map is obtained by a method comprising:
enzymatically fragmenting intact chromatin in a cell;
performing proximity ligation of the fragmented chromatin;
converting the ligated chromatin fragments by a method that distinguishes between unmodified and modified cytosines, wherein modified cytosines are selected from the group consisting of methylated cytosines (mC) and hydroxymethylated cytosines (hmC);
sequencing ligation junctions of the converted ligated chromatin fragments obtained by proximity ligation to determine DNA contacts in the cell, DNA methylation sites, and chromatin cut sites;
phasing the sequenced chromatin fragments onto individual homologs in the cell based on DNA contacts; and
phasing the DNA methylation sites onto the individual homologs to generate a phased genome scale DNA methylation map.

6. The phased genome scale DNA methylation map of claim 5, wherein the method that distinguishes between unmodified and modified cytosines is selected from the group consisting of (i) bisulfite conversion, (ii) Tet-assisted bisulfite conversion, (iii) Tet-assisted conversion with a substituted borane reducing agent, and (iv) protection of hmC followed by Tet-assisted conversion with a substituted borane reducing agent.

7. The phased genome scale DNA protein-binding map for a cell of claim 1, wherein the map is obtained by a method comprising:
enzymatically fragmenting intact chromatin in a cell;
performing proximity ligation of the fragmented chromatin;
performing a method that detects protein binding to the ligated chromatin fragments or chromatin modifications on the ligated chromatin fragments, optionally, with an antibody specific for the chromatin protein or chromatin modification;
sequencing ligation junctions of the ligated chromatin fragments obtained by proximity ligation and immunoprecipitation to determine DNA contacts in the cell, chromatin cut sites, and DNA sites bound by the chromatin protein or having the chromatin modification;
phasing the sequenced chromatin fragments onto individual homologs in the cell based on DNA contacts; and
phasing the DNA sites bound by the chromatin protein or having the chromatin modification onto the individual homologs to generate a phased genome scale protein-binding map.

8. The phased genome scale DNA protein-binding map of claim 7, wherein the method that detects protein binding or chromatin modification is selected from the group consisting of (i) chromatin immunoprecipitation (ChIP) with an antibody specific for the chromatin protein or chromatin modification, (ii) fusion of a methyltransferase with a protein in vivo in order to modify nearby DNA bases (such as DAMid); (iii) antibody-mediated DNA modification or cleavage, such as Cut & Run; and (iv) other methods for marking sites bound by a specific protein.

9. A method for obtaining a phased genome scale nuclease sensitivity map for a cell comprising:
enzymatically fragmenting intact chromatin in a cell;
performing proximity ligation of the fragmented chromatin;
sequencing ligation junctions of the ligated chromatin fragments obtained by proximity ligation to determine DNA contacts in the cell and chromatin cut sites;
phasing the sequenced chromatin fragments onto individual homologs in the cell based on DNA contacts; and
phasing the cut sites from the fragmenting step onto the individual homologs to generate a phased genome scale nuclease sensitivity map.

10. The method of claim 9, further comprising obtaining a phased genome scale DNA methylation map for a cell, said method further comprising:
converting the ligated chromatin fragments by a method that distinguishes between unmodified and modified cytosines, wherein modified cytosines are selected from the group consisting of methylated cytosines (mC) and hydroxymethylated cytosines (hmC);
sequencing ligation junctions of the converted ligated chromatin fragments obtained by proximity ligation to determine DNA contacts in the cell, DNA methylation sites, and chromatin cut sites;
phasing the sequenced chromatin fragments onto individual homologs in the cell based on DNA contacts; and
phasing the DNA methylation sites onto the individual homologs to generate a phased genome scale DNA methylation map.

11. The method of claim 10, wherein the method that distinguishes between unmodified and modified cytosines is selected from the group consisting of (i) bisulfite conversion, (ii) Tet-assisted bisulfite conversion, (iii) Tet-assisted conversion with a substituted borane reducing agent, and (iv) protection of hmC followed by Tet-assisted conversion with a substituted borane reducing agent.

12. The method of claim 9, further comprising obtaining a phased genome scale DNA protein-binding map for a cell, said method further comprising:
performing a method that detects protein binding to the ligated chromatin fragments or chromatin modifications on the ligated chromatin fragments, optionally, with an antibody specific for a chromatin protein or chromatin modification;
sequencing ligation junctions of the ligated chromatin fragments obtained by proximity ligation to determine DNA contacts in the cell, chromatin cut sites, and DNA sites bound by the chromatin protein or having the chromatin modification;
phasing the sequenced chromatin fragments onto individual homologs in the cell based on DNA contacts; and
phasing the DNA sites bound by the chromatin protein or having the chromatin modification onto the individual homologs to generate a phased genome scale ChIP-seq map.

13. The method of claim 12, wherein the method that detects protein binding or chromatin modification is selected from the group consisting of (i) chromatin immunoprecipitation (ChIP) with an antibody specific for the chromatin protein or chromatin modification, (ii) fusion of a methyltransferase with a protein in vivo in order to modify nearby DNA bases (such as DAMid); (iii) antibody-mediated DNA modification or cleavage, such as Cut & Run; and (iv) other methods for marking sites bound by a specific protein.

14. The method of claim 9, further comprising identifying the state of the chromatin fragmented or confirming that the chromatin fragmented was intact, optionally, wherein only fragments from confirmed intact chromatin are used to generate the phased genome scale map.

15. The method of claim 9, further comprising detecting spatial proximity relationships between genomic DNA in a cell, said method further comprising:
identifying the state of the chromatin fragmented using the genome scale nuclease sensitivity map.

16. The method of claim 15, wherein fragments from the least denatured chromatin are used to detect spatial proximity relationships; or
wherein only fragments from confirmed intact chromatin are used to detect spatial proximity relationships; or
wherein the cell was obtained from a sample treated with one or more agents or conditions that causes chromatin to be altered; or
wherein the cell was obtained from a deceased organism.

17-19. (canceled)

20. The phased genome scale DNA methylation map for a cell of claim 1, wherein the map is obtained by a method comprising:
enzymatically fragmenting intact chromatin in a cell;
performing proximity ligation of the fragmented chromatin;
sequencing ligation junctions of the converted ligated chromatin fragments obtained by proximity ligation using a sequencer that can detect DNA methylation to determine DNA contacts in the cell, DNA methylation sites, and chromatin cut sites;
phasing the sequenced chromatin fragments onto individual homologs in the cell based on DNA contacts; and
phasing the DNA methylation sites onto the individual homologs to generate a phased genome scale DNA methylation map.

21. The method of claim 9, further comprising obtaining a phased genome scale DNA methylation map for a cell, said method further comprising:
sequencing ligation junctions of the converted ligated chromatin fragments obtained by proximity ligation using a sequencer that can detect DNA methylation to determine DNA contacts in the cell, DNA methylation sites, and chromatin cut sites;
phasing the sequenced chromatin fragments onto individual homologs in the cell based on DNA contacts; and
phasing the DNA methylation sites onto the individual homologs to generate a phased genome scale DNA methylation map.

22. The method of claim 9, further comprising an annotation of DNA elements located on each homolog of each chromosome of a cell as determined using the map or method; and/or
wherein chromatin is enzymatically fragmented with any nuclease, such as DNase I, micrococcal nuclease (MNase), benzonase, or cyanase, or a restriction enzyme, or a transposase complex.

23. (canceled)

24. The method of claim 9, further comprising identifying chromatin sites bound by a protein on the phased genome using the chromatin cut sites to identify sites protected by bound proteins.

25. The method of claim 24, further comprising determining known DNA motifs in the chromatin sites bound by proteins to determine the proteins bound at the chromatin sites in the diploid genome; and/or determining unknown DNA motifs bound by proteins.

26. (canceled)

27. The method of claim 25, further comprising isolating proteins specific to the unknown DNA motifs by isolating proteins that bind to the DNA motif sequences.

28. The method of claim 9, wherein intact chromatin is enzymatically fragmented in an isolated nuclei from the cell; and/or
wherein the cell is crosslinked; and/or
wherein the sequencing is ligation junction sequencing; and/or
wherein the method further comprises identifying sequence variants on a phased genome; and/or
wherein the method further comprises determining a phased whole genome sequence for the cell based on the determined sequence information.

29-30. (canceled)

31. The method of claim 28, wherein ligation junction sequencing comprises selecting and sequencing approximately 250 base pair fragments using paired end sequencing; or
wherein ligation junction sequencing comprises selecting and sequencing approximately 300 base pair fragments from a single end.

32-34. (canceled)

35. The method of claim 9, wherein the method is used to determine which DNA elements tend to be in physical proximity of other DNA elements; and/or
wherein the method is combined with single cell sequencing in order to map accessibility, methylation, or protein binding on a single chromosomal molecule or homolog rather than in a single cell; and/or
wherein chromatin is maintained intact using one or methods comprising: (1) not using SDS or other detergents prior to ligation; (2) crosslinking for an extended period of time with formaldehyde, using multiple crosslinkers, or not crosslinking at all; (3) avoiding high-temperature steps; and (4) performing in reactions in buffers with physiologic ion concentrations.

36-37. (canceled)


SEQUENCE LISTING (420 sequences)

SEQ ID NO: 1 - protein, 288 aa, synthetic construct
  REGION 1..288: note = Synthetic
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 2 - protein, 183 aa, synthetic construct
  REGION 1..183: note = Synthetic
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS

SEQ ID NO: 3 - protein, 7 aa, Simian virus 40
PKKKRKV

SEQ ID NO: 4 - protein, 10 aa, Simian virus 40
PKKKRKVEAS

SEQ ID NO: 5 - protein, 16 aa, synthetic construct
  REGION 1..16: note = Synthetic
KRPAATKKAGQAKKKK

SEQ ID NO: 6 - protein, 9 aa, Homo sapiens
PAAKRVKLD

SEQ ID NO: 7 - protein, 11 aa, Homo sapiens
RQRRNELKRSP

SEQ ID NO: 8 - protein, 38 aa, Homo sapiens
NQSSNFGPMKGGNFGGRSSGPYGGGGQYFAKPRNQGGY

SEQ ID NO: 9 - protein, 42 aa, Homo sapiens
RMRIZFKNKGKDTAELRRRRVEVSVELRKAKKDEQILKRRNV

SEQ ID NO: 10 - protein, 8 aa, Homo sapiens
VSRKRPRP

SEQ ID NO: 11 - protein, 8 aa, Homo sapiens
PPKKARED

SEQ ID NO: 12 - protein, 8 aa, Homo sapiens
PQPKKKPL

SEQ ID NO: 13 - protein, 12 aa, Mus sp.
SALIKKKKKMAP

SEQ ID NO: 14 - protein, 5 aa, Influenza virus
DRLRR

SEQ ID NO: 15 - protein, 7 aa, Influenza virus
PKQKKRK

SEQ ID NO: 16 - protein, 10 aa, Hepatitis delta virus
RKLKKKIKKL

SEQ ID NO: 17 - protein, 10 aa, Mus sp.
REKKKFLKRR

SEQ ID NO: 18 - protein, 20 aa, Homo sapiens
KRKGDEVDGVDEVAKKKSKK

SEQ ID NO: 19 - protein, 17 aa, Homo sapiens
RKCLQAGMNLEARKTKK

SEQ ID NO: 20 - protein, 19 aa, synthetic construct
  REGION 1..19: note = Synthetic
YGRCCASYAGRKGGCRSYR

SEQ ID NO: 21 - protein, 15 aa, synthetic construct
  REGION 1..15: note = Synthetic
RSYGCCMYCTRSTGG

SEQ ID NO: 22 - protein, 10 aa, synthetic construct
  REGION 1..10: note = Synthetic
CCACTAGRKG

SEQ ID NO: 23 - protein, 35 aa, synthetic construct
  REGION 1..35: note = Synthetic
CTGCAGTKCCNVCHNNYRGCCASYAGRKGGCRSYN

SEQ ID NO: 24 - protein, 34 aa, synthetic construct
  REGION 1..34: note = Synthetic
CTGCAGTKCCNNNNNYNRCCASYAGRKGGCRSYV

SEQ ID NO: 25 - protein, 15 aa, synthetic construct
  REGION 1..15: note = Synthetic
CCRMYAGRTGGCGCY

SEQ ID NO: 26 - protein, 12 aa, synthetic construct
  REGION 1..12: note = Synthetic
NSCAGGGGGCGS

SEQ ID NO: 27 - protein, 7 aa, synthetic construct
  REGION 1..7: note = Synthetic
GGTGGCG

SEQ ID NO: 28 - protein, 15 aa, synthetic construct
  REGION 1..15: note = Synthetic
TTTTTTTTTTTTTTT

SEQ ID NO: 29 - protein, 15 aa, synthetic construct
  REGION 1..15: note = Synthetic
GRCCCCCCGCKGYGH

SEQ ID NO: 30 - protein, 15 aa, synthetic construct
  REGION 1..15: note = Synthetic
CCAGCCTGGGCRACA

SEQ ID NO: 31 - protein, 15 aa, synthetic construct
  REGION 1..15: note = Synthetic
GCCTGTAATCCCAGC

SEQ ID NO: 32 - protein, 13 aa, synthetic construct
  REGION 1..13: note = Synthetic
RGYGCRGTGGCDC

SEQ ID NO: 33 - protein, 15 aa, synthetic construct
  REGION 1..15: note = Synthetic
GCCTCRGCCTCCCAA

SEQ ID NO: 34 - protein, 15 aa, synthetic construct
  REGION 1..15: note = Synthetic
GGAGGCBGRGGCRGG

SEQ ID NO: 35 - protein, 14 aa, synthetic construct
  REGION 1..14: note = Synthetic
GACCCCCYGCTGTG

SEQ ID NO: 36 - protein, 7 aa, synthetic construct
  REGION 1..7: note = Synthetic
MGCCACA

SEQ ID NO: 37 - protein, 15 aa, synthetic construct
  REGION 1..15: note = Synthetic
TGTYGCCCAGGCTGG

SEQ ID NO: 38 - protein, 15 aa, synthetic construct
  REGION 1..15: note = Synthetic
GCCTGTAATCCCAGC

SEQ ID NO: 39 - protein, 13 aa, synthetic construct
  REGION 1..13: note = Synthetic
CNCAGCAGGAGNN

SEQ ID NO: 40 - protein, 12 aa, synthetic construct
  REGION 1..12: note = Synthetic
ARACYCYGTCTC

SEQ ID NO: 41 - protein, 15 aa, synthetic construct
  REGION 1..15: note = Synthetic
YTCAAGYGATYCTCC

SEQ ID NO: 42 - protein, 11 aa, synthetic construct
  REGION 1..11: note = Synthetic
CVCAGCAGGNV

SEQ ID NO: 43 - protein, 14 aa, synthetic construct
  REGION 1..14: note = Synthetic
AAAAAAAMAAAAAA

SEQ ID NO: 44 - protein, 9 aa, synthetic construct
  REGION 1..9: note = Synthetic
YGGGKGKYV

SEQ ID NO: 45 - protein, 15 aa, synthetic construct
  REGION 1..15: note = Synthetic
GCAGTGAGCYRAGAT

SEQ ID NO: 46 - protein, 16 aa, synthetic construct
  REGION 1..16: note = Synthetic
VGACCCCCCGCTGHGM

SEQ ID NO: 47 - protein, 11 aa, synthetic construct
  REGION 1..11: note = Synthetic
RVCAGATGGYN

SEQ ID NO: 48 - protein, 9 aa, synthetic construct
  REGION 1..9: note = Synthetic
AGGAAGTGR

SEQ ID NO: 49 - protein, 12 aa, synthetic construct
  REGION 1..12: note = Synthetic
GMCCCCCCGCTG

SEQ ID NO: 50 - protein, 9 aa, synthetic construct
  REGION 1..9: note = Synthetic
RNTGTGGCG

SEQ ID NO: 51 - protein, 14 aa, synthetic construct
  REGION 1..14: note = Synthetic
NDCACAGCAGGDRG

SEQ ID NO: 52 - protein, 8 aa, synthetic construct
  REGION 1..8: note = Synthetic
SYGGCGCY

SEQ ID NO: 53 - protein, 13 aa, synthetic construct
  REGION 1..13: note = Synthetic
NRACAGATGGYNN

SEQ ID NO: 54 - protein, 9 aa, synthetic construct
  REGION 1..9: note = Synthetic
NCGCCGBMN

SEQ ID NO: 55 - protein, 12 aa, synthetic construct
  REGION 1..12: note = Synthetic
CACCATATGKYR

SEQ ID NO: 56 - protein, 9 aa, synthetic construct
  REGION 1..9: note = Synthetic
RTGCCAACY

SEQ ID NO: 57 - protein, 12 aa, synthetic construct
  REGION 1..12: note = Synthetic
VVCCACGTGGBB

SEQ ID NO: 58 - protein, 10 aa, synthetic construct
  REGION 1..10: note = Synthetic
WVGCGCCAHN

SEQ ID NO: 59 - protein, 15 aa, synthetic construct
  REGION 1..15: note = Synthetic
NNGRACAGATGGYNN

SEQ ID NO: 60 - protein, 10 aa, synthetic construct
  REGION 1..10: note = Synthetic
CCCCCCMYDH

SEQ ID NO: 61 - protein, 11 aa, synthetic construct
  REGION 1..11: note = Synthetic
NNCCACGCGNN

SEQ ID NO: 62 - protein, 10 aa, synthetic construct
  REGION 1..10: note = Synthetic
NGGGCCCCCN

SEQ ID NO: 63 - protein, 6 aa, synthetic construct
  REGION 1..6: note = Synthetic
TCACCA

SEQ ID NO: 64 - protein, 11 aa, synthetic construct
  REGION 1..11: note = Synthetic
CACGTGGCANN

SEQ ID NO: 65 - protein, 11 aa, synthetic construct
  REGION 1..11: note = Synthetic
NVATGGCGGCS

SEQ ID NO: 66 - protein, 16 aa, synthetic construct
  REGION 1..16: note = Synthetic
RWTTTGGCGCCAWWWY

SEQ ID NO: 67 - protein, 11 aa, synthetic construct
  REGION 1..11: note = Synthetic
CACGTGGCANN

SEQ ID NO: 68 - protein, 11 aa, synthetic construct
  REGION 1..11: note = Synthetic
SACGTGGCANN

SEQ ID NO: 69 - protein, 10 aa, synthetic construct
  REGION 1..10: note = Synthetic
AVCATATGBY

SEQ ID NO: 70 - protein, 13 aa, synthetic construct
  REGION 1..13: note = Synthetic
NNVACAGATGGNN

SEQ ID NO: 71 - protein, 13 aa, synthetic construct
  REGION 1..13: note = Synthetic
TGCCCYSRGGGCA

SEQ ID NO: 72 - protein, 10 aa, synthetic construct
  REGION 1..10: note = Synthetic
WMCACCTGKW

SEQ ID NO: 73 - protein, 8 aa, synthetic construct
  REGION 1..8: note = Synthetic
CRCCGMCS

SEQ ID NO: 74 - protein, 5 aa, synthetic construct
  REGION 1..5: note = Synthetic
AGGGG

SEQ ID NO: 75 - protein, 15 aa, synthetic construct
  REGION 1..15: note = Synthetic
GAGACRGRGTYTCRC

SEQ ID NO: 76 - protein, 13 aa, synthetic construct
  REGION 1..13: note = Synthetic
NNGCACCTGCCNN

SEQ ID NO: 77 - protein, 7 aa, synthetic construct
  REGION 1..7: note = Synthetic
CYACYAA

SEQ ID NO: 78 - protein, 11 aa, synthetic construct
  REGION 1..11: note = Synthetic
NNCACCTGCNN

SEQ ID NO: 79 - protein, 10 aa, synthetic construct
  REGION 1..10: note = Synthetic
VCRCCGCMRY

SEQ ID NO: 80 - protein, 8 aa, synthetic construct
  REGION 1..8: note = Synthetic
GCGCCGCC

SEQ ID NO: 81 - protein, 10 aa, synthetic construct
  REGION 1..10: note = Synthetic
BGGGSCCMCC

SEQ ID NO: 82 - protein, 13 aa, synthetic construct
  REGION 1..13: note = Synthetic
TGCCCYBRGGGCA

SEQ ID NO: 83 - protein, 10 aa, synthetic construct
  REGION 1..10: note = Synthetic
MGCCGYCRNN

SEQ ID NO: 84 - protein, 5 aa, synthetic construct
  REGION 1..5: note = Synthetic
AGGGG

SEQ ID NO: 85 - protein, 9 aa, synthetic construct
  REGION 1..9: note = Synthetic
RTGCCCRSB

SEQ ID NO: 86 - protein, 12 aa, synthetic construct
  REGION 1..12: note = Synthetic
NNTGCTGCCCWR

SEQ ID NO: 87 - protein, 14 aa, synthetic construct
  REGION 1..14: note = Synthetic
TTTTGGCGCCAWWW

SEQ ID NO: 88 - protein, 12 aa, synthetic construct
  REGION 1..12: note = Synthetic
NNCCACGTGCNB

SEQ ID NO: 89 - protein, 10 aa, synthetic construct
  REGION 1..10: note = Synthetic
NMGCCGCCDN

SEQ ID NO: 90 - protein, 13 aa, synthetic construct
  REGION 1..13: note = Synthetic
TGCCCYSRGGGCA

SEQ ID NO: 91 - protein, 12 aa, synthetic construct
  REGION 1..12: note = Synthetic
TTTGGCGCCAAA

SEQ ID NO: 92 - protein, 15 aa, synthetic construct
  REGION 1..15: note = Synthetic
TGAGGYCAGGAGTTY

SEQ ID NO: 93 - protein, 13 aa, synthetic construct
  REGION 1..13: note = Synthetic
NNGCACCTGCYNB

SEQ ID NO: 94 - protein, 15 aa, synthetic construct
  REGION 1..15: note = Synthetic
VRBVNTGGGCGCCAM

SEQ ID NO: 95 - protein, 15 aa, synthetic construct
  REGION 1..15: note = Synthetic
GCSGGGCGBGGTGGC

SEQ ID NO: 96 - protein, 5 aa, synthetic construct
  REGION 1..5: note = Synthetic
RGGGG

SEQ ID NO: 97 - protein, 7 aa, synthetic construct
  REGION 1..7: note = Synthetic
CCCCTGA

SEQ ID NO: 98 - protein, 15 aa, synthetic construct
  REGION 1..15: note = Synthetic
GSMMCCYARGGKKBM

SEQ ID NO: 99 - protein, 15 aa, synthetic construct
  REGION 1..15: note = Synthetic
MHARNGGGAGACAMB

SEQ ID NO: 100 - protein, 8 aa, synthetic construct
  REGION 1..8: note = Synthetic
ACCCCTAA

SEQ ID NO: 101 - protein, 9 aa, synthetic construct
  REGION 1..9: note = Synthetic
WAACCGCAA

SEQ ID NO: 102 - protein, 15 aa, synthetic construct
  REGION 1..15: note = Synthetic
AGTGCAGTGGYRYRA

SEQ ID NO: 103 - protein, 20 aa, synthetic construct
  REGION 1..20: note = Synthetic
YDBNYNVCACCTGNMMVMHV

SEQ ID NO: 104 - protein, 8 aa, synthetic construct
  REGION 1..8: note = Synthetic
GCGCCGCC

SEQ ID NO: 105 - protein, 9 aa, synthetic construct
  REGION 1..9: note = Synthetic
NRRGGTCAN

SEQ ID NO: 106 - protein, 11 aa, synthetic construct
  REGION 1..11: note = Synthetic
NVCACCTGCNN

SEQ ID NO: 107 - protein, 17 aa, synthetic construct
  REGION 1..17: note = Synthetic
BHBBKKACGTMMNWNNN

SEQ ID NO: 108 - protein, 11 aa, synthetic construct
  REGION 1..11: note = Synthetic
DCCGCCGCCRY

SEQ ID NO: 109 - protein, 16 aa, synthetic construct
  REGION 1..16: note = Synthetic
NNWGCAACAGGTGDNN

SEQ ID NO: 110 - protein, 12 aa, synthetic construct
  REGION 1..12: note = Synthetic
NSAMCATCTGKT

SEQ ID NO: 111 - protein, 11 aa, synthetic construct
  REGION 1..11: note = Synthetic
NNATGGCCGNN

SEQ ID NO: 112 - protein, 12 aa, synthetic construct
  REGION 1..12: note = Synthetic
VNGCAACAKAWD

SEQ ID NO: 113 - protein, 12 aa, synthetic construct
  REGION 1..12: note = Synthetic
RCGACCACCGAN

SEQ ID NO: 114 - protein, 13 aa, synthetic construct
  REGION 1..13: note = Synthetic
NHSACGTGGCANN

SEQ ID NO: 115 - protein, 10 aa, synthetic construct
  REGION 1..10: note = Synthetic
AHCATRTGDT

SEQ ID NO: 116 - protein, 11 aa, synthetic construct
  REGION 1..11: note = Synthetic
DCCGCCGCCRY

SEQ ID NO: 117 - protein, 8 aa, synthetic construct
  REGION 1..8: note = Synthetic
AGGTGTKA

SEQ ID NO: 118 - protein, 8 aa, synthetic construct
  REGION 1..8: note = Synthetic
VCCCCTDA

SEQ ID NO: 119 - protein, 10 aa, synthetic construct
  REGION 1..10: note = Synthetic
CGSYGCCCCC

SEQ ID NO: 120 - protein, 12 aa, synthetic construct
  REGION 1..12: note = Synthetic
VSGCAGGGCASV

SEQ ID NO: 121 - protein, 8 aa, synthetic construct
  REGION 1..8: note = Synthetic
MGCCGCCR

SEQ ID NO: 122 - protein, 16 aa, synthetic construct
  REGION 1..16: note = Synthetic
NDWKCAACAGGTGKNN

SEQ ID NO: 123 - protein, 11 aa, synthetic construct
  REGION 1..11: note = Synthetic
SNCACCTGSVN

SEQ ID NO: 124 - protein, 8 aa, synthetic construct
  REGION 1..8: note = Synthetic
ATGCGGGY

SEQ ID NO: 125 - protein, 13 aa, synthetic construct
  REGION 1..13: note = Synthetic
NNCTGGGGCCABN

SEQ ID NO: 126 - protein, 11 aa, synthetic construct
  REGION 1..11: note = Synthetic
SAACAGCTGNC

SEQ ID NO: 127 - protein, 10 aa, synthetic construct
  REGION 1..10: note = Synthetic
CRCCGACCAN

SEQ ID NO: 128 - protein, 10 aa, synthetic construct
  REGION 1..10: note = Synthetic
ARCAGCTGCY

SEQ ID NO: 129 - protein, 10 aa, synthetic construct
  REGION 1..10: note = Synthetic
VGCAGCTGCN

SEQ ID NO: 130 - protein, 12 aa, synthetic construct
  REGION 1..12: note = Synthetic
ACACGTGKCANN

SEQ ID NO: 131 - protein, 10 aa, synthetic construct
  REGION 1..10: note = Synthetic
AVCACGTGNY

SEQ ID NO: 132 - protein, 8 aa, synthetic construct
  REGION 1..8: note = Synthetic
CGSCGCCR

SEQ ID NO: 133 - protein, 9 aa, synthetic construct
  REGION 1..9: note = Synthetic
HCCCCTWWN

SEQ ID NO: 134 - protein, 8 aa, synthetic construct
  REGION 1..8: note = Synthetic
SGCCGCCR

SEQ ID NO: 135 - protein, 10 aa, synthetic construct
  REGION 1..10: note = Synthetic
ACCGGAAGTR

SEQ ID NO: 136 - protein, 11 aa, synthetic construct
  REGION 1..11: note = Synthetic
NWCTGACACNN

SEQ ID NO: 137 - protein, 10 aa, synthetic construct
  REGION 1..10: note = Synthetic
RVCACGTGMH

SEQ ID NO: 138 - protein, 10 aa, synthetic construct
  REGION 1..10: note = Synthetic
ACCGGAARTR

SEQ ID NO: 139 - protein, 9 aa, synthetic construct
  REGION 1..9: note = Synthetic
ATGACTCAT

SEQ ID NO: 140 - protein, 13 aa, synthetic construct
  REGION 1..13: note = Synthetic
HDGCAGCAGCWDY

SEQ ID NO: 141 - protein, 12 aa, synthetic construct
  REGION 1..12: note = Synthetic
NNACAGCTGTNN

SEQ ID NO: 142 - protein, 15 aa, synthetic construct
  REGION 1..15: note = Synthetic
HHDCCGCCGACAHND

SEQ ID NO: 143 - protein, 11 aa, synthetic construct
  REGION 1..11: note = Synthetic
BBYTGTGGTTT

SEQ ID NO: 144 - protein, 10 aa, synthetic construct
  REGION 1..10: note = Synthetic
DACCGGAAGY

SEQ ID NO: 145 - protein, 10 aa, synthetic construct
  REGION 1..10: note = Synthetic
ACCGGAAGTR

SEQ ID NO: 146 - protein, 10 aa, synthetic construct
  REGION 1..10: note = Synthetic
NNACGCGCCN

SEQ ID NO: 147 - protein, 15 aa, synthetic construct
  REGION 1..15: note = Synthetic
MARMGGGRGACAMKK

SEQ ID NO: 148 - protein, 14 aa, synthetic construct
  REGION 1..14: note = Synthetic
NNAAACCACAARNN

SEQ ID NO: 149 - protein, 10 aa, synthetic construct
  REGION 1..10: note = Synthetic
ACCGGAARTR

SEQ ID NO: 150 - protein, 11 aa, synthetic construct
  REGION 1..11: note = Synthetic
CDCCGCCGCCR

SEQ ID NO: 151 - protein, 10 aa, synthetic construct
  REGION 1..10: note = Synthetic
YKGGGACCAC

SEQ ID NO: 152 - protein, 6 aa, synthetic construct
  REGION 1..6: note = Synthetic
MTCCAC

SEQ ID NO: 153 - protein, 12 aa, synthetic construct
  REGION 1..12: note = Synthetic
NNACAGCTGTNN

SEQ ID NO: 154 - protein, 9 aa, synthetic construct
  REGION 1..9: note = Synthetic
WAACCGCAA

SEQ ID NO: 155 - protein, 15 aa, synthetic construct
  REGION 1..15: note = Synthetic
AACGAAACCGAAACT

SEQ ID NO: 156 - protein, 12 aa, synthetic construct
  REGION 1..12: note = Synthetic
AAMCCGGAAGTR

SEQ ID NO: 157 - protein, 12 aa, synthetic construct
  REGION 1..12: note = Synthetic
VACCGGAAGTVV

SEQ ID NO: 158 - protein, 11 aa, synthetic construct
  REGION 1..11: note = Synthetic
DVTGASTCATB

SEQ ID NO: 159 - protein, 13 aa, synthetic construct
  REGION 1..13: note = Synthetic
NKATGAGTCATNN

SEQ ID NO: 160 - protein, 7 aa, synthetic construct
  REGION 1..7: note = Synthetic
STCTAGA

SEQ ID NO: 161 - protein, 12 aa, synthetic construct
  REGION 1..12: note = Synthetic
CCGCCGCCRCCD

SEQ ID NO: 162 - protein, 10 aa, synthetic construct
  REGION 1..10: note = Synthetic
GRCACGTGYC

SEQ ID NO: 163 - protein, 14 aa, synthetic construct
  REGION 1..14: note = Synthetic
HCGAAACCGAAACT

SEQ ID NO: 164 - protein, 10 aa, synthetic construct
  REGION 1..10: note = Synthetic
AACAGCTGTT

SEQ ID NO: 165 - protein, 11 aa, synthetic construct
  REGION 1..11: note = Synthetic
DKCMACTTGCM

SEQ ID NO: 166 - protein, 15 aa, synthetic construct
  REGION 1..15: note = Synthetic
HCGAAACCGAAACYA

SEQ ID NO: 167 - protein, 10 aa, synthetic construct
  REGION 1..10: note = Synthetic
RYCACGTGAC

SEQ ID NO: 168 - protein, 10 aa, synthetic construct
  REGION 1..10: note = Synthetic
GRCACGTGYC

SEQ ID NO: 169 - protein, 15 aa, synthetic construct
  REGION 1..15: note = Synthetic
CCDCCDCCACCGCCD

SEQ ID NO: 170 - protein, 12 aa, synthetic construct
  REGION 1..12: note = Synthetic
SSCGCCGCCGCC

SEQ ID NO: 171 - protein, 20 aa, synthetic construct
  REGION 1..20: note = Synthetic
BKNNNNVCACGTGBNNNNMV

SEQ ID NO: 172 - protein, 12 aa, synthetic construct
  REGION 1..12: note = Synthetic
AACCCGGAAGTR

SEQ ID NO: 173 - protein, 10 aa, synthetic construct
  REGION 1..10: note = Synthetic
ACCGGAAGTR

SEQ ID NO: 174 - protein, 13 aa, synthetic construct
  REGION 1..13: note = Synthetic
NVCCGGAAGTGSV

SEQ ID NO: 175 - protein, 12 aa, synthetic construct
  REGION 1..12: note = Synthetic
NVACAGCTGTBN

SEQ ID NO: 176 - protein, 8 aa, synthetic construct
  REGION 1..8: note = Synthetic
MGCCGCCA

SEQ ID NO: 177 - protein, 20 aa, synthetic construct
  REGION 1..20: note = Synthetic
NNNNNNDCCGGAARYNVNNN

SEQ ID NO: 178 - protein, 12 aa, synthetic construct
  REGION 1..12: note = Synthetic
KATGASTCATHN

SEQ ID NO: 179 - protein, 22 aa, synthetic construct
  REGION 1..22: note = Synthetic
NNNNNBRYTTCCGGTNNNNNNN

SEQ ID NO: 180 - protein, 19 aa, synthetic construct
  REGION 1..19: note = Synthetic
DWANCATGASTCATSNTWH

SEQ ID NO: 181 - protein, 11 aa, synthetic construct
  REGION 1..11: note = Synthetic
AACCGGAAATR

SEQ ID NO: 182 - protein, 13 aa, synthetic construct
  REGION 1..13: note = Synthetic
NNGCACCTGTCNB

SEQ ID NO: 183 - protein, 15 aa, synthetic construct
  REGION 1..15: note = Synthetic
CCDCCDCCRCCGCCA

SEQ ID NO: 184 - protein, 19 aa, synthetic construct
  REGION 1..19: note = Synthetic
TGACCTNNAGTRACCYYDN

SEQ ID NO: 185 - protein, 8 aa, synthetic construct
  REGION 1..8: note = Synthetic
CACCGACA

SEQ ID NO: 186 - protein, 9 aa, synthetic construct
  REGION 1..9: note = Synthetic
GCCACGTGD

SEQ ID NO: 187 - protein, 9 aa, synthetic construct
  REGION 1..9: note = Synthetic
CNVMGCCHC

SEQ ID NO: 188 - protein, 10 aa, synthetic construct
  REGION 1..10: note = Synthetic
ACCGGAAGTR

SEQ ID NO: 189 - protein, 8 aa, synthetic construct
  REGION 1..8: note = Synthetic
AGGTGTGA

SEQ ID NO: 190 - protein, 12 aa, synthetic construct
  REGION 1..12: note = Synthetic
CCGCCGCCRCCR

SEQ ID NO: 191 - protein, 11 aa, synthetic construct
  REGION 1..11: note = Synthetic
GRCCRCGCCCH

SEQ ID NO: 192 - protein, 14 aa, synthetic construct
  REGION 1..14: note = Synthetic
RDVCAGGAAGTGVN

SEQ ID NO: 193 - protein, 9 aa, synthetic construct
  REGION 1..9: note = Synthetic
ATGACTCAT

SEQ ID NO: 194 - protein, 10 aa, synthetic construct
  REGION 1..10: note = Synthetic
YGMCAGCTGK

SEQ ID NO: 195 - protein, 11 aa, synthetic construct
  REGION 1..11: note = Synthetic
NRCCACRCCCH

SEQ ID NO: 196 - protein, 11 aa, synthetic construct
  REGION 1..11: note = Synthetic
BCRCTTCCGGB

SEQ ID NO: 197 - protein, 12 aa, synthetic construct
  REGION 1..12: note = Synthetic
NKCCACGTSDHH

SEQ ID NO: 198 - protein, 10 aa, synthetic construct
  REGION 1..10: note = Synthetic
KRTGASTCAT

SEQ ID NO: 199 - protein, 11 aa, synthetic construct
  REGION 1..11: note = Synthetic
YCRCCGACAHN

SEQ ID NO: 200 - protein, 11 aa, synthetic construct
  REGION 1..11: note = Synthetic
VATGACTCATS

SEQ ID NO: 201 - protein, 16 aa, synthetic construct
  REGION 1..16: note = Synthetic
GGYAGCRGCAGCGGYG

SEQ ID NO: 202 - protein, 13 aa, synthetic construct
  REGION 1..13: note = Synthetic
NNDCCAGATGTBN

SEQ ID NO: 203 - protein, 11 aa, synthetic construct
  REGION 1..11: note = Synthetic
BATGCGGGTAC

SEQ ID NO: 204 - protein, 14 aa, synthetic construct
  REGION 1..14: note = Synthetic
NNMMCGAAACCNNV

SEQ ID NO: 205 - protein, 10 aa, synthetic construct
  REGION 1..10: note = Synthetic
MSCGGAAGTR

SEQ ID NO: 206 - protein, 12 aa, synthetic construct
  REGION 1..12: note = Synthetic
NDRCAGCTGYHN

SEQ ID NO: 207 - protein, 9 aa, synthetic construct
  REGION 1..9: note = Synthetic
VCCCCTWTH

SEQ ID NO: 208 - protein, 20 aa, synthetic construct
  REGION 1..20: note = Synthetic
NNNNVTCACGTGAYNNNNNN

SEQ ID NO: 209 - protein, 8 aa, synthetic construct
  REGION 1..8: note = Synthetic
MCACGTGK

SEQ ID NO: 210 - protein, 14 aa, synthetic construct
  REGION 1..14: note = Synthetic
ARCGGGGGACAYGT

SEQ ID NO: 211 - protein, 10 aa, synthetic construct
  REGION 1..10: note = Synthetic
KATGACTCAT

SEQ ID NO: 212 - protein, 15 aa, synthetic construct
  REGION 1..15: note = Synthetic
AGGTCASVNTGMCCY

SEQ ID NO: 213 - protein, 10 aa, synthetic construct
  REGION 1..10: note = Synthetic
DRCAGGTGYD

SEQ ID NO: 214 - protein, 7 aa, synthetic construct
  REGION 1..7: note = Synthetic
CACGTGA

SEQ ID NO: 215 - protein, 13 aa, synthetic construct
  REGION 1..13: note = Synthetic
YMTCCACCGTHDH

SEQ ID NO: 216 - protein, 13 aa, synthetic construct
  REGION 1..13: note = Synthetic
YMTCCACCGTHDH

SEQ ID NO: 217 - protein, 6 aa, synthetic construct
  REGION 1..6: note = Synthetic
CTGTGG

SEQ ID NO: 218 - protein, 10 aa, synthetic construct
  REGION 1..10: note = Synthetic
AMCATATGKY

SEQ ID NO: 219 - protein, 14 aa, synthetic construct
  REGION 1..14: note = Synthetic
GACCCCCCGCRAMG

SEQ ID NO: 220 - protein, 16 aa, synthetic construct
  REGION 1..16: note = Synthetic
NNTGMCACGTGKCANN

SEQ ID NO: 221 - protein, 9 aa, synthetic construct
  REGION 1..9: note = Synthetic
CGTTGCYAY

SEQ ID NO: 222 - protein, 20 aa, synthetic construct
  REGION 1..20: note = Synthetic
NNNNNHRCACCTGYHNNNNN

SEQ ID NO: 223 - protein, 12 aa, synthetic construct
  REGION 1..12: note = Synthetic
WVACAGCAGAYW

SEQ ID NO: 224 - protein, 10 aa, synthetic construct
  REGION 1..10: note = Synthetic
GGCACGTGCC

SEQ ID NO: 225 - protein, 12 aa, synthetic construct
  REGION 1..12: note = Synthetic
RCCACGCCCMCY

SEQ ID NO: 226 - protein, 12 aa, synthetic construct
  REGION 1..12: note = Synthetic
NVACAGCTGTBN

SEQ ID NO: 227 - protein, 11 aa, synthetic construct
  REGION 1..11: note = Synthetic
ACCGGAAGTRV

SEQ ID NO: 228 - protein, 8 aa, synthetic construct
  REGION 1..8: note = Synthetic
AGGTGTGA

SEQ ID NO: 229 - protein, 11 aa, synthetic construct
  REGION 1..11: note = Synthetic
NRCCACGCCCH

SEQ ID NO: 230 - protein, 13 aa, synthetic construct
  REGION 1..13: note = Synthetic
NNACAGATGTTNN

SEQ ID NO: 231 - protein, 6 aa, synthetic construct
  REGION 1..6: note = Synthetic
GGCCRN

SEQ ID NO: 232 - protein, 11 aa, synthetic construct
  REGION 1..11: note = Synthetic
AMCCGGATGTR

SEQ ID NO: 233 - protein, 12 aa, synthetic construct
  REGION 1..12: note = Synthetic
YGCCACGTCAYC

SEQ ID NO: 234 - protein, 10 aa, synthetic construct
  REGION 1..10: note = Synthetic
RTCACGTGAT

SEQ ID NO: 235 - protein, 14 aa, synthetic construct
  REGION 1..14: note = Synthetic
NNCCACTTCCTGNT

SEQ ID NO: 236 - protein, 13 aa, synthetic construct
  REGION 1..13: note = Synthetic
CCGCCGCCGCCGC

SEQ ID NO: 237 - protein, 7 aa, synthetic construct
  REGION 1..7: note = Synthetic
MCCAGCA

SEQ ID NO: 238 - protein, 16 aa, synthetic construct
  REGION 1..16: note = Synthetic
CSCCGCCGCCGCCRCC

SEQ ID NO: 239 - protein, 14 aa, synthetic construct
  REGION 1..14: note = Synthetic
GCRMCAGCTGTYAC

SEQ ID NO: 240 - protein, 14 aa, synthetic construct
  REGION 1..14: note = Synthetic
NNCACTTCCTGTNN

SEQ ID NO: 241 - protein, 12 aa, synthetic construct
  REGION 1..12: note = Synthetic
GRTGACGTCAYC

SEQ ID NO: 242 - protein, 21 aa, synthetic construct
  REGION 1..21: note = Synthetic
NSRRAAMGGAAACCGAAACYR

SEQ ID NO: 243 - protein, 14 aa, synthetic construct
  REGION 1..14: note = Synthetic
NNACAGGAAGTGVN

SEQ ID NO: 244 - protein, 14 aa, synthetic construct
  REGION 1..14: note = Synthetic
NMYTGCAGAGCCCH

SEQ ID NO: 245 - protein, 15 aa, synthetic construct
  REGION 1..15: note = Synthetic
VGSCTAGAGMGGCCS

SEQ ID NO: 246 - protein, 13 aa, synthetic construct
  REGION 1..13: note = Synthetic
NRGACCACCCASV

SEQ ID NO: 247 - protein, 8 aa, synthetic construct
  REGION 1..8: note = Synthetic
DTGACAGS

SEQ ID NO: 248 - protein, 10 aa, synthetic construct
  REGION 1..10: note = Synthetic
KRTGASTCAT

SEQ ID NO: 249 - protein, 11 aa, synthetic construct
  REGION 1..11: note = Synthetic
NNTCGTGACHN

SEQ ID NO: 250 - protein, 12 aa, synthetic construct
  REGION 1..12: note = Synthetic
RCCACGCCCMCY

SEQ ID NO: 251 - protein, 14 aa, synthetic construct
  REGION 1..14: note = Synthetic
YHTYMGCCGCCDYN

SEQ ID NO: 252 - protein, 10 aa, synthetic construct
  REGION 1..10: note = Synthetic
ACCATATGGT

SEQ ID NO: 253 - protein, 11 aa, synthetic construct
  REGION 1..11: note = Synthetic
YCACCAACMNC

SEQ ID NO: 254 - protein, 20 aa, synthetic construct
  REGION 1..20: note = Synthetic
YNANYGGCGCCGYRYVNMBH

SEQ ID NO: 255 - protein, 14 aa, synthetic construct
  REGION 1..14: note = Synthetic
RWMAACAGGAAGTD

SEQ ID NO: 256 - protein, 10 aa, synthetic construct
  REGION 1..10: note = Synthetic
CGCAGCTGCK

SEQ ID NO: 257 - protein, 20 aa, synthetic construct
  REGION 1..20: note = Synthetic
NNNNRRCAGCTGTYNNNNNN

SEQ ID NO: 258 - protein, 22 aa, synthetic construct
  REGION 1..22: note = Synthetic
NNNNNGRCACGTGCCNNNNNNN

SEQ ID NO: 259 - protein, 15 aa, synthetic construct
  REGION 1..15: note = Synthetic
DDWKSHSACGTGGCA

SEQ ID NO: 260 - protein, 14 aa, synthetic construct
  REGION 1..14: note = Synthetic
CCGAAACCGAAACY

SEQ ID NO: 261 - protein, 10 aa, synthetic construct
  REGION 1..10: note = Synthetic
ACCGGAAGTR

SEQ ID NO: 262 - protein, 10 aa, synthetic construct
  REGION 1..10: note = Synthetic
RACATATGTC

SEQ ID NO: 263 - protein, 15 aa, synthetic construct
  REGION 1..15: note = Synthetic
TTCACATAAAAACTA

SEQ ID NO: 264 - protein, 11 aa, synthetic construct
  REGION 1..11: note = Synthetic
NATGACTCATH

SEQ ID NO: 265 - protein, 10 aa, synthetic construct
  REGION 1..10: note = Synthetic
SVYAACCGMC

SEQ ID NO: 266 - protein, 7 aa, synthetic construct
  REGION 1..7: note = Synthetic
CGCGCVN

SEQ ID NO: 267 - protein, 13 aa, synthetic construct
  REGION 1..13: note = Synthetic
NNNDACACGTGDN

SEQ ID NO: 268 - protein, 14 aa, synthetic construct
  REGION 1..14: note = Synthetic
RYAACAGCTGTTRN

SEQ ID NO: 269 - protein, 15 aa, synthetic construct
  REGION 1..15: note = Synthetic
CCDCCDCCACCGMCA

SEQ ID NO: 270 - protein, 10 aa, synthetic construct
  REGION 1..10: note = Synthetic
NVCAGATGNN

SEQ ID NO: 271 - protein, 8 aa, synthetic construct
  REGION 1..8: note = Synthetic
YGCGGCKB

SEQ ID NO: 272 - protein, 12 aa, synthetic construct
  REGION 1..12: note = Synthetic
TTCCCGCCAHWA

SEQ ID NO: 273 - protein, 8 aa, synthetic construct
  REGION 1..8: note = Synthetic
SCGCCGCC

SEQ ID NO: 274 - protein, 10 aa, synthetic construct
  REGION 1..10: note = Synthetic
BACTGACAGT

SEQ ID NO: 275 - protein, 9 aa, synthetic construct
  REGION 1..9: note = Synthetic
BGGSCCCAC

SEQ ID NO: 276 - protein, 15 aa, synthetic construct
  REGION 1..15: note = Synthetic
CCDCCGCCGCCGCCR

SEQ ID NO: 277 - protein, 17 aa, synthetic construct
  REGION 1..17: note = Synthetic
RYGGCGGCGGHGGHGGH

SEQ ID NO: 278 - protein, 16 aa, synthetic construct
  REGION 1..16: note = Synthetic
NVNATGACTCAGCADW

SEQ ID NO: 279 - protein, 9 aa, synthetic construct
  REGION 1..9: note = Synthetic
GGGGCGGGG

SEQ ID NO: 280 - protein, 14 aa, synthetic construct
  REGION 1..14: note = Synthetic
MCWGCCGACAAGSH

SEQ ID NO: 281 - protein, 14 aa, synthetic construct
  REGION 1..14: note = Synthetic
CCAGYYYVADCCRG

SEQ ID NO: 282 - protein, 22 aa, synthetic construct
  REGION 1..22: note = Synthetic
NNNNNNNGGCACGTGBBNNNNN

SEQ ID NO: 283 - protein, 13 aa, synthetic construct
  REGION 1..13: note = Synthetic
NNATGACTCATNN

SEQ ID NO: 284 - protein, 15 aa, synthetic construct
  REGION 1..15: note = Synthetic
NNRCAGGAAGTGGVN

SEQ ID NO: 285 - protein, 10 aa, synthetic construct
  REGION 1..10: note = Synthetic
KKGGGCCCMM

SEQ ID NO: 286 - protein, 13 aa, synthetic construct
  REGION 1..13: note = Synthetic
NBRACAGGTGYAN

SEQ ID NO: 287 - protein, 12 aa, synthetic construct
  REGION 1..12: note = Synthetic
NVACACCTGTNN

SEQ ID NO: 288 - protein, 20 aa, synthetic construct
  REGION 1..20: note = Synthetic
NNNNNNDCCGGAARHNNNNN

SEQ ID NO: 289 - protein, 10 aa, synthetic construct
  REGION 1..10: note = Synthetic
RRGGGGCGKR

SEQ ID NO: 290 - protein, 11 aa, synthetic construct
  REGION 1..11: note = Synthetic
KRRTGASTCAB

SEQ ID NO: 291 - protein, 7 aa, synthetic construct
  REGION 1..7: note = Synthetic
CCCCRCV

SEQ ID NO: 292 - protein, 13 aa, synthetic construct
  REGION 1..13: note = Synthetic
AGGGGAWTCCCCY

SEQ ID NO: 293 - protein, 14 aa, synthetic construct
  REGION 1..14: note = Synthetic
NNACAGGAAGTGNN

SEQ ID NO: 294 - protein, 12 aa, synthetic construct
  REGION 1..12: note = Synthetic
SGACGGCGACGV

SEQ ID NO: 295 - protein, 14 aa, synthetic construct
  REGION 1..14: note = Synthetic
NNVACACCTGTBNN

SEQ ID NO: 296 - protein, 15 aa, synthetic construct
  REGION 1..15: note = Synthetic
HDYCACCGACAHHNN

SEQ ID NO: 297 - protein, 13 aa, synthetic construct
  REGION 1..13: note = Synthetic
NNATGACTCATNN

SEQ ID NO: 298 - protein, 14 aa, synthetic construct
  REGION 1..14: note = Synthetic
NNRGACCACCCASV

SEQ ID NO: 299 - protein, 12 aa, synthetic construct
  REGION 1..12: note = Synthetic
NNRTGAGTCAYN

SEQ ID NO: 300 - protein, 11 aa, synthetic construct
  REGION 1..11: note = Synthetic
SCCCCGCCCCS

SEQ ID NO: 301 - protein, 10 aa, synthetic construct
  REGION 1..10: note = Synthetic
TGGGSCCCAC

SEQ ID NO: 302 - protein, 21 aa, synthetic construct
  REGION 1..21: note = Synthetic
NNNHCAARATGGCTGCCNBNN

SEQ ID NO: 303 - protein, 11 aa, synthetic construct
  REGION 1..11: note = Synthetic
SCCACGCCCMC

SEQ ID NO: 304 - protein, 8 aa, synthetic construct
  REGION 1..8: note = Synthetic
GGSMCCAC

SEQ ID NO: 305 - protein, 10 aa, synthetic construct
  REGION 1..10: note = Synthetic
GRCACGTGCC

SEQ ID NO: 306 - protein, 9 aa, synthetic construct
  REGION 1..9: note = Synthetic
GVTAGCGCA

SEQ ID NO: 307 - protein, 15 aa, synthetic construct
  REGION 1..15: note = Synthetic
GGGGYGAYGACCRCT

SEQ ID NO: 308 - protein, 10 aa, synthetic construct
  REGION 1..10: note = Synthetic
NVCAGCTGBN

SEQ ID NO: 309 - protein, 20 aa, synthetic construct
  REGION 1..20: note = Synthetic
NNNNNRYTTCCGGNNNNNNN

SEQ ID NO: 310 - protein, 15 aa, synthetic construct
  REGION 1..15: note = Synthetic
NNCACTTCCTGTTNN

SEQ ID NO: 311 - protein, 14 aa, synthetic construct
  REGION 1..14: note = Synthetic
ACCGGAAGTAATTA

SEQ ID NO: 312 - protein, 9 aa, synthetic construct
  REGION 1..9: note = Synthetic
RSTAGCGCA

SEQ ID NO: 313 - protein, 10 aa, synthetic construct
  REGION 1..10: note = Synthetic
RTGACGTMAY

SEQ ID NO: 314 - protein, 21 aa, synthetic construct
  REGION 1..21: note = Synthetic
YCDCCDCCDCCGCCGCCRYYD

SEQ ID NO: 315 - protein, 10 aa, synthetic construct
  REGION 1..10: note = Synthetic
HGCTACYGTD

SEQ ID NO: 316 - protein, 10 aa, synthetic construct
  REGION 1..10: note = Synthetic
AMCATATGKT

SEQ ID NO: 317 - protein, 13 aa, synthetic construct
  REGION 1..13: note = Synthetic
NBGCACCTGTMNY

SEQ ID NO: 318 - protein, 13 aa, synthetic construct
  REGION 1..13: note = Synthetic
NKATGACTCATNN

SEQ ID NO: 319 - protein, 18 aa, synthetic construct
  REGION 1..18: note = Synthetic
RTGMCACGCCCCTTTTTG

SEQ ID NO: 320 - protein, 10 aa, synthetic construct
  REGION 1..10: note = Synthetic
ATGAGTCAYM

SEQ ID NO: 321 - protein, 9 aa, synthetic construct
  REGION 1..9: note = Synthetic
GGGSCCCAC

SEQ ID NO: 322 - protein, 8 aa, synthetic construct
  REGION 1..8: note = Synthetic
RRGGTCAN

SEQ ID NO: 323 - protein, 12 aa, synthetic construct
  REGION 1..12: note = Synthetic
HYCACCTGRNNY

SEQ ID NO: 324 - protein, 6 aa, synthetic construct
  REGION 1..6: note = Synthetic
GAARCC

SEQ ID NO: 325 - protein, 15 aa, synthetic construct
  REGION 1..15: note = Synthetic
BGTAAACAGGAAGYR

SEQ ID NO: 326 - protein, 12 aa, synthetic construct
  REGION 1..12: note = Synthetic
DRAGGTGTGAAR

SEQ ID NO: 327 - protein, 15 aa, synthetic construct
  REGION 1..15: note = Synthetic
HDHKSGCGSGAAAWT

SEQ ID NO: 328 - protein, 16 aa, synthetic construct
  REGION 1..16: note = Synthetic
VRRRACAAATGGTNNN

SEQ ID NO: 329 - protein, 11 aa, synthetic construct
  REGION 1..11: note = Synthetic
TGRTAGCGCCR

SEQ ID NO: 330 - protein, 10 aa, synthetic construct
  REGION 1..10: note = Synthetic
YYCACCTAMY

SEQ ID NO: 331 - protein, 10 aa, synthetic construct
  REGION 1..10: note = Synthetic
YYCACCTAMY

SEQ ID NO: 332 - protein, 11 aa, synthetic construct
  REGION 1..11: note = Synthetic
RASCACGTGGT

SEQ ID NO: 333 - protein, 13 aa, synthetic construct
  REGION 1..13: note = Synthetic
GTAAACAGGAWGY

SEQ ID NO: 334 - protein, 12 aa, synthetic construct
  REGION 1..12: note = Synthetic
RCGACCACCGAA

SEQ ID NO: 335 - protein, 13 aa, synthetic construct
  REGION 1..13: note = Synthetic
NYCCCAAGGGANN

SEQ ID NO: 336 - protein, 10 aa, synthetic construct
  REGION 1..10: note = Synthetic
GTGGACCCRS

SEQ ID NO: 337 - protein, 16 aa, synthetic construct
  REGION 1..16: note = Synthetic
RSCGGAAGCAGSTGKN

SEQ ID NO: 338 - protein, 15 aa, synthetic construct
  REGION 1..15: note = Synthetic
SHGRCGCCGVCGSHG

SEQ ID NO: 339 - protein, 9 aa, synthetic construct
  REGION 1..9: note = Synthetic
NNTGCCAAN

SEQ ID NO: 340 - protein, 12 aa, synthetic construct
  REGION 1..12: note = Synthetic
YGCCCBVRGGCA

SEQ ID NO: 341 - protein, 10 aa, synthetic construct
  REGION 1..10: note = Synthetic
NNCACGTGNN

SEQ ID NO: 342 - protein, 10 aa, synthetic construct
  REGION 1..10: note = Synthetic
VVCAGCTGBB

SEQ ID NO: 343 - protein, 12 aa, synthetic construct
  REGION 1..12: note = Synthetic
CYCATTGGCCVV

SEQ ID NO: 344 - protein, 21 aa, synthetic construct
  REGION 1..21: note = Synthetic
KBNBMTAKTGCACCCSNWWBS

SEQ ID NO: 345 - protein, 16 aa, synthetic construct
  REGION 1..16: note = Synthetic
NNDGTGACGTCACHNN

SEQ ID NO: 346 - protein, 12 aa, synthetic construct
  REGION 1..12: note = Synthetic
YGCCCBVRGGCR

SEQ ID NO: 347 - protein, 14 aa, synthetic construct
  REGION 1..14: note = Synthetic
VCMCGCCCACGCVS

SEQ ID NO: 348 - protein, 13 aa, synthetic construct
  REGION 1..13: note = Synthetic
NNCAGACAGCMNN

SEQ ID NO: 349 - protein, 8 aa, synthetic construct
  REGION 1..8: note = Synthetic
TTGACAGS

SEQ ID NO: 350 - protein, 12 aa, synthetic construct
  REGION 1..12: note = Synthetic
HHCACGCGCTNN

SEQ ID NO: 351 - protein, 13 aa, synthetic construct
  REGION 1..13: note = Synthetic
NTGTCGGTANNNN

SEQ ID NO: 352 - protein, 15 aa, synthetic construct
  REGION 1..15: note = Synthetic
WWWTGVCYYTTSRDD

SEQ ID NO: 353 - protein, 9 aa, synthetic construct
  REGION 1..9: note = Synthetic
DGGGCGGGG

SEQ ID NO: 354 - protein, 14 aa, synthetic construct
  REGION 1..14: note = Synthetic
BNCGCACGTGCGNV

SEQ ID NO: 355 - protein, 14 aa, synthetic construct
  REGION 1..14: note = Synthetic
SSCGCCGCTCCSSS

SEQ ID NO: 356 - protein, 13 aa, synthetic construct
  REGION 1..13: note = Synthetic
NNATGACTCATNN

SEQ ID NO: 357 - protein, 15 aa, synthetic construct
  REGION 1..15: note = Synthetic
HGRYGGCGGCGGHGG

SEQ ID NO: 358 - protein, 10 aa, synthetic construct
  REGION 1..10: note = Synthetic
NVCAGCTGBN

SEQ ID NO: 359 - protein, 10 aa, synthetic construct
  REGION 1..10: note = Synthetic
KATGACKCAT

SEQ ID NO: 360 - protein, 14 aa, synthetic construct
  REGION 1..14: note = Synthetic
GGGGCCCWAGGGGG

SEQ ID NO: 361 - protein, 10 aa, synthetic construct
  REGION 1..10: note = Synthetic
AWCAGCTGWT

SEQ ID NO: 362 - protein, 8 aa, synthetic construct
  REGION 1..8: note = Synthetic
TGACGTCA

SEQ ID NO: 363 - protein, 15 aa, synthetic construct
  REGION 1..15: note = Synthetic
TKGCGGCGGMGGHGG

SEQ ID NO: 364 - protein, 16 aa, synthetic construct
  REGION 1..16: note = Synthetic
DCCGGAAGTSRCGTGA

SEQ ID NO: 365 - protein, 15 aa, synthetic construct
  REGION 1..15: note = Synthetic
CCCCACTCCTGGTAC

SEQ ID NO: 366 - protein, 22 aa, synthetic construct
  REGION 1..22: note = Synthetic
NNNNNNRGGTGTGAANDNNNNN

SEQ ID NO: 367 - protein, 10 aa, synthetic construct
  REGION 1..10: note = Synthetic
RRCAGGTGYA

SEQ ID NO: 368 - protein, 8 aa, synthetic construct
  REGION 1..8: note = Synthetic
GGCGGAGW

SEQ ID NO: 369 - protein, 11 aa, synthetic construct
  REGION 1..11: note = Synthetic
GMCACGCCCCC

SEQ ID NO: 370 - protein, 17 aa, synthetic construct
  REGION 1..17: note = Synthetic
DDNTGMCACGTGTCMHH

SEQ ID NO: 371 - protein, 8 aa, synthetic construct
  REGION 1..8: note = Synthetic
GCACGTGC

SEQ ID NO: 372 - protein, 18 aa, synthetic construct
  REGION 1..18: note = Synthetic
RRGGTCAHNNNRRGGTCA

SEQ ID NO: 373 - protein, 8 aa, synthetic construct
  REGION 1..8: note = Synthetic
CCGGAART

SEQ ID NO: 374 - protein, 24 aa, synthetic construct
  REGION 1..24: note = Synthetic
NYTGTGTCCTCABRTGACCTYWBB

SEQ ID NO: 375 - protein, 9 aa, synthetic construct
  REGION 1..9: note = Synthetic
GGGGCGGGG

SEQ ID NO: 376 - protein, 12 aa, synthetic construct
  REGION 1..12: note = Synthetic
SSGGGGMGGGGS

SEQ ID NO: 377 - protein, 10 aa, synthetic construct
  REGION 1..10: note = Synthetic
NTGCCGGCGN

SEQ ID NO: 378 - protein, 11 aa, synthetic construct
  REGION 1..11: note = Synthetic
NRTGACTCABN

SEQ ID NO: 379 - protein, 10 aa, synthetic construct
  REGION 1..10: note = Synthetic
NVCAGCTGBN

SEQ ID NO: 380 - protein, 12 aa, synthetic construct
  REGION 1..12: note = Synthetic
YGCCYBVRGGCA

SEQ ID NO: 381 - protein, 13 aa, synthetic construct
  REGION 1..13: note = Synthetic
SSCGCCGCCGCCG

SEQ ID NO: 382 - protein, 8 aa, synthetic construct
  REGION 1..8: note = Synthetic
SACACCCB

SEQ ID NO: 383 - protein, 12 aa, synthetic construct
  REGION 1..12: note = Synthetic
RRGCGTGACCNN

SEQ ID NO: 384 - protein, 8 aa, synthetic construct
  REGION 1..8: note = Synthetic
SGGCGGGA

SEQ ID NO: 385 - protein, 18 aa, synthetic construct
  REGION 1..18: note = Synthetic
SGAAAGYGAAASCRWWWM

SEQ ID NO: 386 - protein, 12 aa, synthetic construct
  REGION 1..12: note = Synthetic
NNACAGAAGCNN

SEQ ID NO: 387 - protein, 14 aa, synthetic construct
  REGION 1..14: note = Synthetic
YBCCTCCCCCACVB

SEQ ID NO: 388 - protein, 14 aa, synthetic construct
  REGION 1..14: note = Synthetic
YBCCTCCCCCACVB

SEQ ID NO: 389 - protein, 11 aa, synthetic construct
  REGION 1..11: note = Synthetic
CCDCCACCGCC

SEQ ID NO: 390 - protein, 14 aa, synthetic construct
  REGION 1..14: note = Synthetic
HDYCACCGACMANN

SEQ ID NO: 391 - protein, 12 aa, synthetic construct
  REGION 1..12: note = Synthetic
NHAACCTCAANN

SEQ ID NO: 392 - protein, 14 aa, synthetic construct
  REGION 1..14: note = Synthetic
HCGAAARYGAAAVT

SEQ ID NO: 393 - protein, 13 aa, synthetic construct
  REGION 1..13: note = Synthetic
HSACGCTCCTCHN

SEQ ID NO: 394 - protein, 13 aa, synthetic construct
  REGION 1..13: note = Synthetic
HSACGCTCCTCHN

SEQ ID NO: 395 - protein, 11 aa, synthetic construct
  REGION 1..11: note = Synthetic
YHHYWTCACTN

SEQ ID NO: 396 - protein, 12 aa, synthetic construct
  REGION 1..12: note = Synthetic
GATGACGTCAYC

SEQ ID NO: 397 - protein, 14 aa, synthetic construct
  REGION 1..14: note = Synthetic
TGRTGACGTGGCAN

SEQ ID NO: 398 - protein, 20 aa, synthetic construct
  REGION 1..20: note = Synthetic
NNNNRNCGGAAGTNNNNNNN

SEQ ID NO: 399 - protein, 15 aa, synthetic construct
  REGION 1..15: note = Synthetic
CASNATGACTCAGCA

SEQ ID NO: 400 - protein, 8 aa, synthetic construct
  REGION 1..8: note = Synthetic
GGSCCCAC

SEQ ID NO: 401 - protein, 8 aa, synthetic construct
  REGION 1..8: note = Synthetic
GGSCCCAC

SEQ ID NO: 402 - protein, 12 aa, synthetic construct
  REGION 1..12: note = Synthetic
DYCACCGACAHH

SEQ ID NO: 403 - protein, 12 aa, synthetic construct
  REGION 1..12: note = Synthetic
NRGAACAGCCNN

SEQ ID NO: 404 - protein, 12 aa, synthetic construct
  REGION 1..12: note = Synthetic
CGGGGRACACGT

SEQ ID NO: 405 - protein, 22 aa, synthetic construct
  REGION 1..22: note = Synthetic
CYNNNNNAGGTGTGAAWHNYMN

SEQ ID NO: 406 - protein, 22 aa, synthetic construct
  REGION 1..22: note = Synthetic
NDCRNNNAGGTGTGAWWWNNNN

SEQ ID NO: 407 - protein, 14 aa, synthetic construct
  REGION 1..14: note = Synthetic
NDGTCATGTGACHN

SEQ ID NO: 408 - protein, 18 aa, synthetic construct
  REGION 1..18: note = Synthetic
YBVCYBRSCCTCTCTGDG

SEQ ID NO: 409 - protein, 10 aa, synthetic construct
  REGION 1..10: note = Synthetic
AYAGTAGGTS

SEQ ID NO: 410 - protein, 13 aa, synthetic construct
  REGION 1..13: note = Synthetic
GTMAACAGGAWRY

SEQ ID NO: 411 - protein, 8 aa, synthetic construct
  REGION 1..8: note = Synthetic
CSCGCGCG

SEQ ID NO: 412 - protein, 8 aa, synthetic construct
  REGION 1..8: note = Synthetic
RCCGACCA

SEQ ID NO: 413 - protein, 10 aa, synthetic construct
  REGION 1..10: note = Synthetic
RTGGKMCCAY

SEQ ID NO: 414 - protein, 18 aa, synthetic construct
  REGION 1..18: note = Synthetic
NTTDCCWWWWHDGGWAAN

SEQ ID NO: 415 - protein, 20 aa, synthetic construct
  REGION 1..20: note = Synthetic
CCVNNCCACGCCCHNNVVCV

SEQ ID NO: 416 - protein, 8 aa, synthetic construct
  REGION 1..8: note = Synthetic
AGGTGTGA

SEQ ID NO: 417 - protein, 9 aa, synthetic construct
  REGION 1..9: note = Synthetic
TGACACAWW

SEQ ID NO: 418 - protein, 15 aa, synthetic construct
  REGION 1..15: note = Synthetic
NWGMTGACTCAGCAN

SEQ ID NO: 419 - protein, 11 aa, synthetic construct
  REGION 1..11: note = Synthetic
DATGACTCATH

SEQ ID NO: 420 - protein, 14 aa, synthetic construct
  REGION 1..14: note = Synthetic
RCGGGGGACAHGTC